(19) United States
(12) Patent Application Publication
(10) Pub. No.: US 2014/0004209 A1
(43) Pub. Date: Jan. 2, 2014

(54) AUTOPHAGY INDUCER AND INHIBITOR COMBINATION THERAPY FOR THE TREATMENT OF NEOPLASMS

(71) Applicants: Kiu Lin, South San Francisco, CA (US); Michael Degtyarev, South San Francisco, CA (US)

(72) Inventors: Kiu Lin, South San Francisco, CA (US); Michael Degtyarev, South San Francisco, CA (US)

(73) Assignee: GENENTECH, INC., South San Francisco, CA (US)

(21) Appl. No.: 13/997,024

(22) PCT Filed: Dec. 21, 2011
(86) PCT No.: PCT/US11/66449

§ 371 Date: Sep. 19, 2013

Related U.S. Application Data
(60) Provisional application No. 61/426,325, filed on Dec. 22, 2010.

(51) Int. Cl. A61K 31/713; A61K 31/131; A61K 31/7076; A61K 31/52; A61K 33/02

(52) U.S. Cl. CPC A61K 31/713; A61K 31/52; A61K 33/02; A61K 31/7076; A61K 31/131
USPC 424/720; 514/44 A; 514/263.4; 514/47; 514/663

(57) ABSTRACT

The subject matter disclosed herein relates to agents and methods of treating neoplasms with an agent that is a kinase inhibitor and is also an inducer of autophagy in combination with an agent that is an inhibitor of autophagy.

### CROSS REFERENCE TO RELATED APPLICATION

This application claims the benefit of priority of provisional U.S. Application No. 61/426,325 filed Dec. 22, 2010 which is hereby incorporated by reference in its entirety.

### FIELD OF THE INVENTION

The RNAi constructs and combination therapy disclosed herein relate to the treatment of neoplasms.

### BACKGROUND OF INVENTION

Aberrant activation of the class I phosphatidylinositol 3-kinase (PI3K)/Akt pathway has been widely implicated in a variety of cancers. This is not only as a result of abnormal activities of various upstream growth factors and their receptors, but also through direct alterations of the PI3K and Akt isoforms, and more frequently, inactivation of the tumor suppressor phosphatase and tensin homolog (PTEN), a phospholipid phosphatase that negates the activity of PI3K. The three Akt isoforms represent attractive cancer therapeutic targets (Samuels, Y., and K. Ericson, (2006), Oncogenic PI3K and its role in cancer. Curr Opin Oncol. 18:77-82; Stambolic, V., and J. R. Woodgett, (2006), Functional distinctions of protein kinase B/Akt isoforms defined by their influence on cell migration. Trends Cell Biol.) Genetic ablations of the 3 Akt genes in mice have revealed both distinct and overlapping functions of each isoform in normal physiology (Chen, W. S., P. Z. Xu, K. Gottlob, M. L. Chen, K. Sokol, T. Shiyanova, I. Roninson, W. Weng, R. Suzuki, K. Tobe, T. Kadowaki, and N. Hay, (2001), Growth retardation and increased apoptosis in mice with homozygous disruption of the Akt1 gene. Genes Dev. 15:2203-8; Cho, H., J. Mu, J. K. Kim, J. L. Thorvaldsen, Q. Chu, E. B. Crenshaw, 3rd, K. H. Kaestner, M. S. Bartolomei, G. I. Shulman, and M. J. Birnbaum, (2001), Insulin resistance and a diabetes mellitus-like syndrome in mice lacking the protein kinase Akt2 (PKB beta). Science. 292:1728-31; Cho, H., J. L. Thorvaldsen, Q. Chu, F. Feng, and M. J. Birnbaum, (2001), Akt1/PKBalpha is required for normal growth but dispensable for maintenance of glucose homeostasis in mice. J Biol Chem. 276:38349-52. Epub 2001 Aug. 31; Easton, R. M., H. Cho, K. Roovers, D. W. Shineman, M. Mizrahi, M. S. Forman, V. M. Lee, M. Szabolcs, R. de Jong, T. Oltersdorf, T. Ludwig, A. Efstratiadis, and M. J. Birnbaum, (2005), Role for Akt3/protein kinase Bgamma in attainment of normal brain size. Mol Cell Biol. 25:1869-78; Peng, X. D., P. Z. Xu, M. L. Chen, A. Hahn-Windgassen, J. Skeen, J. Jacobs, D. Sundararajan, W. S. Chen, S. E. Crawford, K. G. Coleman, and N. Hay, (2003), Dwarfism, impaired skin development, skeletal muscle atrophy, delayed bone development, and impeded adipogenesis in mice lacking Akt1 and Akt2. Genes Dev. 17:1352-65; Tschopp, O., Z. Z. Yang, D. Brodbeck, B. A. Dummler, M. Hemmings-Mieszczak, T. Watanabe, T. Michaelis, J. Frahm, and B. A. Hemmings, (2005), Essential role of protein kinase B gamma (PKB gamma/Akt3) in postnatal brain development but not in glucose homeostasis. Development. 132:2943-54. Epub 2005 Jun. 1; Yang, Z. Z., O. Tschopp, N. Di-Poi, E. Bruder, A. Baudry, B. Dummler, W. Wahli, and B. A. Hemmings, (2005), Dosage-dependent effects of Akt1/protein kinase Balpha (PKBalpha) and Akt3/PKBgamma on thymus, skin, and cardiovascular and nervous system development in mice. Mol Cell Biol. 25:10407-18) and tumor initiation (Chen, M. L., P. Z. Xu, X. D. Peng, W. S. Chen, G. Guzman, X. Yang, A. Di Cristofano, P. P. Pandolfi, and N. Hay, (2006), The deficiency of Akt1 is sufficient to suppress tumor development in Pten+/− mice. Genes Dev. 20:1569-74; Ju, X., S. Katiyar, C. Wang, M. Liu, X. Jiao, S. Li, J. Zhou, J. Turner, M. P. Lisanti, R. G. Russell, S. C. Mueller, J. Ojeifo, W. S. Chen, N. Hay, and R. G. Pestell, (2007), Akt1 governs breast cancer progression in vivo. Proc Natl Acad Sci USA. 104:7438-43; Maroulakou, I. G., W. Oemler, S. P. Naber, and P. N. Tsichlis, (2007), Akt1 ablation inhibits, whereas Akt2 ablation accelerates, the development of mammary adenocarcinomas in mouse mammary tumor virus (MMTV)-ErbB2/neu and MMTV-polyoma middle T transgenic mice. Cancer Res. 67:167-77; Skeen, J. E., P. T. Bhaskar, C. C. Chen, W. S. Chen, X. D. Peng, V. Nogueira, A. Hahn-Windgassen, H. Kiyokawa, and N. Hay, (2006), Akt deficiency impairs normal cell proliferation and suppresses oncogenesis in a p53-independent and mTORC1-dependent manner. Cancer Cell. 10:269-80). The relative contribution of the three Akt isoforms in maintaining human tumor growth, however, remains elusive.

Human cancers usually co-express two or all three Aid isoforms, and amplification or hyperactivation of each isoform has been documented in different types of cancers (Altomare, D. A., and J. R. Testa, (2005), Perturbations of the AKT signaling pathway in human cancer. Oncogene. 24:7455-64; Stahl, J. M., A. Sharma, M. Cheung, M. Zimmerman, J. Q. Cheng, M. W. Bosenberg, M. Kester, L. Sandirasegarane, and G. P. Robertson, (2004), Deregulated Akt3 activity promotes development of malignant melanoma. Cancer Res. 64:7002-10). Mounting evidence suggests that Akt isoforms may be differentially regulated depending on the external stimuli and the tissue studied, and may regulate distinct aspects of cellular processes in a cell and tissue-specific manner (Dufour, G., M. J. Demers, D. Gagne, A. B. Dydensborg, I. C. Teller, V. Bouchard, I. Degongre, J. F. Beaulieu, J. Q. Cheng, N. Fujita, T. Tsuruo, K. Vallee, and P. H. Vachon, (2004), Human intestinal epithelial cell survival and anoikis. Differentiation state-distinct regulation and roles of protein kinase B/Akt isoforms. J Biol Chem. 279:44113-22. Epub 2004 Aug. 6; Irie, H. Y., R. V. Pearline, D. Grueneberg, M. Hsia, P. Ravichandran, N. Kothari, S, Natesan, and J. S. Brugge, (2005), Distinct roles of Akt1 and Akt2 in regulating cell migration and epithelial-mesenchymal transition. J Cell Biol. 171:1023-34; Kim, D., S. Kim, H. Koh, S. O. Yoon, A. S. Chung, K. S. Cho, and J. Chung, (2001), Akt/PKB promotes cancer cell invasion via increased motility and metalloproteinase production. FASEB J. 15:1953-62; Samuels, Y., L. A. Diaz, Jr., O, Schmidt-Kittler, J. M. Cummins, L. Delong, I. Cheong, C. Rago, D. L. Huso, C. Lengauer, K. W. Kinzler, B. Vogelstein, and V. E. Velculescu. 2005. Mutant PIK3CA promotes cell growth and invasion of human cancer cells. Cancer Cell. 7:561-73; Tanno, S., S. Tanno, Y. Mitsuuchi, D. A. Altomare, G. H. Xiao, and J. R. Testa, (2001), AKT activation up-regulates insulin-like growth factor I receptor expression and promotes invasiveness of human pancreatic cancer cells. Cancer Res. 61:589-93; Yoeli-Lerner, M., G. K. Yiu, I. Rabinovitz, P. Erhardt, S. Jauliac, and A. Toker, (2005), Akt blocks breast cancer cell motility and invasion through the transcription factor NFAT. Mol Cell. 20:539-50).

Akt is well known for its anti-apoptotic activity, leading to its depiction as a survival kinase (Amaravadi, R., and C. B. Thompson, (2005), The survival kinases Akt and Pim as potential pharmacological targets. J Clin Invest. 115:2618-24). However, inhibiting components of the PI3K/Akt pathway often does not induce substantial apoptosis without additional pro-apoptotic insults. This is exemplified in a recent study, where a dual PI3K/mTOR inhibitor that efficiently inhibited phosphorylation of Akt, blocked proliferation of glioma xenografts without the induction of apoptosis (Fan, Q. W., Z. A. Knight, D. D. Goldenberg, W. Yu, K. E. Mostov, D. Stokoe, K. M. Shokat, and W. A. Weiss, (2006), A dual PI3 kinase/mTOR inhibitor reveals emergent efficacy in glioma. Cancer Cell. 9:341-9).

In a recent study, alkylating agents were given in combination with chloroquine (WO 2006/078774). No results were presented on the combination of kinase inhibitors with chloroquine.

### BRIEF SUMMARY OF INVENTION

The subject matter disclosed herein relates to agents and methods of treating neoplasms with an agent that is a kinase inhibitor and is also an inducer of autophagy in combination with an agent that is an inhibitor of autophagy.

### DESCRIPTION OF THE FIGURES

FIG. 1 shows inducible (knockdown) KD of Akt isoforms and their effect on xenograft tumor growth. (A) Immunoblot analysis of Akt isoforms and various downstream proteins in stable PC3 clones expressing the inducible shRNA constructs. Each clone was induced to express the respective shRNA(s) with 1 μg/ml Dox grown under 10% FBS for 7 days. Double arrowheads indicate slight differences in the mobility of the 3 Akt isoforms detected by total and phospho-Akt antibodies, and the mobility shift of IRS 1. FIG. 1B shows the effect of Akt KD on xenograft tumor growth. Representative experiments showing the growth of PC3 xenograft tumors containing the various shRNAs treated with vehicle control (−Dox, filled circles) or Dox (+Dox, open circles) (see details in Table 3). Error bars represent SEM. *, P<0.05; **, P<0.005.

FIG. 2 depicts that Akt KD resulted in cell cycle delay and elevated autophagy without substantial apoptosis. (A) Histological analysis of PC3-shAkt123 tumors treated with Dox or vehicle control for 5, 15, or 21 d as indicated. Tumor tissues were analyzed by IHC using antibodies specific for Ki-67 or by the TUNEL assay. Pathologist's scoring of the signal intensity for each sample is indicated in parentheses. Bars, 100 μm. (B and C) Effect of triple-Akt KD on cell cycle progression under serum starvation (ss) compared with cells grown under 10% FBS. Cells containing shRNAs targeting EGFP or all three Akt isoforms were pretreated for 2 d with or without Dox in medium containing 10% FBS and changed to 0% (B) or 0.5% (C) FBS. Cell cycle profiles were analyzed at the indicated time points after serum withdrawal. Error bars represent SEM (n=3). The percentage of change in each cell cycle phase with Dox versus without Dox treatment is also shown.

FIG. 3 depicts autophagy induced in PC3 and U87MG cells by Akt KD. (A) EM images of PC3 (ad) and U87MG (e-g) cells grown in the absence (a and f) or presence (b-e and g) of Dox-induced Akt 123 KD for 5 d. Arrows, degradative autolysosomes. Double arrows, initial AVs. Arrowhead, phagophore isolation membrane. M, mitochondrion in an AV. Asterisks, glycogen particle clusters. Bars: (a, b, f, and g) 0.5 μm: (c and d) 200 nm; (e) 1 μm. (B) Quantification of the number of AVs per unit cytoplasmic area of 4.5 μm² (n≧64) and the percentage of cytoplasmic area occupied by AV in randomly sampled cytoplasmic areas (n=5 areas of >200 μm²) of PC3 and U87MG cells with and without Dox-induced shAkt 123 expression. Error bars represent SEM. (e) Dox-induced Akt silencing caused degeneration in PC3 and U87MG tumors. (C) (a) PC3 tumors expressing the control EGFP shRNA after 15 d of Dox treatment. The tumor cells contain large nuclei and nucleoli, some lipid droplets (asterisks), and are connected by cell junctions (arrowheads). (b-d) PC3 tumors expressing shAkt 123 after 15 (b and c) or 10 d (d) of Dox treatment. (b) Cells and nuclei in these tumors often appear shrunken. Arrows, AVs. E, eosinophil. (c) Two AVs (arrows) found among dilated RER cisternae in a degenerating tumor cell. (d) Ultrathin cryosection with immunogold labeling of human LAMP1. Label occurs on lysosomes (arrow) and AVs (top inset). Some of the tumor cells also contain human LAMP1-positive dense bodies with a shape reminiscent of microautophagy (bottom inset; de Waal, E. J., H. Vreeling-Sindelarova, J. P. Schellens, J. M. Houtkooper, and J. James, (1986), Quantitative changes in the lysosomal vacuolar system of rat hepatocytes during short-term starvation. A morphometric analysis with special reference to macro- and microautophagy. Cell Tissue Res. 243:641-8). The tumor cells have widened nuclear envelope and ER cisterns (asterisks), which contain small cytoplasmic islands (arrowheads). (e) U87MG tumor after 5 d of vehicle treatment. (f-h) U87MG-shAkt 123 tumor after 5 d of Dox treatment. Arrows, AVs. (h) In some tumor samples, cells with glycogen clusters (asterisks) and glycogen-containing AVs occur. Bars: (a-c) 2 μm: (e and f) 1 μm: (g) 0.5 μm: (d and h) 200 nm.

FIG. 4 depicts lysosomotropic agents accelerated cell death in combination with Akt KD. (A) CQ treatment caused accumulation of GFP-LC3 dots in Dox-treated PC3-shAkt 123 cells. PC3-shAkt 123 cells stably expressing GFP-LC3 were pretreated with or without 1 μg/ml Dox for 6 d and treated with or without 10 μM CQ. GFP fluorescence was imaged after 1 d of CQ treatment. Arrowheads point to representative GFP dots or clumps. Bar, 10 μm. (B) Effect of shAkt123 and 10 μM CQ on LC3 processing, PARP cleavage, and total Akt in PC3-shAkt 123 cells treated with or without Dox or CQ. The ratio of LC3-II to LC3-I and cleaved (Cl) to full-length (FL) PARP was quantified from immunoblots of cell lysates made at days 1 and 2 of CQ treatment. Immunoblots of day 2 samples are shown. Molecular masses are indicated in kilodaltons parenthetically next to each protein. Data are representative of three independent experiments. (C) CQ promoted cell death in PC3 cells induced to express shAkt123, whereas 3-MA pretreatment delayed this effect. PC3-shAkt123 cells were preincubated with 1 μg/ml Dox for 3 d to induce shRNA expression before cells were seeded into fresh medium containing 10 μM CQ or 2.5 nM Ba with or without Dox. 1 mM 3-MA was added with Dox, both during and after the pretreatment. Cell viability was determined at days 2, 3, and 4 under 0.5% (e) or 0% (D) FBS (cells treated with Ba alone under 0% FBS were followed for 2 and 3 d only). The percentage of the annexin V-positive PI-negative population was determined at days 2, 3, and 4 under 0.5% FBS. Caspase-3/7 activity was determined at days 2 and 3 under 0% FBS and expressed as relative fluorescence units (RFU, in thousands) normalized to the same number of cells. Error bars represent SD of three independent experiments.

FIG. 5 depicts CQ accelerated cell death in combination with III-5. (A) PC3 cells were treated with DMSO or 0.5 μM III-5 in the presence or absence of 10 μM CQ under 0.5% FBS. Cell viability was determined by PI exclusion at days 2, 3, and 5 Annexin V staining was analyzed at days 2 and 3 and broken down into PI+ or PI− populations. (B) Time course of cell viability in PC3 cells treated with 0.5 (III-5-0.5) or 20 μM (III-5-20) III-5 with or without 10 μM CQ or 3 mM 3-MA. PC3 cells pretreated with III-5 for 24 h under 1% FBS were split into medium containing 0.5% FBS in the presence or absence of CQ. 3-MA was added immediately before III-5, 24 h before CQ addition. Cell viability was determined by PI exclusion at the indicated time points after CQ addition. Error bars represent SEM (n=3). LC3-II to LC3-I ratios were determined from quantitation of immunoblots. (C) CQ dramatically increased the size and number of MDC+ vacuoles in PC3 cells treated with III-5, whereas 3-MA suppressed this effect. Cells were cultured in medium containing 0.5% FBS and treated with DMSO, 0.5 μM III-5, 10 μM CQ, and 5 mM 3-MA, alone or in combinations as indicated. MDC staining at 48 h is shown. Bar, 10 μm.

FIG. 6 depicts CQ accelerated cell death in combination with II-4. (A) PC3 cells were treated with DMSO or 4 μM II-4 in the presence or absence of 10 μM CQ under 0.5% FBS. Cell viability was determined by PI exclusion over the course of 10 d. Error bars represent SEM. Representative data from one of three independent experiments are shown. (B) Immunoblot analysis of cell lysates collected at the indicated time points from the experiment shown in A. Arrowheads indicate the positions for LC3-I and -II, CathD 43, and CathD 28. Quantifications of the indicated markers are shown in C. CathD 43, the 43-50-kD forms of cathepsin D precursors. CathD 28, the 28-kD cathepsin D heavy chain.

FIG. 7 depicts accumulation of AVOs preceded plasma membrane rupture and correlated with the appearance of apoptotic and anucleated cells with Akt inhibitor (“Akti”), in this example compound II-4, and CQ treatment. (A) PC3 cells treated with DMSO, 10 μM II-4, 10 μM CQ, or both under 5% FBS were followed for 3 d using time-lapse microscopy. Representative images of the cells at the indicated time points are shown. White arrowheads indicate the fusion between two adjacent cells before plasma membrane rupture in cells treated with both agents. Bar, 10 μm. (B) PC3 cells treated with the indicated agents were stained with AO and analyzed by multispectral imaging flow cytometry. (left) Brightfield (BF), nuclei (green), vacuoles (red), and green/red composite images of three representative cells with each treatment are shown. Bars, 10 μm. (middle) plotting AO green intensity versus AO green bright detail area revealed three distinct populations: R2 anucleated cells, R3 apoptotic cells, and R4 live cells. (right) AO red intensity for R4 is plotted on the histogram with an arbitrary gate (R5) drawn to include events with the brightest AO red intensity. R2, R3, and R4 histograms are overlaid in the Akti (II-4)+CQ plot only. (C) Statistics for each population shown in B. *, percentage of total single cells; **, mean fluorescence intensity of R4 live cells; ***, percentage of R4 live cells.

FIG. 8 depicts Akt inhibition induces mitochondrial superoxide and cellular ROS production, which is augmented by CQ. (A) PC3 cells cultured in 0.5% FBS were treated with DMSO, 3 μM II-4, 10 μM CQ, or both, stained with MitoSOX red dye, and examined by fluorescence microscopy. Images at 24 h are shown. Bar, 10 μm. (B) PC3 cells treated as in A were stained with the Image-iT LIVE green ROS Detection kit and examined by fluorescence microscopy at 24 h. Bright field (BF) images of cells are also shown. Bar, 10 μm. (C) Quantification of MitoSOX red and ROS green fluorescence intensities by flow cytometry at 24 h. Cells were treated as in A and B. Error bars represent SEM (n=3).

FIG. 9 depicts CQ selectively accelerated cell death in Akti-treated PTEN-null cells in vitro and enhanced the antitumor efficacy of Akt KD in vivo. (A) PTEN−/− (−/−) MEFs were more sensitive than isogenic PTEN+/+ (+/+) counterparts to the combined treatment with II-4 and CQ. MEFs were treated with 5 μM each of II-4 and CQ under 1% FBS, and cell viability was determined at days 0, 2, and 3 by PI exclusion. Error bars represent SEM (n=3). (B) Mean tumor volumes of PC3 xenograft tumors treated daily with vehicle (Veh), Dox only, CQ only, or both Dox and CQ over a 28-d period. The vehicle and vehicle+CQ groups were followed for up to 18 d before terminated because of weight loss from the tumor burdens. Error bars represent SEM (n=10 tumors in each cohort). (C) Scatterplot of the tumor volumes in the Dox only and Dox+CQ groups on day 28 (P=0.05). Horizontal bars indicate mean tumor volumes. Numbers of tumors with complete remission (CR, dashed line) are indicated for each group. (D) Individual tumor growth plotted as a percentage of tumor volume change compared with day 0 for the Dox only and Dox+CQ cohorts shown in A. Dashed lines indicate −100% change from the starting tumor volumes, i.e., complete tumor regression. Numbers of tumors with smaller (<<0% change) or larger (>0% change) than the starting tumor volumes on day 28 are indicated.

FIG. 10 depicts increased AV accumulation and apoptosis in PC3 tumor with combined Akt123 KD and CQ treatment. (A) (a) EM images of PC3-shAkt123 tumors treated for 5 d with CQ only. Arrows, dense AVs and lysosomes; N, nucleolus. (b) Dox only. Arrows, AVs with a less dense appearance than in a. (c and d) Both Dox and CQ. (c) Numerous dense and enlarged AVs (arrows) accumulate in tumor cells. An apoptotic cell (Ap) is partially surrounded by a macrophage (M). T, tumor cell. (d) Apoptotic nuclei (Ap) among the AV-loaded (arrows) tumor cells. Insets, enlarged images of AVs (a-c) and abnormal mitochondria (*) in each tumor. Bars: (a-c) 2 μm: (d) 1 μm. (B) Quantification of the percentage of cytoplasmic area occupied by AVs in randomly sampled cytoplasmic areas (n=6 areas of >80 μm2). (C) Percentage of apoptotic nuclei among randomly sampled tumor cell nuclei (n=3-4 sets of 100 tumor cell nuclei). (B and C) Error bars represent SEM; *, P<0.0005 compared with the other three groups.

FIG. 11 depicts Akt knockdown by shRNA induces autophagy gene expression. PC3 cells were induced to express shRNA to indicated Akt isoforms for 72 hours by Doxycycline, RNA was extracted from both Dox treated (Dox+) or untreated control (Dox−) cells. Microarray analysis was carried out using Affymetrix chips. Ratios of the expression levels of each autophagy gene from Dox+ and Dox− samples are shown. Data are mean values from 3 independent experiments.

FIG. 12 depicts Akt inhibitors that induce autophagy gene expression. PC3 cells were treated with DMSO vehicle control or various Akt inhibitors, including 1-(1-(4-(5-hydroxy-6-methyl-3-phenylpyrazin-2-yl)benzyl)piperidin-4-yl)-1H-benzo[d]imidazol-2(3H)-one (II-1), 1-(1-(4-(6-hydroxy-5-isobutyl-3-phenylpyrazin-2-yl)benzyl)piperidin-4-yl)-1H-benzo[d]imidazol-2 (3H)-one (II-2), 1-(1-(4-(7-phenyl-1H-imidazo[4,5-g]quinoxalin-6-yl)benzyl)piperidin-4-yl)-1H-benzo[d]imidazol-2 (3H)-one (II-4), (S)-2-(4-chlorophenyl)-1-(4-((5R,7R)-7-hydroxy-5-methyl-6,7-dihydro-5H-cyclopenta[d]pyrimidin-4-yl)piperazin-1-yl)-3-(isopropylamino)propan-1-one (III-4), and (S)-1-(3-(N-(4-(3-phenylisoxazol-5-yl)thiazol-2-yl)thiophene-2-carboxamido)propyl)piperidine-2-carboxamide (IV-1), at 1 or 5 μM for 6 or 24 hours. RNA was extracted from the cells. Microarray analysis was carried out using Affymetrix chips. Expression levels of each autophagy gene are normalized to the DMSO controls. Data are mean values from 3 independent experiments.

FIG. 13 depicts various mTOR, PI3K and Akt inhibitors alone induce increased autophagic vacuole accumulation as measured by side scatter (SSC) in a flow cytometer. Inhibitors include 3-phenyl-2-(4-((4-(5-(pyridin-2-yl)-1H-1,2,4-triazol-3-yl)piperidin-1-yl)methyl)phenyl)-1,6-naphthyridin-5(6H)-one (II-3), benzyl 2-(4-(3-ethylureido)phenyl)-4-(1,4-oxazepan-4-yl)-5H-pyrrolo[3,4-d]pyrimidine-6(7H)-carboxylate (III-1); 1-ethyl-3-(4-(4-morpholino-7-(pyrimidin-2-yl)-5,6,7,8-tetrahydropyrido[3,4-d]pyrimidin-2-yl)phenyl)urea (III-2), (R)-1-(44(2-(2-aminopyrimidin-5-yl)-7-methyl-4-morpholinothieno[3,2-d]pyrimidin-6-yl)methyl)piperazin-1-yl)-2-hydroxypropan-1-one (III-3), 4-(2-(1H-indazol-4-yl)-6-((4-(methylsulfonyl)piperazin-1-yl)methyl)thieno[3,2-d]pyrimidin-4-yl)morpholine (III-6), (S)—N-(4-(benzo[d][1,3]dioxol-5-yl)thiazol-2-yl)-N-(3-(2-formylpiperidin-1-yl)propyl)thiophene-2-carboxamide (IV-2) and Rapamycin.

FIG. 14 depicts CQ (10 μM) increases the number and size of autophagic vacuoles when combined with the various mTOR, PI3K, and Akt inhibitors.

FIG. 15 depicts various mTOR, PI3K and Akt inhibitors alone induce increased autophagic vacuole accumulation as measured by the red to green fluorescent ratio after acridine orange staining.

FIG. 16 depicts CQ (10 μM) increases the accumulation of autophagic vacuoles when combined with the various mTOR, PI3K and Akt inhibitors as measured by the red to green fluorescent ratio after acridine orange staining.

FIG. 17 depicts Akt inhibitor III-4 and PI3K inhibitor III-6 both synergize with CQ to accelerate cell death.

FIG. 18 depicts relative levels of Akt isoforms in cancer cell lines and inducible knockdown of Akt isoforms in U87MG cells and xenograft tumors. (A) Relative expression levels of each Akt isoform in tumor cell lines. Akt proteins in total cell lysates from each cell line were analyzed by Western blot analysis using isoform-specific antibodies and quantified using recombinant proteins of each isoform loaded on the same gel as standards. Data are representative of two independent experiments. (B) Western blot validation of Akt KD in U87MG cells expressing the inducible shRNA constructs. Stable pools of U87MG cells containing the indicated constructs targeting the respective Akt isoforms were induced to express shRNA with 1 mg/ml Dox for 3 days. Total cell lysates were analyzed using the indicated antibodies. GAPDH levels were determined as a loading control. Molecular weights are indicated in kDa parenthetically next to each protein. (C-F) U87 MG xenograft tumors containing the indicated shRNAs were treated with vehicle control or Dox. Each cohort consisted of ten mice. (G) Dox has no effect on the growth of wild-type PC3 cells. (H) PC3 tumor growth retardation induced by a second shAkt1 construct. Error bars represent SEM. *P<0.05; **p<0.005.

FIG. 19 depicts the effect of shGFP and Akt isoform KDs on cell cycle progression, apoptosis and autophagy. (A) Steady-state cell cycle profiles of PC3 stable clones containing the indicated shRNAs with or without Dox treatment (1 μg/ml for 96 hours). Error bars represent standard deviations of 2 independent experiments. (B) Cell cycle profiles in (A) expressed as the percentage of change in each phase with Dox treatment compared to the same clone without Dox treatment. Data are representative of at least 2 independent experiments with at least 0.5×106 cells analyzed for each condition. (C) EM quantification of the number of AVs per unit cytoplasmic area of 4.5 μm² (n=130) of PC3 and U87MG cells showing no significant difference in AV numbers with or without Dox− induced shGFP expression. Error bars represent SEM. (D) Increased punctate LC3 immunofluorescence staining in PC3 and U87MG cells after 5 days of Dox-induced shAkt123 expression compared to untreated controls. Bar, 10 μn. (E) Percentage of PC3-shAkt123 cells with GFP-LC3 dots under the indicated treatments. PC3-shAkt123 cells stably expressing GFP-LC3 were pretreated as in FIG. 4 and the percentage of cells with >5 punctate GFP dots visible was determined. Note that although CQ treatment alone caused perinuclear GFP-LC3 clumps in the cells, these are morphologically different from the widespread cytoplasmic GFP-LC3 dots in Dox treated cells. Error bars represent standard deviation from 2 random fields of at least 15 cells each. (F) CQ treatment causes accumulation of MDC-labeled vacuoles in Dox-treated PC3 cells expressing shAkt123. PC3-shAkt123 cells were incubated in the presence or absence of 1 μg/ml Dox, with or without 10 μM CQ, for 5 days before labeling with MDC. Scale bar: 10 μm. (G) Flow cytometry analysis of acridine orange-stained PC3-shAkt123 cells treated with 1 μg/ml Dox, 10 μM CQ or both. FL1-H indicates intensity of green fluorescence of the nucleus. FL3-H indicates intensity of red fluorescence of the AVOs. Percentage of cells with high FL3-H/FL1-H ratio, characteristic of cells with high levels of AVOs (Paglin et al., 2001), is indicated on the histogram. The data shown are representative of three independent experiments.

FIG. 20. Effect of LAMP2, Atg7, protease inhibitors, cathepsin D siRNA and pepstatin A on PC3 cell viability in combination with PI-103 or Akti-1/2. (A) Immunoblot showing KD of LAMP2 by siRNA oligos a-d and their pool compared to a non-targeting control oligo (siCtrl). (B) Effect of LAMP2 siRNA oligos on PC3 cell viability. PC3 cells were transfected with 80 nM of siRNA under 0.5% FBS, PI-103 (0.5 μM) or DMSO was added 2 days post-transfection and cell viability (PI exclusion) was analyzed 4 days after PI-103 addition. (C) Atg7 immunoblot of PC3 cells transfected with Atg7 siRNA pool (Santa Cruz) or a non-targeting control oligo. (D) Effect of Atg7 siRNA oligos on PC3 cell viability under the different treatments. PC3 cells were transfected with 20 nM of siRNA under 0.5% FBS, Akti-112 (5 μM) or DMSO was added with or without CQ (10 μM) 2 days post-transfection and cell viability (PI exclusion) was analyzed 2 and 3 days after compound addition. *, P<0.05 between the two conditions. (E) PC3 cells treated with the indicated concentrations of zVAD.fmk added together with DMSO, 10 μM CQ, 3 μM Akti-1/2, or both Akti-1/2 and CQ. Cell viability was determined at day 3 by PI exclusion. *, P<0.05; **, P<0.001 between the two conditions. (F) PC3 cells treated with the indicated concentrations of zFA.fmk added together with DMSO, 10 μM CQ, 3 μM Akti-1/2, or both Akti-1/2 and CQ. Cell viability was determined at day 3 by PI exclusion. *, P<0.05; **, P<0.001 between the two conditions. (F) PC3 cells treated with the indicated concentrations of zFA.fmk added together with DMSO, 10 μM CQ, 3 μM Akti-1/2, or both Akti-1/2 and CQ. Cell viability was determined at day 3 by PI exclusion. *, P<0.05 compared to either Akti-1/2 alone or zFA.fmk alone treated cells. (G-H) PC3 cells pre-treated with the indicated concentrations of CA-074-Me (G) or ALLN (H) for 2 hours prior to addition of DMSO, 10 μM CQ, 3 μM Akti-1/2, or both Akti-1/2 and CQ. Cell viability was determined at day 3 by PI exclusion. *, P<0.05 compared to either Akti-1/2 alone or the protease inhibitors alone treated cells. (I) Effect of cathepsin D siRNA oligos on PC3 cell viability under the different treatments. PC3 cells were transfected with 10 nM of cathepsin D siRNA pool (Santa Cruz) or a non-targeting control under 0.5% FBS, Akti-1/2 (5 μM) or DMSO was added with or without CQ (10 μM) 2 days post-transfection and cell viability (PI exclusion) was analyzed 2 days after compound addition. Cathepsin D knockdown was confirmed by immunoblot analysis shown at the upper right corner. (J) Effect of pepstatin A on PC3 cell viability under the different treatments. PC3 cells were treated with DMSO, 5 μM Akti-1/2, 10 μM CQ or both under 0.5% FBS with or without 200 μM pepstatin A. Cell viability (PI exclusion) was analyzed 2 days after compound addition. *, P<0.05 between the two conditions. (B, D-J) Error bars represent SEM (n=3). Molecular weights are indicated in kDa parenthetically next to each protein for immunoblots in A, C & I.

FIG. 21. CQ promoted mitochondrial membrane depolarization and cellular ROS accumulation in combination with Akti-1/2 (A) CQ enhanced Akti-1/2-induced mitochondria depolarization. PC3 cells cultured in 0.5% FBS were treated with DMSO, 3 μM Akti-1/2, 10 μM CQ, or both and stained with the MitoPT dye (Immunochemistry Technologies, LLC) at various time points. Images at 48 h are shown. Healthy mitochondria show punctate red stains of JC-1 aggregates (MitoPT-R), while cells with depolarized mitochondria show diffuse green stains of JC-1 monomers (MitoPT-G). Merged images between red and green channels are also shown (MitoPT-M). Scale bar: 20 μm. (B) Percentage of cells showing red punctate stains and diffuse green stains from treatments described in (A). >86 cells from two random fields of fluorescent images of each treatment were counted. Error bars represent standard deviations between the two fields. (C) 3-MA reduced ROS signals generated by CQ, Akti and Akti+CQ. PC3 cells cultured in 0.5% FBS were pre-treated with 3-MA overnight, then treated with DMSO, 5 μM Akti-1/2, 10 μM CQ, or both for 48 hours and stained with the Image-iT LIVE Green ROS Detection Kit. Total fluorescence intensity from all cells was collected using the Isocyte (Blueshift Biotechnologies), a laser scanning imager. Green fluorescence intensity is collected with a 488 nm laser and a 510-540 nm band pass filter, and normalized to cell number determined by Hoechst staining using a 405 nm laser and a 430-480 nm band pass filter. Error bars, standard deviation between two independent experiments. (D) Representative images of the green ROS signals and bright field (BF) taken under a Nikon TE300 inverted microscope. Akti-1/2 alone first induced a homogeneous increase in ROS level, but by 48 hours the fluorescence became significantly reduced and localized to perinuclear and cytoplasmic vacuoles resembling autolysosomes. Although CQ alone had little effect on ROS levels, combination with Akti-1/2 caused a prolonged increase in vacuolar fluorescence, as well as an increased population of cells with persistent homogeneous fluorescence, many of which showing morphological signs of apoptosis within 48 hours. Arrowheads point to representative cells exhibiting vacuolar fluorescence. Asterisks indicate representative cells with homogeneous green fluorescence and also exhibiting apoptotic morphology. Scale bar: 20 μm.

FIG. 22. NAC rescued cell death induced by Akti+CQ. (A) NAC reduced MitoSOX Red signal induced by the various treatments. PC3 cells were either pretreated for 1 day with 5 mM NAC then washed off (NACpr) and treated with the indicated agents, or pretreated for 1 hour with 5 mM NAC and incubated with the indicated agents in the continuous presence of NAC (NAC). MitoSOX signals were determined 24 hours after Akti-1/2 addition by Flow Cytometry. 5 μM Akti-1/2 and 10 μM CQ were used. Continuous NAC treatment is required for MitoSOX Red signal reduction at this time point. (B) Viability (PI exclusion) of cells treated as in (A) was determined 4 days after Akti-1/2 addition by Flow Cytometry. NAC pretreatment showed a small, insignificant decrease in cell death in the Akti+CQ group, and significant rescue is seen with continuous NAC treatment. (A and B) Error bars represent SEM (n=3). (C) PC3 cells stably expressing GFP-LC3 were treated as in (A) and analyzed by immunoblots at 48 hours after Akti-1/2 addition. β-actin and GAPDH were used as loading controls. Quantifications of p62 and cleaved GFP levels normalized to GAPDH, and LC3-II to LC3-I ratios are shown on the right. Akti caused a reduction in p62 levels, increased LC3-I turnover (both endogenous and GFP-LC3-I) and concomitant LC3-II and cleaved GFP accumulation. CQ increased the level of p62 both with and without Akti, consistent with its blocking of p62 degradation in the autolysosomes. CQ also induced LC3-II and cleaved GFP accumulation due to its blocking of their degradation in the autolysosomes. NAC treatment counteracted all these effects induced by Akti with or without CQ but did not affect Akti's ability to inhibit pAkt or pS6. NACpr showed some but weaker effects than continuous NAC treatment. (D) Time lapse fluorescence images of PC3 cells stably expressing GFP-LC3 treated with DMSO, 3 μM Akti-1/2 in the presence or absence of 5 mM NAC (added 1 hour prior to Akt-1/2) at 2, 11 and 23 hours after Akti-1/2 addition. Arrowheads point to representative cells with visible GFP-LC3 dots. Scale bar: 50 μm.

FIG. 23 depicts the pHUSH vector system used to make shRNAs specifically targeting Akt isoforms. The pHUSH vector system comprises an shRNA expression shuttle plasmid (pShuttle-H1) and a viral vector backbone (pHUSH-GW; GW=Gateway) that contains a TetR-IRES-Puro cassette to enable Tet-regulated shRNA expression. The Akt shRNA vectors were constructed by (1) designing and cloning shRNA sequences into pShuttle-H1 (2) transferring the H1-shRNA cassette into pHUSH-GW by a Gateway (Invitrogen) recombination reaction and (3) packaging the completed H1-pHUSH plasmid as a retrovirus. For each shRNA, a 19 bp siRNA sequence was designed using an appropriate algorithm against the coding sequence of an Akt gene(s). The shRNA sequence was converted into an shRNA hairpin sequence, and then the corresponding double-stranded DNA oligo was synthesized and cloned into pShuttle-H1 as shown. The effectiveness of each shRNA in pShuttle-H1 vector was verified by transient transfection into cells and the degree of knockdown of each Akt isoform examined by western blots. The validated H1-shRNA cassette was then transferred into the pHUSH-GW vector and packaged as a retrovirus (Table 1 summarizes the validated sequences used). Cells stably expressing each shRNA were generated by retroviral infection with single or combination of shRNA-containing viruses. For single Akt isoform knockdowns, cells were infected with one retroviral vector encoding an shRNA construct singly targeting each Akt isoform (constructs 252 & 253 for Akt1, 254 & 255 for Akt2, and 259 & 260 for Akt3) and stable clones were selected using 5 mg/ml puromycin. For dual Akt1 and Akt2 knockdown, a single shRNA targeting both Akt1 and 2 simultaneously (construct 256 & 257) was used. Dual Akt2 and 3 (constructs 255 and 261), or triple Akt1, 2 and 3 (constructs 257 and 261) knockdowns were achieved by co-infecting the cells with two retroviral vectors containing different antibiotic selection markers (puromycin and hygromycin), each encoding one single shRNA, and stable clones were selected using 5 mg/ml puromycin and 300 mg/ml hygromycin. For dual Akt1 and 3 knockdown, either a single shRNA targeting both Akt1 and 3 (construct 258), or co-infection with two shRNA vectors (constructs 253 and 261) was employed (Table 2). All shRNAs shown in Table 2 have been validated in cultured cells. The efficiency and tumor inhibitory effect of shRNAs validated in xenograft models are summarized in Table 3.

FIG. 24 depicts a model of the mechanism of cell death induced by the combination of chloroquine with Akt inhibition. Akt inhibition alone (by shRNA, specific Akt inhibitors or class I PI3K inhibitors) can activate autophagy through multiple mechanisms, including decreased mTORC1 activity downstream of Akt, (Corradetti M N, Guan K L. Upstream of the mammalian target of rapamycin: do all roads pass through mTOR? Oncogene (2006); 25:6347-60), increased activity of FoxO proteins (Zhao J, Brault J J, Schild A, Goldberg A L. Coordinate activation of autophagy and the proteasome pathway by foxO transcription factor, Autophagy (2008); 4:378-80), and decreased glucose and energy metabolism. We also observed an accumulation of abnormal mitochondria and signs of ER stress (unpublished data) in cells with Akt knockdown or inhibition, both of which can induce autophagy. (Yorimitsu T, Klionsky D J. Endoplasmic reticulum stress: a new pathway to induce autophagy. Autophagy (2007); 3:160-2). Abnormal mitochondria can generate a ROS signal, resulting in elevated autophagic removal of the damaged mitochondria and attenuation of the oxidative stress. 3-methyladenine (3-MA) and other non-selective pan-PI3K inhibitors that inhibit class III PI3K, such as wortmannin or LY294002, block the induction of autophagy. Impaired autolysosomal degradation caused by CQ (or other inhibitors of lysosomal enzyme activity) can result in aggregation of deleterious ROS generators that further amplify the ROS damage. Multiple downstream events can lead to both apoptosis-like and non-apoptotic cell death. It is unclear whether the autophagic response induced by Akt inhibition alone can eventually lead to cell death directly in some cells, or require additional insults.

FIG. 25A-E depict AV accumulation in PC3 cells treated with Akt inhibitor, CQ and their combination. (Panels A-C): PC3 cells grown under 0.5% FBS were treated with (A) DMSO control, (B) 10 μM CQ, and (C) 5 μM Akti-1/2 for 1 day. Both CQ and Akti-1/2 alone induced accumulation of AVs (arrows). (Panels D-E): Combined treatment of Akti-1,2 and CQ resulted in accumulation of larger AVs and the appearance apoptotic nuclei. (D) After 1 day of treatment, many cells contained very large AVs (arrows), distended ER cisterns (arrowheads), and appear largely vacuolated (small arrows). (E) After 2 days of treatment, apoptosis became apparent in a large proportion of the cells (asterisk). Scale bars, 2 μm.

FIGS. 26A-B depicts measured ED50 for CQ alone, the AKTi III-4 alone, and their combination, in multiple cell lines. FIG. 26A depicts the compound ratio work table for CQ and the AKTi. FIG. 26B depicts the CI (combination index) by using bar graphs for data obtained by CellTiter-Glo assay at day 4 of treatment with 10% serum. The X-axis shows the cell line and the Y-axis shows the concentration in μM. The combination of the AKTi with CQ significantly lowers the ED50 for both the AKTi and CQ.

FIGS. 27A-B depicts data for the PC3 (PTEN-, p53- and Al) cell line. FIG. 27A depicts the CI values at ED50, ED75 and ED90 when the AKTi III-4 and CQ are combined at varying ratios. The data show combination ratios of III-4:CQ in the ranges of 5:1 to 1:800, respectively, with the lowest ED50, ED75 and ED90 values in the ratios of about 1:1.5 to about 1:200, with alternative ratios in the range of about 1:3 to about 1:50, with additionally alternative ratios of about 1:12 to about 1:50, with particular ratio of about 1:25, respectively. 27B depicts growth inhibition curves of the AKTi III-4 alone, CQ alone, and the AKTi and CQ combination dosed in a 1:25 ratio (about the EC50 ratio). The x-axis indicates III-4 concentration in μM and the y-axis indicates the % of control. The data indicate that the combination inhibits growth in the cell line at lower concentrations of III-4 than either III-4 alone or CQ alone.

FIGS. 28A-B depicts data for the MDA-361.1 (PI2-K mut (E545K), Her2+, HR+ and Luminal) cell line. FIG. 28A depicts the CI values at ED50, ED75 and ED90 when the AKTi III-4 and CQ are combined at varying ratios. The data show combination ratios of III-4:CQ in the ranges of 5:1 to 1:800, respectively, with the lowest ED50, ED75 and ED90 values in the ratios of about 1:1.5 to about 1:200, with alternative ratios in the range of about 1:3 to about 1:25, with particular ratio of about 1:12.5, respectively. FIG. 28B depicts growth inhibition curves of the AKTi III-4 alone, CQ alone, and the AKTi and CQ combination dosed in a 1:12.5 ratio (about the EC50 ratio). The x-axis indicates III-4 concentration in μM and the y-axis indicates the % of control. The data indicate that the combination inhibits growth in the cell line at lower concentrations of III-4 than either III-4 alone or CQ alone.

FIGS. 29A-B depicts data for the MDA-MB-231 (Kras, Braf, p53 mut, Triple- and Basal) cell line. FIG. 29A depicts the CI values at ED50, ED75 and ED90 when the AKTi III-4 and CQ are combined at varying ratios. The data show combination ratios of III-4:CQ in the ranges of 5:1 to 1:800, respectively, with the lowest ED50, ED75 and ED90 values in the ratios of about 2.5:1 to about 1:1:3, with particular ratio of about 1.25:1, respectively. FIG. 29B depicts growth inhibition curves of the AKTi III-4 alone, CQ alone, and the AKTi and CQ combination dosed in a 1.25:1 ratio (about the EC50 ratio). The x-axis indicates III-4 concentration in μM and the y-axis indicates the % of control. The data indicate that the combination inhibits growth in the cell line at lower concentrations of III-4 than either III-4 alone or CQ alone.

FIGS. 30A-B depicts data for the U87MG (PTEN-, PI3K mut (1391M)) cell line. FIG. 30A depicts the CI values at ED50, ED75 and ED90 when the AKTi III-4 and CQ are combined at varying ratios. The data show combination ratios of III-4:CQ in the ranges of 5:1 to 1:800, respectively, with the lowest ED50, ED75 and ED90 values in the ratios of about 2.5:1 to about 1:25, with alternative ratios in the range of about 1.25:1 to about 1:3, with a particular ratio of about 1:1.5, respectively. FIG. 30B depicts growth inhibition curves of the AKTi III-4 alone, CQ alone, and the AKTi and CQ combination dosed in a 1:1.5 ratio (about the EC50 ratio with minimum CI). The x-axis indicates III-4 concentration in μM and the y-axis indicates the % of control. The data indicate that the combination inhibits growth in the cell line at lower concentrations of III-4 than either III-4 alone or CQ alone.

FIGS. 31A-C depicts data for the Panc-1 (Akt2 amp, Kras mut, p53 mut) cell line. FIG. 31A depicts the CI values at ED50, ED75 and ED90 when the AKTi III-4 and CQ are combined at varying ratios. The data show combination ratios of III-4:CQ in the ranges of 5:1 to 1:800, respectively, with the lowest ED50, ED75 and ED90 values in the ratios of about 2.5:1 to about 1:1.3, with a particular ratio of about 1.25:1, respectively. FIG. 31B depicts growth inhibition curves of the AKTi III-4 alone, CQ alone, and the AKTi and CQ combination dosed in a 1.28:1 ratio (about the EC50 ratio with minimum CI). The x-axis indicates III-4 concentration in μM and the y-axis indicates the % of control. The data indicate that the combination inhibits growth in the cell line at lower concentrations of III-4 than either III-4 alone or CQ alone. FIG. 31C depicts growth inhibition curves of the AKTi III-4 alone, CQ alone, and the AKTi and CQ combination dosed in a 1:1.56 ratio (about the EC 10 ratio with minimum CI). The x-axis indicates III-4 concentration in μM and the y-axis indicates the % of control. The data indicate that the combination inhibits growth in the cell line at lower concentrations of III-4 than either III-4 alone or CQ alone.

FIGS. 32A-B and 34A-B depict data showing correlation between Autophagy induction and apoptosis induction when each compound is combined with CQ at 24 hrs after the given compound treatment measured by Acridine Orange staining in 537MEL melanoma cell line and SKBR3Breast cancer cell line, respectively. Control was DMSO. Compounds tested were 1-(1-(4-(7-phenyl-1H-imidazo[4,5-g]quinoxalin-6-yl)benzyl)piperidin-4-yl)-1H-benzo[d]imidazol-2 (3H)-one (II-4), 3-phenyl-2-(4-((4-(5-(pyridin-2-yl)-1H-1,2,4-triazol-3-yl)piperidin-1-yl)methyl)phenyl)-1,6-naphthyridin-5 (6H)-one (II-3), (S)-2-(4-chlorophenyl)-1-(4-((5R,7R)-7-hydroxy-5-methyl-6,7-dihydro-5H-cyclopenta[d]pyrimidin-4-yl)piperazin-1-yl)-3-(isopropylamino)propan-1-one (III-4), 4-(2-(1H-indazol-4-yl)-6-((4-(methylsulfonyl)piperazin-1-yl)methyl)thieno[3,2-d]pyrimidin-4-yl)morpholine (III-6), (R)—N-(2,3-dihydroxypropoxy)-3,4-difluoro-2-(2-fluoro-4-iodophenylamino)benzamide (VII), (S)-1-ethyl-3-(4-(4-(3-methylmorpholino)-7-(pyrimidin-2-yl)-5,6,7,8-tetrahydropyrido[3,4-d]pyrimidin-2-yl)phenyl)urea (III-2a) and rapamycin.

FIGS. 33A-G and 35A-G show dose response curve of apoptosis induction in 537MEL melanoma and SKBR3Breast cancer cells, respectively, treated with the indicated compound alone or with 10 μM CQ, measured by Annezin V (AnnV) and Propidium iodide (PI) staining.

In above Figures, a positive correlation is shown between autophagy induction and apoptosis induction when the compound is combined with CQ. In the case of the compound that does not induce autophagy (VII), no synergistic apoptosis induction is shown when combined with CQ.

### DETAILED DESCRIPTION OF THE INVENTION

### Definitions

The term “neoplasms” encompasses “cancer” and “cancerous,” which refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. In one embodiment, the neoplasm is other than a glycolysis dependent cancer. In another embodiment, the neoplasm is prostate, breast, glioma or pancreatic cancer. In another embodiment, the neoplasm is prostate, breast or ovarian cancer. In another embodiment, the neoplasm comprises PTEN or PI3K mutations. In another embodiment, the neoplasm is resistant to inhibitors of the Aid kinase pathway.

The term “alkyl” as used herein refers to a saturated linear or branched-chain monovalent hydrocarbon radical of one to twelve carbon atoms, wherein the alkyl radical may be optionally substituted independently with one or more substituents described below. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like.

The term “alkenyl” refers to linear or branched-chain monovalent hydrocarbon radical of two to twelve carbon atoms with at least one site of unsaturation, i.e., a carbon-carbon, sp² double bond, wherein the alkenyl radical may be optionally substituted independently with one or more substituents described herein, and includes radicals having “cis” and “trans” orientations, or alternatively, “E” and “Z” orientations. Examples include, but are not limited to, ethylenyl or vinyl (—CH═CH₂), allyl (—CH₂CH═CH₂), and the like.

The term “alkynyl” refers to a linear or branched monovalent hydrocarbon radical of two to twelve carbon atoms with at least one site of unsaturation, i.e., a carbon-carbon, sp triple bond, wherein the alkynyl radical may be optionally substituted independently with one or more substituents described herein. Examples include, but are not limited to, ethynyl (—C≡CH), propynyl (propargyl, —CH₂C≡CH), and the like.

The terms “carbocycle”, “carbocyclyl”, “carbocyclic ring” and “cycloalkyl” refer to a monovalent non-aromatic, saturated or partially unsaturated ring having 3 to 12 carbon atoms as a monocyclic ring or 7 to 12 carbon atoms as a bicyclic ring. Bicyclic carbocycles having 7 to 12 atoms can be arranged, for example, as a bicyclo [4,5], [5,5], [5,6] or [6,6] system, and bicyclic carbocycles having 9 or 10 ring atoms can be arranged as a bicyclo [5,6] or [6,6] system, or as bridged systems such as bicyclo[2.2.1]heptane, bicyclo[2.2.2]octane and bicyclo[3.2.2]nonane. Examples of monocyclic carbocycles include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, cyclododecyl, and the like.

“Aryl” or “aromatic” means a monovalent aromatic hydrocarbon radical of 6-20 carbon atoms derived by the removal of one hydrogen atom from a single carbon atom of a parent aromatic ring system. Some aryl groups are represented in the exemplary structures as “Ar”. Aryl includes bicyclic radicals comprising an aromatic ring fused to a saturated, partially unsaturated ring, or aromatic carbocyclic or heterocyclic ring. Typical aryl groups include, but are not limited to, radicals derived from benzene (phenyl), substituted benzenes, naphthalene, anthracene, biphenyl, indenyl, indanyl, 1,2-dihydronapthalene, 1,2,3,4-tetrahydronapthyl, and the like. Aryl groups are optionally substituted independently with one or more substituents described herein.

The terms “heterocycle,” “heterocyclyl” and “heterocyclic ring” are used interchangeably herein and refer to a saturated, a partially unsaturated (i.e., having one or more double and/or triple bonds within the ring) or aromatic carbocyclic radical of 3 to 20 ring atoms in which at least one ring atom is a heteroatom selected from nitrogen, oxygen and sulfur, the remaining ring atoms being C, where one or more ring atoms is optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo [4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. The term “heterocycle” includes heterocycloalkoxy. “Heterocyclyl” also includes radicals where heterocycle radicals are fused with a saturated, partially unsaturated ring, or aromatic carbocyclic or heterocyclic ring. Examples of heterocyclic rings include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro moieties are also included within the scope of this definition. Examples of a heterocyclic group wherein 2 ring carbon atoms are substituted with oxo (═O) moieties are pyrimidinonyl and 1,1-dioxo-thiomorpholinyl. The heterocycle groups herein are optionally substituted independently with one or more substituents described herein.

The term “heteroaryl” or “heteroaromatic” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The heterocycle or heteroaryl groups may be carbon (carbon-linked), nitrogen (nitrogen-linked) or oxygen (oxygen-linked) attached where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

The terms “treat” and “treatment” refer to both therapeutic treatment and prophylactic or preventative measures, which prevent or slow down (lessen) an undesired physiological change or disorder, such as the growth, development or spread of cancer. In one embodiment, the term “treat” and “treatment” refer to therapeutic treatment, which or slows down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

As used herein, “glycolysis dependent cancer” is meant to refer to cancer that is characterized by cancer cells that rely on glucose metabolism for essentially all of their energy needs excluding energy that may be obtained by autophagy. Cancer cells of glycolysis dependent cancer may be capable of some level of non-glycolytic metabolism but such level does not prevent the cancer cells from undergoing cell death by apoptosis or autophagy in the absence of a glucose energy source. There are numerous methods of determining whether or not a cancer is dependent upon glycolysis. Samples of tumors can be excised and examined in vitro by any one of several well known assays to determine if the cells are dependent on glycolysis. Such methods can determine whether or not the cells utilize aerobic or anaerobic glycolysis. FDG-PETscan technology uses high levels of glucose uptake as a marker for detection. The cancer cells that take up the detectable glucose derivative 18-fluoro-2-deoxyglucose can be located on a computer image of the patient's anatomy. Those cancers which can be detected by FDG-PETscan technology have a high likelihood of being dependent on glycolysis.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and/or stop) cancer cell infiltration into peripheral organs; inhibit (i.e., slow to some extent and/or stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The term “mammal” includes, but is not limited to, humans, mice, rats, guinea pigs, monkeys, dogs, cats, horses, cows, pigs, and sheep, and poultry.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. Acids which are generally considered suitable for the formation of pharmaceutically useful or acceptable salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1 19; P. Gould, International J. of Pharmaceutics (1986) 33 201 217; Anderson et al, The Practice of Medicinal Chemistry (1996), Academic Press, New York; Remington's Pharmaceutical Sciences, 18th ed., (1995) Mack Publishing Co., Easton Pa.; and in The Orange Book (Food & Drug Administration, Washington, D.C. on their website). These disclosures are incorporated herein by reference thereto.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The phrase “pharmaceutically acceptable” indicates that the substance or composition is compatible chemically and/or toxicologically, with the other ingredients comprising a formulation, and/or the mammal being treated therewith.

The phrase “substantially corresponding to” means that a sequence has inconsequential variations from the known or target sequence. In one example, a sequence has about 80% homology with a known or target sequence. In another example, a sequence has 85% homology. In another example, a sequence has 90, 91, 92, 93, 94, 95, 96, 97, 98, 99 or above % homology. Methods are known in the art for determining % homology.

A “solvate” refers to a physical association or complex of one or more solvent molecules and a compound of the invention. The compounds of the invention may exist in unsolvated as well as solvated forms. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water. This physical association involves varying degrees of ionic and covalent bonding, including hydrogen bonding. In certain instances the solvate will be capable of isolation, for example when one or more solvent molecules are incorporated in the crystal lattice of the crystalline solid. Preparation of solvates is generally known, for example, M. Caira et al, J. Pharmaceutical Sci., 93(3), 601 611 (2004). Similar preparations of solvates, hemisolvate, hydrates and the like are described by E. C. van Tonder et al, AAPS PharmSciTech., 5(1), article 12 (2004); and A. L. Bingham et al, Chem. Commun., 603 604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I.R. spectroscopy, show the presence of the solvent (or water) in the crystals as a solvate (or hydrate).

The term “synergistic” as used herein refers to a therapeutic combination which is more effective than the additive effects of the two or more single agents. A determination of a synergistic interaction between a kinase inhibitor that induces autophagy and one or more inhibitor of autophagy may be based on the results obtained from the assays described herein. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together. The combinations provided herein have been evaluated, and the data can be analyzed utilizing a standard program for quantifying synergism, additivism, and antagonism. An example of a program used for calculating synergism is that described by Chou and Talalay, in “New Avenues in Developmental Cancer Chemotherapy,” Academic Press, 1987, Chapter 2.

As used herein, “autophagy inhibitor” is meant to refer to composition which decreases the level of autophagy in a cell undergoing autophagy in its presence compared to the level of autophagy in a cell undergoing autophagy in its absence. Autophagy is a catabolic process of bulk lysosomal degradation and recycling of cytoplasmic material and organelles, characterized by the appearance of autophagic vacuoles in the cytoplasm, leading to self-digestion of cytoplasmic organelles and other constituents in the lysosomal compartments. While autophagy may be capable of ultimate cell killing when allowed to reach its limit, autophagy can provide a temporary survival mechanism for cells under stress conditions, but can also make cells vulnerable to several forms of cell death under specific circumstances. Inhibiting autophagy can either promote or inhibit cell death depending on the conditions and agents used (Amaravadi, R. K., D. Yu, J. J. Lum, T. Bui, M. A. Christophorou, G. I. Evan, A. Thomas-Tikhonenko, and C. B. Thompson, (2007), Autophagy inhibition enhances therapy-induced apoptosis in a Myc-induced model of lymphoma. J Clin Invest. 117:326-336; Kroemer, G., and M. Jaattela. 2005. Lysosomes and autophagy in cell death control. Nat Rev Cancer. 5:886-97; Levine, B., and J. Yuan, (2005), Autophagy in cell death: an innocent convict. J Clin Invest. 115:2679-2688; Lockshin, R. A., and Z. Zakeri, (2004), Apoptosis, autophagy, and more. Int J Biochem Cell Biol. 36:2405-19). Autophagy is a catabolic process that has distinct phases. These include, induction, sequestration, fusion and degradation phases. Inhibitors of autophagy can inhibit one or more of the phases. In an embodiment, autophagy inhibitors inhibit the later stages of autophagy. In one example, autophagy inhibitors inhibit the sequestration, fusion and degradation phases of autophagy. In one example, autophagy inhibitors inhibit the fusion and degradation phases of autophagy. In one example, autophagy inhibitors inhibit the degradation phase of autophagy. Useful inhibitors of autophagy include siRNA; antisense RNA; agents that inhibit the expression or function of LAMP2, LAMP1 or an autophagy (Atg) gene (e.g., Atg1, Atg4, Atg8, Atg5, Atg7 or Atg12); 3-methyladenine; lysosomotropic agents, which can also be antiparasitic, such as chloroquine, hydroxychloroquine or suramin, a vacuolar proton-ATPase inhibitor, such as Bafilomycin A1, an agent acting on the circulatory system, such as Amiodarone or Perhexylene, a cytotoxic agent, such as Vinblastine, an agent influencing lipid metabolism, an antibiotic, such as monensin, or a hormone, such as, Glucagon or estradiol, lysosomotropic agents, such as ammonium chloride, cAMP or methylamine, ATPase inhibitors, protease inhibitors, lysosomal protease inhibitors such as cathepsin inhibitors and cathepsin knockdown, as well as LAMP knockdown, e.g. LAMP1 and LAMP2. In another embodiment, modulators of lysosomal activity can be combined with kinase inhibitors that induce autophagy to provide a combination therapy for neoplasms. Lysosomes are organelles that contain digestive enzymes (acid hydrolases). Such enzymes include lipase, which digests lipids, carbohydrates, which digest carbohydrates (e.g., sugars), proteases, which digest proteins, nucleases, which digest nucleic acids, and phosphoric acid monoesters. In one example, the modulator acts to inhibit lysosomal activity. In an embodiment, the combination provides a synergistic effect.

### Kinase Inhibitors

There are hundreds of kinases, but not all kinase inhibitors also induce autophagy. For example, inhibitors of the bRaf and MEK kinase do not induce autophagy. In one example, the MEK inhibitor (R)—N-(2,3-dihydroxypropoxy)-3,4-difluoro-2-(2-fluoro-4-iodophenylamino)benzamide (VII) does not induce an increase in autophagy. In another example, CQ does not induce apoptosis in cancer cells (for example, melanoma and breast cancers) when combined with compound VII. Described herein are assays to determine whether a kinase inhibitor also induces autophagy Inhibitors of kinases that induce autophagy include inhibitors of Akt (eg. Akt-1, Akt-2 and Akt-3), PI3K, mTOR, PDK1 and p70S6K. The Akt kinase inhibitor can be a pan-Akt inhibitor, an allosteric Akt inhibitor or a selective inhibitor of Akt-1, Akt-2 or Akt-3.

In one embodiment, the Aid kinase inhibitor is a compound of Formula I:

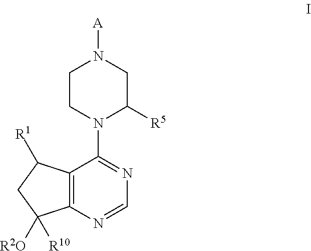

and tautomers, resolved enantiomers, resolved diastereomers, solvates, and salts thereof, wherein,

### R¹ is H, Me, Et and CF₃;

### R² is H or Me; R⁵ is H or Me;

### A is:

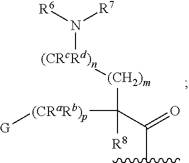

wherein G is phenyl optionally substituted by one to four R⁹ groups or a 5-6 membered heteroaryl optionally substituted by a halogen;
R⁶ and R⁷ are independently H, OCH₃, (C₃-C₆ cycloalkyl)-(CH₂), (C₃-C₆ cycloalkyl)-(CH₂CH₂), V—(CH₂)₀₋₁ wherein V is a 5-6 membered heteroaryl, W—(CH₂)₁₋₂ wherein W is phenyl optionally substituted with F, Cl, Br, I, OMe, CF₃ or Me, C₃-C₆-cycloalkyl optionally substituted with C₁-C₃ alkyl or O(C₁-C₃ alkyl), hydroxy-(C₃-C₆-cycloalkyl), fluoro-(C₃-C₆-cycloalkyl), CH(CH₃)CH(OH)phenyl, 4-6 membered heterocycle optionally substituted with F, OH, C₁-C₃ alkyl, cyclopropylmethyl or C(═O)(C₁-C₃ alkyl), or C₁-C₆-alkyl optionally substituted with one or more groups independently selected from OH, oxo, 0(C₁-C₆-alkyl), CN, F, NH₂, NH(C₁-C₆-alkyl), N(C₁-C₆-alkyl)₂, cyclopropyl, phenyl, imidazolyl, piperidinyl, pyrrolidinyl, morpholinyl, tetrahydrofuranyl, oxetanyl or tetrahydropyranyl, or R⁶ and R⁷ together with the nitrogen to which they are attached form a 4-7 membered heterocyclic ring optionally substituted with one or more groups independently selected from OH, halogen, oxo, CF₃, CH₂CF₃, CH₂CH₂OH, O(C₁-C₃ alkyl), C(═O)CH₃, NH₂, NHMe, N(Me)₂, S(O)₂CH₃, cyclopropylmethyl and C₁-C₃ alkyl;
R^{a} and R^{b} are H, or R^{a} is H, and R^{b} and R⁶ together with the atoms to which they are attached form a 5-6 membered heterocyclic ring having one or two ring nitrogen atoms;
R^{c} and R^{d} are H or Me, or R^{c} and R^{d} together with the atom to which they are attached from a cyclopropyl ring;
R⁸ is H, Me, F or OH, or R⁸ and R⁶ together with the atoms to which they are attached form a 5-6 membered heterocyclic ring having one or two ring nitrogen atoms;
each R⁹ is independently halogen, C₁-C₆-alkyl, C₃-C₆-cycloalkyl, O—(C₁-C₆-alkyl), CF₃, OCF₃, S(C₁-C₆-alkyl), CN, OCH₂-phenyl, CH₂O-phenyl, NH₂, NH—(C₁-C₆-alkyl), N—(C₁-C₆-alkyl)₂, piperidine, pyrrolidine, CH₂F, CHF₂, OCH₂F, OCHF₂, OH, SO₂(C₁-C₆-alkyl), C(O)NH₂, C(O)NH(C₁-C₆-alkyl), and C(O)N(C₁-C₆-alkyl)₂;

### R¹⁹ is H or Me; and

m, n and p are independently 0 or 1.

Another embodiment includes Akt inhibitors of Formula I, wherein R¹ is methyl; R², R⁵ and R¹⁰ are H; G is phenyl optionally substituted with 1-3 R⁹; R⁹ is halogen, C₁-C₃ alkyl, NC, CF₃, OCF₃ OCH₃ or OCH₂Phenyl; R_{c} and R_{d} are H or methyl; m, n and p are 0 or 1; and R⁸ is H or methyl.

Another embodiment includes Akt inhibitors of Formula I, including the compounds:

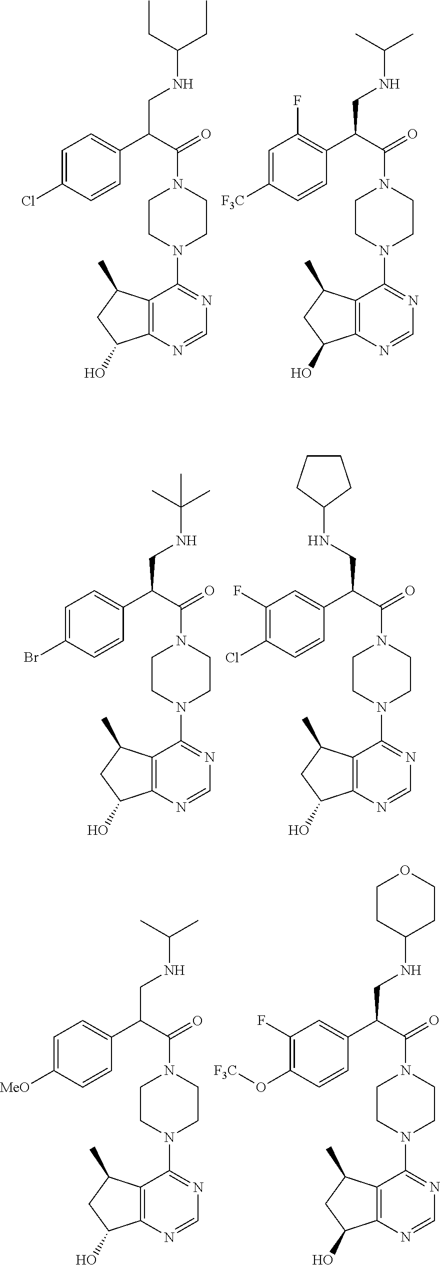

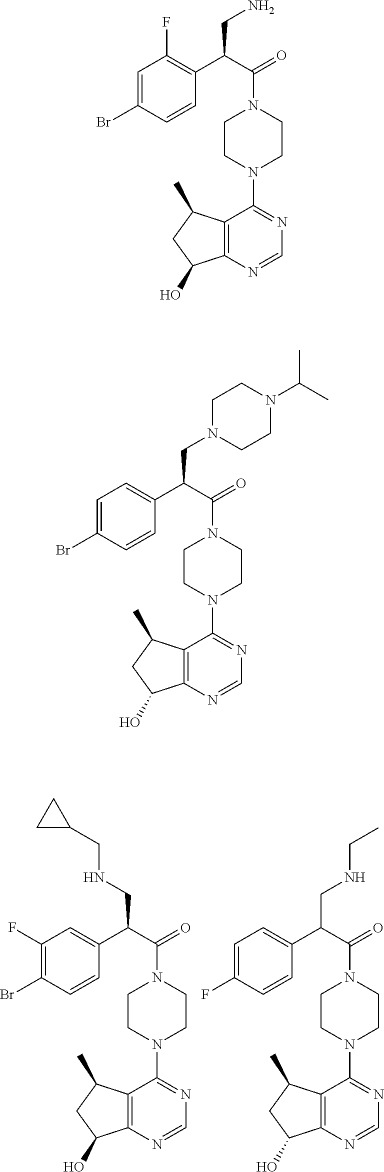

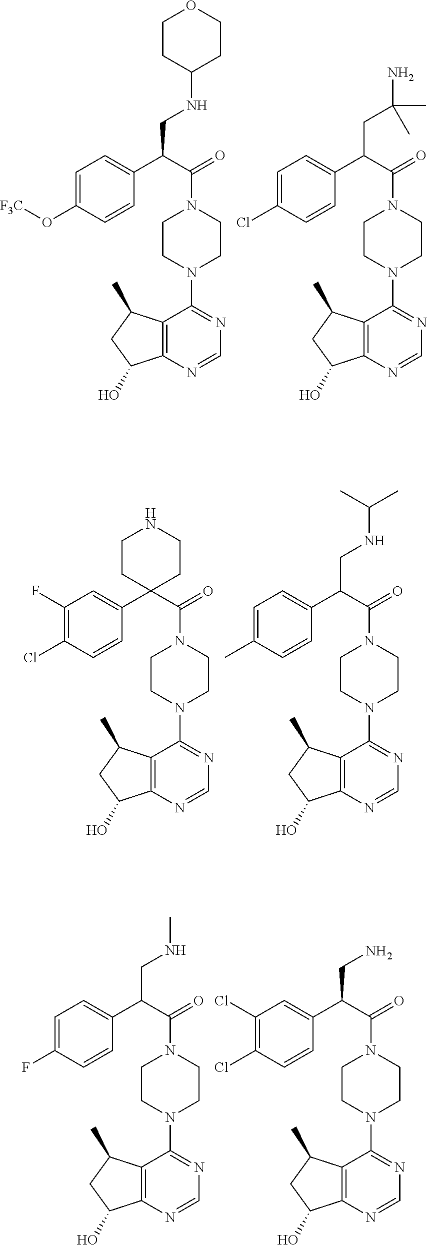

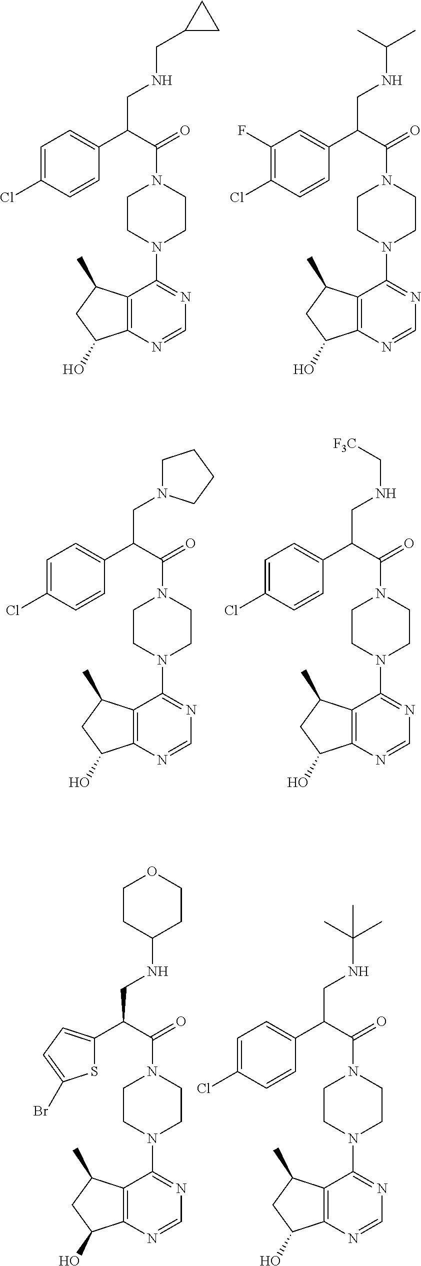

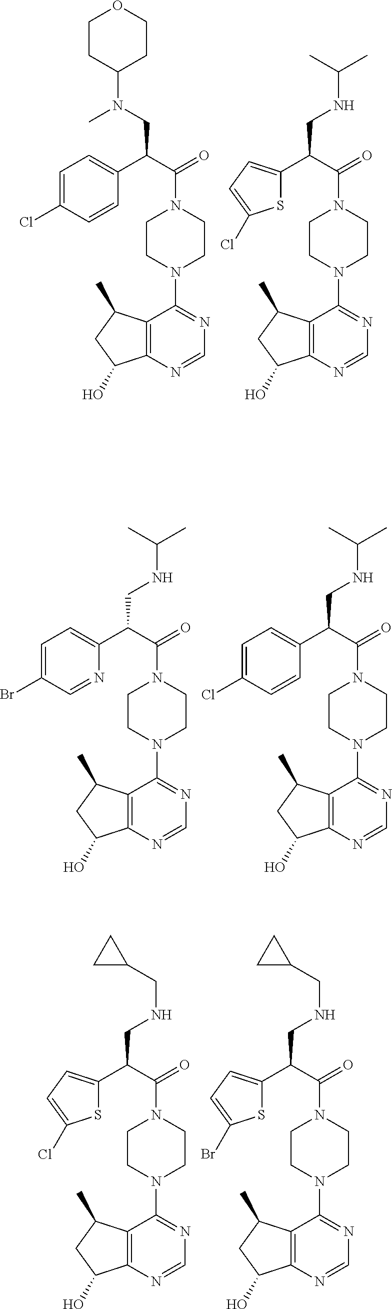

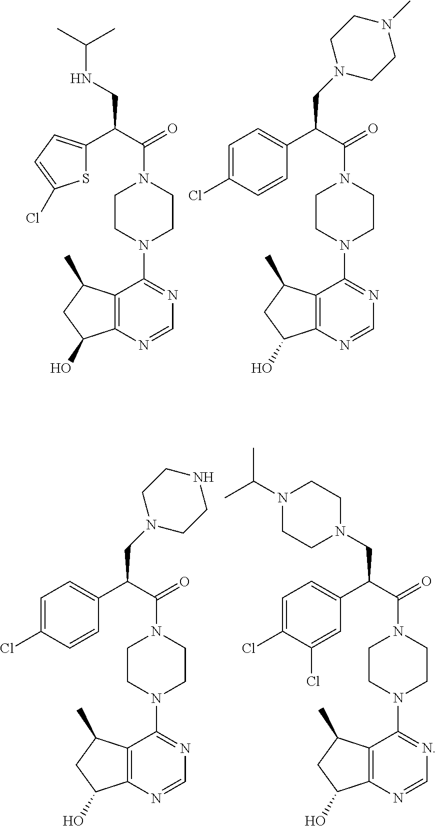

Preparation of Formula I Compounds

Compounds of Formula I may be prepared according to methods described in U.S. patent application Ser. No. 11/773,949, filed Jul. 5, 2007, entitled “Hydroxylated and Methoxylated Pyrimidyl Cyclopentanes as AKT Protein Kinase Inhibitors,” which is incorporated by reference herein, for all purposes.

Compounds of Formula I may be prepared singly or as compound libraries comprising at least 2, for example 5 to 1,000 compounds, or 10 to 100 compounds. Libraries of compounds of Formula I may be prepared by a combinatorial ‘split and mix’ approach or by multiple parallel syntheses using either solution phase or solid phase chemistry.

For illustrative purposes, Schemes 1-4 show a general method for preparing the compounds of Formula I as well as key intermediates. Those skilled in the art will appreciate that other synthetic routes may be used. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

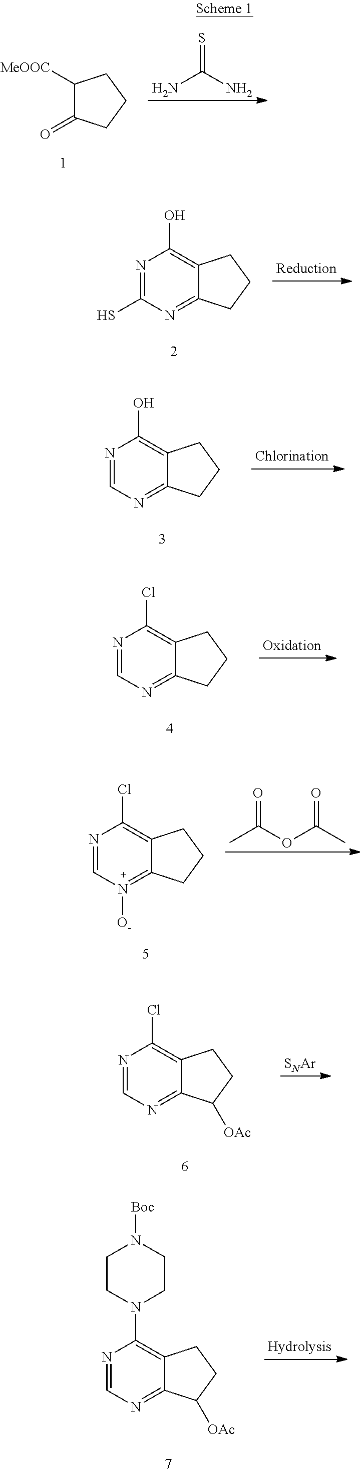

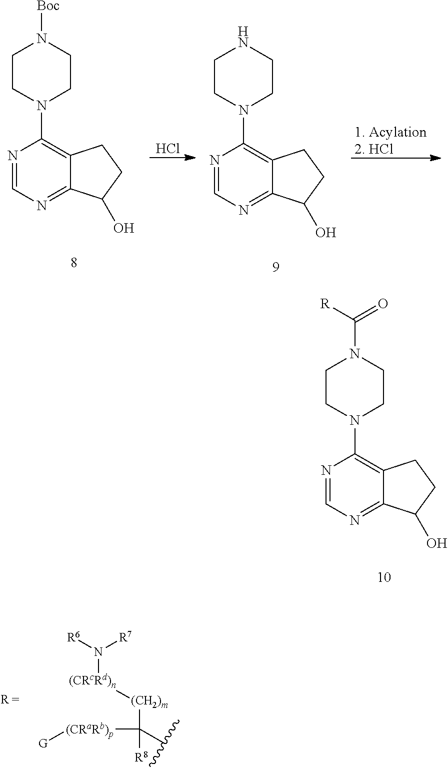

Scheme 1 shows a method of preparing compound 10 of Formula I wherein R¹ is H, R² is OH and R⁵ is H. Formation of pyrimidine 2 can be accomplished by the reaction of the keto ester 1 with thiourea in the presence of a base such as KOH in an appropriate solvent, such as ethanol. After reduction of the mercapto group of compound 2 under standard reducing conditions (e.g., Raney Ni and NH₄OH) to provide compound 3, the hydroxypyrimidine 3 can be chlorinated under standard conditions (e.g., POCl₃ in DIEA/DCE) to provide compound 4. Compound 4 is then oxidized under standard conditions (e.g., MCPBA in an appropriate solvent such as CHCl₃) to give the pyrimidine-oxide 5. Treatment of the pyrimidine-oxide with acetic anhydride gives the rearrangement product 6. Compound 7 is obtained by reacting compound 6 with an appropriately substituted piperidine under standard S_{N}Ar reaction conditions to provide compound 7. Compound 7 is hydrolyzed to provide compound 8, which is then deprotected to yield the intermediate 9. Acylation of the piperazinyl cyclopenta[d]pyrimidine 9 with an appropriated amino acid in the presence of a coupling reagent such as HBTU, followed by deprotection if necessary, gives compound 10 of Formula I.

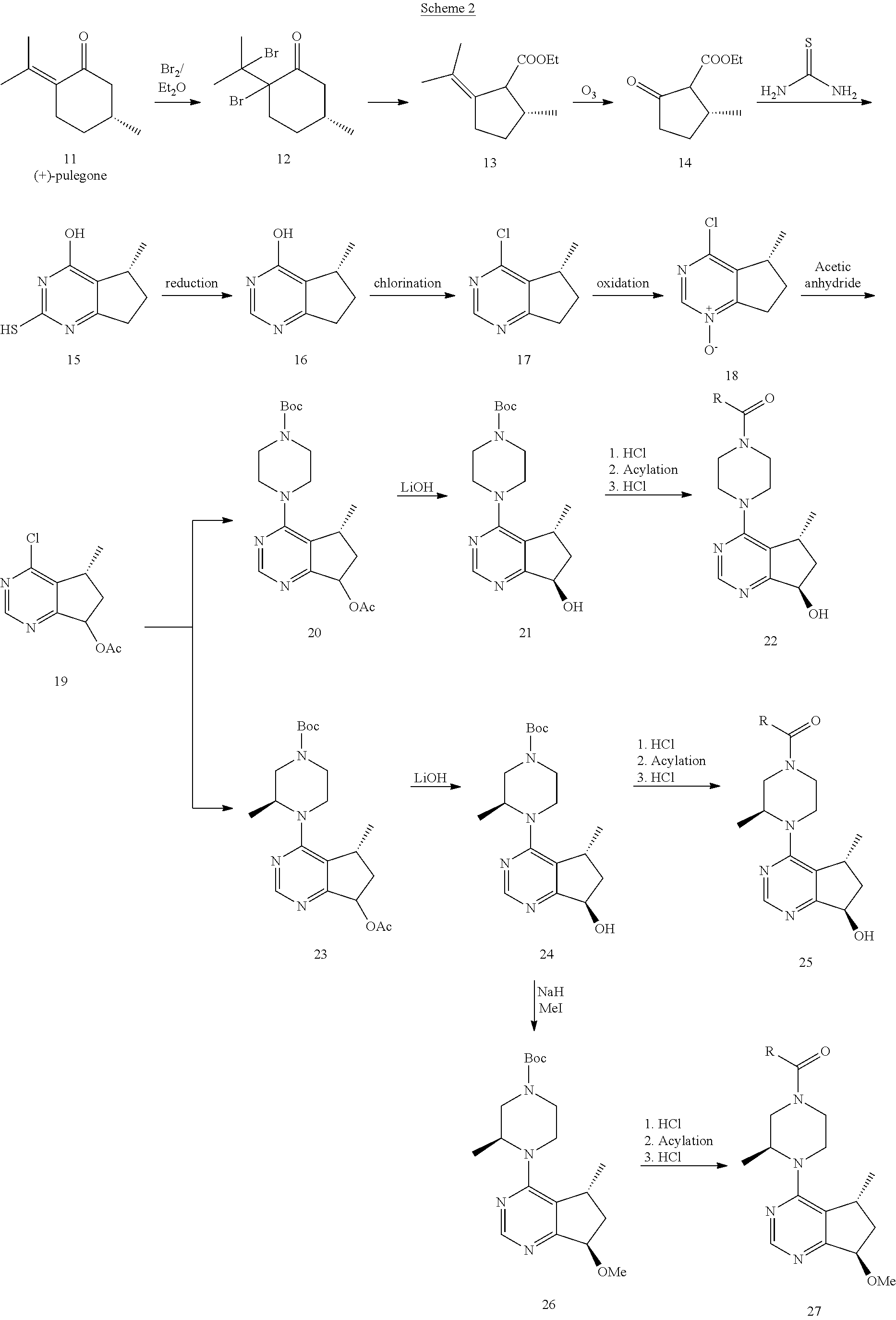

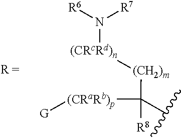

Scheme 2 shows a method of preparing compounds 22, 25 and 27 of Formula I wherein R¹, R² and R⁵ are methyl. According to Scheme 2, bromination of (+)-pulegone 11 with bromine gives the dibromide 12. The treatment of the dibromide 12 with a base such as sodium ethoxide provides the pulegenate 13. Ozonolysis of the pulegenate 13 gives the ketoester 14. Treatment of the keto ester 14 with thiourea in the presence of a base such as KOH in ethanol, followed by reduction of the mercapto group under standard conditions (e.g. Raney Ni catalyst in ammonia) affords the hydroxypyrimidine 16. Chlorination of the hydroxypyrimidine 16 under standard conditions (e.g., POCl₃) provides the 4-chloropyrimidine 17. The oxidation of the 4-chloropyrimidine 17 with an oxidizing agent such as MCPBA or hydrogen peroxide provides the N-oxide 18. Rearrangement of the N-oxide 18 with acetic anhydride yields the intermediate 19. Compound 19 is reacted with the desired piperazine according to the procedure described in Scheme 1 to provide compound 20 where R⁵ is H and 23 where R⁵ is Me. Compounds 20 and 23 are subjected to chiral separation using HPLC with chiral stationary and then hydrolyzed upon treatment with a base such as lithium hydroxide to provide compounds 21 and 24, respectively. After deprotection, compounds 21 and 24 are then reacted with the appropriate amino acid to provide compounds 22 and 25, respectively.

Alternatively, the 7-hydroxy group of compound 24 may be alkylated with alkylation reagent such as alkyl halide in the presence of a base such as NaH or KOH to provide compound 26 where R² is Me. After deprotection, compound 26 is then reacted with the appropriate amino acid to provide compound 27.

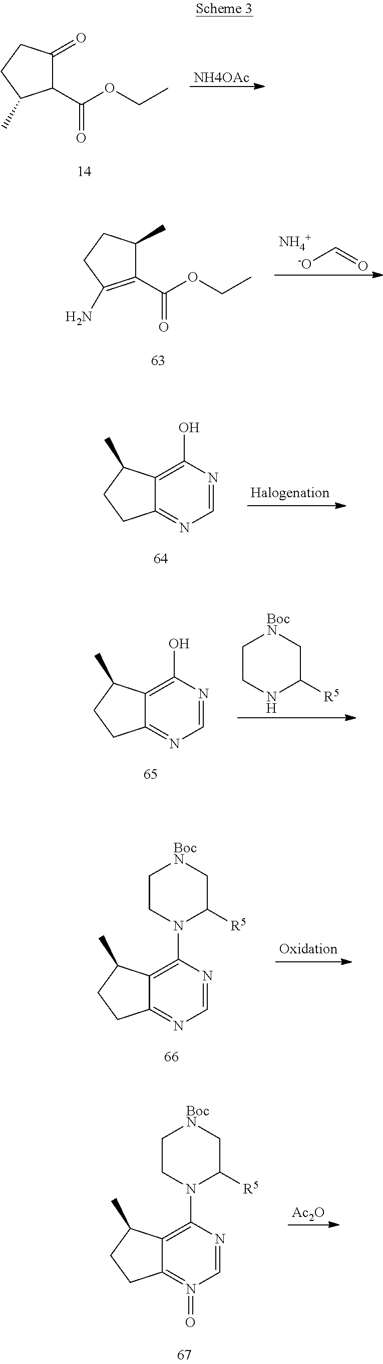

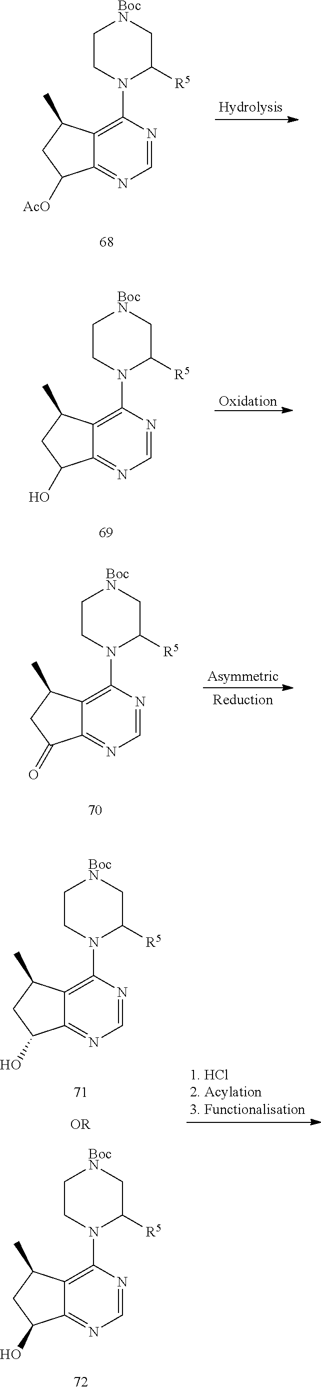

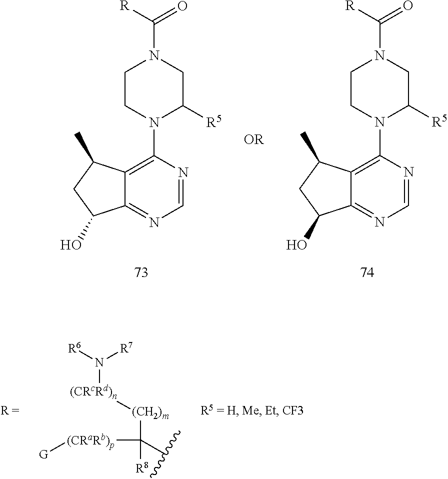

Scheme 3 shows an alternative method of preparing compounds 73 and 74. According to Scheme 3, amination of 14 using an ammonia synthon gives 63. Pyrimidine formation using, for example, ammonium formate in the presence of formamide at 50° C.-250° C. and/or at high pressure gives the bicyclic unit 64. Activation of 64 using, for example, POCl₃ or SOCl₂ gives the activated pyrimidine 65. Displacement of this leaving group, using a suitable protected/substituted piperidine at 0° C. to 150° C. gives the piperidine 66. Oxidation, using, for example, m-chloroperoxybenzoic acid (“MCPBA” or “m-CPBA”) or Oxone® at −20° C. to 50° C. gives the N-oxide 67. Treatment with an acylating agent (eg. acetic anhydride) followed by heating (40° C. to 200° C.) causes rearrangement to give 68. Hydrolysis, using, for example LiOH or NaOH at 0° C. to 50° C. gives the alcohol 69. Oxidation, using for example, Swern conditions, MnO₄ or pyridine-SO₃ complex at appropriate temperatures gives the ketone 70. Asymmetric reduction using, for example, a catalytic chiral catalyst in the presence of hydrogen, the CBS catalyst or a borohydride reducing agent in the presence of a chiral ligand gives rise to either the (R) or the (S) stereochemistry at the alcohol 71 or 72. Alternatively, a non-chiral reducing agent could be used (eg. H₂, Pd/C), allowing the methyl group on the cyclopentane unit to provide facial selectivity and ultimately diastereoselectivity. If the reduction gives a lower diastereoselctivity, the diastereomers could be separated by (for example) chromatography, crystallization or derivitization. Finally deprotection of the Boc-group, using, for example, acid at 0° C. to 50° C., acylation using an appropriately functionalized amino acid and final functionalization of the amine of this amino acid (eg. removal of any protecting group, alkylation, reductive amination or acylation to introduce new substituents) gives rise to the final compounds 73 and 74.

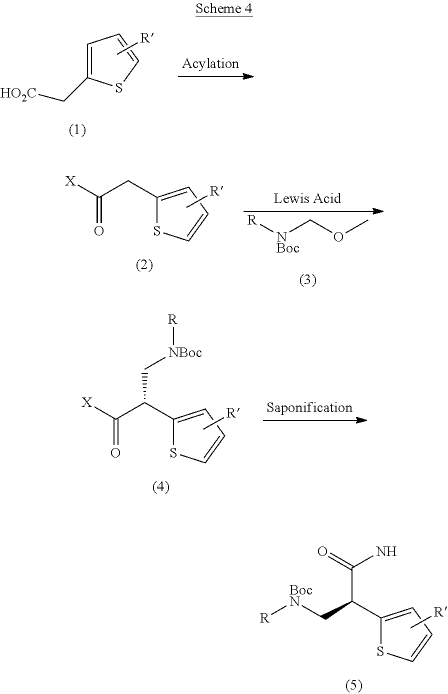

Introduction of a chiral auxiliary (e.g. Evans oxazolidinone, etc.) to compound (1) may be accomplished by standard acylation procedures to give the conjugate (2). For example, treatment of the acid with an activating agent (e.g. COCl₂) or mixed anhydride formation (e.g. 2,2-dimethylpropanoyl chloride) in the presence of an amine base at −20° C. to 100° C. followed by treatment with the appropriate chiral auxiliary (X) gives compound (2). The stereochemistry and choice of the chiral auxiliary may determine the stereochemistry of the newly created chiral center and the diastereoselectivity. Treatment of compound (2) with a Lewis acid (eg. TiCl₄) at low temperature (e.g. −20° C. to −100° C.) and an amine base (e.g. Hunig's base) followed by the use of an appropriately substituted imminium ion precursor (3) at low temperature then gives rise to compound (4). The temperature, Lewis acid and chiral auxiliary may all be expected to influence the diastereoselectivity of the addition adduct. Finally, saponification under mild conditions (e.g. LiOH/H₂O at −10° C. to 30° C.) gives rise to the desired acid (5).

In another embodiment, the kinase inhibitor is an Aid inhibitor of the following formula:

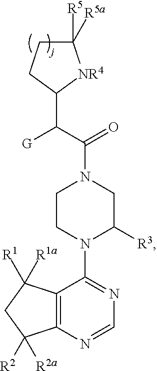

stereoisomers, tautomers or pharmaceutically acceptable salts thereof, wherein:

G is phenyl optionally substituted with one to three R^{a} groups or a 5-6 membered heteroaryl optionally substituted by a halogen;

R¹ and R^{1a} are independently selected from H, Me, CF₃, CHF₂ or CH₂F;

R² is H, F or —OH;

R^{2a} is H;

R³ is H;

R⁴ is H, or C₁-C₄ alkyl optionally substituted with F, —OH or —O(C₁-C₃ alkyl);

R⁵ and R^{5a} are independently selected from H and C₁-C₄ alkyl, or R⁵ and R^{5a} together with the atom to which they are attached form a 5-6 membered cycloalkyl or 5-6 membered heterocycle, wherein the heterocycle has an oxygen heteroatom;

each R^{a} is independently halogen, C₁-C₆-alkyl, C₃-C₆-cycloalkyl, —O—(C₁-C₆-alkyl), CF₃, —OCF₃, S(C₁-C₆-alkyl), CN, —OCH₂-phenyl, NH₂, —NO₂, —NH—(C₁-C₆-alkyl), —N—(C₁-C₆-alkyl)₂, piperidine, pyrrolidine, CH₂F, CHF₂, —OCH₂F, —OCHF₂, —OH, —SO₂(C₁-C₆-alkyl), C(O)NH₂, C(O)NH(C₁-C₆-alkyl), and C(O)N(C₁-C₆-alkyl)₂; and

j is 1 or 2.

Another embodiment includes Aid inhibitor compounds, including:

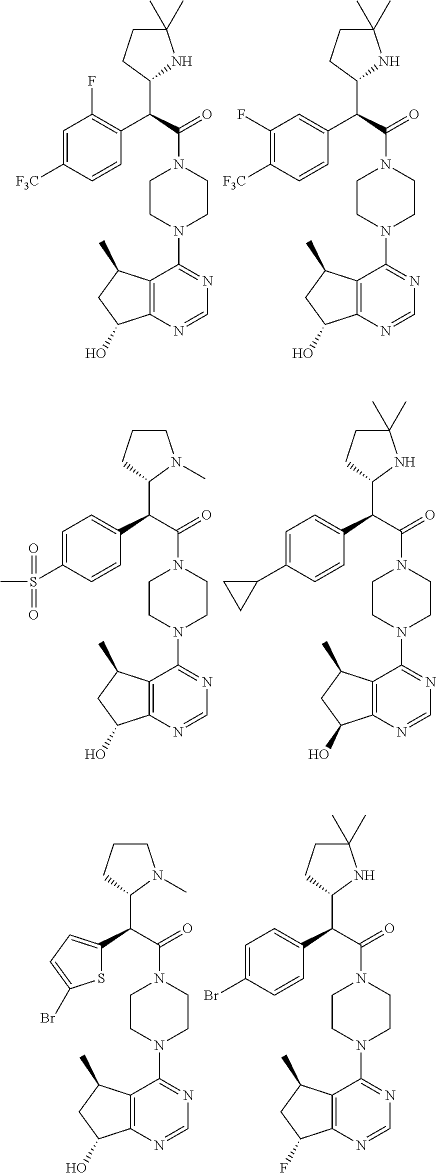

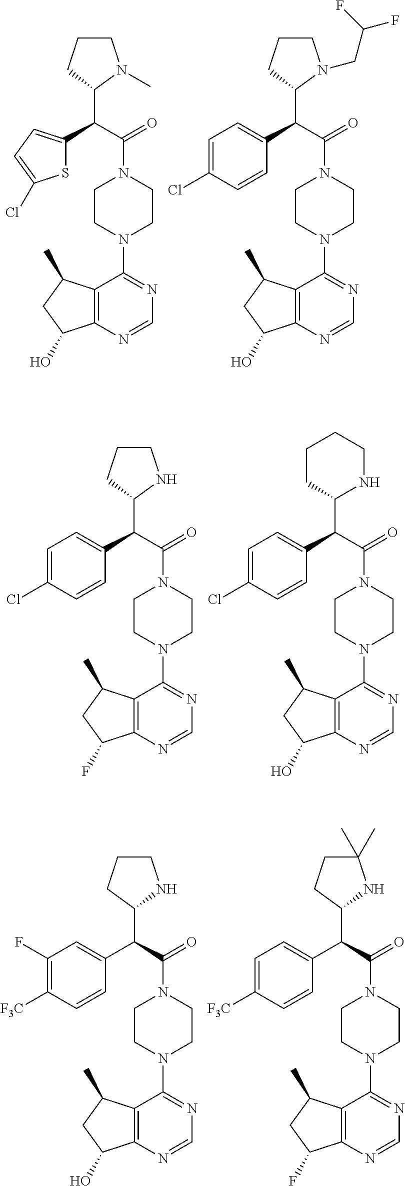

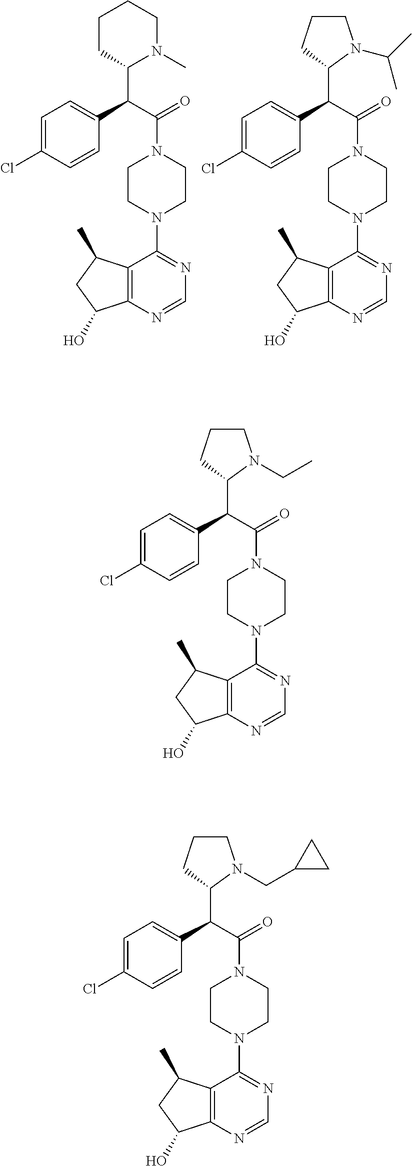

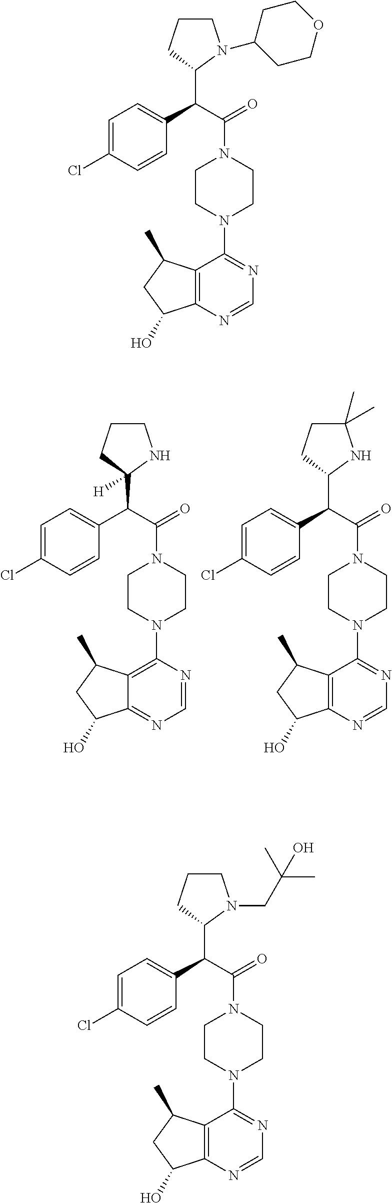

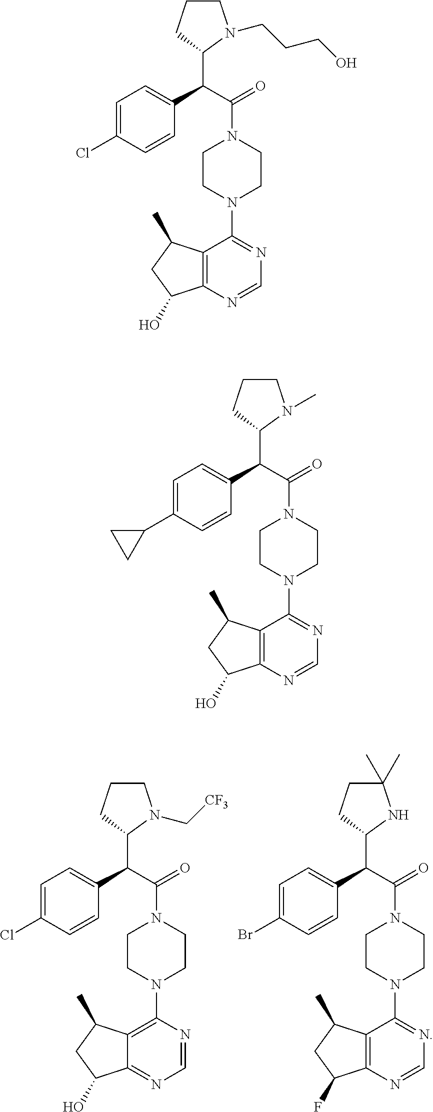 [SMILES: C[C@@H]1C[C@@H](O)c2ncnc(N3CCN(C(=O)[C@@H](c4ccc(Cl)cc4)[C@@H]4CCCN4CCCO)CC3)c21 ; C[C@@H]1C[C@@H](O)c2ncnc(N3CCN(C(=O)[C@@H](c4ccc(C5CC5)cc4)[C@@H]4CCCN4C)CC3)c21 ; C[C@@H]1C[C@@H](O)c2ncnc(N3CCN(C(=O)[C@@H](c4ccc(Cl)cc4)[C@@H]4CCCN4CC(F)(F)F)CC3)c21 ; C[C@@H]1C[C@H](F)c2ncnc(N3CCN(C(=O)[C@@H](c4ccc(Br)cc4)[C@@H]4CCC(C)(C)N4)CC3)c21]

In one embodiment, the kinase inhibitor is an Akt inhibitor compound of Formula II:

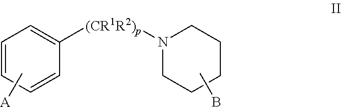

wherein, R¹ and R² are independently hydrogen, C₁-C₅ alkyl, hydroxyl, C₁₋₅ alkoxy or amine; p is an integer from 1 to 6; A is a 5-14 carbon cyclic, bicyclic or tricyclic aromatic or heteroaromatic ring, which can be optionally substituted with halogen, OH, amino, dialkylamino, monoalkylamino, C₁-C₆-alkyl or phenyl, which is optionally substituted with halogen, OH, C₁-C₃ alkyl or cyclopropylmethyl; and in one embodiment A has one of the following structures:

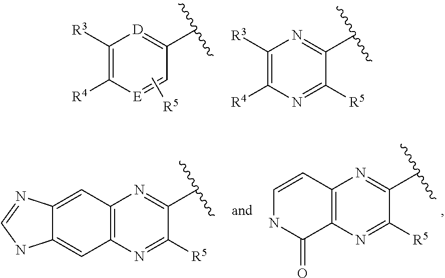

wherein D and E are independently —CH or N;
wherein R³ and R⁴ are each independently hydrogen, halogen, OH, amino, dialkylamino, monoalkylamino or C₁-C₆-alkyl, which is optionally substituted with halogen, OH, C₁-C₃ alkyl or cyclopropylmethyl;
R⁵ is a 5 or 6 membered aromatic or heteroaromatic ring optionally substituted with halogen, OH, amino, dialkylamino, monoalkylamino or C₁-C₆-alkyl, which is optionally substituted with halogen, OH, C₁-C₃ alkyl or cyclopropylmethyl; in one embodiment R⁵ is phenyl;
B is an aromatic, heteroaromatic, cyclic or heterocyclic ring having the formula:

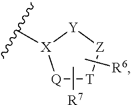

wherein, Q, T, X and Y are each independently selected from the group consisting of —CH, —CH₂, C═O, N or O;

### Z is —CH, —CH₂, C═O, N, O or —C═C—;

R⁶ and R⁷ are independently selected from the group consisting of hydrogen, halogen, carbonyl and a 5 or 6 membered aromatic or heteroaromatic ring optionally substituted with halogen, OH, amino, dialkylamino, monoalkylamino or C₁-C₆-alkyl, which is optionally substituted with halogen, OH, C₁-C₃ alkyl or cyclopropylmethyl; in one embodiment R⁶ or R⁷ is pyridinyl, or R⁶ and R⁷ are taken together to form a 5-6 membered aromatic, heteroaromatic, cyclic or heterocyclic ring, which can be optionally substituted with halogen, OH, amino, dialkylamino, monoalkylamino or C₁-C₆-alkyl, which is optionally substituted with halogen, OH, C₁-C₃ alkyl or cyclopropylmethyl; in one embodiment, B has one of the following structures:

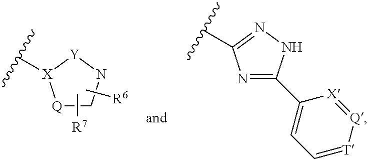

wherein X, Y, Q, R⁶ and R⁷ are as described above, and X′, Q′ and T′ are —CH or N.

In another embodiment, AKT inhibitors include compounds having the formula:

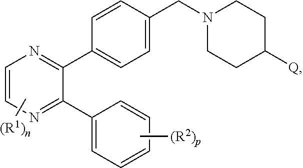

wherein: a is 0 or 1; b is 0 or 1; m is 0, 1 or 2; n is 0, 1 or 2; p is 0, 1 or 2; r is 0 or 1; s is 0 or 1;

Q is selected from: —NR⁷R⁸,

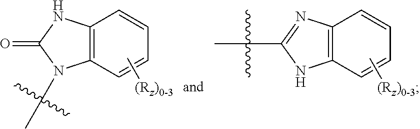

R¹ is independently selected from (C═O)ₐO_{b}C₁-C₆ alkyl, (C═O)ₐO_{b}aryl, C₂-C₆ alkenyl, C₂-C₆ alkynyl, (C═O)ₐO_{b}heterocyclyl, (C═O)ₐO_{b}C₃-C₆ cycloalkyl, CO₂H, halogen, CN, OH, O_{b}C₁-C₆ perfluoroalkyl, Oₐ(C═O)_{b}NR⁷R⁸, NR^{c}(C═O)NR⁷R⁸, S(O)ₘR^{a}, S(O)₂NR⁷R⁸, NR^{c}S(O)ₘR^{a}, oxo, CHO, NO₂, NR^{c}(C═O)O_{b}R^{a}, O(C═O)O_{b}C₁-C₆ alkyl, O(C═O)O_{b}C₃-C₆ cycloalkyl, O(C═O)O_{b}aryl, and O(C═O)O_{b}-heterocycle, wherein said alkyl, aryl, alkenyl, alkynyl, heterocyclyl, and cycloalkyl are optionally substituted with one or more substituents selected from R^{z};

R² is independently selected from C₁-C₆ alkyl, aryl, heterocyclyl, CO₂H, halo, CN, OH and S(O)₂NR⁷R⁸, wherein said alkyl, aryl and heterocyclyl are optionally substituted with one, two or three substituents selected from R^{z};

R⁷ and R⁸ are independently selected from H, (C═O)O_{b}C₁-C₁₀ alkyl, (C═O)O_{b}C₃-C₈ cycloalkyl, (C═O)O_{b}aryl, (C═O)O_{b}heterocyclyl, C₁-C₁₀ alkyl, aryl, C₂-C₁₀ alkenyl, C₂-C₁₀ alkynyl, heterocyclyl, C₃-C₈ cycloalkyl, SO₂R^{a} and (C═O)NR^{b}₂, wherein said alkyl, cycloalkyl, aryl, heterocylyl, alkenyl, and alkynyl is optionally substituted with one or more substituents selected from R^{z}, or

R⁷ and R⁸ can be taken together with the nitrogen to which they are attached to form a monocyclic or bicyclic heterocycle with 5-7 members in each ring and optionally containing, in addition to the nitrogen, one or two additional heteroatoms selected from N, O and S, said monocyclic or bicyclic heterocycle optionally substituted with one or more substituents selected from R^{z};

R^{z} is selected from: (C═O)ᵣOₛ(C₁-C₁₀)alkyl, Or(C₁-C₃)perfluoroalkyl, (C₀-C₆)alkylene-S(O)ₘR^{a}, oxo, OH, halo, CN, (C═O)ᵣOₛ(C₂-C₁₀)alkenyl, (C═O)ᵣOₛ(C₂-C₁₀) alkynyl, (C═O)ᵣOₛ(C₃-C₆)cycloalkyl, (C═O)ᵣOₛ(C₀-C₆)alkylene-aryl, (C═O)ᵣOₛ(C₀-C₆) alkylene-heterocyclyl, (C═O)ᵣOₛ(C₀-C₆)alkylene-N(R^{b})₂, C(O)R^{a}, (C₀-C₆)alkylene-CO₂R^{a}, C(O)H, (C₀-C₆)alkylene-CO₂H, C(O)N(R^{b})₂, S(O)ₘR^{a}, and S(O)₂N(R^{b})₂NR^{c}(C═O)O_{b}R^{a}, O(C═O)O_{b}C₁-C₁₀ alkyl, O(C═O)O_{b}C₃-C₈ cycloalkyl, O(C═O)O_{b}aryl, and O(C═O)O_{b}-heterocycle, wherein said alkyl, alkenyl, alkynyl, cycloalkyl, aryl, and heterocyclyl are optionally substituted with up to three substituents selected from R^{b}, OH, (C₁-C₆)alkoxy, halogen, CO₂H, CN, O(C═O)C₁-C₆ alkyl, oxo, and N(R^{b})₂;

R^{a} is (C₁-C₆)alkyl, (C₃-C₆)cycloalkyl, aryl or heterocyclyl; and

R^{b} is H, (C₁-C₆)alkyl, aryl, heterocyclyl, (C₃-C₆)cycloalkyl, (C═O)OC₁-C₆ alkyl, (C═O)C₁-C₆ alkyl or S(O)₂R^{a};

R^{c} is selected from: H, C₁-C₆ alkyl, aryl, C₂-C₆ alkenyl, C₂-C₆ alkynyl, heterocyclyl, C₃-C₈ cycloalkyl and C₁-C₆ perfluoroalkyl, wherein said alkyl, cycloalkyl, aryl, heterocylyl, alkenyl, and alkynyl is optionally substituted with one or more substituents selected from R^{z};

or a pharmaceutically acceptable salt or a stereoisomer thereof.

In another embodiment, AKT inhibitors include:

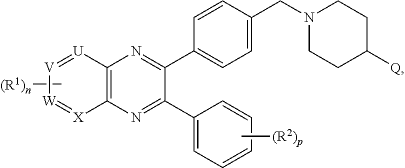

wherein a is 0 or 1; b is 0 or 1; m is 0, 1 or 2; n is 0, 1, 2 or 3; p is 0, 1 or 2; r is 0 or 1; s is 0 or 1; u, v, w and x are independently selected from: CH and N, provided that only one of u, v, w and x may be N;

Q is selected from: —NR⁵R⁶,

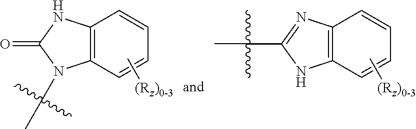

R¹ is independently selected from (C═O)ₐO_{b}C₁-C₆ alkyl, (C═O)ₐO_{b}aryl, C₂-C₆ alkenyl, C₂-C₆ alkenyl, (C═O)ₐO_{b}heterocyclyl, (C═O)ₐO_{b}C₃-C₆ cycloalkyl, CO₂H, halogen, CN, OH, O_{b}C₁-C₆ perfluoroalkyl, Oₐ(C═O)_{b}NR⁷R⁸, NR^{c}(C═O)NR⁷R⁸, S(O)ₘR^{a}, S(O)₂NR⁷R⁸, NR^{c}S(O)ₘR^{a}, oxo, CHO, NO₂, NR^{c}(C═O)O_{b}R^{a}, O(C═O)O_{b}C₁-C₆ alkyl, O(C═O)O_{b}C₃-C₆ cycloalkyl, O(C═O)O_{b}aryl, and O(C═O)O_{b}-heterocycle, wherein said alkyl, aryl, alkenyl, alkynyl, heterocyclyl, and cycloalkyl are optionally substituted with one or more substituents selected from R^{z};

R² is independently selected from C₁-C₆ alkyl, aryl, heterocyclyl, CO₂H, halo, CN, OH and S(O)₂NR⁷R⁸, wherein said alkyl, aryl and heterocyclyl are optionally substituted with one, two or three substituents selected from R^{z};

R⁷ and R⁸ are independently selected from H, (C═O)O_{b}C₁-C₁₀ alkyl, (C═O)O_{b}C₃-C₈ cycloalkyl, (C═O)O_{b}aryl, (C═O)O_{b}heterocyclyl, C₁-C₁₀ alkyl, aryl, C₂-C₁₀ alkenyl, C₂-C₁₀ alkynyl, heterocyclyl, C₃-C₈ cycloalkyl, SO₂R^{a} and (C═O)NR^{b}₂, wherein said alkyl, cycloalkyl, aryl, heterocylyl, alkenyl, and alkynyl is optionally substituted with one or more substituents selected from R^{z}, or

R⁷ and R⁸ can be taken together with the nitrogen to which they are attached to form a monocyclic or bicyclic heterocycle with 5-7 members in each ring and optionally containing, in addition to the nitrogen, one or two additional heteroatoms selected from N, O and S, said monocyclic or bicyclic heterocycle optionally substituted with one or more substituents selected from R^{z};

R^{z} is selected from: (C═O)ᵣOₐ(C₁-C₁₀) alkyl, Or(C₁-C₃)perfluoroalkyl, (C₀-C₆)alkylene-S(O)ₘR^{a}, oxo, OH, halo, CN, (C═O)ᵣOₐ(C₂-C₁₀) alkenyl, (C═O)ᵣOₐ(C₂-C₁₀) alkynyl, (C═O)ᵣOₛ(C₃-C₆) cycloalkyl, (C═O)ᵣOₐ(C₀-C₆) alkylene-aryl, (C═O)ᵣOₐ(C₀-C₆) alkylene-heterocyclyl, (C═O)ᵣOₐ(C₀-C₆) alkylene-N(R^{b})₂, C(O)R^{a}, (C₀-C₆)alkylene-CO₂R^{a}, C(O)H, (C₀-C₆)alkylene-CO₂H, C(O)N(R^{b})₂, S(O)ₘR^{a}, and S(O)₂N(R^{b})₂NR^{c}(C═O)O_{b}R^{a}, O(C═O)O_{b}C₁-C₁₀ alkyl, O(C═O)O_{b}C₃-C₈ cycloalkyl, O(C═O)O_{b}aryl, and O(C═O)O_{b}-heterocycle, wherein said alkyl, alkenyl, alkynyl, cycloalkyl, aryl, and heterocyclyl are optionally substituted with up to three substituents selected from R^{b}, OH, (C₁-C₆)alkoxy, halogen, CO₂H, CN, O(C═O)C₁-C₆ alkyl, oxo, and N(R^{b})₂;

R^{a} is (C₁-C₆)alkyl, (C₃-C₆)cycloalkyl, aryl or heterocyclyl; and

R^{b} is H, (C₁-C₆)alkyl, aryl, heterocyclyl, (C₃-C₆)cycloalkyl, (C═O)OC₁-C₆ alkyl, (C═O)C₁-C₆ alkyl or S(O)₂R^{a};

R^{c} is selected from: H, C₁-C₆ alkyl, aryl, C₂-C₆ alkenyl, C₂-C₆ alkynyl, heterocyclyl, C₃-C₈ cycloalkyl and C₁-C₆ perfluoroalkyl, wherein said alkyl, cycloalkyl, aryl, heterocylyl, alkenyl, and alkynyl is optionally substituted with one or more substituents selected from R^{z};

or a pharmaceutically acceptable salt or a stereoisomer thereof.

In another embodiment, AKT inhibitors include:

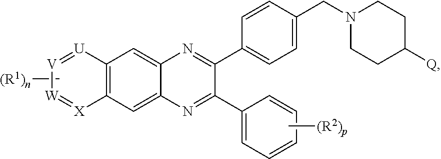

wherein a is 0 or 1; b is 0 or 1; m is 0, 1 or 2; n is 0, 1, 2 or 3; p is 0, 1 or 2; r is 0 or 1; s is 0 or 1; u, v, and x are independently selected from CH and N; W is a bond, CH or N;

Q is selected from: —NR⁵R⁶,

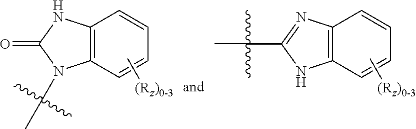

R¹ is independently selected from (C═O)ₐO_{b}C₁-C₆ alkyl, (C═O)ₐO_{b}aryl, C₂-C₆ alkenyl, C₂-C₆ alkynyl, (C═O)ₐO_{b}heterocyclyl, (C═O)ₐO_{b}C₃-C₆ cycloalkyl, CO₂H, halogen, CN, OH, O_{b}C₁-C₆ perfluoroalkyl, Oₐ(C═O)_{b}NR⁷R⁸, NR^{c}(C═O)NR⁷R⁸, S(O)ₘR^{a}, S(O)₂NR⁷R⁸, NR^{c}S(O)ₘR^{a}, oxo, CHO, NO₂, NR^{c}(C═O)O_{b}R^{a}, O(C═O)O_{b}C₁-C₆ alkyl, O(C═O)O_{b}C₃-C₆ cycloalkyl, O(C═O)O_{b}aryl, and O(C═O)O_{b}-heterocycle, wherein said alkyl, aryl, alkenyl, alkynyl, heterocyclyl, and cycloalkyl are optionally substituted with one or more substituents selected from R^{z};

R² is independently selected from C₁-C₆ alkyl, aryl, heterocyclyl, CO₂H, halo, CN, OH and S(O)₂NR⁷R⁸, wherein said alkyl, aryl and heterocyclyl are optionally substituted with one, two or three substituents selected from R^{z};

R⁷ and R⁸ are independently selected from H, (C═O)O_{b}C₁-C₁₀ alkyl, (C═O)O_{b}C₃-C₈ cycloalkyl, (C═O)O_{b}aryl, (C═O)O_{b}heterocyclyl, C₁-C₁₀ alkyl, aryl, C₂-C₁₀ alkenyl, C₂-C₁₀ alkynyl, heterocyclyl, C₃-C₈ cycloalkyl, SO₂R^{a} and (C═O)NR^{b}₂, wherein said alkyl, cycloalkyl, aryl, heterocylyl, alkenyl, and alkynyl is optionally substituted with one or more substituents selected from R^{z}, or

R⁷ and R⁸ can be taken together with the nitrogen to which they are attached to form a monocyclic or bicyclic heterocycle with 5-7 members in each ring and optionally containing, in addition to the nitrogen, one or two additional heteroatoms selected from N, O and S, said monocyclic or bicyclic heterocycle optionally substituted with one or more substituents selected from R^{z};

R^{z} is selected from: (C═O)ᵣOₛ(C₁-C₁₀) alkyl, Or(C₁-C₃)perfluoroalkyl, (C₀-C₆)alkylene-S(O)ₘR^{a}, oxo, OH, halo, CN, (C═O)ᵣOₛ(C₂-C₁₀) alkenyl, (C═O)ᵣOₛ(C₂-C₁₀) alkynyl, (C═O)ᵣOₛ(C₃-C₆) cycloalkyl, (C═O)ᵣOₛ(C₀-C₆) alkylene-aryl, (C═O)ᵣOₛ(C₀-C₆) alkylene-heterocyclyl, (C═O)ᵣOₛ(C₀-C₆) alkylene-N(R^{b})₂, C(O)R^{a}, (C₀-C₆)alkylene-CO₂R^{a}, C(O)H, (C₀-C₆)alkylene-CO₂H, C(O)N(R^{b})₂, S(O)ₘR^{a}, and S(O)₂N(R^{b})₂NR^{c}(C═O)O_{b}R^{a}, O(C═O)O_{b}C₁-C₁₀ alkyl, O(C═O)O_{b}C₃-C₈ cycloalkyl, O(C═O)O_{b}aryl, and O(C═O)O_{b}-heterocycle, wherein said alkyl, alkenyl, alkynyl, cycloalkyl, aryl, and heterocyclyl are optionally substituted with up to three substituents selected from R^{b}, OH, (C₁-C₆)alkoxy, halogen, CO₂H, CN, O(C═O)C₁-C₆ alkyl, oxo, and N(R^{b})₂;

R^{a} is (C₁-C₆)alkyl, (C₃-C₆)cycloalkyl, aryl or heterocyclyl; and

R^{b} is H, (C₁-C₆)alkyl, aryl, heterocyclyl, (C₃-C₆)cycloalkyl, (C═O)OC₁-C₆ alkyl, (C═O)C₁-C₆ alkyl or S(O)₂R^{a};

R^{c} is selected from: H, C₁-C₆ alkyl, aryl, C₂-C₆ alkenyl, C₂-C₆ alkynyl, heterocyclyl, C₃-C₈ cycloalkyl and C₁-C₆ perfluoroalkyl, wherein said alkyl, cycloalkyl, aryl, heterocylyl, alkenyl, and alkynyl is optionally substituted with one or more substituents selected from R^{z};

or a pharmaceutically acceptable salt or a stereoisomer thereof.

Exemplary AKT inhibitors include:

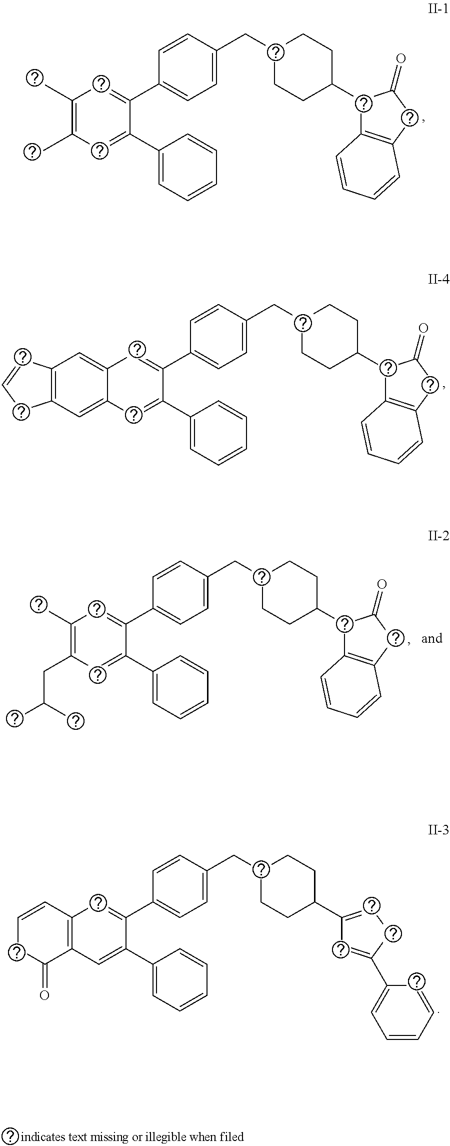 [SMILES: Cc1cc(-c2ccccc2)c(-c2ccc(CC3CCC(C4C(=O)Cc5ccccc54)CC3)cc2)cc1C ; O=C1Cc2ccccc2C1C1CCC(Cc2ccc(-c3cc4cc5c(cc4cc3-c3ccccc3)CC=C5)cc2)CC1 ; Cc1cc(-c2ccc(CC3CCC(C4C(=O)Cc5ccccc54)CC3)cc2)c(-c2ccccc2)cc1CC(C)C ; O=C1CC=Cc2cc(-c3ccc(CC4CCC(C5=CCC(c6ccccc6)=C5)CC4)cc3)c(-c3ccccc3)cc21]

In one embodiment, the kinase inhibitor is an Akt-1 selective inhibitor, and is a compound of Formula IV:

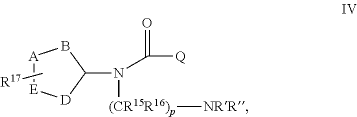

wherein, A, B, D and E are independently S, —CH, O or N, wherein depending on A, B, D and E, the ring shown in formula IV can be aromatic, heteroaromatic, cyclic or heterocyclic;
p is an integer from 1 to 6;
R¹⁵ and R¹⁶ are independently selected from the group consisting of hydrogen, halogen, OH, amino, dialkylamino, monoalkylamino and C₁-C₆-alkyl;
Q is a 5-6 membered aromatic or heteroaromatic ring; in one embodiment Q the following structure:

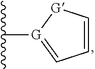

wherein, G and G′ are independently N, S or —C═C—;
R′ and R″ are taken together with the N to which they are bound to form a 5, 6 or 7 member heterocyclic ring, which can be optionally substituted with halogen, OH, amino, dialkylamino, monoalkylamino, C₁-C₆-alkyl, and, as an example, has the following structure, which depending on G′ can be heteroaromatic or heterocyclic, which can further contain the above-listed substituents:

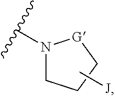

wherein G′ is as described above,
J is an unsubstituted or substituted amide;
R¹⁷ is a 5-14 membered aromatic or heteroaromatic ring system, which can be optionally substituted; in one embodiment R¹⁷ has one of the following structures:

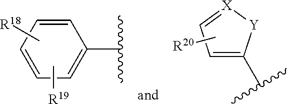

wherein, X and Y, independently, are N, O, S or —CH;
R¹⁸, R¹⁹ and R²⁰ are independently selected from the group consisting of halogen, OH, amino, dialkylamino, monoalkylamino, C₁-C₆-alkyl or phenyl, which is optionally substituted with halogen, OH, C₁-C₃ alkyl or cyclopropylmethyl; or R¹⁸ and R¹⁹ are taken together to form an aromatic, heteroaromatic, cyclic or heterocyclic ring.

Compounds of Formula IV include:

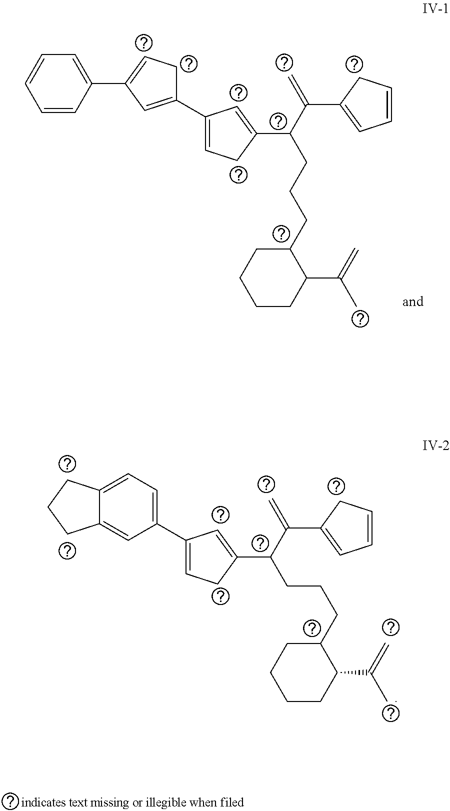

Another embodiment includes AKT inhibitors such as perifosine having the formula:

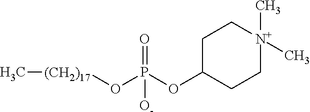

Another embodiment includes AKT inhibitors such as oligonucleotides, including antisense oligonucleotides having the sequences: 5′ ccagcccccaccagtccact 3′,5′ cgccaaggagatcatgcagc 3′,5′ gctgcatgatctccttggcg 3′,5′ agatagctggtgacagacag 3′,5′ cgtggagagatcatctgagg 3′,5′ tcgaaaaggtcaagtgctac 3′,5′ tggtgcagcggcagcggcag 3′ and 5′ ggcgcgagcgcgggcctagc 3′.

In one embodiment, the kinase inhibitor is a compound of Formula III. In one example, compounds of Formula III include PI3-k inhibitors. In another example, compounds of Formula III include mTOR inhibitors. Compounds of Formula III have the formula:

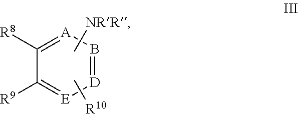

wherein, A, B, D and E are independently —CH or N;
R⁸ and R⁹ are taken together to form a 5 or 6 membered aromatic, heteroaromatic, cyclic or heterocyclic ring, which can be optionally substituted. For example, R⁸ and R⁹ can be taken together with the carbons in formula III to which they are attached to form a 9-10 member bicyclic ring system. Embodiments of the bicyclic ring systems include the following structures, wherein [symbol] indicates a bond in the formula III ring:

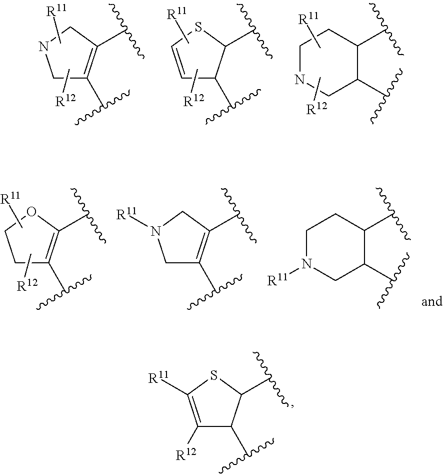

wherein R¹¹ and R¹² are independently selected from the group consisting of hydrogen, halogen, OH, amino, dialkylamino, monoalkylamino, C₁-C₆-alkyl, —C(═O)O—(CR^{y}R^{z})ₙ—W or phenyl, which is optionally substituted with halogen, OH, C₁-C₃ alkyl or cyclopropylmethyl, wherein W is C₅₋₁₂ aryl or heteroaryl, R^{y} and R^{z} are independently hydrogen, halogen, —OH or C₁₋₆ alkyl; or R¹¹ and R¹² are taken together to form a 5-14 membered aromatic or heteroaromatic ring. For example, R¹¹ and R¹² can be taken together with the carbons to which they are attached and the ring in Formula III above to form a 12-14 member tricyclic ring system, and in one embodiment has the following structure:

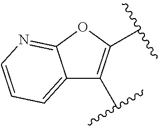 [SMILES: *c1oc2ncccc2c1*]

R′ and R″ are taken together with the N to which they are bound to form a 5, 6 or 7 member heterocyclic ring, which can be optionally substituted with halogen, OH, amino, dialkylamino, monoalkylamino, C₁-C₆-alkyl, having one of the following structures, which can further contain the above-listed substituents:

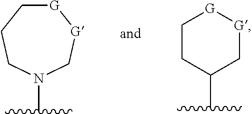

wherein, G and G′ are independently C, O or N;
R¹⁰ is an aromatic or heteroaromatic ring, having the structure:

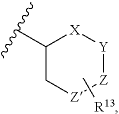

wherein, X, Y, Z and Z′ are independently —CH or N;
R¹³ is hydrogen, halogen, OH, amino, dialkylamino, monoalkylamino, C₁-C₆-alkyl or —N—(C═O)—N—R¹⁴, wherein R¹⁴ is C₁-C₆-alkyl. An example of R¹⁰ is:

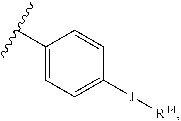

wherein, J is —N—(C═O)—N—, and R¹⁴ is C₁-C₆-alkyl.

An example compound of Formula III includes the PI3-k inhibitor:

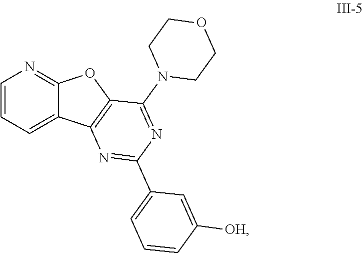 [SMILES: Oc1cccc(-c2nc(N3CCOCC3)c3oc4ncccc4c3n2)c1]

Another embodiment includes mTOR inhibitors having the following formula:

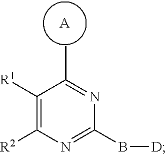

stereoisomers, tautomers or a pharmaceutically acceptable salt thereof, wherein:

A is a ring selected from the group consisting of morpholin-4-yl, 3,4-dihydro-2H-pyran-4-yl, 3,6-dihydro-2H-pyran-4-yl, tetrahydro-2H-pyran-4-yl, 1,4-oxazepan-4-yl, piperidin-1-yl, and is optionally substituted with from 1 to 2 substituents selected from the group consisting of —C(O)OR^{a}, —C(O)NR^{a}R^{b}, —NR^{a}R^{b}, —OR^{a}, —SR^{a}, —S(O)₂R^{c}, —S(O)R^{c}, —R^{c}, halogen, —NO₂, —CN and —N₃, wherein R^{a} and R^{b} are each independently selected from hydrogen, C₁₋₆ alkyl, C₁₋₆ haloalkyl, C₂₋₆ alkenyl and C₃₋₆ cycloalkyl, or R^{a} and R^{b}, together with the nitrogen atom to which each is attached, are combined to form a 3- to 6-membered ring, and R^{e} is selected from C₁₋₆ alkyl, C₁₋₆ haloalkyl, C₂₋₆ alkenyl, C₃₋₆ cycloalkyl;

R¹ and R² are combined with the atoms to which they are attached to form an optionally substituted pyrrolidine, piperidine or homopiperidine ring, wherein the nitrogen atom of said pyrrolidine, piperidine or homopiperidine ring is substituted by the group:

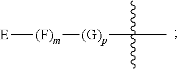

wherein E is hydrogen, C₆₋₁₀ aryl, C₅₋₁₀ heteroaryl, C₃₋₁₀ cycloalkyl, C₃₋₁₀ heterocycloalkyl, C₁₋₆ alkyl or C₁₋₆ heteroalkyl; and wherein E is optionally substituted with 1 to 5 substituents selected from halogen, C₁₋₆ alkyl, —NR^{d}R^{e}, —SR^{d}, —OR^{d}, —C(O)OR^{d}, —C(O)NR^{d}R^{e}, —C(O)R^{d}, —NR^{d}C(O)R^{e}, —OC(O)R^{f}, —NR^{d}C(O)NR^{d}R^{e}, —OC(O)NR^{d}R^{e}, —C(═NOR^{d})NR^{d}R^{e}, —NR^{d}C(═N—CN)NR^{d}R^{e}, —NR^{d}S(O)₂NR^{d}R^{e}, —S(O)₂R^{d}, —S(O)₂NR^{d}R^{e}, —R^{f}, —NO₂, —N₃, ═O, —CN, —(CH₂)₁₋₄—NR^{d}R^{e}, —(CH₂)₁₋₄—SR^{d}, —(CH₂)₁₋₄—OR″, —(CH₂)₁₋₄—C(O)OR^{d}, —(CH₂)₁₋₄—C(O)NR^{d}R^{e}, —(CH₂)₁₋₄—C(O)R^{d}, —(CH₂)₁₄—NR^{d}C(O)R^{e}, —(CH₂)₁₋₄—OC(O)R^{f}, —(CH₂)₁₋₄—NR^{d}C(O)NR^{d}R^{e}, —(CH₂)₁₋₄—OC(O)NR^{d}R^{e}, —(CH₂)₁₋₄—C(═NOR^{d})NR^{d}R^{e}, —(CH₂)₁₋₄—NR^{d}C(═N—CN)NR^{d}R^{e}, —(CH₂)₁₋₄—NR^{d}S(O)₂NR^{d}R^{e}, —(CH₂)₁₋₄—S(O)₂R^{d}, —(CH₂)₁₋₄—S(O)₂NR^{d}R^{e}, —(CH₂)₁₋₄—NO₂, —(CH₂)₁₋₄—N₃ or —(CH₂)₁₋₄—CN; wherein R^{d} and R^{e} are each independently selected from hydrogen, C₁₋₆ alkyl, C₁₋₆ haloalkyl, C₂₋₆ alkenyl, C₂₋₆ alkynyl, C₃₋₇ cycloalkyl, C₃₋₇ heterocycloalkyl, phenyl and —(CH₂)₁₋₄-phenyl, or R^{d} and R^{e}, when attached to the same nitrogen atom are combined to form a 3- to 6-membered ring; R^{f} is selected from C₁₋₆ alkyl, C₁₋₆ haloalkyl, C₂₋₆ alkenyl, C₂₋₆ alkynyl, C₃₋₇ cycloalkyl, C₃₋₇ heterocycloalkyl, phenyl and —(CH₂)₁₋₄-phenyl;

F is a member selected from the group consisting of C₁₋₆ alkylene, C₂₋₆ alkenylene, C₂₋₆ alkynylene and C₁₋₆ heteroalkylene; wherein F is independently substituted with from 0 to 3 substituents selected from the group consisting of halogen, —NR^{g}R^{h}, —SR^{g}, —OR^{g}, —C(O)OR^{g}, —C(O)NR^{g}R^{h}, —NR^{g}C(O)Rⁱ, —OC(O)Rⁱ, —NR^{g}C(O)NR^{g}R^{h}, —OC(O)NR^{g}R^{h}, NR^{g}S(O)₂NR^{g}R^{h}, —S(O)₂R^{g}, —S(O)₂NR^{g}R^{h}, —Rⁱ, —NO₂, N₃, ═O, —CN, —(CH₂)₁₋₄—NR^{g}R^{h}, —(CH₂)₁₋₄—SR^{g}, —(CH₂)₁₋₄—OR^{g}, —(CH₂)₁₋₄—C(O)OR^{g}, —(CH₂)₁₋₄—C(O)NR^{g}R^{h}, —(CH₂)₁₋₄—C(O)R^{g}, —(CH₂)₁₋₄—NR^{g}C(O)R^{h}, —(CH₂)₁₋₄—OC(O)Rⁱ, —(CH₂)₁₋₄—NR^{g}C(O)NR^{g}R^{h}, —(CH₂)₁₄—OC(O)NR^{g}R^{h}, —(CH₂)₁₋₄—NR^{g}S(O)₂NR^{g}R^{h}, —(CH₂)₁₋₄—S(O)₂R^{g}, —(CH₂)₁₋₄—S(O)₂NR^{g}R^{h}, —(CH₂)₁₋₄—NO₂, —(CH₂)₁₋₄—N₃ and —(CH₂)₁₋₄—CN; wherein R^{g} and R^{h} are each independently selected from hydrogen, C₁₋₆ alkyl, C₁₋₆ haloalkyl, C₁₋₆ heteroalkyl, C₃₋₇ cycloalkyl, C₃₋₇ heterocycloalkyl, phenyl and —(CH₂)₁₋₄-phenyl, and optionally R^{g} and R^{h}, when attached to the same nitrogen atom are combined to form a 3- to 6-membered ring; Rⁱ is selected from C₁₋₆ alkyl, C₁₋₆ haloalkyl, C₃₋₇ cycloalkyl, C₃₋₇ heterocycloalkyl, phenyl and —(CH₂)₁₋₄-phenyl;

G is a member selected from the group consisting of —C(O)—, —OC(O)—, —NHC(O)—, —NHC(═NOH)—, —S(O)₂— and —NHS(O)₂—;

m and p are each independently an integer from 0 to 1, wherein if m and p are both the integer 0, then E is not C₁₋₆ alkyl or C₁₋₆ heteroalkyl;

wherein pyrrolidine, piperidine or homopiperidine ring formed by combining R¹ and R² is further substituted with from 0 to 5 substituents selected from the group consisting of halogen, —NR^{j}R^{k}, —SR^{k}, —OR^{j}, —C(O)OR^{j}, —C(O)NR^{j}R^{k}, —NHC(O)R^{j}, —OC(O)R^{j}, —R^{m}, —CN and ═O, wherein R^{j} and R^{k} are each independently selected from hydrogen, C₁₋₆ alkyl, C₁₋₆ haloalkyl, C₂₋₆ alkenyl, C₂₋₆ alkynyl, C₃₋₅ cycloalkyl and C₃₋₅ heterocycloalkyl, and R^{j} and R^{k}, when attached to the same nitrogen atom, are optionally combined to form a 3- to 6-membered ring; and R^{m} is selected from C₁₋₆ alkyl, C₁₋₆ haloalkyl, C₂₋₆ alkenyl, C₂₋₆ alkynyl, C₃₋₅ cycloalkyl and C₃₋₅ heterocycloalkyl;

B is selected from the group consisting of phenylene, pyridylene, pyrimidylene, pyridazinylene and pyrazinyline and is substituted with from 0 to 4 substituents selected from halogen, —CN, —N₃, —NO₂, —C(O)ORⁿ, —C(O)NRⁿR^{o}, —NRⁿC(O)R^{o}, —NRⁿC(O)NRⁿR^{o}, —ORⁿ, —NRⁿR^{o} and R^{p}; wherein Rⁿ and R^{o} are independently selected from hydrogen and C₁₋₄ alkyl, C₁₋₄ haloalkyl, C₁₋₄ heteroalkyl, C₃₋₇ cycloalkyl and C₃₋₇ heterocycloalkyl, or when attached to the same nitrogen atom, Rⁿ and R^{o} are optionally are combined to form a 3- to 6-membered ring; R^{p} is C₁₋₄ alkyl, C₁₋₄ haloalkyl, C₃₋₇ cycloalkyl and C₃₋₇ heterocycloalkyl, wherein any two substituents, not including the D group, located on adjacent atoms of B are optionally combined to form a 5- to 6-membered carbocyclic, heterocyclic, aryl or heteroaryl ring; and

D is a member selected from the group consisting of —NR³C(O)NR⁴R⁵, —NR⁴R⁵, —C(O)NR⁴R⁵, —OC(O)OR⁴, —OC(O)NR⁴R⁵, —NR³C(═N—CN)NR⁴R⁵, —NR³C(═N—OR⁴)NR⁴R⁵, —NR³C(═N—NR⁴)NR⁴R⁵, —NR³C(O)R⁴, —NR³C(O)OR⁴, —NR³S(O)₂NR⁴R⁵ and —NR³S(O)₂R⁴, wherein R³ is selected from the group consisting of hydrogen, C₁₋₆ alkyl, C₁₋₆ haloalkyl and C₂₋₆ alkenyl; R⁴ and R⁵ are each independently selected from the group consisting of hydrogen, C₁₋₆ alkyl, C₁₋₆ haloalkyl, C₂₋₆ alkenyl, C₂₋₆ alkynyl, C₃₋₁₀ cycloalkyl, C₃₋₁₀ heterocycloalkyl, C₆₋₁₀ aryl and C₅₋₁₀ heteroaryl, and R⁴ and R⁵, when attached to the same nitrogen atom, are optionally combined to form a 5- to 7-membered heterocyclic or heteroaryl ring; and wherein R³, R⁴ and R⁵ are further substituted with from 0 to 3 substituents independently selected from the group consisting of halogen, —NO₂, —CN, —NR^{q}R^{r}, —OR^{q}, —SR^{q}, —C(O)OR^{q}, —C(O)NR^{q}R^{r}, —NR^{q}C(O)R^{r}, —NR^{q}C(O)OR^{s}, —(CH₂)₁₋₄—NR^{q}R^{r}, —(CH₂)₁₋₄—OR^{q}, —(CH₂)₁₋₄—SR^{q}, —(CH₂)₁₋₄—C(O)OR^{q}, —(CH₂)₁₋₄—C(O)NR^{q}R^{r}, —(CH₂)₁₋₄—NR^{q}C(O)R^{r}, —(CH₂)₁₋₄—NR^{q}C(O)OR^{r}, —(CH₂)₁₋₄—CN, —(CH₂)₁₋₄—NO₂, —S(O)R^{r}, —S(O)₂R^{r}, ═O, and —R^{s}; wherein R^{q} and R^{r} is selected from hydrogen, C₁₋₆ alkyl, C₁₋₆ haloalkyl, C₂₋₆ alkenyl, C₂₋₆ alkynyl, C₁₋₆ heteroalkyl, C₃₋₇ cycloalkyl, C₃₋₇ heterocycloalkyl, C₆₋₁₀ aryl, C₅₋₁₀ heteroaryl; and R^{s}, at each occurrence, is independently selected from C₁₋₆ alkyl. C₁₋₆ haloalkyl, C₁₋₆ heteroalkyl, C₃₋₇ cycloalkyl, C₃₋₇ heterocycloalkyl, C₆₋₁₀ aryl and C₅₋₁₀ heteroaryl; and wherein the D group and a substituent located on an adjacent atom of the B ring are optionally combined to form a 5- to 6-membered heterocyclic or heteroaryl ring.

In certain embodiments:

A is a ring selected from the group consisting of morpholin-4-yl, 3,4-dihydro-2H-pyran-4-yl, 3,6-dihydro-2H-pyran-4-yl, tetrahydro-2H-pyran-4-yl, 1,4-oxazepan-4-yl, piperidin-1-yl, optionally substituted by C₁-C₆ alkyl;

B is selected from the group consisting of phenylene and pyrimidylene;

D is —NR³C(O)NR⁴R⁵, —NR⁴R⁵, —C(O)NR⁴R⁵, —OC(O)OR⁴, —OC(O)NR⁴R⁵, —NR³C(═N—CN)NR⁴R⁵, —NR³C(═N—OR⁴)NR⁴R⁵, —NR³C(═N—NR⁴)NR⁴R⁵, —NR³C(O)R⁴, —NR³C(O)OR⁴, —NR³S(O)₂NR⁴R⁵ or —NR³S(O)₂R⁴, wherein R³ is hydrogen or C₁₋₆ alkyl; R⁴ and R⁵ are each independently hydrogen, C₁₋₆ alkyl, C₁₋₆ haloalkyl or C₃₋₁₀ cycloalkyl, or R⁴ and R⁵ are combined to form a 5- or 6-membered heterocyclic ring;

R¹ and R² are combined with the atoms to which they are attached to form an substituted pyrrolidine, piperidine or homopiperidine ring, wherein the nitrogen atom of said ring is substituted by the group:

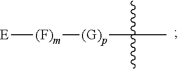

wherein E is hydrogen, C₆ aryl, C₅₋₆ heteroaryl, C₁₋₆ alkyl or C₅₋₆ heterocycloalkyl,; and wherein E is optionally substituted with 1 to 5 substituents selected from halogen, C₁₋₆ alkyl, —NR^{d}R^{e}, —SR^{d}, —OR^{d}, —C(O)OR^{d}, —C(O)NR^{d}R^{e}, —C(O)R^{d}, —NR^{d}C(O)R^{e}, —OC(O)R^{f}, —NR^{d}C(O)NR^{d}R^{e}, —OC(O)NR^{d}R^{e}, —C(═NOR^{d})NR^{d}R^{e}, —NR^{d}C(═N—CN)NR^{d}R^{e}, —NR^{d}S(O)₂NR^{d}R^{e}, —S(O)₂R^{d}, —S(O)₂NR^{d}R^{e}, —R^{f}, —NO₂, —N₃, ═O, —CN, —(C₂)₁₋₄—NR^{d}R^{e}, —(CH₂)₁₋₄—SR^{d}, —(CH₂)₁₋₄—OR^{d}, —(CH₂)₁₋₄—C(O)OR^{d}, —(CH₂)₁₋₄—C(O)NR^{d}R^{e}, —(CH₂)₁₋₄—C(O)R^{d}, —(CH₂)₁₋₄—NR^{d}C(O)R^{e}, —(CH₂)₁₋₄—OC(O)R^{f}, —(CH₂)₁₋₄—NR^{d}C(O)NR^{d}R^{e}, —(CH₂)₁₋₄—OC(O)NR^{d}R^{e}, —(CH₂)₁₋₄—C(═NOR^{d})NR^{d}R^{e}, —(CH₂)₁₋₄—NR^{d}C(═N—CN)NR^{d}R^{e}, —(CH₂)₁₋₄—NR^{d}S(O)₂NR^{d}R^{e}, —(CH₂)₁₋₄—S(O)₂R^{d}, —(CH₂)₁₋₄—S(O)₂NR^{d}R^{e}, —(CH₂)₁₋₄—NO₂, —(CH₂)₁₋₄—N₃ or —(CH₂)₁₋₄—CN; wherein R^{d} and R^{e} are each independently selected from hydrogen, C₁₋₆ alkyl, C₁₋₆ haloalkyl, C₂₋₆ alkenyl, C₂₋₆ alkynyl, C₃₋₇ cycloalkyl, C₃₋₇ heterocycloalkyl, phenyl and —(CH₂)₁₋₄-phenyl, or R^{d} and R^{e}, when attached to the same nitrogen atom are combined to form a 3- to 6-membered ring; R^{f} is selected from C₁₋₆ alkyl, C₁₋₆ haloalkyl, C₃₋₇ cycloalkyl, C₃₋₇ heterocycloalkyl, phenyl and —(CH₂)₁₋₄-phenyl;

F is C₁₋₆ alkylene;

G is —C(O)—, —OC(O)—, —NHC(O)—, —NHC(═NOH)—, —S(O)₂— or —NHS(O)₂—; and

m and p are independently 0 or 1.

Another embodiment includes mTOR inhibitor compounds, including:

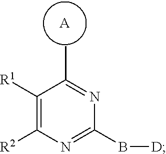

stereoisomers, tautomers, and pharmaceutically acceptable salts thereof, wherein A is a 5- to 8-membered heterocyclic ring having from 1 to 3 heteroatoms independently selected from N, O and S as ring vertices, and having from 0 to 2 double bonds; wherein the A ring is further substituted with from 0 to 5 R^{A} substituents selected from the group consisting of C(O)OR^{a}, —C(O)NR^{a}R^{b}, —NR^{a}R^{b}, —OC(O)R^{c}, —OR^{a}, —SR^{a}, —S(O)₂R^{c}, —S(O)R^{c}, —R^{c}, —(CH₂)₁₋₄—NR^{a}R^{b}, —(CH₂)₁₋₄—NR^{a}C(O)R^{c}, —(CH₂)₁₋₄—OR^{a}, —(CH₂)₁₋₄—SR^{a}, —(CH₂)₁₋₄—S(O)₂R^{c}, —(CH₂)₁₋₄—S(O)R^{c}, halogen, —NO₂, —CN and —N₃, wherein R^{a} and R^{b} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄(phenyl), and optionally R^{a} and R^{b}, together with the nitrogen atom to which each is attached, are combined to form a 3- to 7-membered heterocyclic ring comprising 1 to 2 heteroatoms selected from N, O and S; R^{c} is selected from C₁₋₆ alkyl, C₁₋₆ haloalkyl, C₁₋₆ heteroalkyl, C₂₋₆ alkenyl, C₂₋₆ alkynyl, C₃₋₆ cycloalkyl, phenyl and —(CH₂)₁₋₄ (phenyl); and any two substituents attached to the same atom in the 5- to 8-membered heterocyclic ring are optionally combined to form a 3- to 5-membered carbocyclic or a 3 to 5-membered heterocyclic ring; R¹ and R² are combined with the atoms to which they are attached to form a 5- to 8-membered monocyclic or bridged bicyclic heterocyclic ring comprising —O— as one of the ring vertices; wherein the 5- to 8-membered monocyclic or bridged-bicyclic heterocyclic ring formed by combining R¹ and R² further optionally comprises one additional heteroatom selected from the group consisting of N, O and S, and is substituted with from 0 to 5 R^{R} substituents selected from the group consisting of halogen, —NR^{j}R^{k}, —SR^{j}, —OR^{j}, —C(O)OR^{j}, —C(O)NR^{j}R^{k}, —NHC(O)R^{j}, —OC(O)R^{j}, —R^{m}, —CN, ═O, ═S, ═N—CN, —(CH₂)₁₋₄—CN, —(CH₂)₁₋₄—OR^{j}, —(CH₂)₁₋₄—NR^{j}R^{k}, —C₁₋₄ alkylene-OR^{j}, —C₁₋₄ alkylene-R^{m}, —C₂₋₄ alkenylene-R^{m}, —C₂₋₄ alkynylene-R^{m}, —C₁₋₄ alkylene-C₁₋₉ heteroaryl, C₂₋₄ alkenylene-C₁₋₉ heteroaryl, C₂₋₄ alkynylene-C₁₋₉ heteroaryl, C₁₋₄ alkylene-C₆₋₁₀ aryl, C₂₋₄ alkynylene-C₆₋₁₀ aryl and C₂₋₄ alkynylene-C₆₋₄₀ aryl, wherein R^{j} and R^{k} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl, pyridyl and —(CH₂)₁₋₄—(Ph), and R^{j} and R^{k}, when attached to the same nitrogen atom, are optionally combined to form a 3- to 6-membered heterocyclic ring comprising 1 to 2 heteroatoms selected from N, O and S; and R^{m} is selected from C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl and —(CH₂)₁₋₄—(Ph), and wherein a C₃₋₇ cycloalkyl, C₂₋₆ heterocycloalkyl, C₁₋₉ heteroaryl or C₆₋₁₀ aryl portion of a R^{R} substituent is substituted with from 0 to 3 substituents selected from the group consisting of F, Cl, Br, I, —NH(C₁₋₄ alkyl), —N(diC₁₋₄ alkyl), O(C₁₋₄ alkyl), C₁₋₆ alkyl, C₁₋₆ heteroalkyl, —C(O)O(C₁₋₄ alkyl), —C(O)NH(C₁₋₄alkyl), —C(O)N(diC₁₋₄ alkyl), —NO₂, —CN; wherein when R¹ and R² are combined to form a monocyclic 5- to 8-membered heterocyclic ring then any two R^{R} substitutents attached to the same atom or adjacent carbon atoms in said 5- to 8-membered heterocyclic ring are optionally combined to form a 3- to 7-membered cycloalkyl ring or a 3- to 7-membered heterocycloalkyl ring comprising 1 to 2 heteroatoms selected from N, O and S as ring vertices; B is a member selected from the group consisting of phenylene and 5- to 6-membered heteroarylene, and is substituted with from 0 to 4 R^{B} substituents selected from halogen, —CN, —N₃, —NO₂, —C(O)ORⁿ, —C(O)NRⁿR^{o}, —NR¹¹C(O)R^{o}, —NRⁿC(O)NRⁿR^{o}, —ORⁿ, —NRⁿR^{o}, —(CH₂)₁₋₄—C(O)ORⁿ, —(CH₂)₁₋₄—C(O)NRⁿR^{o}, —(CH₂)₁₋₄—ORⁿ, —(CH₂)₁₋₄—NRⁿR^{o}, —(CH₂)₁₋₄—SR^{p} and R^{p}; wherein Rⁿ and R^{o} are independently selected from hydrogen and C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl and —(CH₂)₁₋₄-(phenyl) or when attached to the same nitrogen atom, Rⁿ and R^{o} are optionally are combined to form a 3- to 6-membered heterocyclic ring comprising 1 to 2 heteroatoms selected from N, O and S; R^{p} is C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₆ heterocycloalkyl, phenyl and —(CH₂)₁₋₄-(phenyl), wherein any two substituents, not including the D group, located on adjacent atoms of B are optionally combined to form a 5- to 6-membered carbocyclic, heterocyclic, aryl or heteroaryl ring; D is a member selected from the group consisting of —NR³C(O)NR⁴R⁵, —NR⁴R⁵, —C(O)NR⁴R⁵, —OC(O)OR⁴, —OC(O)NR⁴R⁵, —NR³C(═N—CN)NR⁴R⁵, —NR³C(═N—OR⁴)NR⁴R⁵, —NR³C(═N—NR⁴)NR⁴R⁵, —NR³C(O)R⁴, —NR³C(O)OR⁴, —NR³S(O)₂NR⁴R⁵, —NR³S(O)₂R⁴, —NR³C(═S)NR⁴R⁵ and —S(O)₂R⁴R⁵, wherein R³ is selected from the group consisting of hydrogen, C₁₋₆ alkyl, C₁₋₆ haloalkyl and C₂₋₆ alkenyl; R⁴ and R⁵ are each independently selected from the group consisting of hydrogen, C₁₋₆ alkyl, C₁₋₆ haloalkyl, C₁₋₆ alkylamino-C(═O)—, C₂₋₆ alkenyl, C₂₋₆ alkynyl, C₃₋₁₀ cycloalkyl, C₂₋₉ heterocycloalkyl, C₆₋₁₀ aryl and C₁₋₉ heteroaryl, and R⁴ and R⁵, when attached to the same nitrogen atom, are optionally combined to form a 5- to 7-membered heterocyclic or 5- to 6-membered heteroaryl ring comprising 1 to 3 heteroatoms selected from N, O and S; and wherein R³, R⁴ and R⁵ are further substituted with from 0 to 3 R^{D} substituents independently selected from the group consisting of halogen, —NO₂, —CN, —NR^{q}R^{r}, —OR^{q}, —SR^{q}, —C(O)OR^{q}, —C(O)NR^{q}R^{r}, —NR^{q}C(O)R^{r}, —NR^{q}C(O)OR^{s}, —(CH₂)₁₋₄—NR^{q}R^{r}, —(CH₂)₁₋₄—OR^{q}, —(CH₂)₁₋₄—SR^{q}, —(CH₂)₁₋₄—C(O)OR^{q}, —(CH₂)₁₋₄—C(O)NR^{q}R^{r}, —(CH₂)₁₋₄—NR^{q}C(O)R^{r}, —(CH₂)₁₋₄—NR^{q}C(O)OR^{r}, —(CH₂)₁₋₄—CN, —(CH₂)₁₋₄—NO₂, —S(O)R^{r}, —S(O)₂R^{r}, —(CH₂)₁₋₄R^{s}, ═O, and —R^{s}; wherein R^{q} and R^{r} is selected from hydrogen, C₁₋₆ alkyl, C₁₋₆ haloalkyl, C₂₋₆ alkenyl, C₂₋₆ alkynyl, C₁₋₆ heteroalkyl, C₃₋₇ cycloalkyl, C₂₋₆ heterocycloalkyl, C₆₋₁₀ aryl, C₁₋₉ heteroaryl; and R^{s}, at each occurrence, is independently selected from C₁₋₆ alkyl, C₁₋₆ haloalkyl, C₃₋₇ cycloalkyl, C₂₋₆ heterocycloalkyl, C₆₋₁₀ aryl and C₁₋₉ heteroaryl; and wherein the D group and a substituent located on an adjacent atom of the B ring are optionally combined to form a 5- to 6-membered heterocyclic or heteroaryl ring optionally substituted with 1 to 2 R^{D} substituents.

Another embodiment includes mTOR inhibitor compounds, including:

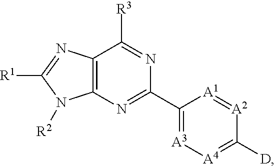

stereoisomers, tautomers, and pharmaceutically acceptable salts thereof, wherein R¹ is selected from the group consisting of 6- to 10-membered aryl, 5- to 9-membered heteroaryl, 3- to 12-membered heterocycloalkyl, 3- to 12-membered cycloalkyl, wherein R¹ is substituted with from 0 to 5 R^{R1} substituents selected from the group consisting of halogen, F, Cl, Br, I, —NR^{a}R^{b}, —SR^{a}, —OR^{a}, —C(O)OR^{a}, —C(O)NR^{a}R^{b}, —C(O)R^{a}, —NR^{a}C(O)R^{b}, —OC(O)R^{c}, —NR^{a}C(O)NR^{a}R^{b}, —OC(O)NR^{a}R^{b}, —NR^{a}S(O)₂NR^{a}R^{b}, —S(O)₂R^{a}, —S(O)₂NR^{a}R^{b}, —R^{c}, —NO₂, —N₃, ═O, —CN, R^{c1}, —X¹—NR^{a}R^{b}, —X¹—SR^{a}, —X¹—OR^{a}, —X¹—C(O)OR^{a}, —X¹—C(O)NR^{a}R^{b}, —X¹—C(O)R^{a}, —X¹—NR^{a}C(O)R^{b}, —X¹—OC(O)R^{a}, —X¹—NR^{a}C(O)NR^{a}R^{b}, —X¹—OC(O)NR^{a}R^{b}, —X¹—NR^{a}S(O)₂NR^{a}R^{b}, —X¹—S(O)₂R^{a}, —X¹—S(O)₂NR^{a}R^{b}, —X¹—NO₂, —X¹—N₃, —X¹—CN, and X¹—R^{c1}; wherein R^{a} and R^{b} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₇ heterocycloalkyl, phenyl and —(CH₂)₁₋₄-phenyl, optionally R^{a} and R^{b}, when attached to the same nitrogen atom are combined to form a 3- to 6-membered heterocyclic ring comprising 1 to 2 heteroatoms selected from N, O and S; R^{c} is selected from C₁₋₆ alkyl, C₁₋₆ haloalkyl, C₂₋₆ alkenyl, C₂₋₆ alkynyl, C₃₋₇ cycloalkyl, C₂₋₇ heterocycloalkyl, phenyl and —(CH₂)₁₋₄-phenyl; X¹ is selected from the group consisting of C₁₋₄ alkylene, C₂₋₄ alkenylene and C₂₋₄ alkynylene; and R^{c1} is selected from the group consisting of phenyl, 2-pyridyl, 3-pyridyl, 4-pyridyl, 2-imidazolyl, 2-indolyl, 1-naphthyl, 2-naphthyl, 2-thienyl, 3-thienyl, 2-pyrrolyl, 2-furanyl and 3-furanyl, and wherein R^{c1} is substituted with from 0 to 3 substituents selected from F, Cl, Br, I, —NR^{a}R^{b}, —SR^{a}, —OR^{a}, —S(O)₂R^{a}, —S(O)₂NR^{a}R^{b}, —NO₂, —N₃, ═O, —CN, pyridyl, C₁₋₆ alkyl, C₂₋₆ alkenyl, C₂₋₆ alkynyl and C₁₋₆ heteroalkyl; R² is selected from the group consisting of hydrogen, C₁₋₆ alkyl, C₂₋₆ alkenyl, C₂₋₆ alkynyl, C₁₋₆ heteroalkyl, a 6- to 10 membered aryl, 5- to 10-membered heteroaryl, a 3- to 12-membered heterocycloalkyl, a 3- to 12 membered cycloalkyl, -L-C₆₋₁₀ aryl, -L-C₁₋₉ heteroaryl, -L-C₃₋₁₂ cycloalkyl and -L-C₂₋₁₂ heterocycloalkyl, wherein L is selected from C₁₋₆ alkylene, C₂₋₆ alkenylene, C₂₋₆ alkynylene and C₁₋₆ heteroalkylene, and wherein R² is substituted with from 0 to 5 R^{R2} substituents selected from the group consisting of halogen, F, Cl, Br, I, —NR^{d}R^{e}, —SR^{d}, —OR^{d}, —C(O)OR^{d}, —C(O)NR^{d}R^{e}, —C(O)R^{d}, —NR^{d}C(O)R^{e}, —OC(O)R^{f}, —NR^{d}C(O)NR^{d}R^{e}, —OC(O)NR^{d}R^{e}, —NR^{d}S(O)₂NR^{d}R^{e}, —S(O)₂R^{d}, —S(O)₂NR^{d}R^{e}, —R^{f}, —NO₂, —N₃, ═O, —CN, —X²—NR^{d}R^{e}, —X²—SR^{d}, —X²—OR^{d}, —X²—C(O)OR^{d}, —X²—C(O)NR^{d}R^{e}, —X²—C(O)R^{d}R^{e}, —X²—NR^{d}C(O)R^{e}, —X²—OC(O)R^{d}, —X²—NR^{d}C(O)NR^{d}R^{e}, —X²—OC(O)NR^{d}R^{e}, —X²—NR^{d}S(O)₂NR^{d}R^{e}, —X²—S(O)₂R^{d}, —X²—S(O)₂NR^{d}R^{e}, —X²—N₂, —X²—N₃ and —X²—CN; wherein R^{d} and R^{e} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₇ heterocycloalkyl, phenyl and —(CH₂)₁₋₄-phenyl, optionally R^{d} and R^{e}, when attached to the same nitrogen atom are combined to form a 3- to 6-membered heterocyclic ring comprising 1 to 2 heteroatoms selected from N, O and S; R^{f} is selected from C₁₋₆ alkyl, C₁₋₆ haloalkyl, C₂₋₆ alkenyl, C₂₋₆ alkynyl, C₃₋₇ cycloalkyl, C₂₋₇ heterocycloalkyl, phenyl and —(CH₂)₁₋₄-phenyl; and X² is selected from the group consisting of C₁₋₄ alkylene, C₂₋₄ alkenylene and C₂₋₄ alkynylene; R³ is a 5- to 12-membered monocyclic or bridged heterocycloalkyl ring, wherein the R³ group is substituted with from 0 to 3 R^{R3} substituents selected from the group consisting of —C(O)OR^{g}, —C(O)NR^{g}R^{h}, —NR^{g}R^{h}, —OR^{g}, —SR^{g}, —S(O)₂Rⁱ, —S(O)Rⁱ, —Rⁱ, halogen, F, Cl, Br, I, —NO₂, —CN and —N₃, wherein R^{g} and R^{h} are each independently selected from hydrogen, C₁₋₆ alkyl, C₁₋₆ haloalkyl, C₁₋₆ heteroalkyl, C₂₋₆ alkenyl and C₃₋₆ cycloalkyl, wherein optionally R^{g} and R^{h}, together with the nitrogen atom to which each is attached, are combined to form a 3- to 6-membered heterocyclic ring comprising 1 to 2 heteroatoms selected from N, O and S, and Rⁱ is selected from C₁₋₆ alkyl, C₁₋₆ haloalkyl, C₂₋₆ alkenyl, C₃₋₆ cycloalkyl; and if R³ is a monocyclic heterocycloalkyl ring then any two R^{R3} groups attached to the same atom of R³ is optionally combined to form at 3- to 7-membered carbocyclic or 3- to 7-membered heterocyclic ring comprising 1 to 2 atoms selected from N, O and S as ring vertices; A¹, A², A³ and A⁴ are each a member independently selected from N, C(R^{A}) or C(H), wherein at least three of A¹, A², A³ and A⁴ is each independently C(H) or C(R^{A}), wherein R^{A} at each occurrence is independently selected from the group consisting of F, Cl, Br, I, —NO₂, —CN, C₁₋₄ alkyl, C₂₋₄ alkenyl, C₂₋₄ alkynyl, or any two R^{A} groups attached to adjacent atoms are optionally combined to form a C₂₋₆ heterocyclic ring comprising from 1 to 2 heteroatoms selected from N, O and S as ring vertices, C₃₋₇ cycloalkyl ring, a C₁₋₅ heteroaryl ring comprising from 1 to 4 heteroatoms selected from N, O and S as ring vertices, or phenyl ring; and D is a member selected from the group consisting of —NR⁴C(O)NR⁵R⁶, —NR⁵R⁶, —C(O)NR⁵R⁶, —OC(O)OR⁵, —OC(O)NR⁵R⁶, —NR⁴C(═N—CN)NR⁵R⁶, —NR⁴C(═N—OR⁵)NR⁵R⁶, —NR⁴C(═N—NR⁵)NR⁵R⁶, —NR⁴C(O)R⁵, —NR⁴C(O)OR⁵, —NR⁴S(O)₂NR⁵R⁶ and —NR⁴S(O)₂R⁵, wherein R⁴ is selected from the group consisting of hydrogen, C₁₋₆ alkyl, C₁₋₆ haloalkyl and C₂₋₆ alkenyl; R⁵ and R⁶ are each independently selected from the group consisting of hydrogen, C₁₋₆ alkyl, C₁₋₆ haloalkyl, C₂₋₆ alkenyl, C₂₋₆ alkynyl, C₃₋₁₀ cycloalkyl, C₂₋₁₀ heterocycloalkyl, C₆₋₁₀ aryl and C₁₋₉ heteroaryl, and R⁵ and R⁶, when attached to the same nitrogen atom, are optionally combined to form a 5- to 7-membered heterocyclic or a 5- to 9-membered heteroaryl ring comprising 1 to 3 heteroatoms selected from N, O and S as ring vertices and substituted with 0-3 R^{D} substituents; and wherein R⁴, R⁵ and R⁶ are further substituted with from 0 to 3 R^{D} substituents, wherein R^{D} is independently selected from the group consisting of halogen, F, Cl, Br, I, —NO₂, —CN, —NR^{j}R^{k}, —OR^{j}, —SR^{j}, —C(O)OR^{j}, —C(O)NR^{j}R^{k}, —NR^{j}C(O)R^{k}, —NR^{j}C(O)OR^{m}, —X³—NR^{j}R^{k}, —X³—OR^{j}, —X³—SR^{j}, —X³—C(O)OR^{j}, —X³—C(O)NR^{j}R^{k}, —X³—NR^{j}C(O)R^{k}, —X³—NR^{j}C(O)OR^{k}, —X³—CN, —X³—NO₂, —S(O)R^{m}, —S(O)₂R^{m}, ═O, and —R^{m}; wherein R^{j} and R^{k} is selected from hydrogen, C₁₋₆ alkyl, C₁₋₆ haloalkyl, C₂₋₆ alkenyl, C₂₋₆ alkynyl, C₁₋₆ heteroalkyl, C₃₋₇ cycloalkyl, C₃₋₇ heterocycloalkyl, C₆₋₁₀ aryl, C₁₋₉ heteroaryl; and R^{m}, at each occurrence, is independently selected from C₁₋₆ alkyl, C₁₋₆ haloalkyl, C₃₋₇ cycloalkyl, C₃₋₇ heterocycloalkyl, C₆₋₁₀ aryl and C₁₋₉ heteroaryl; X³ is selected from the group consisting of C₁₋₄ alkylene, C₂₋₄ alkenylene and C₂₋₄ alkynylene; and wherein D and a R^{A} substituent attached to an atom that is adjacent to the atom to which D is attached are optionally combined to form an optionally substituted 5- to 6-membered heterocyclic or heteroaryl ring substituted with from 0 to 4 R^{D} substituents.

Another embodiment includes mTOR inhibitor compounds, including:

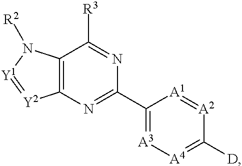

stereoisomers, tautomers, and pharmaceutically acceptable salts thereof, wherein Y¹ and Y² is each independently N or C(R¹), but Y¹ and Y² are not both N or are not both C(R¹), wherein R¹ is selected from the group consisting of hydrogen, C₁₋₆ alkyl, C₂₋₆ alkenyl, C₂₋₆ alkynyl, C₁₋₆ heteroalkyl, 6- to 10-membered aryl, 5- to 9-membered heteroaryl, 3- to 12-membered heterocycloalkyl, 3- to 12-membered cycloalkyl, wherein R¹ is substituted with from 0 to 5 R^{R1} substituents selected from the group consisting of halogen, F, Cl, Br, I, —NR^{a}R^{b}, —SR^{a}, —OR^{a}, —C(O)OR^{a}, —C(O)NR^{a}R^{b}, —C(O)R^{a}, —NR^{a}C(O)R^{b}, —OC(O)R^{c}, —NR^{a}C(O)NR^{a}R^{b}, —OC(O)NR^{a}R^{b}, —NR^{a}S(O)₂NR^{a}R^{b}, —S(O)₂R^{a}, —S(O)₂NR^{a}R^{b}, —R^{c}, —NO₂, —N₃, ═O, —CN, R^{c1}, —X¹—NR^{a}R^{b}, —X¹—SR^{a}, —X¹—OR^{a}, —X¹—C(O)OR^{a}, —X¹—C(O)NR^{a}R^{b}, —X¹—C(O)R^{a}, —X¹—NR^{a}C(O)R^{b}, —X¹—OC(O)R^{a}, —X¹—NR^{a}C(O)NR^{a}R^{b}, —X¹—OC(O)NR^{a}R^{b}, —X¹—NR^{a}S(O)₂NR^{a}R^{b}, —X¹—S(O)₂R^{a}, —X¹—S(O)₂NR^{a}R^{b}, —X¹—NO₂, —X¹—N₃, —X¹—CN, and X¹—R^{c1}; wherein R^{a} and R^{b} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₇ heterocycloalkyl, phenyl and —(CH₂)₁₋₄-phenyl, optionally R^{a} and R^{b}, when attached to the same nitrogen atom are combined to form a 3- to 6-membered heterocyclic ring comprising 1 to 2 heteroatoms selected from N, O and S; R^{c} is selected from C₁₋₆ alkyl, C₁₋₆ haloalkyl, C₂₋₆ alkenyl, C₂₋₆ alkynyl, C₃₋₇ cycloalkyl, C₂₋₇ heterocycloalkyl, phenyl and —(CH₂)₁₋₄-phenyl; X¹ is selected from the group consisting of C₁₋₄ alkylene, C₂₋₄ alkenylene and C₂₋₄ alkynylene; and R^{c1} is selected from the group consisting of phenyl, 2-pyridyl, 3-pyridyl, 4-pyridyl, 2-imidazolyl, 2-indolyl, 1-naphthyl, 2-naphthyl, 2-thienyl, 3-thienyl, 2-pyrrolyl, 2-furanyl and 3-furanyl, and wherein R^{c1} is substituted with from 0 to 3 substituents selected from F, Cl, Br, I, —NR^{a}R^{b}, —SR^{a}, —OR^{a}, —S(O)₂R^{a}, —S(O)₂NR^{a}R^{b}, —NO₂, —N₃, ═O, —CN, pyridyl, C₁₋₆ alkyl, C₂₋₆ alkenyl, C₂₋₆ alkynyl and C₁₋₆ heteroalkyl; R² is selected from the group consisting of hydrogen, C₁₋₆ alkyl, C₂₋₆ alkenyl, C₂₋₆ alkynyl, C₁₋₆ heteroalkyl, -L-C₆₋₁₀ aryl, -L-C₁₋₉ heteroaryl, -L-C₃₋₁₂ cycloalkyl and -L-C₂₋₁₂ heterocycloalkyl, wherein L is selected from C₁₋₆ alkylene, C₂₋₆ alkenylene, C₂₋₆ alkynylene and C₁₋₆ heteroalkylene, and wherein R² is substituted with from 0 to 5 R^{R2} substituents selected from the group consisting of halogen, F, Cl, Br, I, —NR^{d}R^{e}, —SR^{d}, —OR^{d}, —C(O)OR^{d}, —C(O)NR^{d}R^{e}, —C(O)R^{d}, —NR^{d}C(O)R^{e}, —OC(O)R^{f}, —NR^{d}C(O)NR^{d}R^{e}, —OC(O)NR^{d}R^{e}, —NR^{d}S(O)₂NR^{d}R^{e}, —S(O)₂R^{d}, —S(O)₂NR^{d}R^{e}, —R^{f}, —NO₂, —N₃, ═O, —CN, —X²—NR^{d}R^{e}, —X²—SR^{d}, —X²—OR^{d}, —X²—C(O)OR^{d}, —X²—C(O)NR^{d}R^{e}, —X²—C(O)R^{d}, —X²—NR^{d}C(O)R^{e}, —X²—OC(O)R^{d}, —X²—NR^{d}C(O)NR^{d}R^{e}, —X²—OC(O)NR^{d}R^{e}, —X²—NR^{d}S(O)₂NR^{d}R^{e}, —X²—S(O)₂R^{d}, —X²—S(O)₂NR^{d}R^{e}, —X²—NO₂, —X²—N₃ and —X²—CN; wherein R^{d} and R^{e} are each independently selected from hydrogen, C₁₋₆ alkyl, C₁₋₆ haloalkyl, C₁₋₆ heteroalkyl, C₂₋₆ alkenyl, C₂₋₆ alkynyl, C₃₋₇ cycloalkyl, C₂₋₇ heterocycloalkyl, phenyl and —(CH₂)₁₋₄-phenyl, optionally R^{d} and R^{e}, when attached to the same nitrogen atom are combined to form a 3- to 6-membered heterocyclic ring comprising 1 to 2 heteroatoms selected from N, O and S; R^{f} is selected from C₁₋₆ alkyl, C₁₋₆ haloalkyl, C₂₋₆ alkenyl, C₂₋₆ alkynyl, C₃₋₇ cycloalkyl, C₂₋₇ heterocycloalkyl, phenyl and —(CH₂)₁₋₄-phenyl; and X² is selected from the group consisting of C₁₋₄ alkylene, C₂₋₄ alkenylene and C₂₋₄ alkynylene; R³ is a 5- to 12-membered monocyclic or bridged heterocycloalkyl ring, wherein the R³ group is substituted with from 0 to 3 R^{R3} substituents selected from the group consisting of —C(O)OR^{g}, —C(O)NR^{g}R^{h}, —NR^{g}R^{h}, —OR^{g}, —SR^{g}, —S(O)₂Rⁱ, —S(O)Rⁱ, —Rⁱ, halogen, F, Cl, Br, I, —NO₂, —CN and —N₃, wherein R^{g} and R^{h} are each independently selected from hydrogen, C₁₋₆ alkyl, C₁₋₆ haloalkyl, C₁₋₆ heteroalkyl, C₂₋₆ alkenyl and C₃₋₆ cycloalkyl, wherein optionally R^{g} and R^{h}, together with the nitrogen atom to which each is attached, are combined to form a 3- to 6-membered heterocyclic ring comprising 1 to 2 heteroatoms selected from N, O and S, and Rⁱ is selected from C₁₋₆ alkyl, C₁₋₆ haloalkyl, C₂₋₆ alkenyl, C₃₋₆ cycloalkyl; and when R³ is a monocyclic heterocycloalkyl ring then any two R^{R3} groups attached to the same atom of R³ is optionally combined to form at 3- to 7-membered carbocyclic or 3- to 7-membered heterocyclic ring comprising 1 to 2 atoms selected from N, O and S as ring vertices; A¹, A², A³ and A⁴ are each a member independently selected from N, C(R^{A}) or C(H), wherein at least three of A¹, A², A³ and A⁴ is each independently C(H) or C(R^{A}), wherein R^{A} at each occurrence is independently selected from the group consisting of F, Cl, Br, I, —NO₂, —CN, C₁₋₄ alkyl, C₂₋₄ alkenyl, C₂₋₄ alkynyl, or any two R^{A} groups attached to adjacent atoms are optionally combined to form a C₂₋₆ heterocyclic ring comprising from 1 to 2 heteroatoms selected from N, O and S as ring vertices, C₃₋₇ cycloalkyl ring, a C₁₋₅ heteroaryl ring comprising from 1 to 4 heteroatoms selected from N, O and S as ring vertices, or phenyl ring; and D is a member selected from the group consisting of —NR⁴C(O)NR⁵R⁶, —NR⁵R⁶, —C(O)NR⁵R⁶, —OC(O)OR⁵, —OC(O)NR⁵R⁶, —NR⁴C(═N—CN)NR⁵R⁶, —NR⁴C(═N—OR⁵)NR⁵R⁶, —NR⁴C(═N—NR⁵)NR⁵R⁶, —NR⁴C(O)R⁵, —NR⁴C(O)OR⁵, —NR⁴S(O)₂NR⁵R⁶ and —NR⁴S(O)₂R⁵, wherein R⁴ is selected from the group consisting of hydrogen, C₁₋₆ alkyl, C₁₋₆ haloalkyl and C₂₋₆ alkenyl; R⁵ and R⁶ are each independently selected from the group consisting of hydrogen, C₁₋₆ alkyl, C₁₋₆ haloalkyl, C₂₋₆ alkenyl, C₂₋₆ alkynyl, C₃₋₁₀ cycloalkyl, C₂₋₁₀ heterocycloalkyl, C₆₋₁₀ aryl and C₁₋₉ heteroaryl, and R⁵ and R⁶, when attached to the same nitrogen atom, are optionally combined to form a 5- to 7-membered heterocyclic or a 5- to 9-membered heteroaryl ring comprising 1 to 3 heteroatoms selected from N, O and S as ring vertices and substituted with 0-3 R^{D} substituents; and wherein R⁴, R⁵ and R⁶ are further substituted with from 0 to 3 R^{D} substituents, wherein R^{D} is independently selected from the group consisting of halogen, F, Cl, Br, I, —NO₂, —CN, —NR^{j}R^{k}, —OR^{j}, —SR^{j}, —C(O)OR^{j}, —C(O)NR^{j}R^{k}, —NR^{j}C(O)R^{k}, —NR^{j}C(O)OR^{m}, —X³—NR^{j}R^{k}, —X³—OR^{j}, —X³—SR^{j}, —X³—C(O)OR^{j}, —X³—C(O)NR^{j}R^{k}, —X³—NR^{j}C(O)R^{k}, —X³—NR^{j}C(O)OR^{k}, —X³—CN, —X³—NO₂, —S(O)R^{m}, —S(O)₂R^{m}, ═O, and —R^{m}; wherein R^{j} and R^{k} is selected from hydrogen, C₁₋₆ alkyl, C₁₋₆ haloalkyl, C₂₋₆ alkenyl, C₂₋₆ alkynyl, C₁₋₆ heteroalkyl, C₃₋₇ cycloalkyl, C₃₋₇ heterocycloalkyl, C₆₋₁₀ aryl, C₁₋₉ heteroaryl; and R^{m}, at each occurrence, is independently selected from C₁₋₆ alkyl, C₁₋₆ haloalkyl, C₃₋₇ cycloalkyl, C₃₋₇ heterocycloalkyl, C₆₋₁₀ aryl and C₁₋₉ heteroaryl; X³ is selected from the group consisting of C₁₋₄ alkylene, C₂₋₄ alkenylene and C₂₋₄ alkynylene; and wherein D and a R^{A} substituent attached to an atom that is adjacent to the atom to which D is attached are optionally combined to form an optionally substituted 5- to 6-membered heterocyclic or heteroaryl ring substituted with from 0 to 4 R^{D} substituents.

Another embodiment includes mTOR inhibitor compounds, including:

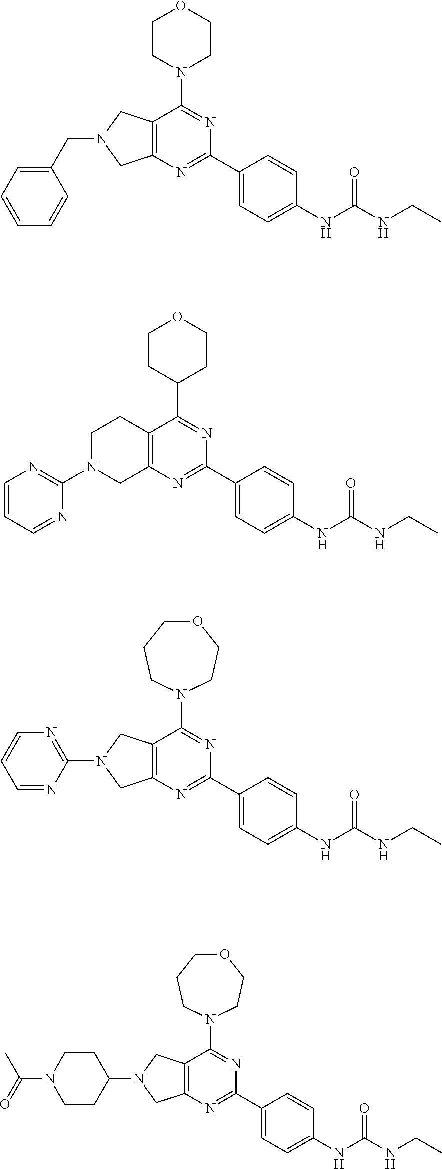 [SMILES: CCNC(=O)Nc1ccc(-c2nc3c(c(N4CCOCC4)n2)CN(Cc2ccccc2)C3)cc1 ; CCNC(=O)Nc1ccc(-c2nc3c(c(C4CCOCC4)n2)CCN(c2ncccn2)C3)cc1 ; CCNC(=O)Nc1ccc(-c2nc3c(c(N4CCCOCC4)n2)CN(c2ncccn2)C3)cc1 ; CCNC(=O)Nc1ccc(-c2nc3c(c(N4CCCOCC4)n2)CN(C2CCN(C(C)=O)CC2)C3)cc1]

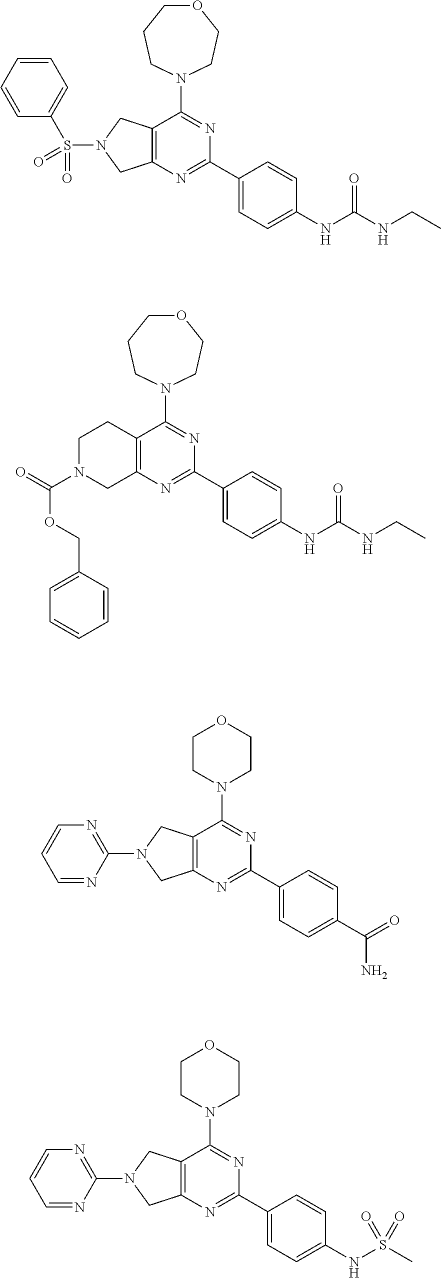 [SMILES: CCNC(=O)Nc1ccc(-c2nc3c(c(N4CCCOCC4)n2)CN(S(=O)(=O)c2ccccc2)C3)cc1 ; CCNC(=O)Nc1ccc(-c2nc3c(c(N4CCCOCC4)n2)CCN(C(=O)OCc2ccccc2)C3)cc1 ; NC(=O)c1ccc(-c2nc3c(c(N4CCOCC4)n2)CN(c2ncccn2)C3)cc1 ; CS(=O)(=O)Nc1ccc(-c2nc3c(c(N4CCOCC4)n2)CN(c2ncccn2)C3)cc1]

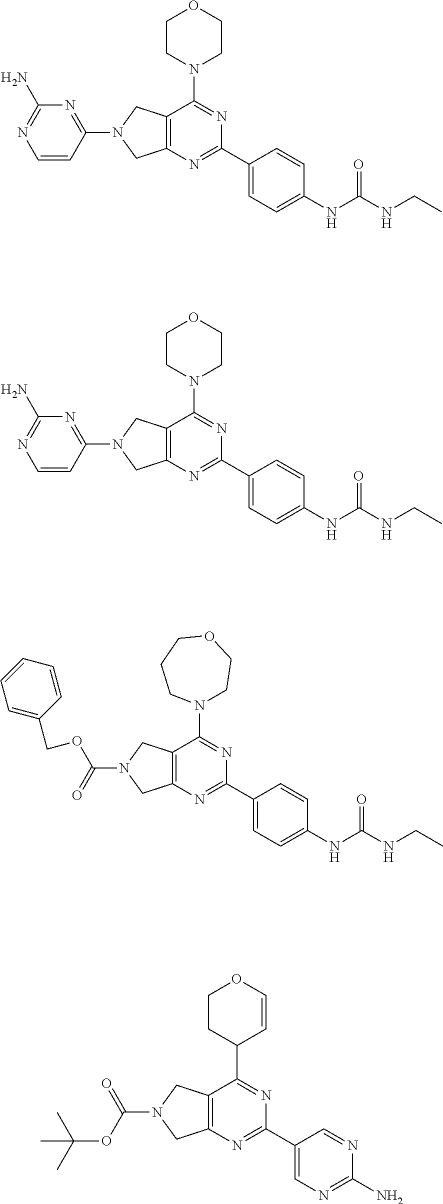

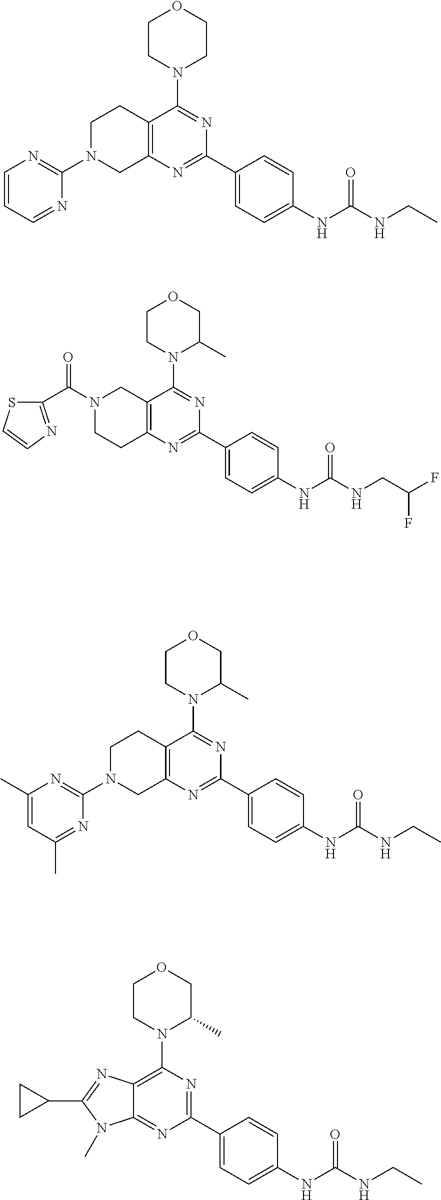

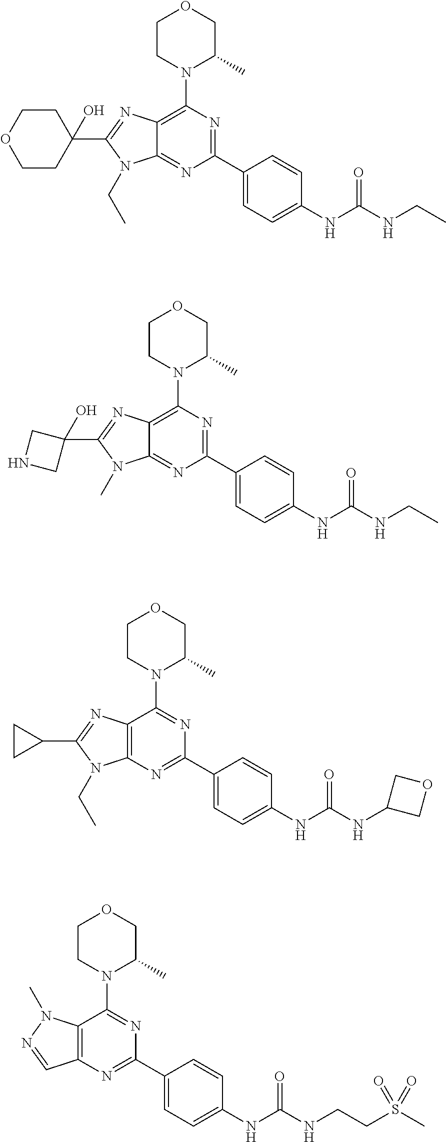 [SMILES: CCNC(=O)Nc1ccc(-c2nc(N3CCOC[C@@H]3C)c3nc(C4(O)CCOCC4)n(CC)c3n2)cc1 ; CCNC(=O)Nc1ccc(-c2nc(N3CCOC[C@@H]3C)c3nc(C4(O)CNC4)n(C)c3n2)cc1 ; CCn1c(C2CC2)nc2c(N3CCOC[C@@H]3C)nc(-c3ccc(NC(=O)NC4COC4)cc3)nc21 ; C[C@H]1COCCN1c1nc(-c2ccc(NC(=O)NCCS(C)(=O)=O)cc2)nc2cnn(C)c12]

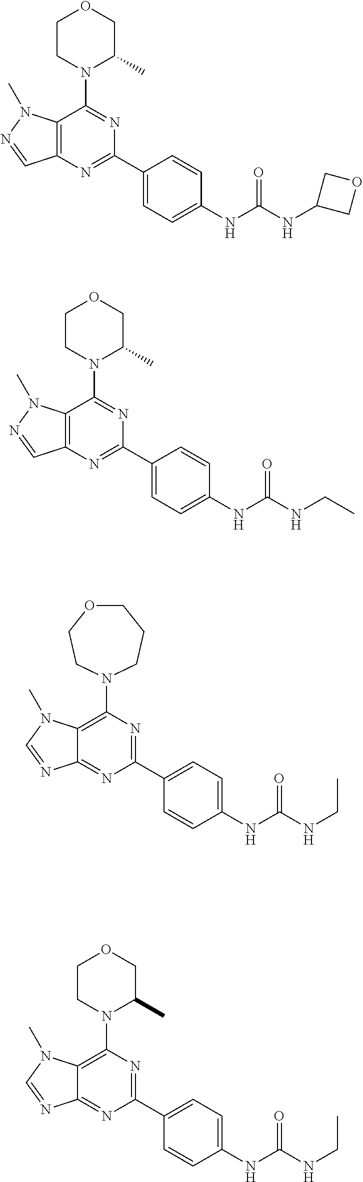 [SMILES: C[C@H]1COCCN1c1nc(-c2ccc(NC(=O)NC3COC3)cc2)nc2cnn(C)c12 ; CCNC(=O)Nc1ccc(-c2nc(N3CCOC[C@@H]3C)c3c(cnn3C)n2)cc1 ; CCNC(=O)Nc1ccc(-c2nc(N3CCCOCC3)c3c(ncn3C)n2)cc1 ; CCNC(=O)Nc1ccc(-c2nc(N3CCOC[C@H]3C)c3c(ncn3C)n2)cc1]

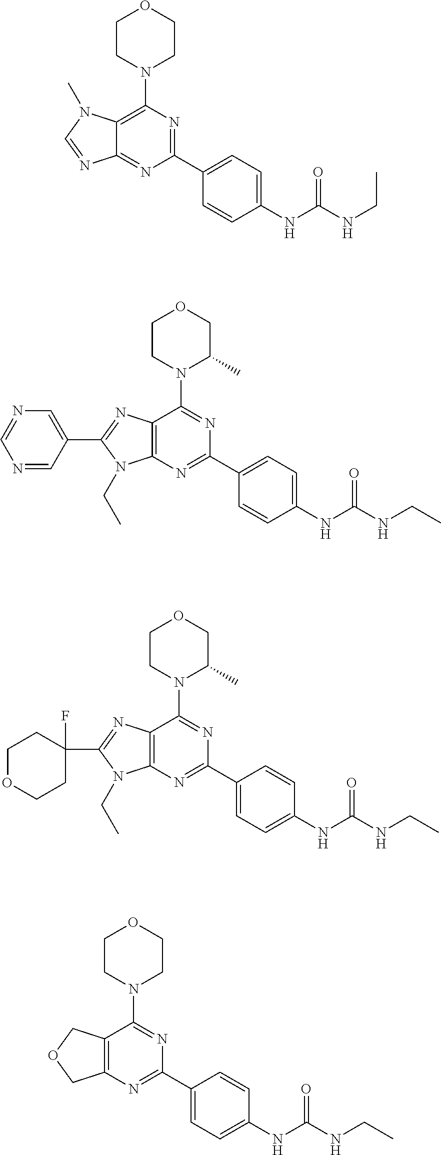 [SMILES: CCNC(=O)Nc1ccc(-c2nc(N3CCOCC3)c3c(ncn3C)n2)cc1 ; CCNC(=O)Nc1ccc(-c2nc(N3CCOC[C@@H]3C)c3nc(-c4cncnc4)n(CC)c3n2)cc1 ; CCNC(=O)Nc1ccc(-c2nc(N3CCOC[C@@H]3C)c3nc(C4(F)CCOCC4)n(CC)c3n2)cc1 ; CCNC(=O)Nc1ccc(-c2nc3c(c(N4CCOCC4)n2)COC3)cc1]

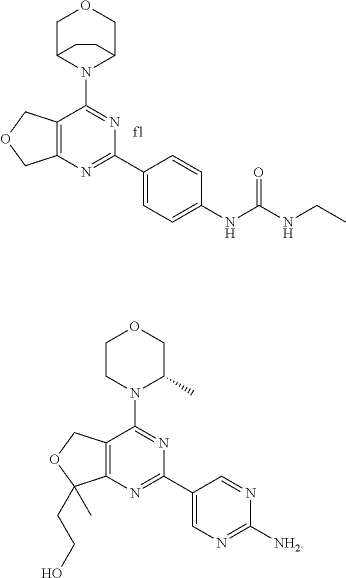 [SMILES: CCNC(=O)Nc1ccc(-c2nc3c(c(N4C5CCC4COC5)n2)COC3)cc1 ; C[C@H]1COCCN1c1nc(-c2cnc(N)nc2)nc2c1COC2(C)CCO]

Another embodiment includes the mTOR inhibitor, rapamycin:

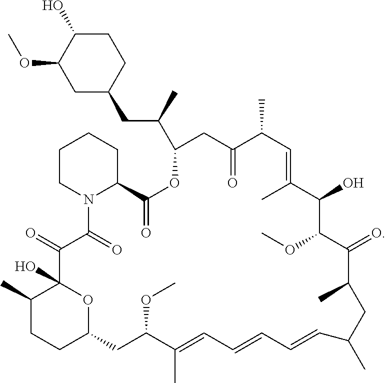 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)CC(C)/C=C/C=C/C=C/1C]

Another embodiment includes PI3-k inhibitor compounds of the following formula:

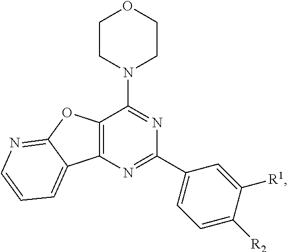

or pharmaceutically acceptable salts thereof, wherein:

R¹ and R² are independently selected from hydrogen, halogen, C₁₋₆ alkyl, —NR^{d}R^{e}, —SR^{d}, —OR^{d}, —C(O)OR^{d}, —C(O)NR^{d}R^{e}, —C(O)R^{d}, —NR^{d}C(O)R^{e}, —OC(O)R^{f}, —NR^{d}C(O)NR^{d}R^{e}, —OC(O)NR^{d}R^{e}, —C(═NOR^{d})NR^{d}R^{e}, —NR^{d}C(═N—CN)NR^{d}R^{e}, —NR^{d}S(O)₂NR^{d}R^{e}, —S(O)₂R^{d}, —S(O)₂NR^{d}R^{e}, —R^{f}, —NO₂, —N₃, ═O, —CN, —(CH₂)₁₋₄—NR^{d}R^{e}, —(CH₂)₁₋₄—SR^{d}, —(CH₂)₁₋₄—OR^{d}, —(CH₂)₁₋₄—C(O)OR^{d}, —(CH₂)₁₋₄—C(O)NR^{d}R^{e}, —(CH₂)₁₋₄—C(O)R^{d}, —(CH₂)₁₋₄—NR^{d}C(O)R^{e}, —(CH₂)₁₋₄—OC(O)R^{f}, —(CH₂)₁₋₄—NR^{d}C(O)NR^{d}R^{e}, —(CH₂)₁₋₄—OC(O)NR^{d}R^{e}, —(CH₂)₁₋₄—C(═NOR^{d})NR^{d}R^{e}, —(CH₂)₁₋₄—NR^{d}C(═N—CN)NR^{d}R^{e}, —(CH₂)₁₋₄—NR^{d}S(O)₂NR^{d}R^{e}, —(CH₂)₁₋₄—S(O)₂R^{d}, —(CH₂)₁₋₄—S(O)₂NR^{d}R^{e}, —(CH₂)₁₋₄—NO₂, —(CH₂)₁₋₄—N₃ or —(CH₂)₁₋₄—CN; wherein R^{d} and R^{e} are each independently selected from hydrogen, C₁₋₆ alkyl, C₁₋₆ haloalkyl, C₂₋₆ alkenyl, C₂₋₆ alkynyl, C₃₋₂₇ cycloalkyl, C₃₋₇ heterocycloalkyl, phenyl and —(CH₂)₁₋₄-phenyl, or R^{d} and R^{e}, when attached to the same nitrogen atom are combined to form a 3- to 6-membered ring; R^{f} is selected from C₁₋₆ alkyl, C₁₋₆ haloalkyl, C₃₋₇ cycloalkyl, C₃₋₇ heterocycloalkyl, phenyl and —(CH₂)₁₋₄-phenyl; or

R¹ and R² are taken together with the atoms to which they are attached to form a fused 5- or 6-membered heterocyclyl or heteroaryl ring, optionally substituted by oxo, halogen, C₁-C₃ alkyl or CF₃.

Example PI3-k inhibitors include the following:

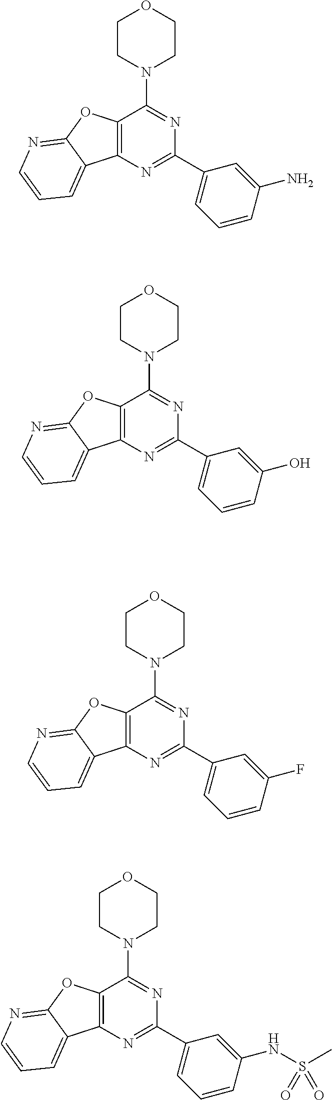 [SMILES: Nc1cccc(-c2nc(N3CCOCC3)c3oc4ncccc4c3n2)c1 ; Oc1cccc(-c2nc(N3CCOCC3)c3oc4ncccc4c3n2)c1 ; Fc1cccc(-c2nc(N3CCOCC3)c3oc4ncccc4c3n2)c1 ; CS(=O)(=O)Nc1cccc(-c2nc(N3CCOCC3)c3oc4ncccc4c3n2)c1]

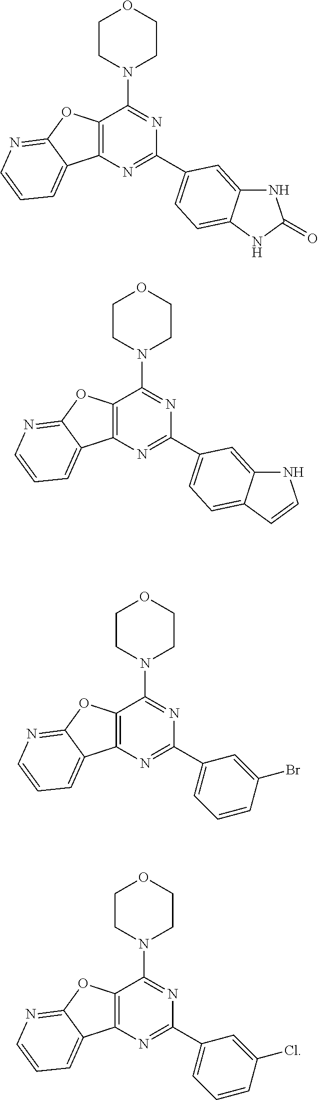 [SMILES: O=c1[nH]c2ccc(-c3nc(N4CCOCC4)c4oc5ncccc5c4n3)cc2[nH]1 ; c1cnc2oc3c(N4CCOCC4)nc(-c4ccc5cc[nH]c5c4)nc3c2c1 ; Brc1cccc(-c2nc(N3CCOCC3)c3oc4ncccc4c3n2)c1 ; Clc1cccc(-c2nc(N3CCOCC3)c3oc4ncccc4c3n2)c1]

In one embodiment, the kinase inhibitor is a PI3K kinase inhibitor of Formulas V and VI:

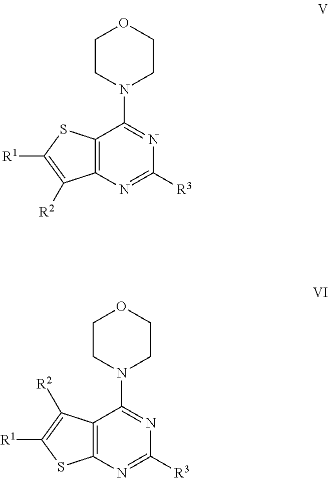

or stereoisomers, geometric isomers, tautomers, or pharmaceutically acceptable salts thereof, where:

R¹ is selected from H, F, Cl, Br, I, CN, —(CR¹⁴R¹⁵)ₘNR¹⁰R¹¹, —C(R¹⁴R¹⁵)ₙNR¹²C(═Y)R¹⁰, —(CR¹⁴R¹⁵)ₙNR¹²S(O)₂R¹⁰, —(CR¹⁴R¹⁵)ₘOR¹⁰, —(CR¹⁴R¹⁵)ₙS(O)₂R¹⁰, —(CR¹⁴R¹⁵)ₙS(O)₂NR¹⁰R¹¹, —C(OR¹⁰)R¹¹R¹⁴, —C(═Y)R¹⁰, —C(═Y)OR¹⁰, —C(═Y)NR¹⁰R¹¹, —C(═Y)NR¹²OR¹⁰, —C(═O)NR¹²S(O)₂R¹⁰, —C(═O)NR¹²(CR¹⁴R¹⁵)ₘNR¹⁰R¹¹, —NO₂, —NR¹²C(═Y)R¹¹, —NR¹²C(═Y)OR¹¹, —NR¹²C(═Y)NR¹⁰R¹¹, —NR¹²S(O)₂R¹⁰, —NR¹²SO₂NR¹⁰R¹¹, —SR¹⁰, —S(O)₂R¹⁰, —S(O)₂NR¹⁰R¹¹, —SC(Y)R¹⁰, —SC(═Y)OR¹⁰, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, and C₁-C₂₀ heteroaryl;

R² is selected from H, F, Cl, Br, I, CN, CF₃, —NO₂, —C(═Y)R¹⁰, —C(═Y)OR¹⁰, —C(═Y)NR¹⁰R¹¹, —(CR¹⁴R¹⁵)ₘNR¹⁰R¹¹, —(CR¹⁴R¹⁵)ₙOR¹⁰, —(CR¹⁴R¹⁵)ₜ—NR¹²C(═O)(CR¹⁴R¹⁵)NR¹⁰R¹¹, —NR¹²C(═Y)R¹⁰, —NR¹²C(═Y)OR¹⁰, —NR¹²C(═Y)NR¹⁰R¹¹, —NR¹²SO₂R¹⁰, OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, and C₁-C₂₀ heteroaryl;

R³ is a carbon linked monocyclic heteroaryl, a carbon linked fused bicyclic C₃-C₂₀ heterocyclyl, or a carbon linked fused bicyclic C₁-C₂₀ heteroaryl, where the monocyclic heteroaryl, fused bicyclic C₃-C₂₀ heterocyclyl, and fused bicyclic C₁-C₂₀ heteroaryl are optionally substituted with one or more groups selected from F, Cl, Br, I, —CN, —NR¹⁰R¹¹, —OR¹⁰, —C(O)R¹⁰, —NR¹⁰C(O)R¹¹, —N(C(O)R¹¹)₂, —NR¹⁰C(O)NR¹⁰R¹¹, NR¹²S(O)₂R¹⁰, —C(═O)OR¹⁰, —C(═O)NR¹⁰R¹¹, C₁-C₁₂ alkyl and (C₁-C₁₂ alkyl)-OR¹⁰;

R¹⁰, R¹¹ and R¹² are independently H, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl,

or R¹⁰ and R¹¹ together with the nitrogen to which they are attached form a C₂-C₂₀ heterocyclic ring optionally substituted with one or more groups independently selected from oxo, (CH₂)ₘOR¹², NR¹²R¹², CF₃, F, Cl, Br, I, SO₂R¹², C(═O)R¹², NR¹²C(═Y)R¹², NR¹²S(O)₂R¹², C(═Y)NR¹²R¹², C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl and C₁-C₂₀ heteroaryl;

R¹⁴ and R¹⁵ are independently selected from H, C₁-C₁₂ alkyl, or —(CH₂)ₙ-aryl,

or R¹⁴ and R¹⁵ together with the atoms to which they are attached form a saturated or partially unsaturated C₃-C₁₂ carbocyclic ring; where said alkyl, alkenyl, alkynyl, carbocyclyl, heterocyclyl, aryl, and heteroaryl, are optionally substituted with one or more groups independently selected from F, Cl, Br, I, CN, CF₃, —NO₂, oxo, R¹⁰, —C(═Y)R¹⁰, —C(═Y)OR¹⁰, —C(═Y)NR¹⁰R¹¹, —(CR¹⁴R¹⁵)ₙNR¹⁰R¹¹, —(CR¹⁴R¹⁵)ₙOR¹⁰, —NR¹⁰R¹¹, —NR¹²C(═Y)R¹⁰, —NR¹²C(═^{Y})OR¹¹, —NR¹²C(═^{Y})NR¹⁰R¹¹, —(CR¹⁴R¹⁵)ₘNR¹²SO₂R¹⁰, ═NR¹², OR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), —SR¹⁰, —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, and C₁-C₂₀ heteroaryl;

Y is O, S, or NR¹²;

m is 0, 1, 2, 3, 4, 5 or 6; and

n is 1, 2, 3, 4, 5 or 6.

Example PI3-k inhibitors include the following:

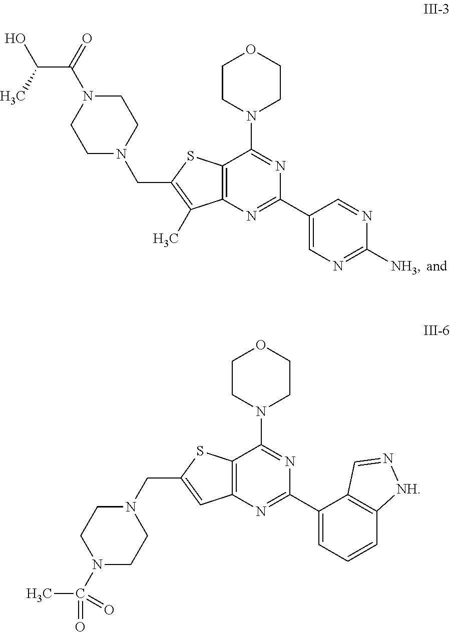

Preparation of Formulae V and VI Compounds

The Formula V and VI compounds may be synthesized by synthetic routes that include processes analogous to those well-known in the chemical arts, and including WO 2006/046031, which is incorporated herein by reference in its entirety, for all purposes. Starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, N.Y. (1967-1999 ed.), or Beilsteins Handbuch der organischen Chemie, 4, Aufl. ed. Springer-Verlag, Berlin, including supplements (also available via the Beilstein online database).

Formulae V and VI compound may be prepared using procedures to prepare other thiophenes, furans, pyrimidines (U.S. Pat. No. 6,608,053; U.S. Pat. No. 6,492,383; U.S. Pat. No. 6,232,320; U.S. Pat. No. 6,187,777; U.S. Pat. No. 3,763,156; U.S. Pat. No. 3,661,908; U.S. Pat. No. 3,475,429; U.S. Pat. No. 5,075,305; US 2003/220365; GB 1393161; WO 93/13664); and other heterocycles, which are described in: Comprehensive Heterocyclic Chemistry, Editors Katritzky and Rees, Pergamon Press, 1984.

Formulae V and VI compounds may be converted into a pharmaceutically acceptable salt, and a salt may be converted into the free compound, by conventional methods. Examples of pharmaceutically acceptable salts include salts with inorganic acids such as hydrochloric acid, hydrobromic acid, hydroiodic acid, sulphuric acid, nitric acid and phosphoric acid; and organic acids such as methanesulfonic acid, benzenesulphonic acid, formic acid, acetic acid, trifluoroacetic acid, propionic acid, oxalic acid, malonic acid, succinic acid, fumaric acid, maleic acid, lactic acid, malic acid, tartaric acid, citric acid, ethanesulfonic acid, aspartic acid and glutamic acid. The salt may be a mesylate, a hydrochloride, a phosphate, a benzenesulphonate or a sulphate. Salts may be mono-salts or bis-salts. For example, the mesylate salt may be the mono-mesylate or the bis-mesylate.

Formulae V and VI compounds and salts may also exist as hydrates or solvates.

Protection of functional groups (e.g., primary or secondary amine) of intermediates may be necessary in preparing Formulae V and VI compounds. The need for such protection will vary depending on the nature of the remote functionality and the conditions of the preparation methods. Suitable amino-protecting groups include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

For illustrative purposes, Schemes 5-11 show general methods for preparing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

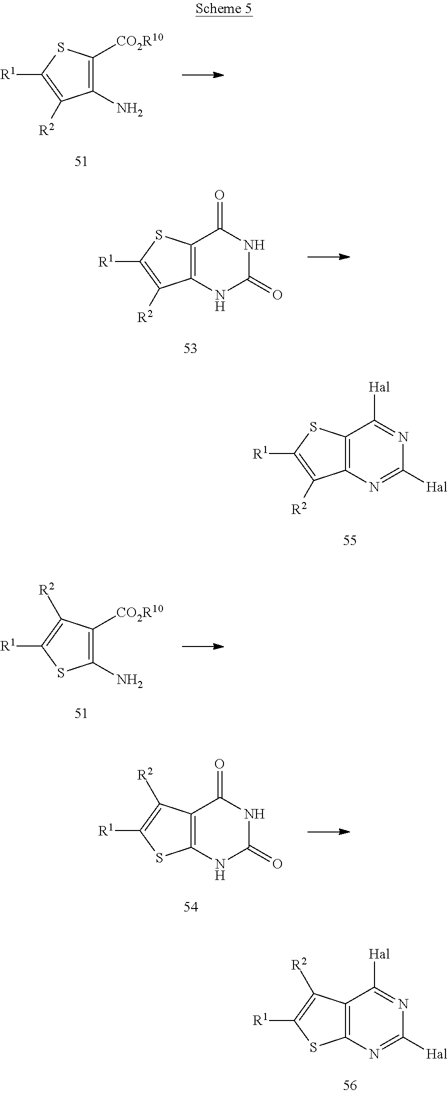

Scheme 5 shows a general method for preparation of the thienopyrimidine intermediates 55 and 56 from 2-carboxyester, 3-amino thiophene, and 2-amino, 3-carboxy ester thiophene reagents, respectively 51 and 52, wherein Hal is Cl, Br, or I; and R¹, R², and R¹⁰ are as defined for Formulae V and VI compounds, or precursors or prodrugs thereto.

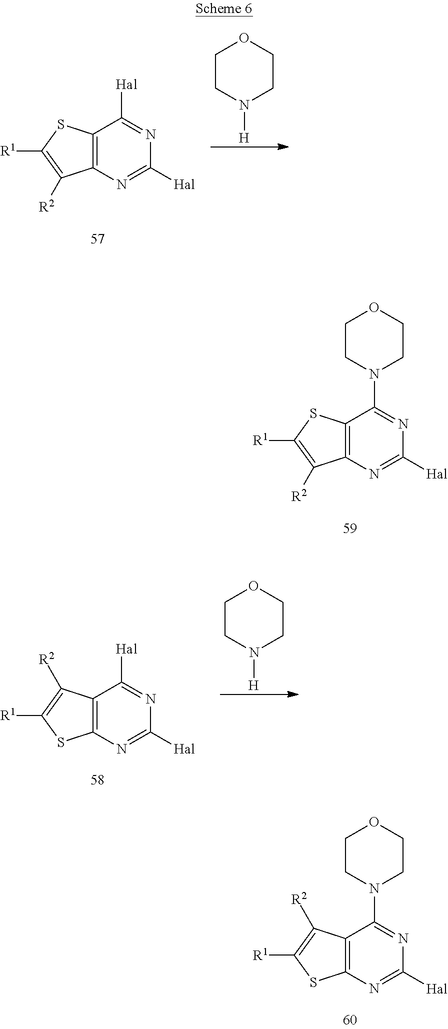

Scheme 6 shows a general method for selectively displacing a 4-halide from bis-halo thienopyrimidine intermediates 57 and 58 with morpholine under basic conditions in an organic solvent to prepare 2-halo, 4-morpholino thienopyrimidine compounds 59 and 60 respectively, wherein Hal is Cl, Br, or I; and R¹ and R² are as defined for Formulae V and VI compounds, or precursors or prodrugs thereto.

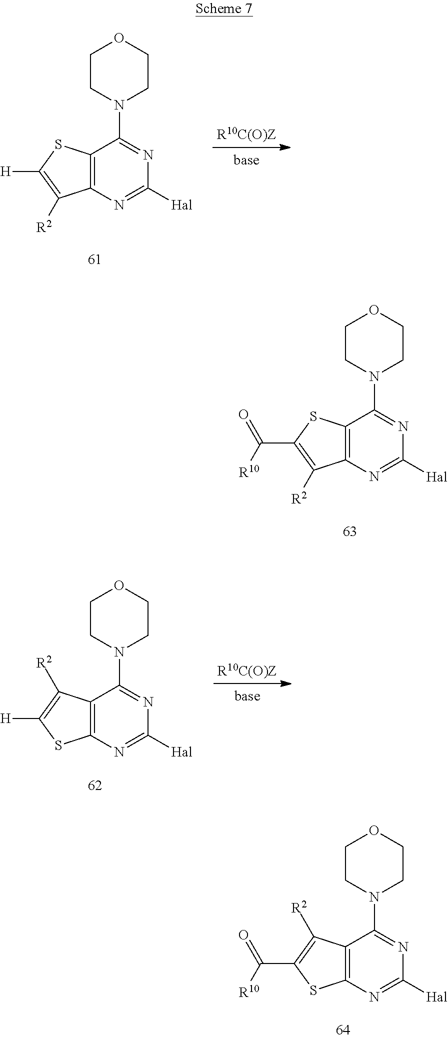

Scheme 7 shows a general method for derivatizing the 6-position of 2-halo, 4-morpholino, 6-hydrogen thienopyrimidine compounds 61 and 62 where R¹ is H. Treating 61 or 62 with a lithiating reagent to remove the 6 position proton, followed by adding an acylating reagent R¹⁰C(O)Z where Z is a leaving group, such as halide, NHS ester, carboxylate, or dialkylamino, gives 2-halo, 4-morpholino, 6-acyl thienopyrimidine compounds 63 and 64, wherein Hal is Cl, Br, or I; and R² and R¹⁰ are as defined for Formulae V and VI compounds, or precursors or prodrugs thereto. An example of R¹⁰C(O)Z to prepare 6-formyl compounds (R¹⁰═H) is N,N′-dimethylformamide (DMF).

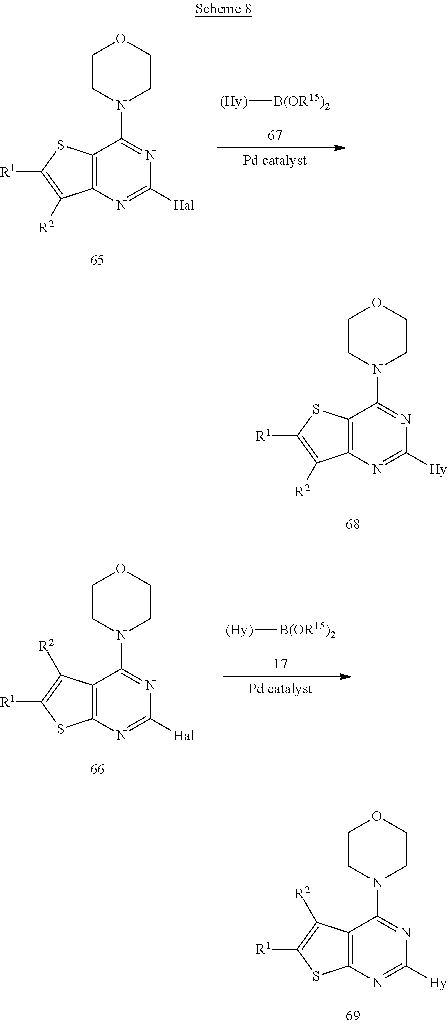

Scheme 8 shows a general method for Suzuki-type coupling of a 2-halo pyrimidine intermediate (65 and 66) with a monocyclic heteroaryl, fused bicyclic heterocyclyl or fused bicyclic heteroaryl boronate acid (R¹⁵═H) or ester (R¹⁵=alkyl) reagent 67 to prepare the 2-substituted (Hy), 4-morpholino thienopyrimidine compounds (68 and 69) of Formulae V and VI wherein Hal is Cl, Br, or I; and R¹ and R² are as defined for Formulae V and VI compounds, or precursors or prodrugs thereto. For reviews of the Suzuki reaction, see: Miyaura et al. (1995) Chem. Rev. 95:2457-2483; Suzuki, A. (1999) J. Organomet. Chem. 576:147-168; Suzuki, A. in Metal-Catalyzed Cross-Coupling Reactions, Diederich, F., Stang, P. J., Eds., VCH, Weinheim, DE (1998), pp 49-97. The palladium catalyst may be any that is typically used for Suzuki-type cross-couplings, such as PdCl₂(PPh₃)₂, Pd(PPh₃)₄, Pd(OAc)₂, PdCl₂(dppf)-DCM, Pd₂(dba)₃/Pt-Bu)₃ (Owens et al (2003) Bioorganic & Med. Chem. Letters 13:4143-4145; Molander et al (2002) Organic Letters 4(11):1867-1870; U.S. Pat. No. 6,448,433).

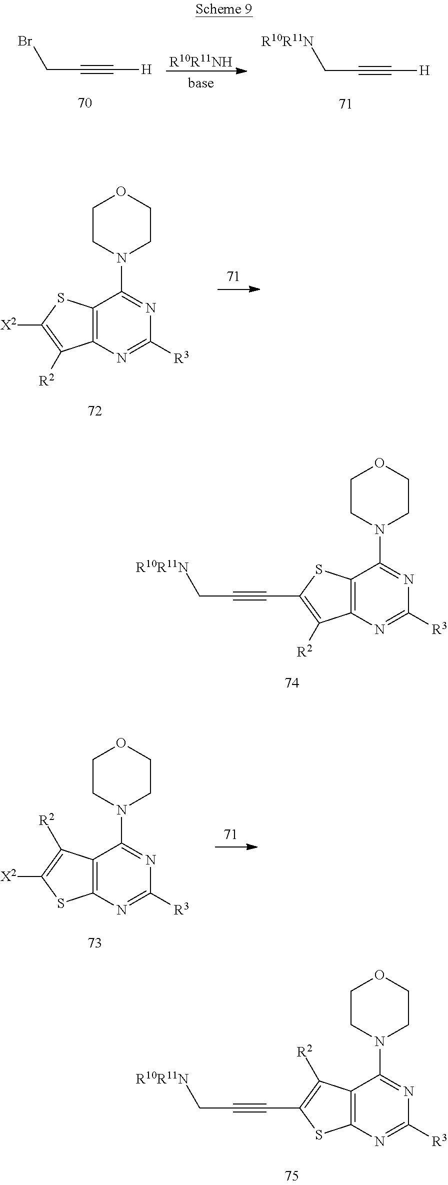

Scheme 9 shows a general method for the synthesis of alkynes 71, which can be used to prepare alkynylated derivatives of compounds 72 and 73. Propargylic amines 71 may be prepared by reaction of propargyl bromide 70 with an amine of the formula R¹⁰R¹¹NH (wherein R¹⁰ and R¹¹ are independently selected from H, alkyl, aryl and heteroaryl, or R¹⁰ and R¹¹ together with the nitrogen to which they are attached form a heterocyclic ring) in the presence of an appropriate base (Cs₂CO₃ or the like). For reviews of alkynyl amines and related syntheses see Booker-Milburn, K. I., Comprehensive Organic Functional Group Transformations (1995), 2:1039-1074; and Viehe, H. G., (1967) Angew. Chem., Int. Ed. Eng., 6(9):767-778. Alkynes 71 may subsequently be reacted with intermediates 72 (X²=bromo or iodo) or 73 (via Sonogashira coupling), to provide compounds 74 and 75, respectively, wherein R² and R³ are as defined for Formulae V and VI compounds, or precursors or prodrugs thereto.

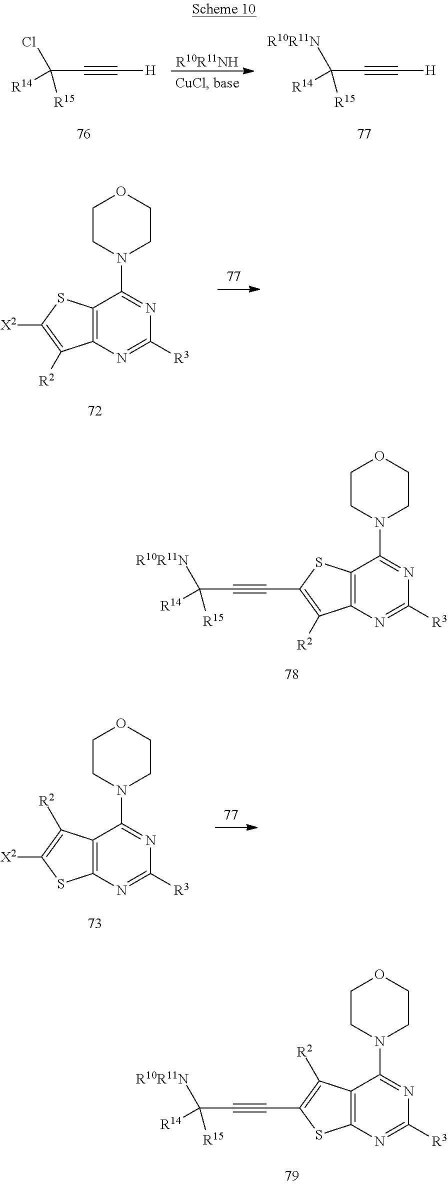

Scheme 10 shows a general method for the synthesis of alkynes 77, which can be used to prepare alkynylated derivatives of compounds 72 and 73. Gem-dialkyl propargylic amines 77 may be prepared using methods described by Zaragoza et al (2004) J. Med. Chem., 47:2833. According to Scheme 6, gem-dialkyl chloride 76 (R¹⁴ and R¹⁵ are independently methyl, ethyl or other alkyl group) can be reacted with an amine of the formula R¹⁰R¹¹NH (wherein R¹⁰ and R¹¹ are independently selected from H, alkyl, aryl and heteroaryl, or R¹⁰ and R¹¹ together with the nitrogen to which they are attached form a heterocyclic ring) in the presence of CuCl and an appropriate base (e.g. TEA or the like) to provide the alkyne 77. Alkyne 77 can be reacted with intermediates 72 or 73 (via Sonogashira coupling) to provide compounds 78 and 79, respectively, wherein R² and R³ are as defined for Formulae V and VI compounds, or precursors or prodrugs thereto.

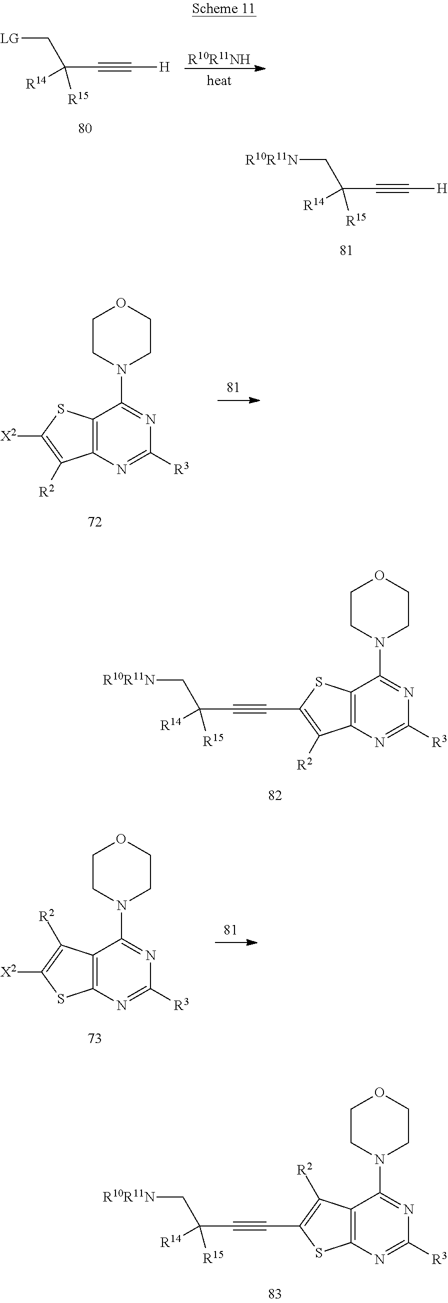

Scheme 11 shows a general scheme for the synthesis of alkynes 81, which can be used to prepare alkynylated derivatives of compounds 72 and 73. But-3-yn-1-amines 81 (wherein R¹⁴ and R¹⁵ are independently H, alkyl, aryl, heteroaryl, or R¹⁴ and R¹⁵ together with the carbon atom to which they are attached form a carbocyclic or heterocyclic ring) can be prepared from reaction of alkynes 80 (LG=tosylate or other leaving group) with an amine of the formula R¹⁰R¹¹NH (wherein R¹⁰ and R¹¹ are independently selected from H, alkyl, aryl and heteroaryl, or R¹⁰ and R¹¹ together with the nitrogen to which they are attached form a heterocyclic ring) using the protocol described by Olomucki M. et al (1960) Ann. Chim. 5:845. Alkynes 81 can subsequently be reacted with intermediates 72 or 73 (via Sonogashira coupling), according to the descriptions provided for Schemes 5 and 6 to provide compounds 82 and 83, respectively, wherein R² and R³ are as defined for Formulae V and VI compounds, or precursors or prodrugs thereto.

A pharmaceutically acceptable salt of a thienopyrimidine compound of Formula I to VI may be prepared using conventional techniques. Typically the process comprises treating the thienopyrimidine of Formula I as defined above with a suitable acid in a suitable solvent.

In the process of the invention as defined above, both the amination step and the Pd-mediated cross-coupling step take place under conventional conditions. The palladium catalyst may be any that is typically used for Suzuki-type cross-couplings, such as PdCl₂(PPh₃)₂. The reducing agent is typically a borohydride, such as NaBH(OAc)₃, NaBH₄ or NaCNBH₄.

### Methods of Treating Neoplasms

An embodiment includes a method of treating a neoplasm in a mammal comprising, administering a combination of (i) an inhibitor of a kinase, wherein said inhibitor induces autophagy, and (ii) an inhibitor of autophagy in an amount effective to treat said neoplasm. The inhibitor of a kinase and the inhibitor of autophagy can be administered together or separately, at the same time or at different times. In an embodiment, the inhibitor of kinase that induces autophagy and said inhibitor of autophagy are present in synergistically effective amounts.

In another embodiment, the method of treating a neoplasm in a mammal comprising, administering a combination of (i) an inhibitor of a kinase, wherein said inhibitor induces autophagy, and (ii) an inhibitor of autophagy in an amount effective to treat said neoplasm, further comprises administering a protease inhibitor. Protease inhibitors are well known in the art. In one embodiment, the protease inhibitor inhibits lysosomal cysteine protease activity or aspartic proteases, such as pepstatin A. The inhibitor of a kinase, inhibitor of autophagy and the protease inhibitor can be administered singly, or in any combination together or separately, at the same time or at different times.

Methods of blocking or reducing relapse tumor growth or a relapse cancer cell growth are also provided. In certain embodiments of the invention, the subject was, or is concurrently undergoing cancer therapy. The administration of the combination therapy described herein blocks or reduces relapse tumor growth or relapse cancer cell growth.

Another embodiment provides, a method of inducing apoptosis in a cancer cell comprising administering to said cell (i) an inhibitor of a kinase, wherein said inhibitor induces autophagy, and (ii) an inhibitor of autophagy in an amount effective to induce said apoptosis. In one example, the effective amount of said kinase inhibitor and/or inhibitor of autophagy produces a synergistic apoptosis inducing effect. In another example, the effective amount of said kinase and/or said inhibitor of autophagy has an ED50, ED75 or ED90 that is lower than the ED50, ED75 or ED90 of the kinase inhibitor or inhibitor of autophagy alone. In one example, the kinase inhibitor and inhibitor of autophagy are given in ratios in the range of about 2:1 to about 1:50, alternatively about 1.25:1 to about 1:12, alternatively about 1:1 to about 1:5. In one example, III-4 is dosed in combination with CQ in a ratio of about 1:25, 1:12.5, 1:1.5, or 1.3:1.

When any variable occurs more than one time in any constituent or in Formula I, II, III, IV, V or VI, its definition on each occurrence is independent of its definition at every other occurrence. Also combinations of substituents and/or variables are permissible only if such combinations result in allowable valences.

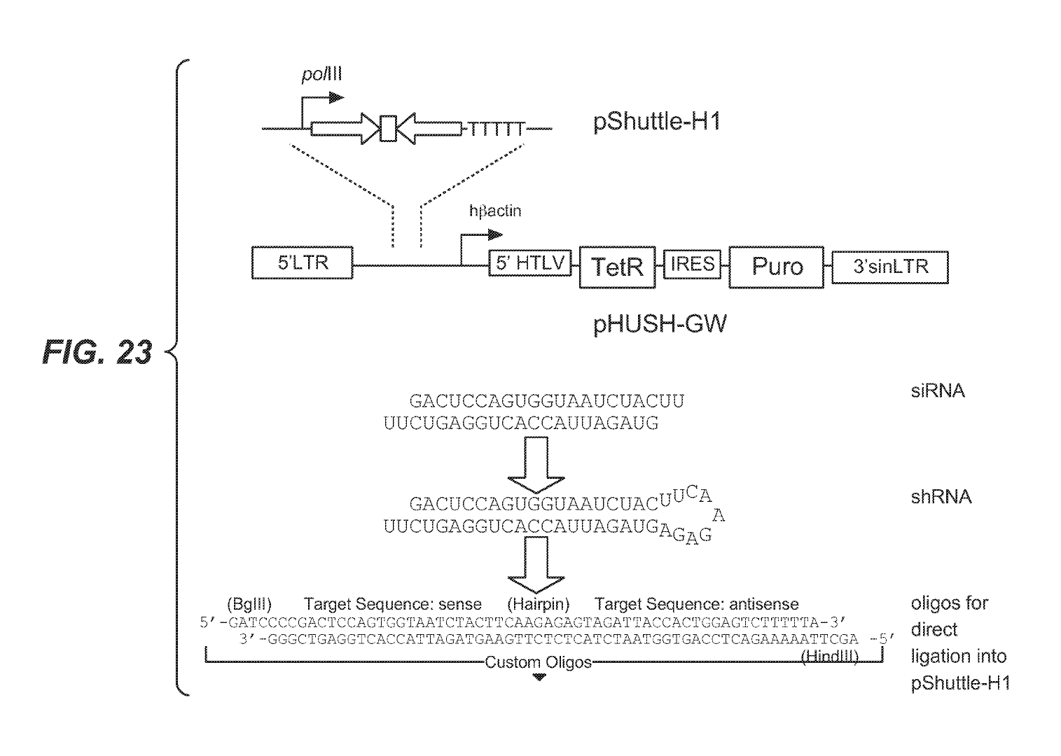

The method of treating a neoplasm described herein can comprise administering an inhibitor of kinase that induces autophagy wherein the inhibitor is an RNA interference (RNAi) construct in combination with an inhibitor of autophagy. FIG. 23 shows that such an RNAi construct can comprise RNA, DNA or DNA that is transcribed to RNA. Preferably, the use of an RNAi construct in the present methods in combination with an autophagy inhibitor results in a synergistic killing or inhibitory effect on a neoplasm.

### RNA Constructs

In another embodiment, the subject matter disclosed herein relates to RNAi constructs described herein. The RNAi constructs are useful inhibitors of Akt.

As used herein, an RNAi construct includes shRNA, siRNA, DNA directed shRNA and siRNA, as well as the DNA itself, DNA oligos and vectors described herein. In certain embodiments, siRNA or shRNA is transcribed from an RNAi construct comprising a nucleic acid sequence substantially corresponding to a target sequence in one or more Akt genes. Preferably, the sequence is selected from SEQ ID Nos: 39-48 and combinations thereof. When introduced to a cell, these RNAi constructs are capable of reducing the expression of one or more Akt proteins. Reducing the expression of a protein means that the expression is lower in a cell than it would be if the RNAi construct had not been introduced. Methods for detecting levels of expression are described herein or known in the art. The RNAi constructs can reduce the expression of AKT isoforms including Akt1, Akt2, Akt3 and combinations thereof.

The RNAi constructs can comprise one or more DNA sequences substantially corresponding to a sequence selected from the group consisting of SEQ ID Nos: 1-18. DNA sequences can be synthesized and cloned into a shuttle as described herein. In another embodiment, an RNAi construct capable of reducing the expression of one or more Akt proteins comprises a RNA sequence substantially corresponding to a sequence selected from the group consisting of SEQ ID Nos: 19-38 and combinations thereof. In another embodiment, the RNAi constructs can comprise a sense RNA strand and a substantially complementary antisense RNA strand, wherein the antisense strand comprises one or more sequence substantially corresponding to a sequence selected from SEQ ID Nos: 20, 22, 24, 26, 28, 30, 32, 34, 36 and 38, wherein the sense and antisense strands are annealed as a RNA duplex. The duplex can comprise a sense strand comprising one or more sequences substantially corresponding to a sequence selected from the group consisting of SEQ ID Nos: 19, 21, 23, 25, 27, 29, 31, 33, 35 and 37. The sense and antisense strands can be annealed to form the duplex in the pair combinations that include the following: SEQ ID Nos: 19:20, 21:22, 23:24, 25:26, 27:28, 29:30, 31:32, 33:34, 35:36 and 37:38 and combinations that include more than one of the pairs. The RNAi construct can contain a hairpin that covalently links the sense strand and the antisense strand.

In another embodiment, RNAi constructs that are capable of reducing the expression of one or more Akt proteins are described herein. Non-limiting examples of such RNAi constructs include a construct comprising a nucleotide sequence substantially corresponding to SEQ ID No: 32, and additionally comprising a sequence substantially corresponding to a sequence selected from the group consisting of SEQ ID Nos: 22, 26 and 36. Another non-limiting example includes a nucleotide sequence substantially corresponding to SEQ ID No: 31, and additionally a sequence substantially corresponding to a sequence selected from the group consisting of SEQ ID Nos: 21, 25 and 35. Other combinations which lower expression of target Akt isoforms can be readily obtained from the present disclosure.

Another embodiment is directed to an RNAi construct capable of reducing the expression of an Akt gene, wherein the construct is a substrate for a Dicer. Yet another embodiment is directed to an isolated nucleotide or nucleic acid sequence as described herein. An RNAi construct as described herein can be prepared by any known method. (McIntyre, G J, and Fanning G C, BMC Biotechnology (2006), 6:1).

### Pharmaceutical Formulations

Pharmaceutical compositions or formulations of the present invention include combinations of compounds of Formula I to VI, and other compounds described herein, a inhibitor of autophagy, and one or more pharmaceutically acceptable carrier, glidant, diluent, or excipient.

The compounds of Formula I to VI, and other compounds described herein, and inhibitors of autophagy of the present invention may exist in unsolvated as well as solvated forms with pharmaceutically acceptable solvents such as water, ethanol, and the like, and it is intended that the invention embrace both solvated and unsolvated forms.

The compounds of Formula I to VI, and other compounds described herein, and inhibitors of autophagy of the present invention may also exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Pharmaceutical compositions encompass both the bulk composition and individual dosage units comprised of more than one (e.g., two) pharmaceutically active agents including a Formula I to VI compound and a inhibitor of autophagy selected from the lists of the additional agents described herein, along with any pharmaceutically inactive excipients, diluents, carriers, or glidants. The bulk composition and each individual dosage unit can contain fixed amounts of the aforesaid pharmaceutically active agents. The bulk composition is material that has not yet been formed into individual dosage units. An illustrative dosage unit is an oral dosage unit such as tablets, pills, capsules, and the like. Similarly, the herein-described method of treating a patient by administering a pharmaceutical composition of the present invention is also intended to encompass the administration of the bulk composition and individual dosage units.

Pharmaceutical compositions also embrace isotopically-labeled compounds of the present invention which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Certain isotopically-labeled compounds of the present invention (e.g., those labeled with ³H and ¹⁴C) are useful in compound and/or substrate tissue distribution assays. Tritiated (³H) and carbon-14 (¹⁴C) isotopes are useful for their ease of preparation and detectability. Further, substitution with heavier isotopes such as deuterium (²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

The compounds of Formula I to VI, and other compounds described herein, and inhibitors of autophagy are formulated in accordance with standard pharmaceutical practice for use in a therapeutic combination for therapeutic treatment (including prophylactic treatment) of hyperproliferative disorders in mammals including humans. The invention provides a pharmaceutical composition comprising a Formula I to VI compound in association with one or more pharmaceutically acceptable carrier, glidant, diluent, or excipient.

Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration. For example, a compound of Formula I to VI, or another compound described herein, having the desired degree of purity may optionally be mixed with pharmaceutically acceptable diluents, carriers, excipients or stabilizers (Remington's Pharmaceutical Sciences (1995) 18th edition, Mack Publ. Co., Easton, Pa.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8.

The pharmaceutical formulation is preferably sterile. In particular, formulations to be used for in vivo administration must be sterile. Such sterilization is readily accomplished by filtration through sterile filtration membranes.

The pharmaceutical formulation ordinarily can be stored as a solid composition, a lyophilized formulation or as an aqueous solution.

The pharmaceutical formulations of the invention will be dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

As a general proposition, the initial pharmaceutically effective amount of the compound of Formula I to VI, or another compound described herein, administered orally or parenterally per dose will be in the range of about 0.01-100 mg/kg, namely about 0.1 to 20 mg/kg of patient body weight per day, with the typical initial range of compound used being 0.3 to 15 mg/kg/day.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition, (1995) Mack Publ. Co., Easton, Pa.

Sustained-release preparations of the compounds of Formula I to VI, and other compounds described herein, may be prepared. Suitable examples of sustained-release preparations include semipermeable matrices of solid hydrophobic polymers containing a compound of Formula I, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D (−) 3-hydroxybutyric acid.

The pharmaceutical formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences 18^{th} Ed. (1995) Mack Publishing Co., Easton, Pa. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of compounds of Formula I to VI, and other compounds described herein, and inhibitors of autophagy suitable for oral administration may be prepared as discrete units such as pills, hard or soft e.g., gelatin capsules, cachets, troches, lozenges, aqueous or oil suspensions, dispersible powders or granules, emulsions, syrups or elixirs each containing a predetermined amount of a compound of Formula I to VI, or another compound described herein, and a inhibitor of autophagy. Such formulations may be prepared according to any method known to the art for the manufacture of pharmaceutical compositions and such compositions may contain one or more agents including sweetening agents, flavoring agents, coloring agents and preserving agents, in order to provide a palatable preparation. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablet excipients of a pharmaceutical formulation of the invention may include: Filler (or diluent) to increase the bulk volume of the powdered drug making up the tablet; Disintegrants to encourage the tablet to break down into small fragments, ideally individual drug particles, when it is ingested and promote the rapid dissolution and absorption of drug; Binder to ensure that granules and tablets can be formed with the required mechanical strength and hold a tablet together after it has been compressed, preventing it from breaking down into its component powders during packaging, shipping and routine handling; Glidant to improve the flowability of the powder making up the tablet during production; Lubricant to ensure that the tableting powder does not adhere to the equipment used to press the tablet during manufacture. They improve the flow of the powder mixes through the presses and minimize friction and breakage as the finished tablets are ejected from the equipment; Antiadherent with function similar to that of the glidant, reducing adhesion between the powder making up the tablet and the machine that is used to punch out the shape of the tablet during manufacture; Flavor incorporated into tablets to give them a more pleasant taste or to mask an unpleasant one, and Colorant to aid identification and patient compliance.

Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions of this invention may be constituted from known ingredients in a known manner, including a mixture of at least one emulsifier with a fat or an oil, or with both a fat and an oil. Preferably, a hydrophilic emulsifier is included together with a lipophilic emulsifier which acts as a stabilizer. Together, the emulsifier(s) with or without stabilizer(s) make up an emulsifying wax, and the wax together with the oil and fat comprise an emulsifying ointment base which forms the oily dispersed phase of cream formulations. Emulsifiers and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

Aqueous suspensions of the pharmaceutical formulations of the invention contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

Pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may be a solution or a suspension in a non-toxic parenterally acceptable diluent or solvent, such as a solution in 1,3-butanediol or prepared from a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below.

Formulations suitable for vaginal administration may be presented as pessaries, tampons, creams, gels, pastes, foams or spray formulations containing in addition to the active ingredient such carriers as are known in the art to be appropriate.

It is further contemplated that an agent of the invention (e.g., DNA, RNAi, shRNA, siRNA, kinase inhibitor, chemotherapeutic agent or anti-cancer agent) can be introduced to a subject by gene therapy. Gene therapy refers to therapy performed by the administration of a nucleic acid to a subject. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 (1986)). The oligonucleotides can be modified to enhance their uptake, e.g. by substituting their negatively charged phosphodiester groups by uncharged groups. For general reviews of the methods of gene therapy, see, for example, Goldspiel et al. Clinical Pharmacy 12:488-505 (1993); Wu and Wu Biotherapy 3:87-95 (1991); Tolstoshev Ann. Rev. Pharmacol. Toxicol. 32:573-596 (1993); Mulligan Science 260:926-932 (1993); Morgan and Anderson Ann. Rev. Biochem. 62:191-217 (1993); and May TIBTECH 11:155-215 (1993). Methods commonly known in the art of recombinant DNA technology which can be used are described in Ausubel et al. eds. (1993) Current Protocols in Molecular Biology, John Wiley & Sons, NY; and Kriegler (1990) Gene Transfer and Expression, A Laboratory Manual, Stockton Press, NY.

In one embodiment, the RNAi constructs or DNA for forming the RNA constructs of the invention are delivered to cell(s) for treatment, and may be delivered in combination with inhibitors of autophagy. There are two major approaches to getting the DNA/RNA (optionally contained in a vector) into the patient's cells; in vivo and ex vivo. For in vivo delivery the DNA/RNA is injected directly into the patient, usually at the site where the DNA/RNA is required. For ex vivo treatment, the patient's cells are removed, the DNA/RNA is introduced into these isolated cells and the modified cells are administered to the patient either directly or, for example, encapsulated within porous membranes which are implanted into the patient (see, e.g., U.S. Pat. Nos. 4,892,538 and 5,283,187). There are a variety of techniques available for introducing nucleic acids into viable cells. The techniques vary depending upon whether the oligonucleotide is transferred into cultured cells in vitro, or in vivo in the cells of the intended host. Techniques suitable for the transfer of oligonucleotides into mammalian cells in vitro include the use of liposomes, electroporation, microinjection, cell fusion, DEAE-dextran, the calcium phosphate precipitation method, etc. A commonly used vector for ex vivo delivery of the gene is a retroviral vector.

Example in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). For review of the currently known gene marking and gene therapy protocols see Anderson et al., Science 256:808-813 (1992). See also WO 93/25673 and the references cited therein. Examples of using viral vectors in gene therapy can be found in Clowes et al. J. Clin. Invest. 93:644-651 (1994); Kiem et al. Blood 83:1467-1473 (1994); Salmons and Gunzberg Human Gene Therapy 4:129-141 (1993); Grossman and Wilson Curr. Opin. in Genetics and Devel. 3:110-114 (1993); Bout et al. Human Gene Therapy 5:3-10 (1994); Rosenfeld et al. Science 252:431-434 (1991); Rosenfeld et al. Cell 68:143-155 (1992); Mastrangeli et al. J. Clin. Invest. 91:225-234 (1993); and Walsh et al. Proc. Soc. Exp. Biol. Med. 204:289-300 (1993).

In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Combination Therapy

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and may be lowered due to the combined action (synergy) of the newly identified agent and other inhibitors of autophagy or treatments.

In a particular embodiment of anti-cancer therapy, a compound of Formula I to VI, or other compounds described herein, or a stereoisomer, geometric isomer, tautomer, solvate, metabolite, or pharmaceutically acceptable salt thereof, is combined with an inhibitor of autophagy, and further combined with surgical therapy and radiotherapy. The amounts of the compound(s) of Formula I to VI, or other compounds described herein, and the inhibitor(s) of autophagy, and the relative timings of administration will be selected in order to achieve the desired combined therapeutic effect. In an embodiment, the therapeutic effect is a synergistic effect.

The compounds of the invention may be administered by any route appropriate to the condition to be treated. Suitable routes include oral, parenteral (including subcutaneous, intramuscular, intravenous, intraarterial, inhalation, intradermal, intrathecal, epidural, and infusion techniques), transdermal, rectal, nasal, topical (including buccal and sublingual), vaginal, intraperitoneal, intrapulmonary and intranasal. Topical administration can also involve the use of transdermal administration such as transdermal patches or iontophoresis devices. Formulation of drugs is discussed in Remington's Pharmaceutical Sciences, 18^{th} Ed., (1995) Mack Publishing Co., Easton, Pa. Other examples of drug formulations can be found in Liberman, H. A. and Lachman, L., Eds., Pharmaceutical Dosage Forms, Marcel Decker, Vol 3, 2^{nd} Ed., New York, N.Y. For local immunosuppressive treatment, the compounds may be administered by intralesional administration, including perfusing or otherwise contacting the graft with the inhibitor before transplantation. It will be appreciated that the preferred route may vary with for example the condition of the recipient. Where the compound is administered orally, it may be formulated as a pill, capsule, tablet, etc. with a pharmaceutically acceptable carrier, glidant, or excipient. Where the compound is administered parenterally, it may be formulated with a pharmaceutically acceptable parenteral vehicle or diluent, and in a unit dosage injectable form, as detailed below.

A dose to treat human patients may range from about 10 mg to about 1000 mg of Formula I to VI compound. A typical dose may be about 100 mg to about 300 mg of the compound. A dose may be administered once a day (QID), twice per day (BID), or more frequently, depending on the pharmacokinetic (PK) and pharmacodynamic (PD) properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

### Articles of Manufacture

Kits of combinations of inhibitors of a kinase that induces autophagy and inhibitors of autophagy are also provided. In certain embodiments, a kit includes inhibitors of a kinase that induce autophagy and inhibitors of autophagy, a pharmaceutically acceptable carrier, vehicle, or diluent, and a container. Instructions for use can also be included.

In another embodiment of the invention, an article of manufacture, or “kit”, containing compounds of Formulae I to VI useful for the treatment of the diseases and disorders described above is provided. In one embodiment, the kit comprises a container comprising a compound of Formula I, or a stereoisomer, geometric isomer, tautomer, solvate, metabolite, or pharmaceutically acceptable salt thereof. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a compound of Formula I to VI or a formulation thereof which is effective for treating the condition and may have a sterile access port (for example, the container may be an intravenous solution bag or a vial having a stopper pierceable by a hypodermic injection needle). At least one active agent in the composition is a compound of Formula I to VI, or a compound described herein. The label or package insert indicates that the composition is used for treating the condition of choice, such as cancer. In one embodiment, the label or package inserts indicates that the composition comprising a compound of Formula I to VI, or a compound described herein, can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the compound of Formula I to VI, or a compound described herein, and, if present, the second pharmaceutical formulation. For example, if the kit comprises a first composition comprising a compound of Formula I to VI, or a compound described herein, and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of a compound of Formula I to VI, or a compound described herein, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with a compound of Formula I to VI, or a compound described herein, contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Where the kit comprises a composition of Formula I to VI, or a compound described herein, and a second therapeutic agent, i.e. the inhibitor of autophagy, the kit may comprise a container for containing the separate compositions such as a divided bottle or a divided foil packet; however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

Kinase Inhibition that Induces Autophagy

Data provided herein, show that Akt inhibition, and likewise, inhibition of other certain kinases, does not always induce a clear apoptotic response. Autophagy is a readily detectable response to pan-Akt knockdown or inhibition, Akt-isoform selective knockdown or inhibition, or small molecule inhibitors of the Akt, PI3K, mTOR, PDK1 or p70S6K pathways. Kinase-inhibition-induced autophagy may sensitize tumor cells to agents targeting this lysosomal degradation pathway. Indeed, agents that block the lysosomal degradation function could precipitate cell death when combined with kinase inhibitors that induce autophagy and promote complete tumor remissions in preclinical models Inhibiting, slowing or blocking the autophagic response may be a promising strategy to increase the therapeutic efficacy of kinase inhibitors that induce autophagy, e.g., Akt, PI3K, mTOR, PDK-1 and p70S6K inhibitors.

Autophagy is a more sensitive response to Akt inhibition, for example, than apoptosis in cancer cell lines. Numerous reports have documented deregulation of the phosphatidylinositol 3-kinase (PI3K)/Akt pathway in a variety of cancers, leading not only to uncontrolled growth and proliferation, but also to resistance to various cell death stimuli. (Manning B D, Cantley L C. AKT/PKB signaling: navigating downstream. Cell 2007; 129:1261-74; Samuels Y, Ericson K. Oncogenic PI3K and its role in cancer. Curr Opin Oncol 2006; 18:77-82). Thus, targeting, for example Akt, the serine/threonine kinase at the central node of this pathway, or other kinases for which inhibition induces autophagy, may inhibit both growth and survival of the malignant cells.

Although Akt is believed to play a critical role in protecting cells from programmed cell death following various pro-apoptotic insults (Manning B D, et al.; Downward J. Mechanisms and consequences of activation of protein kinase B/Akt. Curr Opin Cell Biol 1998; 10:262-7), it remains to be determined whether apoptosis is a prevailing response to inhibiting Akt activity alone. RNA interference techniques that specifically knockdown each of the three Akt isoforms as well as specific inhibitors result in a significant proportion of cancer cell lines examined do not readily undergo apoptosis even when all three Akt isoforms are greatly reduced. (Koseoglu S, Lu Z, Kumar C, Kirschmeier P, Zou J. AKT1, AKT2 and AKT3-dependent cell survival is cell line-specific and knockdown of all three isoforms selectively induces apoptosis in 20 human tumor cell lines. Cancer Biol Ther 2007; 6:755-62). This is consistent with the report that only a small portion of total Akt activity is required for apoptosis inhibition in mouse embryonic fibroblast (MEF) cells. (Liu X, Shi Y, Birnbaum M J, Ye K, De Jong R, Oltersdorf T, Giranda V L, Luo Y. Quantitative analysis of anti-apoptotic function of Akt in Akt1 and Akt2 double knock-out mouse embryonic fibroblast cells under normal and stressed conditions. J Biol Chem 2006; 281:31380-8). The sensitivity of cancer cells to apoptosis induction upon Akt inhibition is likely dependent on both their genetic background and environmental conditions. For example, although a higher level of activated Akt may suggest a relative reliance of tumor cells on this pathway and may be a slightly better predictor of apoptotic response to Akt inhibition, resistance to apoptosis is also observed in cells with Akt activation, including those with loss of phosphatase and tensin homolog (PTEN), a tumor suppressor that negatively regulates PI3K/Akt activity. (Koseoglu S, et al). It is conceivable that apoptosis can be suppressed by multiple mechanisms in advanced cancer cells as a result of their evolution through stringent selection pressure.

In contrast, although both PTEN-null cell lines PC3 and U87MG are resistant to apoptosis in response to inducible shRNA knockdown of all three Akt isoforms (shAkt123), data show significantly elevated autophagy in both cell lines. Autophagy appears to be a more sensitive response to reduced Akt activity caused by either specific shRNA knockdown or selective inhibitors of the pathway in a variety of cell lines, whether or not apoptosis is induced in these cells (Degtyarev and Lin, this work and unpublished data).

Blocking autophagic degradation accelerated cell death in combination with Akt inhibition. Autophagy has been implicated both as a mechanism of cell death and as a cytoprotective process, depending on the circumstances and cellular contexts. (Scarlatti F, Granata R, Meijer A J, Codogno P. Does autophagy have a license to kill mammalian cells? 2008). However, PC3 cells expressing shAkt123 can survive for a prolonged period of time without significant loss of viability, even under reduced serum conditions. When grown as xenograft tumors, although continuous expression of shAkt123 could effectively inhibit tumor growth initially, most of the tumors failed to regress completely and eventually overcame the inhibition and rebounded within 2-3 weeks. These suggest that autophagy induced by inhibiting Akt alone does not effectively eliminate cancer cells under these conditions.

Because autophagy is a more sensitive response to Akt knockdown or small molecule inhibitors, blocking effective autophagy could accelerate cell death in combination with Akt inhibition. The lysosomotropic agent chloroquine (CQ) significantly accelerated death rate in cells either expressing shAkt123 or treated with relatively specific small molecule inhibitors of the pathway, PI-103 (a PI3K/mTOR inhibitor that is 1,000× more potent on class I than class III PI3K) (Knight Z A, Gonzalez B, Feldman M E, Zunder E R, Goldenberg D D, Williams O, Loewith R, Stokoe D, Balla A, Toth B, Balla T, Weiss W A, Williams R L, Shokat K M. A pharmacological map of the PI3-K family defines a role for p110alpha in insulin signaling. Cell 2006; 125:733-47) and Akti-1/2 (a selective dual Akt1,2 inhibitor) (Barnett S F, Defeo-Jones D, Fu S, Hancock P J, Haskell K M, Jones R E, Kahana J A, Kral A M, Leander K, Lee L L, Malinowski J, McAvoy E M, Nahas D D, Robinson R G, Huber H E. Identification and characterization of pleckstrin-homology-domain-dependent and isoenzyme-specific Aid inhibitors. Biochem J 2005; 385:399-408), both of which induced overt autophagy. Similar results were obtained with Bafilomycin A1, an inhibitor of vacuolar proton pump (V—H⁺-ATPase) that impairs lysosomal acidification. (Yamamoto A, Tagawa Y, Yoshimori T, Moriyama Y, Masaki R, Tashiro Y. Bafilomycin A1 prevents maturation of autophagic vacuoles by inhibiting fusion between autophagosomes and lysosomes in rat hepatoma cell line, H-4-II-E cells. Cell Struct Funct 1998; 23:33-42). Synergistic growth inhibitory effect of CQ and Akt inhibitors have also been observed in an expanded panel of cancer cell lines.

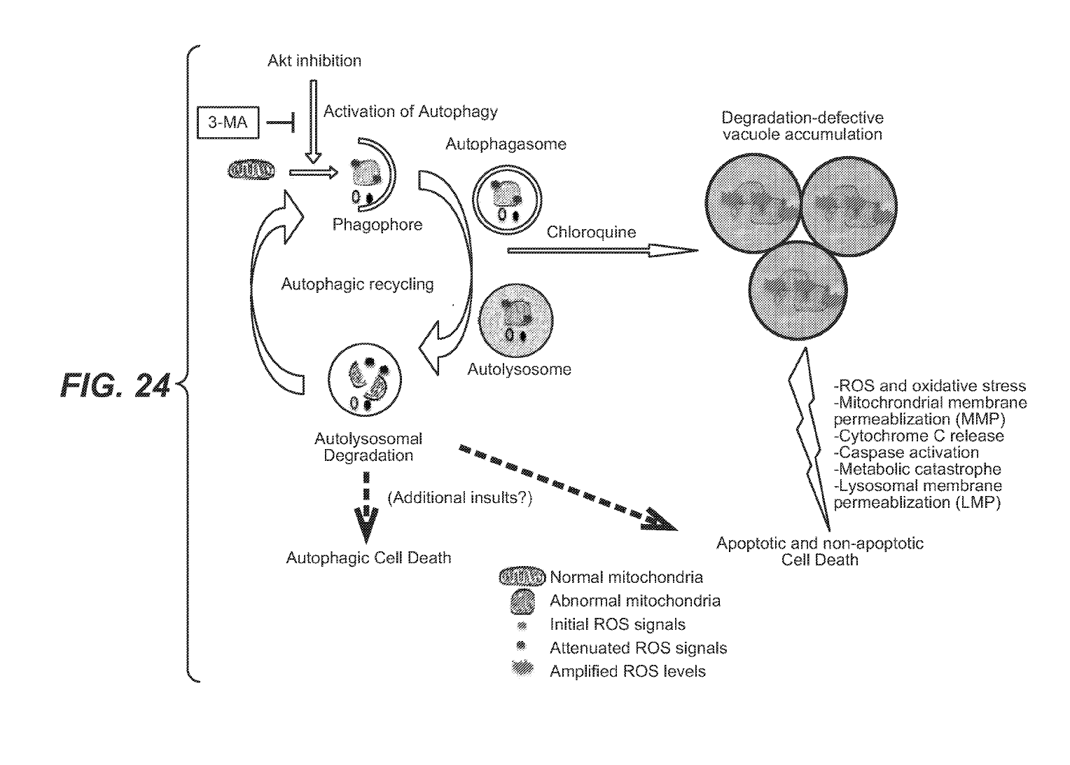

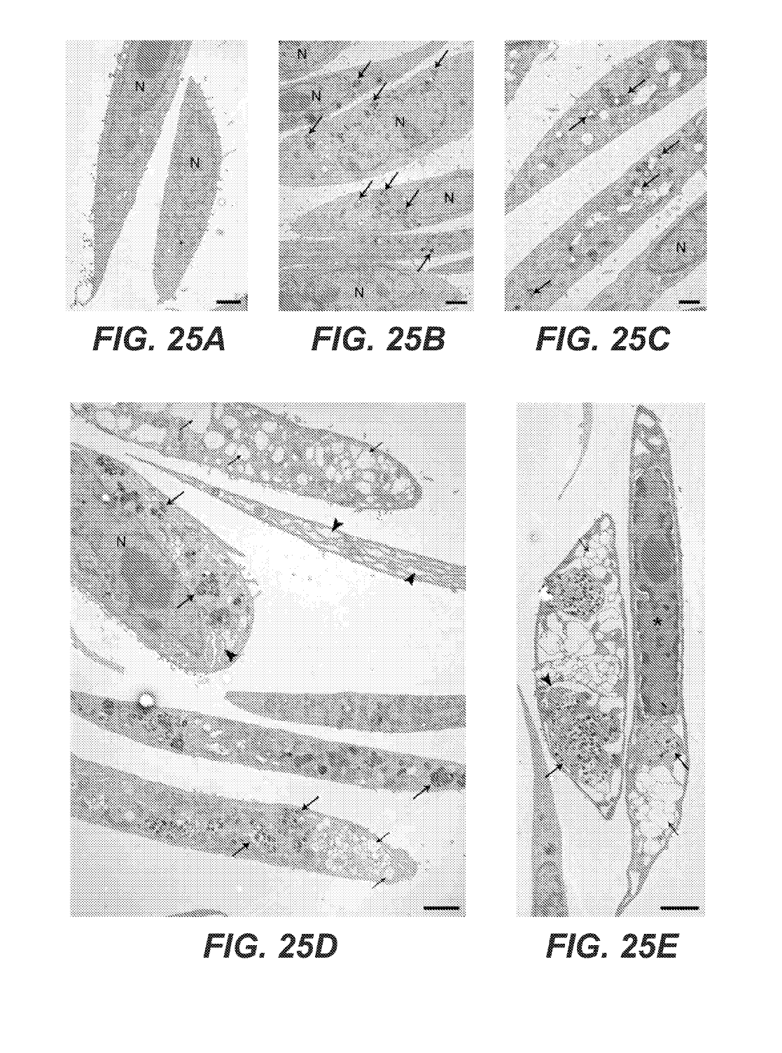

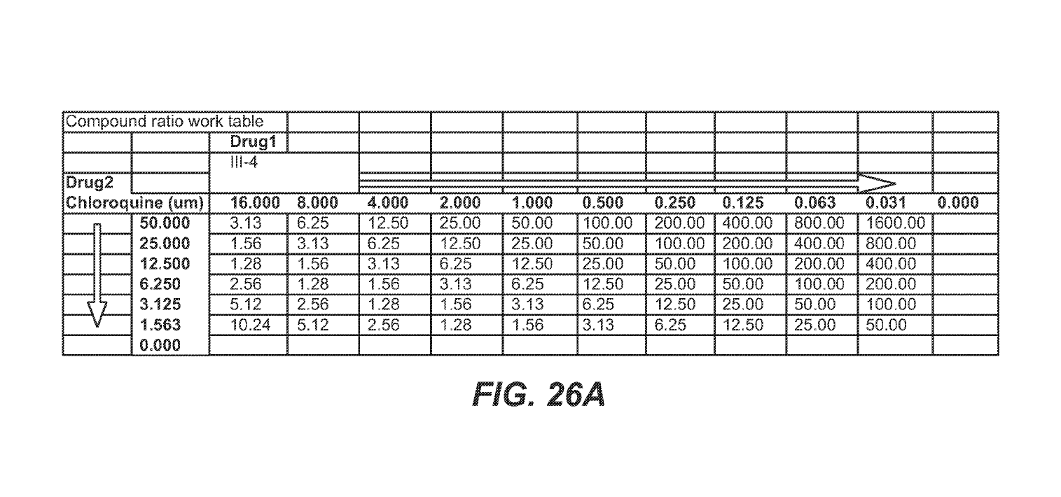

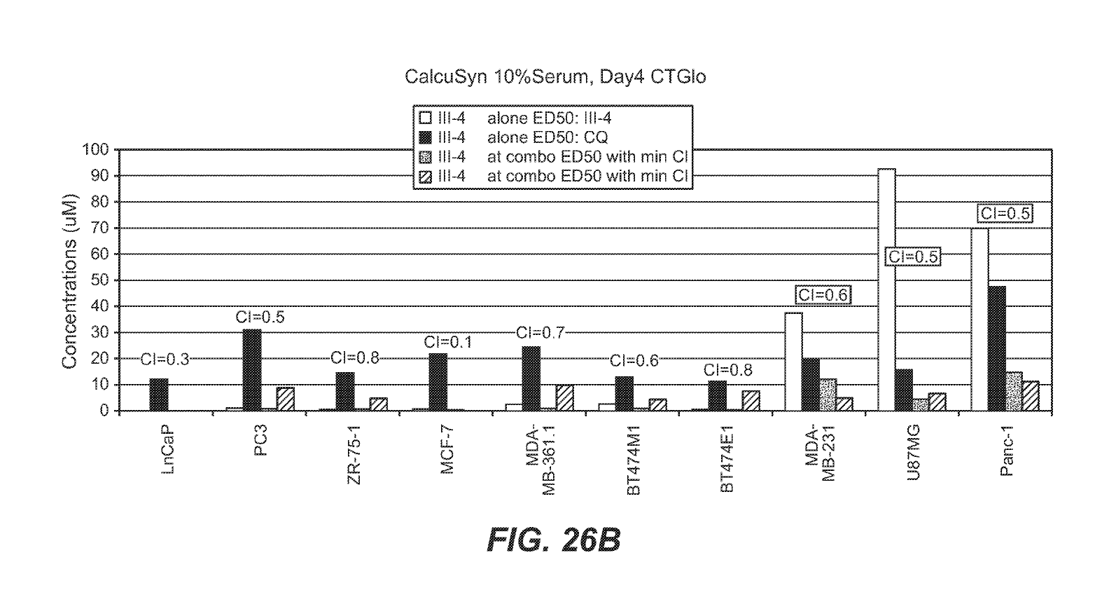

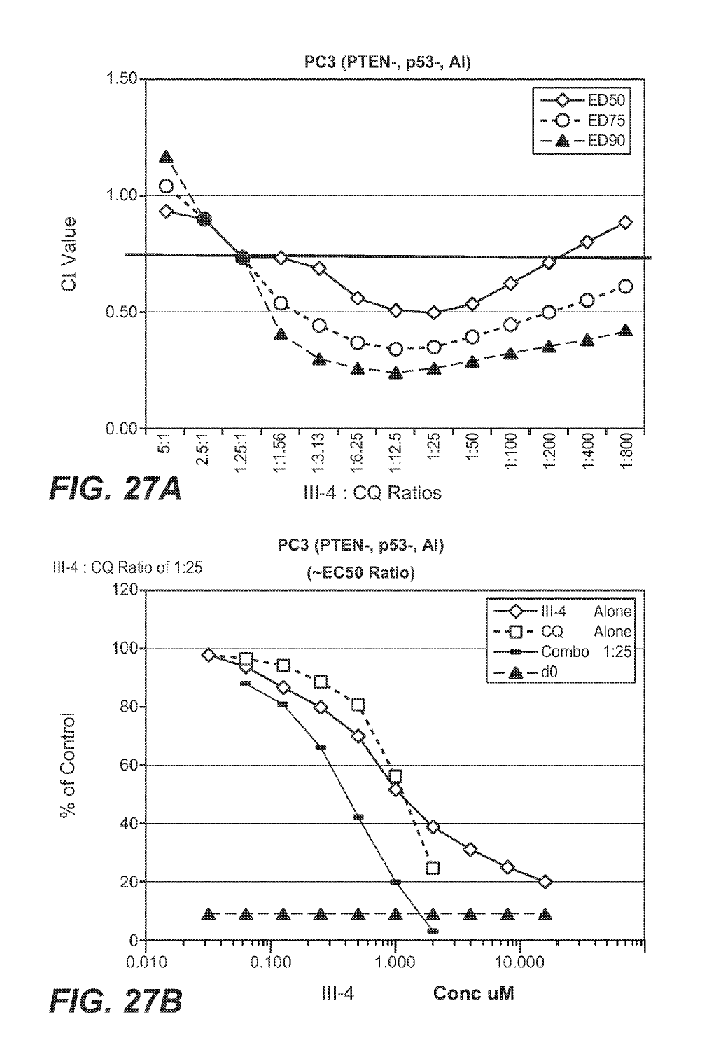

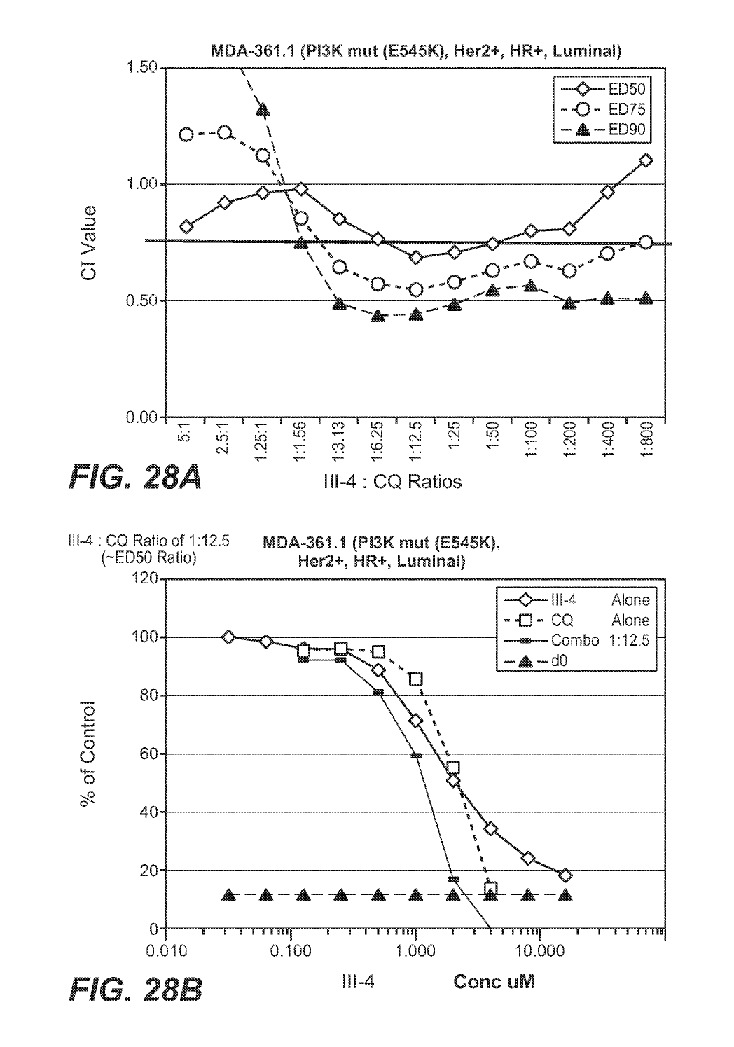

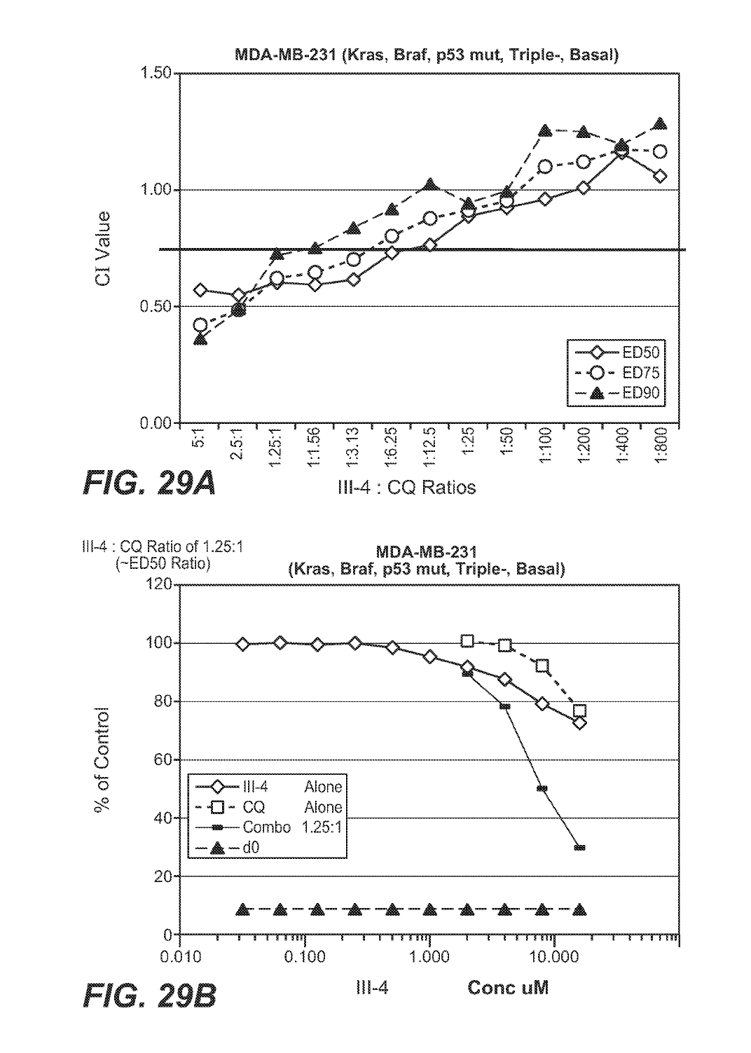

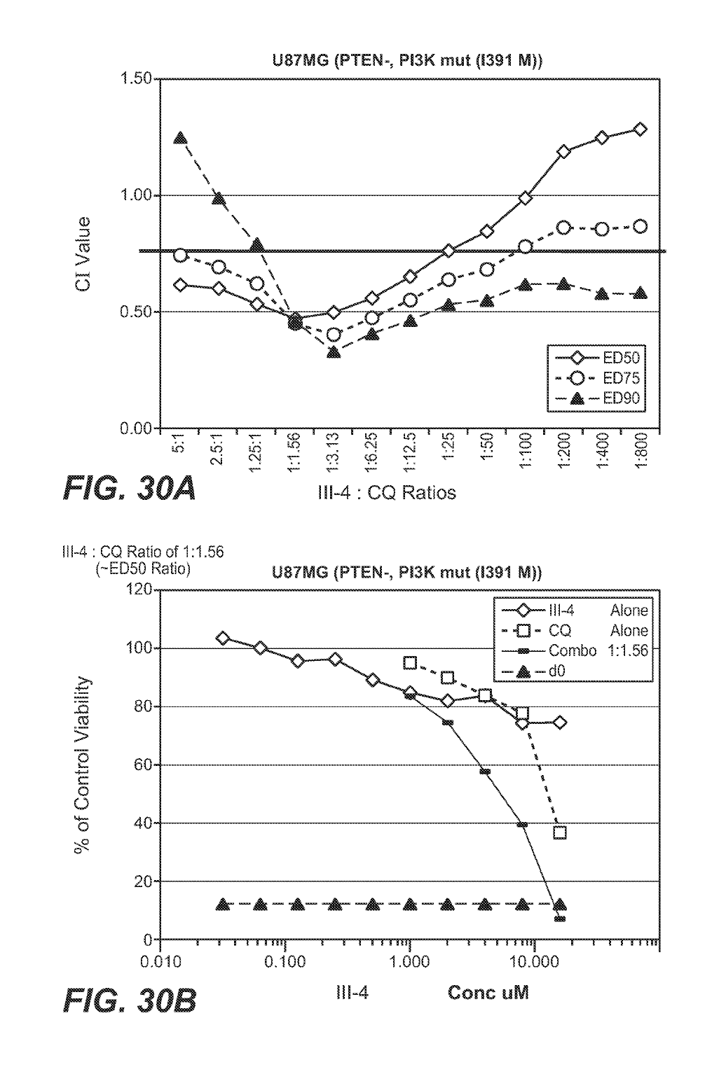

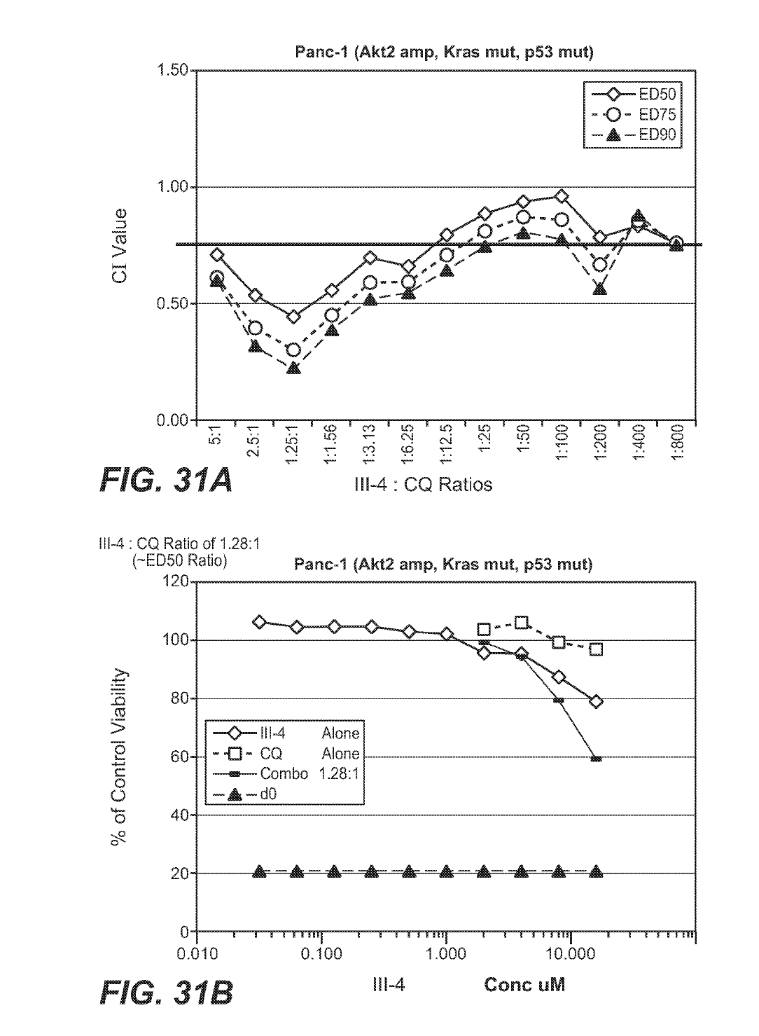

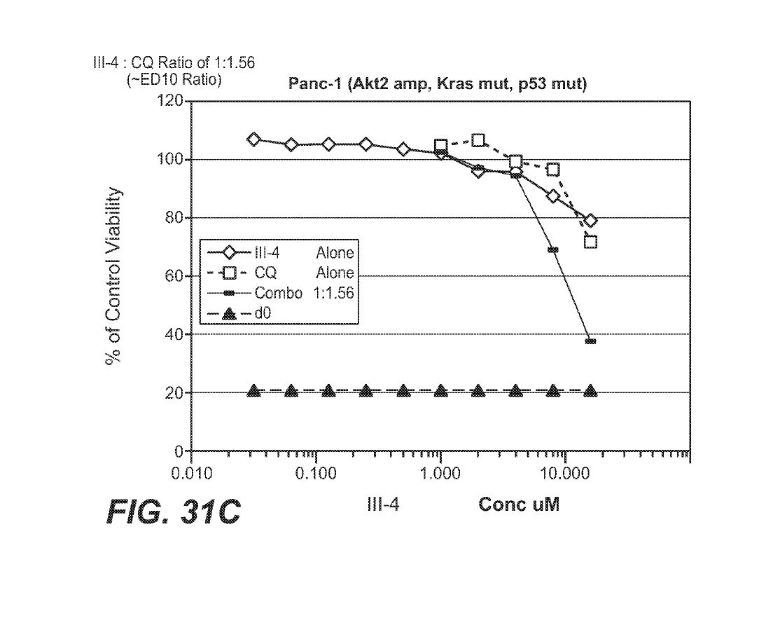

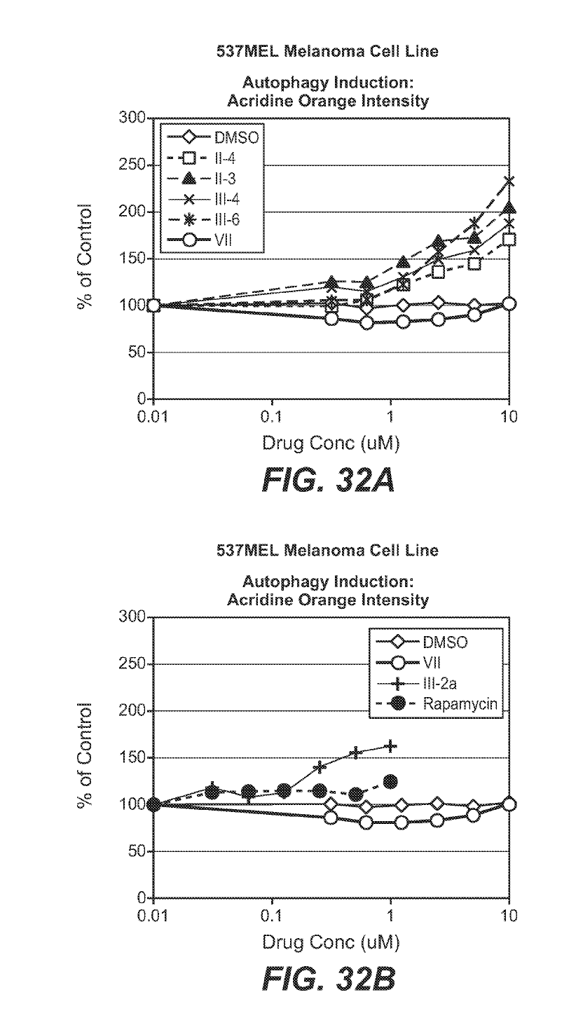

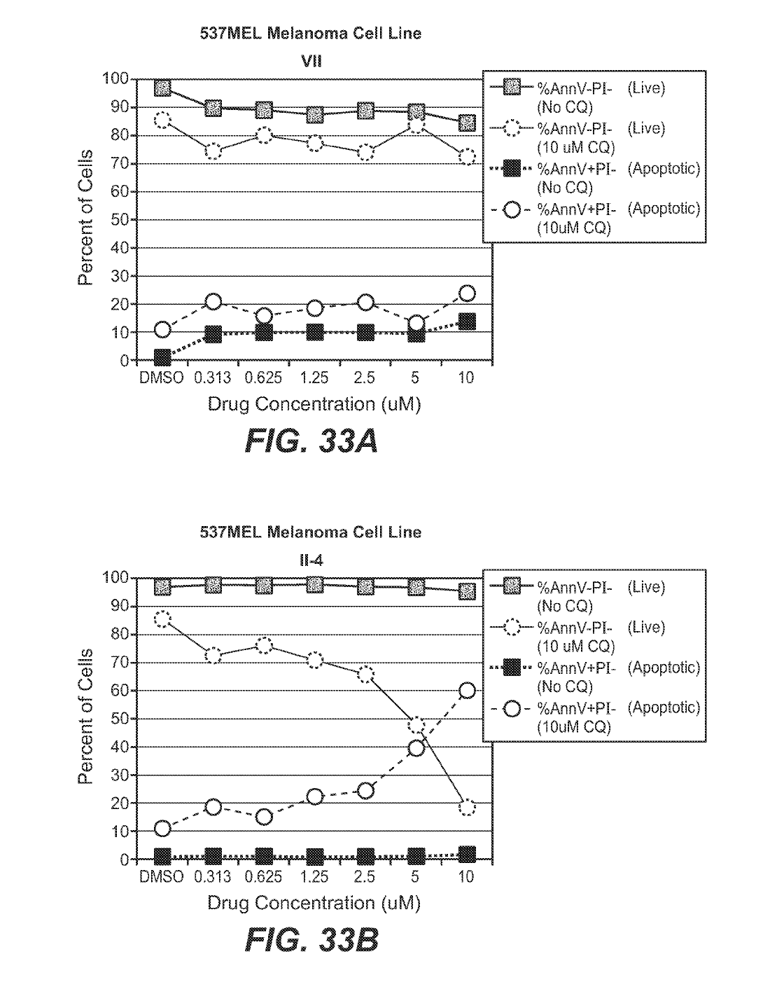

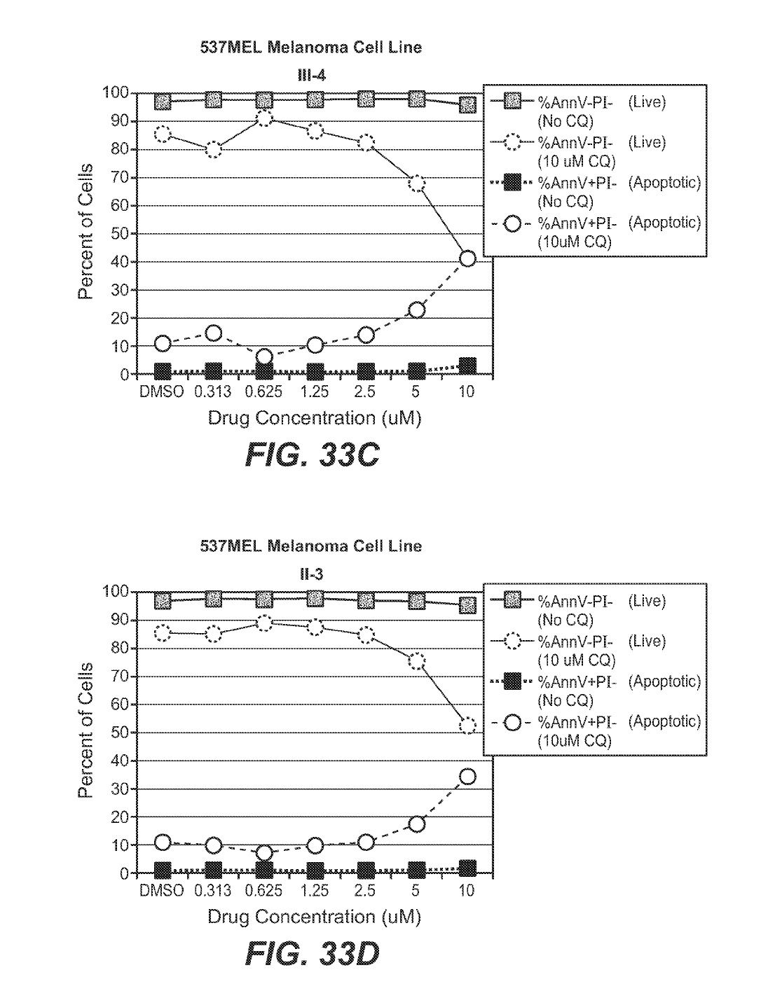

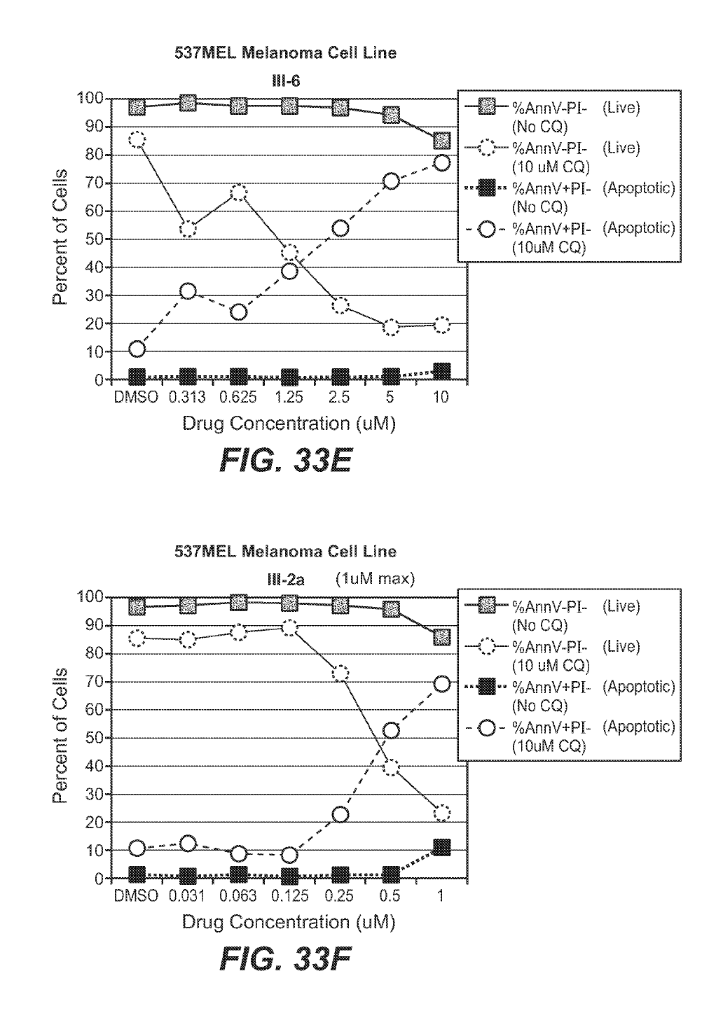

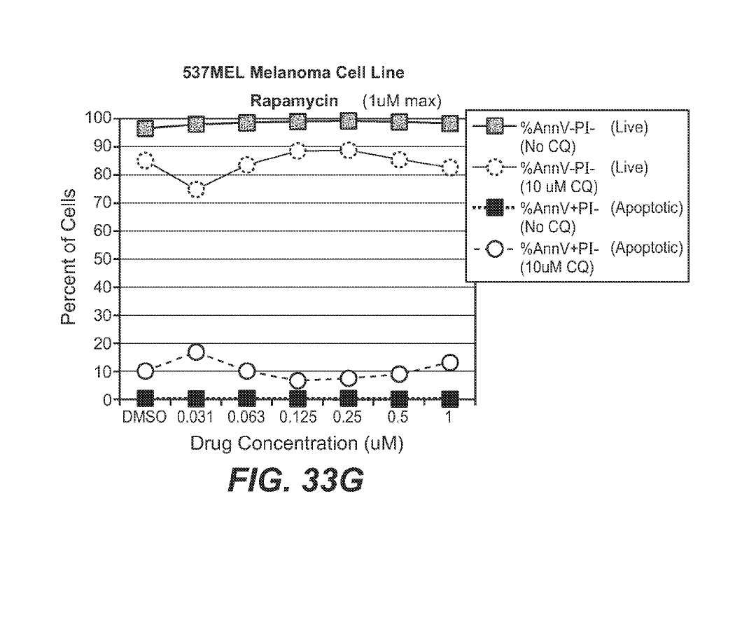

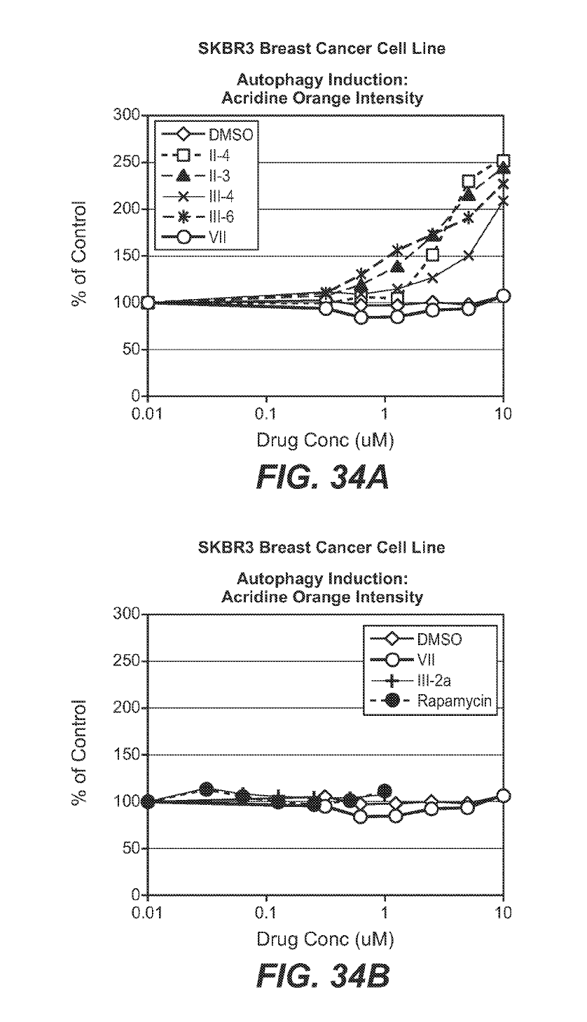

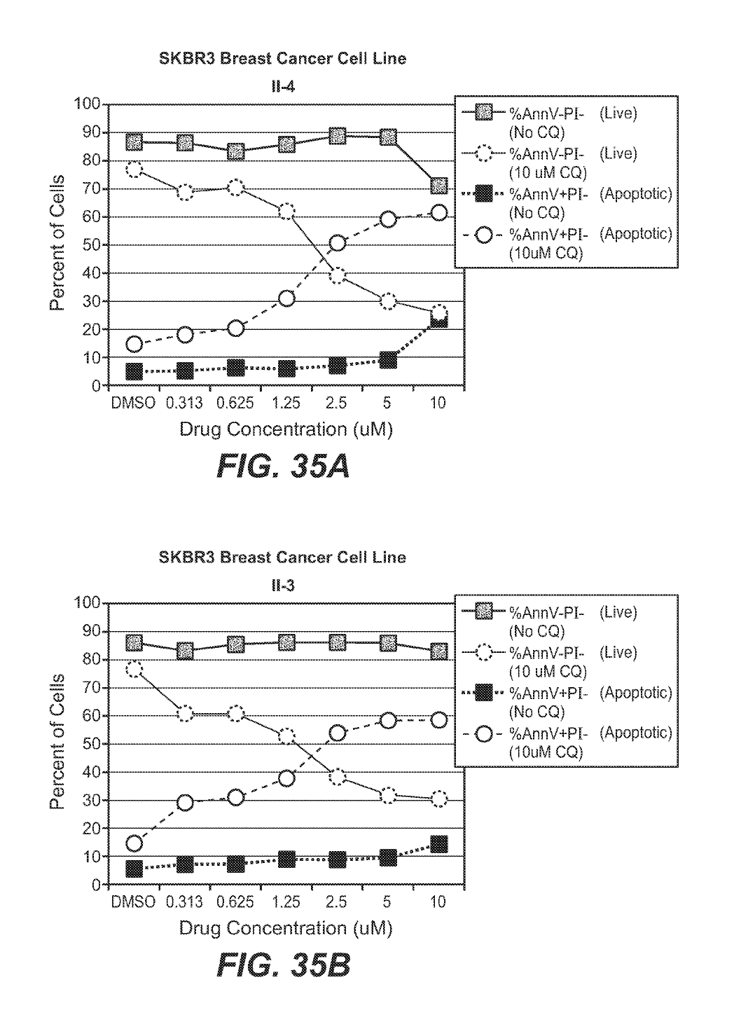

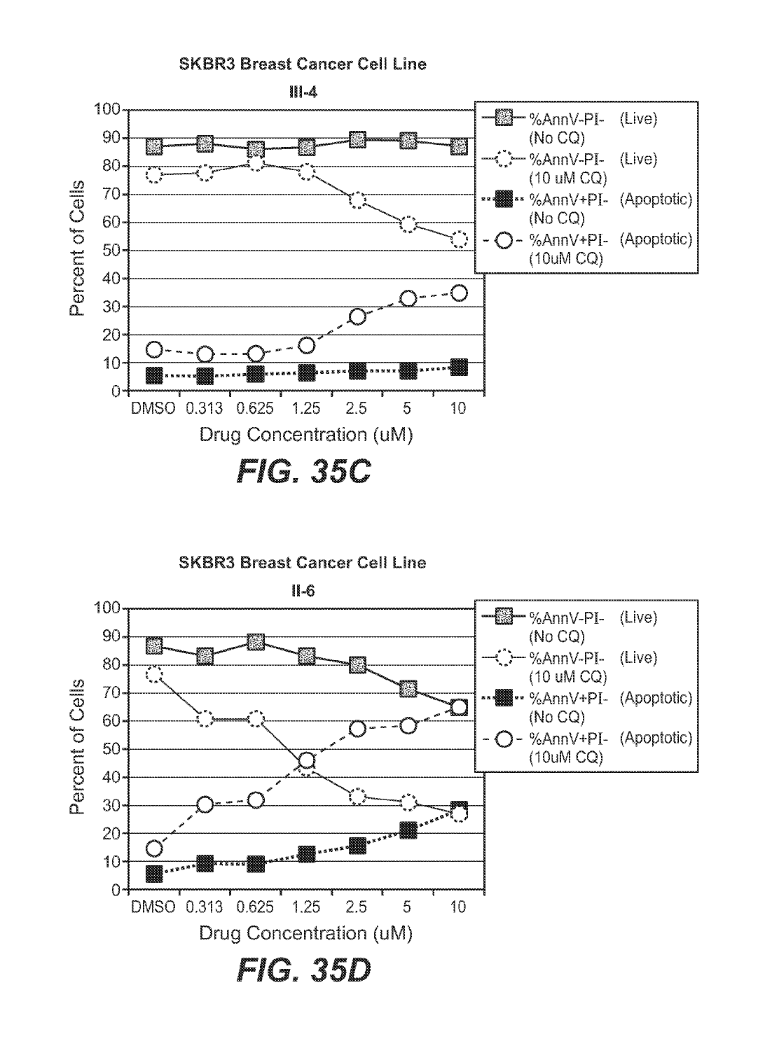

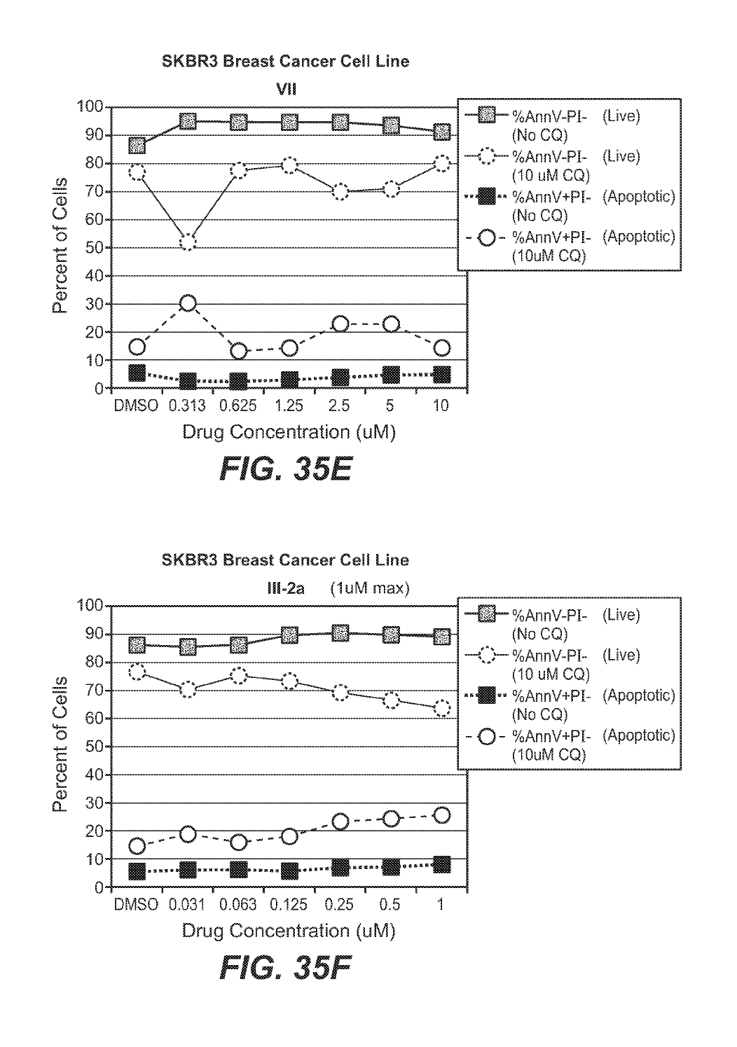

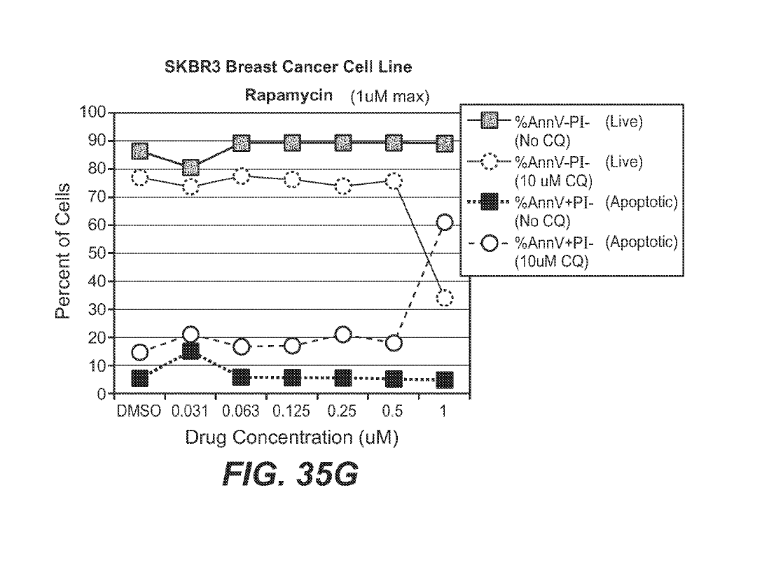

The onset of cell death in cells treated with both CQ and Akt pathway inhibitors was preceded by an accumulation of enlarged autolysosome-like vacuoles. Although both Akt inhibition and CQ alone induced accumulation of autophagic vacuoles (AVs) (FIG. 25 A-C), further analysis revealed that Akt inhibition alone resulted in increased production and maturation of cathepsin D and lysosomal activity, whereas CQ blocked the maturation of cathepsin D, leading to accumulation of very large and likely degradation-defective vacuoles in cells with Akt inhibition. This coincided with activation of caspase 3 and a dramatic increase in apoptotic nuclei (FIG. 25 D,E). There was an increase in mitochondrial depolarization and reactive oxygen species (ROS) generation in cells treated with Akt inhibitor alone. Cytoplasmic ROS generation was attenuated within 48 hours with Akt inhibitor alone, but CQ co-treatment caused a prolonged ROS accumulation both in the cytoplasm and in the vacuoles. Cytoplasmic translocation of cathepsin D due to increased lysosomal membrane permeability (LMP) and the subsequent degradation of cytoplasmic ROS scavenger thioredoxin was previously proposed as a mechanism of ROS accumulation and cell death induction by CQ in combination with another autophagy inducer. (Carew J S, Nawrocki S T, Kahue C N, Zhang H, Yang C, Chung L, Houghton J A, Huang P, Giles F J, Cleveland J L. Targeting autophagy augments the anticancer activity of the histone deacetylase inhibitor SAHA to overcome Bcr-Abl-mediated drug resistance. Blood 2007; 110:313-22). Although LMP may still be a possible downstream event contributing to cell death, no significant increase in thioredoxin degradation prior to the onset of cell death in our system (Degtyarev and Lin, unpublished data) was observed. In contrast, cathepsin D knockdown or inhibition of lysosomal protease activity was not able to protect cells from CQ/Akti-1/2-induced cell death, but rather had a similar effect to CQ in promoting cell death when combined with Akti-1/2. This is consistent with the opposite effects of CQ and Akti-1/2 on cathepsin D maturation, and suggests that preserving (an elevated) lysosomal degradation activity may be critical for cell survival in the presence of elevated autophagic activity induced by Akt inhibition. Taken together, these findings support a model whereby limited mitochondrial depolarization caused by Akt inhibition resulted in an increase in ROS signal that promoted autophagy (Scherz-Shouval R, Shvets E, Fass E, Shorer H, Gil L, Elazar Z. Reactive oxygen species are essential for autophagy and specifically regulate the activity of Atg4. Embo J 2007; 26:1749-60), which in turn removed the damaged mitochondria and alleviated the oxidative stress. Impairment of autolysosomal digestion caused by CQ resulted in aggregation of deleterious oxidative products, which could further amplify the ROS damage (Moore M N, Viarengo A, Donkin P, Hawkins A J. Autophagic and lysosomal reactions to stress in the hepatopancreas of blue mussels. Aquat Toxicol 2007; 84:80-91). Multiple downstream events, including caspase activation and possibly LMP can lead to both apoptosis-like and non-apoptotic cell death (FIG. 24). Not to be bound by theory, FIG. 24 depicts a model of the mechanism of cell death by Akt inhibition in combination with an autophagy inhibitor (chloroquine).

Dosing of a pan-Akt inhibitor will likely be limited by its side effects, most notably metabolic effects due to inhibition of insulin signaling. (Amaravadi et al, 2005). Our data suggest that at least in cancer models like the PTEN-null PC3 xenograft tumors, complete elimination of tumor cells may not be achievable with continuous pan-Akt knockdown alone. However, combined treatment with CQ significantly increased the incidence of complete tumor remission in xenograft models, although CQ alone had no significant effect. This suggests that autophagy induction through Akt inhibition can sensitize tumors to this relatively non-toxic drug, clinically approved for other indications.

Inappropriate inhibition of autophagy could result in loss of its tumor suppression function or may cause up-regulation of alternative survival pathways. (Levine B. Cell biology: autophagy and cancer. Nature 2007; 446:745-7; Wang Y, Singh R, Massey A C, Kane S S, Kaushik S, Grant T, Xiang Y, Cuervo A M, Czaja M J. Loss of macroautophagy promotes or prevents fibroblast apoptosis depending on the death stimulus. J Biol Chem 2008; 283:4766-77). Data herein suggest that the accumulation of defective autolysosomes is required for CQ's effect. One potential advantage of blocking degradation while allowing the autophagic sequestration to occur is that this may result in the formation of more toxic ROS generators in defective autolysosomes such as lipofuscin (Terman A, Gustafsson B, Brunk U T. The lysosomal-mitochondrial axis theory of postmitotic aging and cell death. Chem Biol Interact 2006; 163:29-37; Moore M N, Viarengo A, Donkin P, Hawkins A J. Autophagic and lysosomal reactions to stress in the hepatopancreas of blue mussels. Aquat Toxicol 2007; 84:80-91), therefore leading to a more rapid cell death induction. In addition, cells may not find an easy escape after they are already well engaged in an autophagic response.

The PI3K/Akt pathway is crucial to many aspects of cell growth and survival with multiple components targeted by genomic aberrations more frequently than any other pathway in human cancer, making it an attractive target for cancer therapy. Critical questions underlying the clinical outcomes of Akt inhibitors are the degree of selectivity between the three isoforms needed, and the effects on tumor cell growth and survival expected from inhibiting these kinases. The recently described allosteric Akt inhibitors with unprecedented selectivity towards Akt1 and Akt2 provide valuable tools to begin addressing these questions (Barnett, S. F., M. T. Bilodeau, and C. W. Lindsley, (2005), The Akt/PKB family of protein kinases: a review of small molecule inhibitors and progress towards target validation. Curr Top Med Chem. 5:109-25). However, to date small molecule inhibitors are limited in the degree of specificity that can be achieved, and the in vivo efficacy of the reported compounds were not evaluated due to poor pharmacological properties. In addition, Akt3 selective compounds have not been reported.

RNA interference is a powerful method for suppressing gene expression. Using a Dox− inducible shRNA approach, we are able to achieve specific KD of each Akt isoform, both individually and in all possible combinations, to evaluate the requirement of each isoform in the maintenance of tumor growth in vivo. Data provided herein results suggest that in both Pten− androgen-independent prostate cancer model PC3 and glioblastoma model U87MG, Akt1 is the most important isoform in maintaining tumor growth. This is in concert with the recent report that Akt1 deficiency can markedly decrease the incidence of tumors in Pten+/− mice, both in tissues where Akt1 is the predominantly expressed isoform and in those where Akt1 is not (Chen et al., 2006). However, in the mouse genetic study Akt1 was ablated prior to the development of tumors in Pten+/− mice, whereas in the present study we allowed the tumors to establish before Akt1 KD was induced. Thus, reducing Akt1 activity not only prevents tumors from developing, but also inhibits the growth of established tumors with PTEN deficiency in human cancer models.

Additional KD of Akt2 and Akt3 resulted in a more consistent and pronounced inhibition of tumor growth. This suggests that Akt2 and Akt3 activities can partially compensate for the reduced Akt1 activity in maintaining tumor growth. This is consistent with the more effective inhibition of downstream targets observed with combined Akt KDs. Taking together the recent reports of increased invasiveness associated with inhibiting Akt1 alone that could be counteracted by simultaneous KD of Akt2, highly selective Akt1 inhibition may not be desirable. The data provided herein suggest that partial KD of all three isoforms can be more effective in tumor growth inhibition. A plausible scenario would be to inhibit all three Akt isoforms but with different degrees of activity KD, thus preserving a crucial level of isoform activity for their normal physiological functions, while achieving the maximum inhibitory effect on tumor growth and progression.

One of the most prominent functions of Akt is to mediate cell survival. Constitutively active Akt has been reported to protect cells from programmed cell death following various pro-apoptotic insults. Whether apoptosis is a primary response to Akt inhibition is however less clear, especially in cancer cells where apoptosis is often suppressed due to various genetic alterations. Previous experiments using small molecule inhibitors of the PI3K/Akt pathway often generate conflicting results that are obscured by the inevitable non-specific effects of these compounds. Data provided herein indicate that under normal cell culture conditions, specific KD of any or all three isoforms of Akt can result in cell cycle delay without promoting significant apoptosis. This is consistent with a recent report that only a small portion of total Akt activity is required for apoptosis inhibition under normal growth condition (Liu, X., Y. Shi, M. J. Birnbaum, K. Ye, R. De Jong, T. Oltersdorf, V. L. Giranda, and Y. Luo, (2006), Quantitative analysis of anti-apoptotic function of Akt in Akt1 and Akt2 double knock-out mouse embryonic fibroblast cells under normal and stressed conditions. J Biol Chem. 281:31380-8). In contrast, significantly increased autophagy was observed in both PC3 and U87MG cells with Akt KDs, suggesting that autophagy is a more sensitive response to reduced Akt activity. This is further demonstrated by using relatively specific inhibitors of the pathway, including a dual PI3K/mTOR inhibitor and a dual Akt1,2 inhibitor.

Although the molecular mechanisms of Akt inhibition-induced autophagy remains to be further elucidated, several possibilities exist. First, inhibiting Akt can lead to inhibition of mTOR, which is a known inhibitor of autophagy. Interestingly, a constitutively active form of Akt was shown to suppress the induction of autophagy by rapamycin (Takeuchi, H., Y. Kondo, K. Fujiwara, T. Kanzawa, H. Aoki, G. B. Mills, and S. Kondo, (2005), Synergistic augmentation of rapamycin-induced autophagy in malignant glioma cells by phosphatidylinositol 3-kinase/protein kinase B inhibitors. Cancer Res. 65:3336-46), raising the possibility that the effect of Akt on autophagy may not be completely mediated through the raptor-mTOR activity downstream of Akt, or that the effect of rapamycin may be mediated at least in part through inhibiting Akt, e.g. through inhibition of the assembly of mTORC2 after prolonged treatment (Sarbassov, D. D., S. M. Ali, S. Sengupta, J. H. Sheen, P. P. Hsu, A. F. Bagley, A. L. Markhard, and D. M. Sabatini, (2006), Prolonged rapamycin treatment inhibits mTORC2 assembly and Akt/PKB. Mol Cell. 22:159-68. Epub 2006 Apr. 6). Second, it is possible that other signaling outputs of Akt, such as glucose uptake and metabolism, or cell cycle regulation, can also contribute to autophagy regulation independent of mTOR. Of note, Akt inhibition stabilizes p27kip1, which was recently shown to mediate autophagy under growth factor withdrawal (Liang, J., S. H. Shao, Z. X. Xu, B. Hennessy, Z. Ding, M. Larrea, S. Kondo, D. J. Dumont, J. U. Gutterman, C. L. Walker, J. M. Slingerland, and G. B. Mills, (2007), The energy sensing LKB1-AMPK pathway regulates p27(kip1) phosphorylation mediating the decision to enter autophagy or apoptosis. Nat Cell Biol. 9:218-24). Third, data provided herein indicate that Akt inhibition induces mitochondria membrane depolarization and increased ROS generation. It was recently shown that starvation stimulates formation of ROS in the mitochondria, which serves as a signal to activate autophagy (Scherz-Shouval, R., E. Shvets, E. Fass, H. Shorer, L. Gil, and Z. Elazar, (2007), Reactive oxygen species are essential for autophagy and specifically regulate the activity of Atg4. Embo J. 26:1749-60). It is conceivable that Akt inhibition can induce autophagy via a similar mechanism, and elevated autophagy in turn recycles these damaged mitochondria and prevents the accumulation of ROS to a detrimental level.

Although excessive autophagy may lead to cell killing when allowed to reach its limit, inhibiting Akt alone is apparently very ineffective in cell killing even under reduced serum conditions in the PTEN-null cancer cell lines that we examined. Under the in vivo tumor growth conditions, autophagy may be a potential mechanism by which Akt inhibition restricts tumor growth, but may also provide temporary relief from the metabolic and oxidative stress imposed by Akt inhibition, which may allow resistance to occur. Indeed, most tumors treated with Akt KD alone became resistant and rebound after initial regression or stasis Inhibiting autophagy at an early stage may prevent this temporary protective effect, but may also counteract the possible tumor inhibitory effect of autophagy. Blocking autophagy completion at a late stage might avoid this counteracting effect. Indeed, combination of Akt inhibition with lysosomotropic agents resulted in excessive accumulation of degradation-defective autolysosome-like vacuoles that cannot be cleared, resulting in accelerated cell death. This combination can not only sabotage the ROS scavenger and self-renewal functions of autophagy, but also promote the rupture of defective autolysosomes and the release of lysosomal contents into the cytosol, further augmenting the oxidative stress and mitochondrial damage, leading to eventual cell death. Recently, it was reported that inhibition of autophagy that was induced as an adaptive survival response to therapy could enhance apoptosis in a Myc-induced mouse model of lymphoma (Amaravadi et al., 2007). As reported herein, autophagy induced by Akt/PI3K inhibition can be exploited using lysosomotropic agents to promote the remission of PTEN-null human tumor xenografts. Since this effect is expected to correlate positively with the degree of autophagy induced by a given treatment, creative combination of lysosomotropic agents with agents that induce extensive autophagy, such as inhibitors of the Akt pathway, may profoundly affect their anti-cancer efficacy. Degenhardt et al. proposed that autophagy inhibition by Akt overexpression could lead to necrosis in the center of tumors while the surrounding tumor cells might respond with accelerated growth as a result of combined effect of necrosis-induced inflammatory response and Akt-stimulated proliferation. In the presence of Akt inhibition, however, accelerated cell death caused by CQ-induced late autophagy inhibition might enable completely eliminate tumor cells before they have time to grow back due to possible inflammatory response, because tumor cell proliferation is greatly reduced. Data provided herein show that cells with PTEN deficiency are more sensitive to this combination than cells with intact PTEN, suggesting that a reasonable therapeutic window might be achieved. CQ has already found therapeutic efficacy in several diseases and is well tolerated (Gustafsson, L. L., O. Walker, G. Alvan, B. Beermann, F. Estevez, L. Gleisner, B. Lindstrom, and F. Sjoqvist, (1983), Disposition of chloroquine in man after single intravenous and oral doses. Br J Clin Pharmacol. 15:471-9; Hagihara, N., S. Walbridge, A. W. Olson, E. H. Oldfield, and R. J. Youle, (2000), Vascular protection by chloroquine during brain tumor therapy with Tf-CRM107. Cancer Res. 60:230-4). Given the lengthening list of anti-cancer agents reported to induce autophagy, CQ and other lysosomotropic agents may find promising new therapeutic values in cancer therapy.

### EXAMPLES

### Materials and Methods

Cell Culture and Reagents: The PTEN^{-/-} and PTEN^{+/+} MEFs were maintained as previously described (Sun, H., R. Lesche, D. M. Li, J. Liliental, H. Zhang, J. Gao, N. Gavrilova, B. Mueller, X. Liu, and H. Wu, (1999), PTEN modulates cell cycle progression and cell survival by regulating phosphatidylinositol 3,4,5,-trisphosphate and Akt/protein kinase B signaling pathway. Proc Natl Acad Sci USA. 96:6199-204). The PC3 and U87MG cells were maintained at 37° C. and 5% CO₂ in DMEM/Ham's F-12 (1:1) containing 10% tetracycline-free fetal bovine serum. II-4 was from Calbiochem (Akt inhibitor VIII) (Barnett, S. F., D. Defeo-Jones, S. Fu, P. J. Hancock, K. M. Haskell, R. E. Jones, J. A. Kahana, A. M. Kral, K. Leander, L. L. Lee, J. Malinowski, E. M. McAvoy, D. D. Nahas, R. G. Robinson, and H. E. Huber, (2005), Identification and characterization of pleckstrin-homology-domain-dependent and isoenzyme-specific Akt inhibitors. Biochem J. 385:399-408). To inhibit autophagy, cells were treated with 5-10 μM chloroquine, 2.5 nM Bafilomycin A1 or 1 mM 3-MA (all from Sigma) and analyzed at the indicated time points. Image-iT LIVE Green Reactive Oxygen Species Detection Kit was purchased from Molecular Probes. MitoPT Mitochondria Permeability Transition Detection Kit was purchased from Immunochemistry Technologies, LLC.

Inducible shRNA constructs and generation of inducible-shRNA clones: The pHUSH tetracycline-inducible retrovirus gene transfer vector has been described elsewhere (Gray, D., A. M. Jubb, D. Hogue, P. Dowd, N. Kljavin, S. Yi, W. Bai, G. Frantz, Z. Zhang, H. Koeppen, F. J. de Sauvage, and D. P. Davis, (2005), Maternal embryonic leucine zipper kinase/murine protein serine-threonine kinase 38 is a promising therapeutic target for multiple cancers. Cancer Res. 65:9751-61; Hoeflich, K. P., D. C. Gray, M. T. Eby, J. Y. Tien, L. Wong, J. Bower, A. Gogineni, J. Zha, M. J. Cole, H. M. Stern, L. J. Murray, D. P. Davis, and S. Seshagiri, (2006), Oncogenic BRAF is required for tumor growth and maintenance in melanoma models. Cancer Res. 66:999-1006; US 2007/0026002, herein incorporated by reference in its entirety). The complementary double-stranded shRNA oligos were inserted into this vector system using a shuttle vector followed by a Gateway recombination reaction (Invitrogen) as previously described (Grunwald, V., L. DeGraffenried, D. Russel, W. E. Friedrichs, R. B. Ray, and M. Hidalgo, (2002), Inhibitors of mTOR reverse doxorubicin resistance conferred by PTEN status in prostate cancer cells. Cancer Res. 62:6141-5; See also FIG. 23 herein). The shRNA sequences used in this study are summarized in Table 2. All constructs were verified by sequencing. Retrovirus infection was performed as described (Gray et al., 2005; Hoeflich et al., 2006). For single Akt isoform knockdowns, cells were infected with one retroviral vector encoding an shRNA construct singly targeting each Akt isoform (constructs 252 & 253 for Akt1, 254 & 255 for Akt2, and 259 & 260 for Akt3) and stable clones were selected using 5 μg/ml puromycin. For dual Akt1 and Akt2 KD, a single shRNA targeting both Akt1 and 2 simultaneously (construct 256 & 257) was used. Dual Akt2 and 3 (constructs 255 and 261), or triple Akt1, 2 and 3 (constructs 257 and 261) knockdowns were achieved by co-infecting the cells with two retroviral vectors containing different antibiotic selection markers (puromycin and hygromycin), each encoding one single shRNA, and stable clones were selected using 5 μg/ml puromycin and 300 μg/ml hygromycin. For dual Akt1 and 3 KD, either a single shRNA targeting both Akt1 and 3 (construct 258), or co-infection with two shRNA vectors (constructs 253 and 261) were employed.

Western blot analysis, immunofluorescence, 1HC and TUNEL assay: For Western blot analysis, total protein lysates were subjected to SDS-PAGE and transferred to nitrocellulose. Antibodies used were: anti-Akt1, anti-Akt2, anti-Akt3, anti-total-Akt, anti-p-Akt (Ser473), anti-p-Akt (Thr308), anti-p-S6 (Ser235/236), anti-PARP and anti-cleaved caspase-3 (Cell Signaling Technology); anti-p-PRAS40 (Invitrogen); anti-p27^{Kip1} (Santa Cruz Biotechnology); anti-LC3 (Novus); anti-LAMP2 and anti-Cathepsin D (BD Biosciences); and anti-GAPDH (Advanced Immunochemical Inc.). Primary antibodies were detected using IR Dye 800-conjugated (Rockland) and Alexa-Fluoro 680-conjugated (Molecular Probes) species-selective secondary antibodies. Detection and quantification were performed using an Odyssey infrared scanner (LICOR) using the manufacturer's software. For immunofluorescence staining, cells were fixed in 3% paraformaldehyde and permeabilized with 0.01% digitonin in PBS, followed by a rabbit polyclonal anti-LC3 (Abgent) primary antibody detected with a cy3-conjugated anti-rabbit secondary antibody (Jackson Immunoresearch). For IHC, formalin-fixed, paraffin-embedded specimens were collected. 5-μm-thick paraffin-embedded sections were stained using an anti-Ki-67 (MIB-1, DakoCytomation) antibody with the Dako ARK kit (Dako Corporation). Tissues were counterstained with haematoxylin, dehydrated, and mounted. In all cases, antigen retrieval was performed with the Dako Target Retrieval Kit as per manufacturer's instructions. For TUNEL assay, formalin-fixed, paraffin-embedded sections were stained using an in situ cell death detection kit (POD; Roche Diagnostic) according to the manufacturer's instructions.

Xenograft study: Six- to 8-week-old female athymic nude nu/nu mice were purchased from Charles River Laboratories and maintained in Genentech's conventional animal facility. Mice were injected in the right flank with 5˜7.5×10⁶ cells resuspended in 200 μl Hank's Balanced Salt Solution (Invitrogen). When tumors reached a mean volume of 100˜300 mm³ the mice with similarly-sized tumors were grouped into treatment cohorts. Mice received 5% sucrose or 5% sucrose plus 1 mg/ml Dox in drinking water for control and KD cohorts, respectively. Amber-colored water bottles were used and were changed 3 times per week. CQ is dissolved in 0.9% physiological saline, filter-sterilized and administered at 45 mg/kg through either intraperitoneal or subcutaneous routes. Tumors were measured with calipers and mice weighed twice per week. Mice whose tumors reached 2000 mm³ or lost more than 20% body weight were euthanized. Between 8˜10 mice were used for each treatment group. Statistical significance was analyzed using the JMP software (SAS Institute, Inc.).

Electron Microscopy: Cells were grown to monolayer in plastic flasks and fixed in half-strength Karnovsky fixative (2% paraformaldehyde, 2.5% glutaraldehyde, 0.025% CaCl₂.2H₂O and 0.1 M sodium cacodylate buffer, pH 7.4); tumors were cut into small cubes (˜1 mm³) and fixed by immersion in the same fixative or in the fixative used for immunoelectron microscopy. Cells and tissues were postfixed with 1% OsO₄ and 1% K₄Ru(II)(CN)₆ or 1.5% K₃Fe(CN)₆, dehydrated in ethanol and embedded in Epon. Ultrathin sections were stained with uranyl acetate and lead citrate. Numbers of AV were counted on systematically sampled cytoplasmic areas of 4.5 μm² (n≧64 per condition). The percent AV area was measured by means of a square mesh grid laid over ≧5 sets of systematically sampled micrographs with each set covering a cytoplasmic area ≧80 μm². The average percent of apoptotic nuclei in tumor tissues was calculated from the number of apoptotic nuclei in 3 to 4 sets of 100 systematically counted tumor cell nuclei.

Immunoelectron microscopy. Small tumor blocks were fixed by immersion in 2% paraformaldehyde, 0.2% glutaraldehyde in 0.1 M phosphate buffer, pH 7.4 for 5 h at 4° C. After rinsing with PBS, the blocks were embedded in 12% gelatin, cryoprotected with 2.3 M sucrose, and frozen in liquid nitrogen. Ultrathin cryosections were cut at −120° C., picked up with 1% methylcellulose, 1.2 M sucrose, thawed and collected on copper grids. After washing with PBS containing 0.02 M glycine, sections were incubated with rabbit anti-human LAMP1 antibodies (gift of M. Fukuda, (Carlsson, S. R., J. Roth, F. Piller, and M. Fukuda. 1988. Isolation and characterization of human lysosomal membrane glycoproteins, h-lamp-1 and h-lamp-2. Major sialoglycoproteins carrying polylactosaminoglycan. J Biol Chem. 263:18911-9) or with rat monoclonal anti-mouse LAMP-1 antibody ID4B (T. August, Developmental Studies Hybridoma Bank, Iowa City, Iowa), followed by a secondary rabbit anti-rat IgG antibody (Dako). These were subsequently labeled with Protein A conjugated to 10 nm colloidal gold particles, and contrasted with a 1.8% methylcellulose, 0.6% uranyl acetate mixture.

Cell viability and cell cycle analysis. Cell number and viability was measured using trypan blue exclusion assay using a Vi-Cell Analyzer (Beckman Coulter), or labeled with 1 μg/ml PI in PBS/1% BSA followed by cytofluorometric analysis with a fluorescence-activated cell sorter (FACS) (Becton Dickinson). FITC-conjugated Annexin V was used for the assessment of phosphatidylserine exposure by FACS analysis. Caspase activation was analyzed using a Caspase-Glo 3/7 Assay kit (Promega). For cell cycle analysis, cells were fixed with drop-wise addition of chilled 70% ethanol, washed with PBS and resuspended in staining solution containing 50 μg/ml PI and 60 units of RNAse A. DNA content was analyzed by flow cytometry using the FlowJo and ModFit software (Becton Dickinson).

Multispectral imaging flow cytometry: Cells treated with various agents were stained with Acridine Orange and analyzed by the ImageStream system (Amnis Corporation, Seattle, Wash.) using the IDEAS image analysis program. The DNA AOGreen image and the vacuolar AO Red image were first compensated into separate channels, and then the percentage of apoptotic/anucleate cells (based on AO nuclear morphology and intensity) and vacuolated cells (AO Red+) were quantified. Plotting AO Green Intensity vs AO Green bright detail area revealed three distinct populations: R2 anucleated cells (low AO Green labeling, higher area due to masking of diffuse cytoplasm); R3 apoptotic cells (intermediate to low AO Green, very low AO Green detail area due to presence of small, bright condensated nuclear fragments); R4 live cells (intact bright nucleus). AORed Intensity is plotted on the second histogram with an arbitrary gate (R5) drawn to include events with the brightest AO Red intensity.

Time-lapse video microscopy: Cells cultured in 24-well plates were imaged on an Olympus IX81 inverted microscope under environmental control (37° C. and 5% CO₂) for 3 days. Imaging started 6 hours after the addition of the compounds and was taken at 1-hour intervals.

### Example 1

### Inducible shRNA KD of Akt Isoforms Inhibited the Growth of PTEN-Null Human Tumor Xenografts in a Dose- and Isoform Dependent Manner

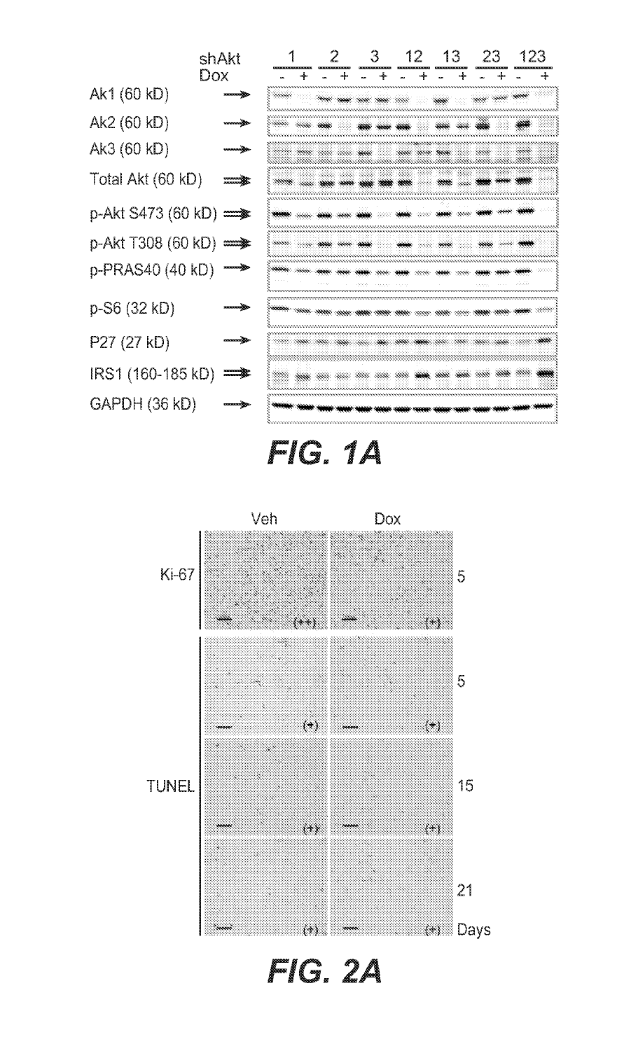

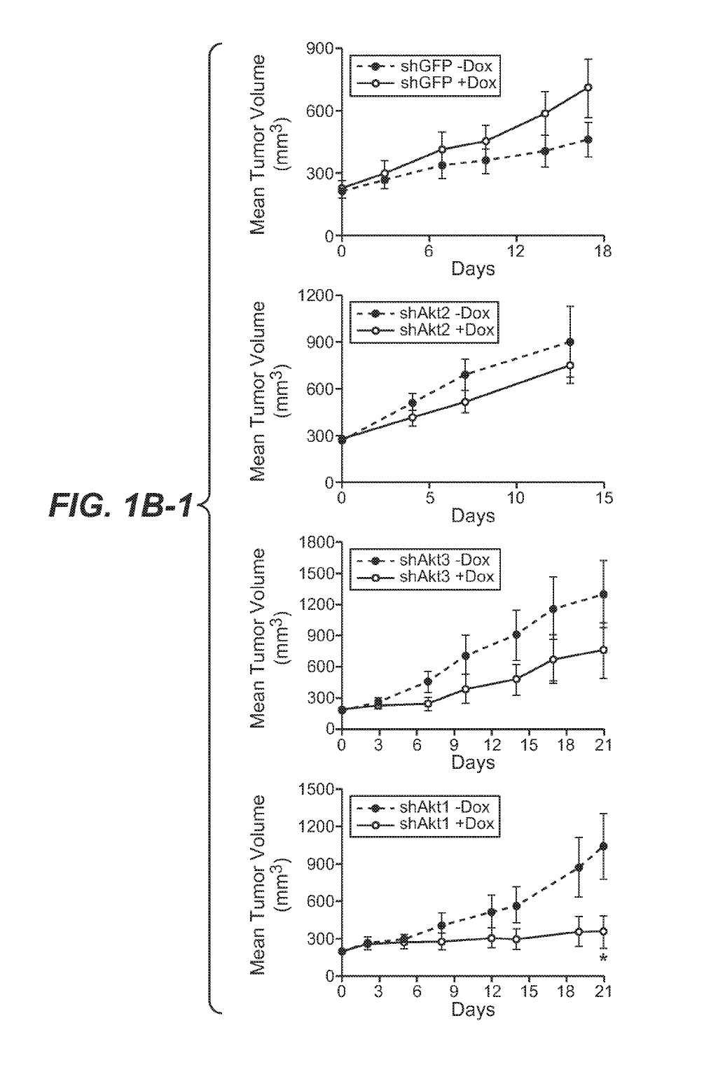

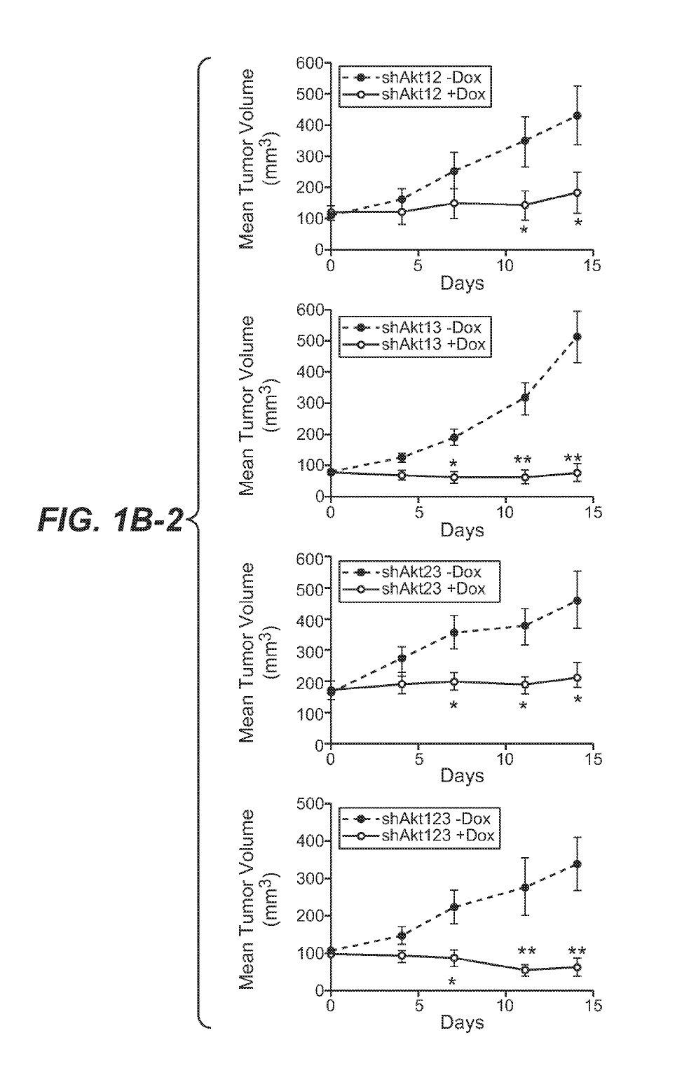

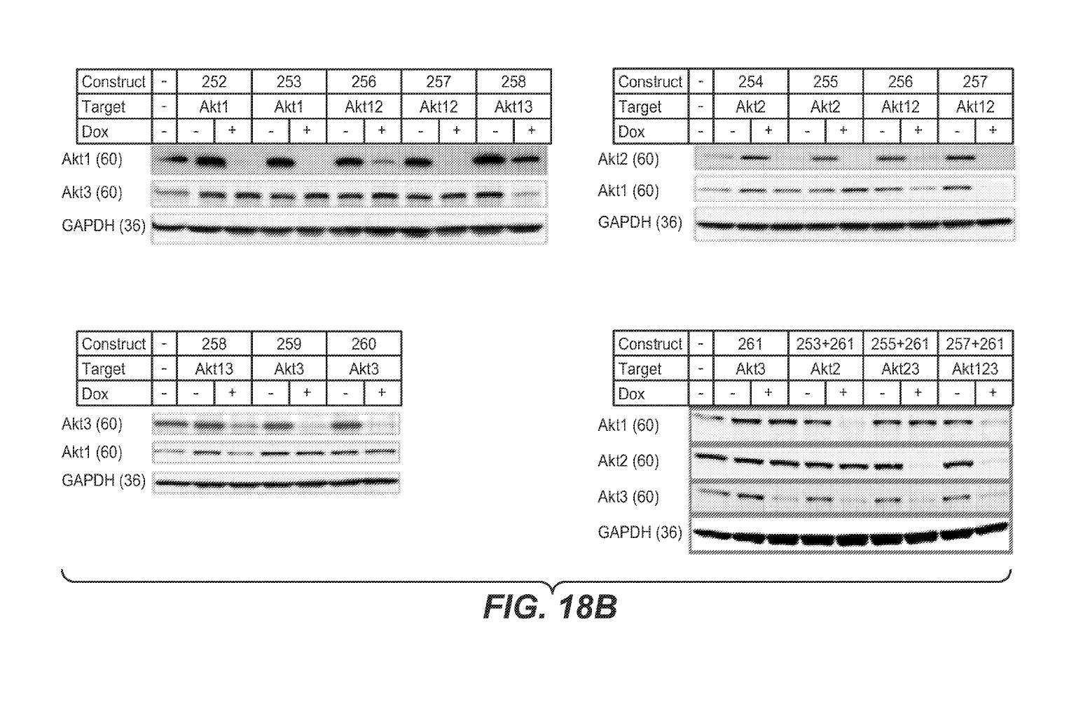

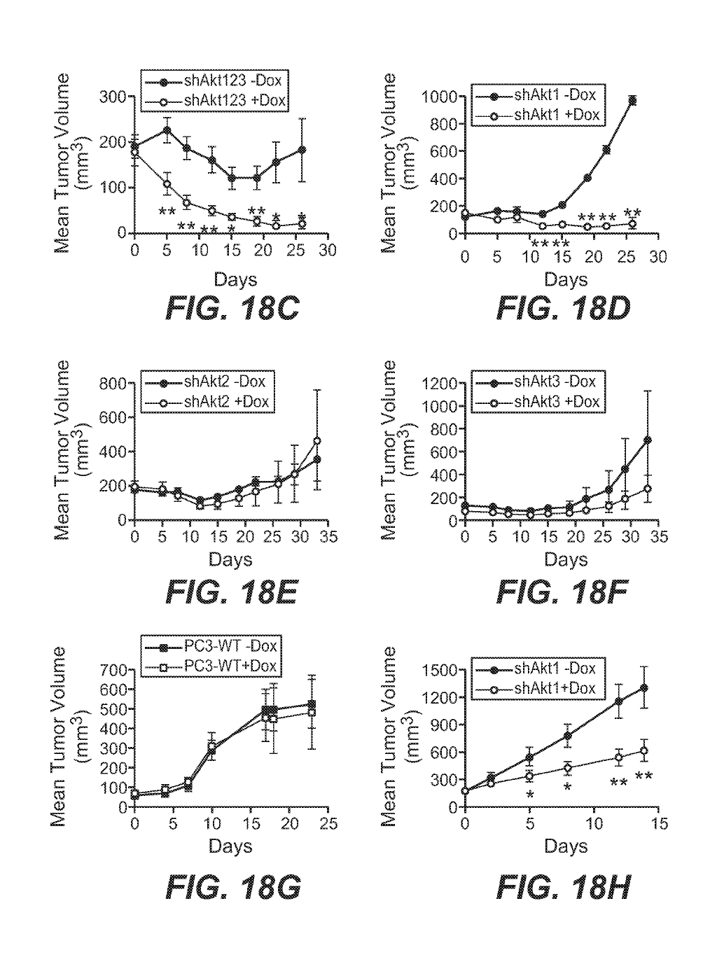

To determine the relative contribution of the three Akt isoforms in maintaining tumor growth, we used a tet-inducible shRNA KD method using a recently described retroviral vector system, a tet-inducible plasmid vector for H1 or U6 short hairpin (Gray et al., 2005; Hoeflich et al., 2006). We chose the PTEN-null human prostate cancer cell line PC3 and the glioma cell line U87MG (Li, J., C. Yen, D. Liaw, K. Podsypanina, S. Bose, S. I. Wang, J. Puc, C. Miliaresis, L. Rodgers, R. McCombie, S. H. Bigner, B. C. Giovanella, M. Ittmann, B. Tycko, H. Hibshoosh, M. H. Wigler, and R. Parsons, (1997), PTEN, a putative protein tyrosine phosphatase gene mutated in human brain, breast, and prostate cancer. Science. 275:1943-7). Both lines express all three Akt isoforms; in PC3 cells, Akt1 protein is expressed at approximately two times the level of Akt2, with Akt3 contributing to <10% of total Akt, whereas in U87MG cells, all three Akt proteins are expressed at equivalent levels (FIG. 18 A). Stable clones of PC3 and U87MG cells were generated harboring inducible shRNA constructs targeting all possible single and combined Akt isoforms (Table 2). Each Akt-targeting shRNA (shAkt) caused ˜75-99% KD of the corresponding Akt mRNA and proteins upon doxycycline (Dox) induction (FIG. 1 A, FIG. 18 B, and Table 3). Decreased steady-state phosphorylation of downstream targets PRAS40 and S6, up-regulation of p27^{Kip1}, and feedback stabilization of IRS1 were observed to varying degrees in response to the KDs, with the strongest effects observed in cells with all three Akt KDs (FIG. 1 A).

We next examined the effect of Akt KDs on the ability of PC3 cells to maintain the growth of established tumors in vivo. Dox-induced KD of Akt2 (shAkt2) or Akt3 (shAkt3) alone did not result in significant inhibition of tumor growth (FIG. 1 B and Table 3). In contrast, two different shRNA constructs targeting Akt1 (shAkt1) both showed significant tumor growth inhibition, each in two out of three independent clones. Tumor growth retardation or stasis was typically observed in these clones (FIG. 1 B, FIG. 18 H, and Table 3). Simultaneous KD of Akt1,2 (shAkt12) or Akt1,3 (shAkt13) also inhibited tumor growth, with almost all tumor growth halted and tumor regression observed in several of the Dox-treated mice. Interestingly, KD of both Akt2 and Akt3 (shAkt23) also resulted in significant tumor growth inhibition, with no tumor volume doubling during the 2 wk of Dox treatment, suggesting that Akt1 activity alone is not sufficient to maintain optimal tumor growth. Finally, triple-Akt KD (shAktI23) most effectively inhibited tumor growth, with consistent tumor regression observed during the first 2 wk of treatment. Similar results were observed in U87MG cells, which express similar levels of the three Akt isoforms. Among the three single KDs, only shAkt1 showed significant tumor stasis, and tumor regression was again observed with triple-Akt KD (FIG. 18, C-F). Thus, KD of Akt1 alone can inhibit tumor growth in both PC3 and U87MG xenografts, and this Akt1 dependency is not simply a total Akt dose effect. More pronounced tumor growth inhibition and regression, however, occurs in tumors with KD of all three Akt isoforms.

TABLE 1
DNA oligo sequences used in Akt shRNA vectors (pShuttle-H1 and pHUSH-GW)

| | | | DNA OLIGO LIGATED INTO PSHUTTLE-H1 (ALL IN 5′-3′ | |
|---|---|---|---|---|
| Target | Construct | | DIRECTION, THE 19 BP TARGET SEQUENCES ARE | SEQ ID |
| gene | # | | BOLDED) | No. |
| Akt1 | 252 | sense | 5′-GATCCCCTGACCATGAACGAGTTTGATTCAAGAGA | 1 |
| | | | TCAAACTCGTTCATGGTCATTTTTTGGAAA-3′ | |
| | | Anti- | 5′-AGCTTTTCCAAAAAATGACCATGAACGAGTTTGATC | 2 |
| | | sense | TCTTGAATCAAACTCGTTCATGGTCAGGG-3′ | |
| Akt1 | 253 | sense | 5′-GATCCCCGTGGACCACTGTCATCGAATTCAAGAGA | 3 |
| | | | TTCGATGACAGTGGTCCACTTTTTTGGAAA-3′ | |
| | | Anti- | 5′-AGCTTTTCCAAAAAAGTGGACCACTGTCATCGAATC | 4 |
| | | sense | TCTTGAATTCGATGACAGTGGTCCACGGG-3′ | |
| Akt2 | 254 | sense | 5′-GATCCCCCCTGGAGGCCACGGTACTTTTCAAGAGA | 5 |
| | | | AAGTACCGTGGCCTCCAGGTTTTTTGGAAA-3′ | |
| | | Anti- | 5′-AGCTTTTCCAAAAAACCTGGAGGCCACGGTACTTTC | 6 |
| | | sense | TCTTGAAAAGTACCGTGGCCTCCAGGGGG-3′ | |
| Akt2 | 255 | sense | 5′-GATCCCCTGACTTCGACTATCTCAAATTCAAGAGA | 7 |
| | | | TTTGAGATAGTCGAAGTCATTTTTTGGAAA-3′ | |
| | | Anti- | 5′-AGCTTTTCCAAAAAATGACTTCGACTATCTCAAATC | 8 |
| | | sense | TCTTGAATTTGAGATAGTCGAAGTCAGGG-3′ | |
| Akt3 | 259 | sense | 5′-GATCCCCGAATTGTAGTCCAACTTCATTCAAGAGA | 9 |
| | | | TGAAGTTGGACTACAATTCTTTTTTGGAAA-3′ | |
| | | Anti- | 5′-AGCTTTTCCAAAAAAGAATTGTAGTCCAACTTCATC | 10 |
| | | sense | TCTTGAATGAAGTTGGACTACAATTCGGG-3′ | |
| Akt3 | 260, 261 | sense | 5′-GATCCCCGCACTTTTGGGAAAGTTATTTCAAGAGA | 11 |
| | | | ATAACTTTCCCAAAAGTGCTTTTTTGGAAA-3′ | |
| | | Anti- | 5′-AGCTTTTCCAAAAAAGCACTTTTGGGAAAGTTATTC | 12 |
| | | sense | TCTTGAAATAACTTTCCCAAAAGTGCGGG-3′ | |
| Akt 1 & | 256 | sense | 5′-GATCCCCGCTACTACGCCATGAAGATTTCAAGAGA | 13 |
| Akt2 | | | ATCTTCATGGCGTAGTAGCTTTTTTGGAAA-3′ | |
| | | Anti- | 5′-AGCTTTTCCAAAAAAGCTACTACGCCATGAAGATTC | 14 |
| | | sense | TCTTGAAATCTTCATGGCGTAGTAGCGGG-3′ | |
| Akt 1 & | 257 | sense | 5′-GATCCCCAGGTGCTGGAGGACAATGATTCAAGAGA | 15 |
| Akt2 | | | TCATTGTCCTCCAGCACCTTTTTTTGGAAA-3′ | |
| | | Anti- | 5′-AGCTTTTCCAAAAAAAGGTGCTGGAGGACAATGA | 16 |
| | | sense | TCTCTTGAATCATTGTCCTCCAGCACCTGGG-3′ | |
| Akt 1 & | 258 | sense | 5′-GATCCCCCTACAACCAGGACCATGAGTTCAAGAGA | 17 |
| Akt2 | | | CTCATGGTCCTGGTTGTAGTTTTTTGGAAA-3′ | |
| | | Anti- | 5′-AGCTTTTCCAAAAAACTACAACCAGGACCATGAGT | 18 |
| | | sense | CTCTTGAACTCATGGTCCTGGTTGTAGGGG-3′ | |

TABLE 2
Summary of Akt shRNA constructs used and effective targeting sequences

| | | | | | | Seq |
|---|---|---|---|---|---|---|
| | Construct | | | | Effective siRNA 19 bp | ID |
| Target | # | Selection^{a} | Target sequence^{b} | | core sequence^{c} | No. |
| Akt1 | 252 | Pur | 5′-TGACCATGAA | sense | UGACCAUGAACGAGUUUGA | 19 |
| | | | CGAGTTTGA | | | |
| | | | | anti-sense | UCAAACUCGUUCAUGGUCA | 20 |
| Akt1 | 253 | Pur | 5′-GTGGACCAC | sense | GUGGACCACUGTCAUCGAA | 21 |
| | | | TGTCATCGAA | | | |
| | | | | anti-sense | UUCGAUGACAGUGGUCCAC | 22 |
| Akt2 | 254 | Pur | 5′-CCTGGAGGC | sense | CCUGGAGGCCACGGUACUU | 23 |
| | | | C ACGGTACTT | | | |
| | | | | anti-sense | AAGUACCGUGGCCUCCAGG | 24 |
| Akt2 | 255 | Pur | 5′-TGACTTCGAC | sense | UGACUUCGACUAUCUCAAA | 25 |
| | | | T ATCTCAAA | | | |
| | | | | anti-sense | UUUGAGAUAGUCGAAGUCA | 26 |
| Akt3 | 259 | Pur | 5′-GAATTGTAGT | sense | GAAUUGUAGUCCAACUUCA | 27 |
| | | | C CAACTTCA | | | |
| | | | | anti-sense | UGAAGUUGGACUACAAUUC | 28 |
| Akt3 | 260 | Pur | 5′-GCACTTTTGG | sense | GCACUUUUGGGAAAGUUAU | 29 |
| | | | GAAAGTTAT | | | |
| | | | | anti-sense | AUAACUUUCCCAAAAGUGC | 30 |
| Akt3 | 261 | Hyg | 5′-GCACTTTTGG | sense | GCACUUUUGGGAAAGUUAU | 31 |
| | | | GAAAGTTAT | | | |
| | | | | anti-sense | AUAACUUUCCCAAAAGUGC | 32 |
| Akt12 | 256 | Pur | 5′-GCTACTACGC | sense | GCUACUACGCCAUGAAGAU | 33 |
| | | | CATGAAGAT | | | |
| | | | | anti-sense | AUCUUCAUGGCGUAGUAGC | 34 |
| Akt12 | 257 | Pur | 5′-AGGTGCTGGA | sense | AGGUGCUGGAGGACAAUGA | 35 |
| | | | GGACAATGA | | | |
| | | | | antisense | AGGUGCUGGAGGACAAUGA | 36 |
| Akt13 | 258 | Pur | 5′-CTACAACCAG | sense | CUACAACCAGGACCAUGAG | 37 |
| | | | GACCATGAG | | | |
| | | | | anti-sense | CUCAUGGUCCUGGUUGTAG | 38 |

| | | | | | |
|---|---|---|---|---|---|
| Akt13 | 253 + 261 | Pur + Hyg | 253 and 261 | sense | 253 and 261 core sequences |
| | | | target sequences | | |
| | | | | anti-sense | 253 and 261 core sequences |
| Akt23 | 255 + 261 | Pur + Hyg | 255 and 261 | sense | 255 and 261 core sequences |
| | | | target sequences | | |
| | | | | anti-sense | 255 and 261 core sequences |
| Akt123 | 257 + 261 | Pur + Hyg | 257 and 261 | sense | 257 and 261 core sequences |
| | | | target sequences | | |
| | | | | anti-sense | 257 and 261 core sequences |

^{a}Antibiotic selection used to establish stable expression of the shRNA. Pur, puromycin; Hyg, Hygromycin.
^{b}Sense sequence in the target (all in 5′-3′ direction).
^{c}siRNA duplexes or shRNA hairpins containing these 19 bp core sequences should also be effective against the indicated genes (both sense and sense sequences are in 5′-3′ direction)

TABLE 3
Summary of Akt knockdown efficiency and effect on xenograft
tumor growth for various PC3-Akt shRNA clones

| | | | | | | | | | | % TGI | | % TGI |
|---|---|---|---|---|---|---|---|---|---|---|---|---|
| Target | Construct | Clone | Treatment | % Akt1^{a} | % Akt2^{a} | % Akt3^{a} | n^{b} | P^{c} | R^{d} | (d14)^{e} | DRS^{f} | (DRS)^{e} |
| Akt1 | 252 | 2 | Dox− | 100 | 100 | 100 | 8 | 6 | 0 | | | |
| | | | Dox+ | 21.9 ± 1.1 | 93.8 ± 10.2 | 151.0 ± 58.3 | 8 | 8 | 0 | −14 | >21 | — |
| | 252 | 9 | Dox− | 100 | 100 | 100 | 8 | 5 | 0 | | | |
| | | | Dox+ | 22.3 ± 2.3 | 122.7 ± 11.3 | 120.3 ± 8.6 | 8 | 2 | 1 | 72 | 21 | 81* |
| | 252 | 10 | Dox− | 100 | 100 | 100 | 8 | 8 | 0 | | | |
| | | | Dox+ | 11.2 ± 2.6 | 89.1 ± 13.9 | 106.8 ± 3.2 | 8 | 5 | 0 | 52 | 20 | 58* |
| | 253 | 17 | Dox− | 100 | 100 | 100 | 8 | 7 | 0 | | | |
| | | | Dox+ | 11.6 ± 1.2 | 80.1 ± 9.7 | 106.1 ± 4.3 | 8 | 2 | 3 | 97* | 13 | 97* |
| | 253 | 25 | Dox− | 100 | 100 | 100 | 8 | 5 | 1 | | | |
| | | | Dox+ | 13.9 ± 5.0 | 82.3 ± 1.3 | 92.9 ± 5.3 | 8 | 7 | 0 | 26 | >21 | — |
| | 253 | 29 | Dox− | 100 | 100 | 100 | 10 | 10 | 0 | | | |
| | | | Dox+ | 5.9 ± 0.1 | 70.3 ± 5.6 | 98.0 ± 1.0 | 10 | 8 | 0 | 56* | 5 | 56* |
| Akt2 | 255 | 4 | Dox− | 100 | 100 | 100 | 8 | 7 | 0 | | | |
| | | | Dox+ | 88.0 ± 16.5 | 11.6 ± 2.4 | 116.4 ± 32.0 | 8 | 5 | 2 | −7 | >21 | — |
| | 255 | 23 | Dox− | 100 | 100 | 100 | 10 | 10 | 0 | | | |
| | | | Dox+ | 105.0 ± 6.1 | 10.4 ± 1.3 | 106.3 ± 8.0 | 10 | 9 | 0 | 22 | >21 | — |
| Akt3 | 260 | 6 | Dox− | 100 | 100 | 100 | 8 | 6 | 1 | | | |
| | | | Dox+ | 101.0 ± 4.0 | 95.5 ± 10.5 | 10.0 ± 4.0 | 8 | 4 | 1 | 45 | >21 | — |
| Akt12 | 257 | 4 | Dox− | 100 | 100 | 100 | 8 | 3 | 2 | | | |
| | | | Dox+ | 5.7 ± 0.9 | 13.2 ± 1.1 | 129.6 ± 11.3 | 8 | 0 | 5 | 137* | 14 | 137* |
| | 257 | 6 | Dox− | 100 | 100 | 100 | 8 | 7 | 0 | | | |
| | | | Dox+ | 6.6 ± 0.3 | 12.3 ± 1.6 | 128.9 ± 18.3 | 8 | 1 | 4 | 80* | 11 | 90* |
| Akt13 | 253 + 261 | 2 | Dox− | 100 | 100 | 100 | 8 | 8 | 0 | | | |
| | | | Dox+ | 5.1 ± 2.0 | 66.0 ± 2.6 | 18.0 ± 3.7 | 8 | 1 | 3 | 102* | 7 | 120* |
| Akt23 | 255 + 261 | 8 | Dox− | 100 | 100 | 100 | 8 | 6 | 0 | | | |
| | | | Dox+ | 96.8 ± 18.8 | 6.3 ± 0.3 | 9.9 ± 2.1 | 8 | 0 | 0 | 82* | 7 | 93* |
| Akt123 | 257 + 261 | 10 | Dox− | 100 | 100 | 100 | 10 | 8 | 2 | | | |
| | | | Dox+ | 1.2 ± 0.3 | 9.5 ± 0.7 | 4.5 ± 2.1 | 10 | 0 | 6 | 116* | 6 | 135* |
| | 257 + 261 | 12 | Dox− | 100 | 100 | 100 | 8 | 6 | 0 | | | |
| | | | Dox+ | 9.0 ± 3.6 | 21.7 ± 2.9 | 12.7 ± 0.9 | 8 | 0 | 5 | 116* | 7 | 110* |
| | 257 + 261 | 12 | Dox− | 100 | 100 | 100 | 10 | 5 | 0 | | | |
| | | | Dox+ | 9.0 ± 3.6 | 21.7 ± 2.9 | 12.7 ± 0.9 | 10 | 1 | 4 | 106* | 10 | 129* |
| eGFP | 310 | 3 | Dox− | 100 | 100 | 100 | 8 | 6 | 1 | | | |
| | | | Dox+ | 93.7 ± 13.2 | 95.0 ± 6.4 | 87.3 ± 4.7 | 8 | 7 | 0 | −90 | >21 | — |
| ^{a}Percentage of message level after 72 hours of Dox treatment compared to untreated control determined by real-time quantitative RT-PCR (Taqman). Data represent mean ± SEM of at least 3 independent experiments. | | | | | | | | | | | | |
| ^{b}Number of tumors analyzed in each cohort. | | | | | | | | | | | | |
| ^{c}Number of tumors progressed by d14, defined by tumor volume >2 fold of the initial size at the start of treatment. | | | | | | | | | | | | |
| ^{d}Number of tumors regressed by d14, defined by tumor volume <50% of the initial size at the start of treatment. | | | | | | | | | | | | |
| ^{e}Percentage of tumor growth inhibition (TGI) at day 14 or the first day when significant difference was achieved, calculated as % TGI = % [Vc(dx − d0) − Vt(dx − d0)]/Vc(dx − d0) * 100, where Vc(dx − d0) is the difference in mean tumor volume of the control cohort (Vc) between the day of analysis (dx) and the day when treatment started (d0), and Vt(dx − d0) is the difference in mean tumor volume of the treated cohort (Vt) between the day of analysis and the day when treatment started. % TGI >100 indicates tumor regression. | | | | | | | | | | | | |
| ^{f}Day to Reach Significance, number of days taken after treatment before significant difference between the control and the treatment group is achieved. | | | | | | | | | | | | |
| *P < 0.05 compared to sucrose vehicle treated group, determined by Student's t test. | | | | | | | | | | | | |

### Example 2

### Akt KDs Induced Cell Cycle Delay without Significant Apoptosis

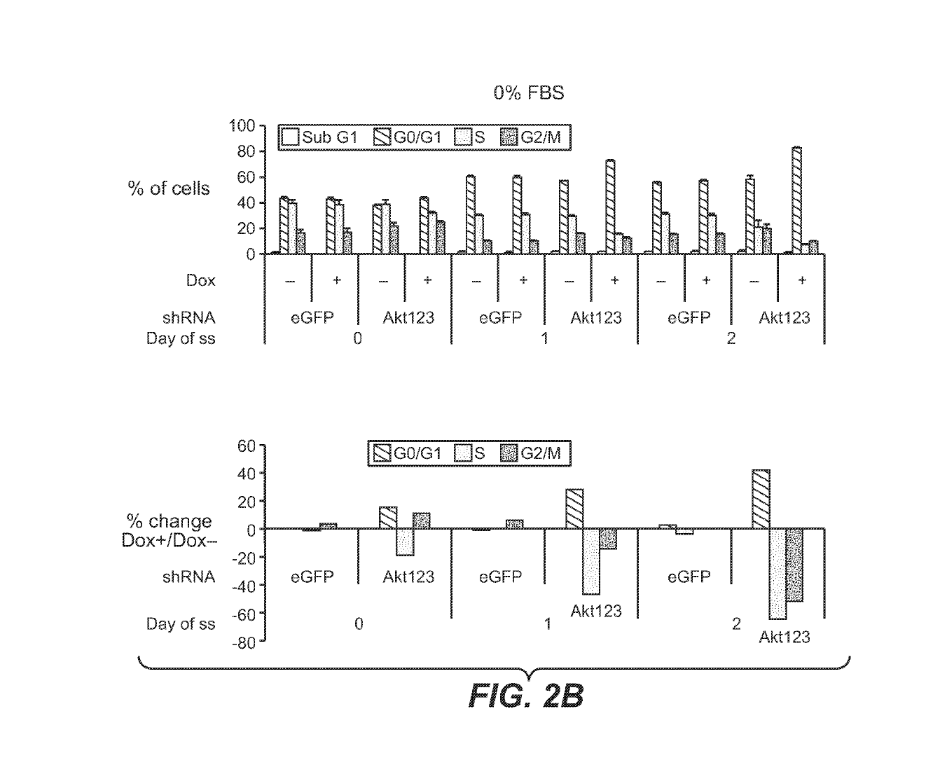

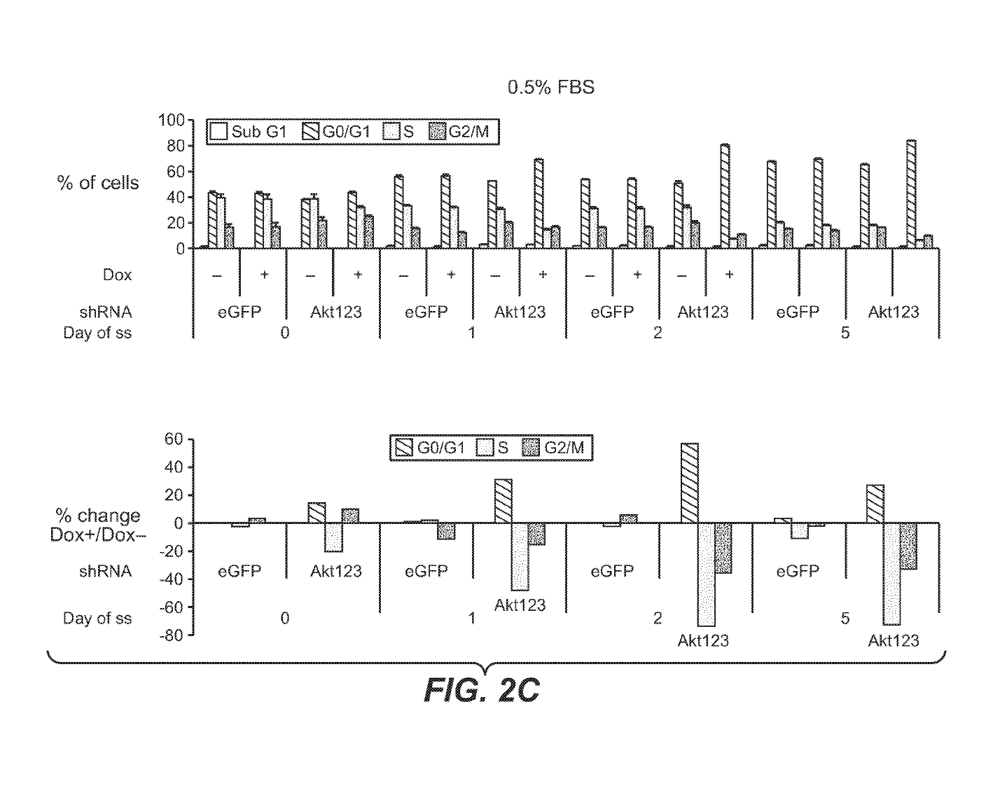

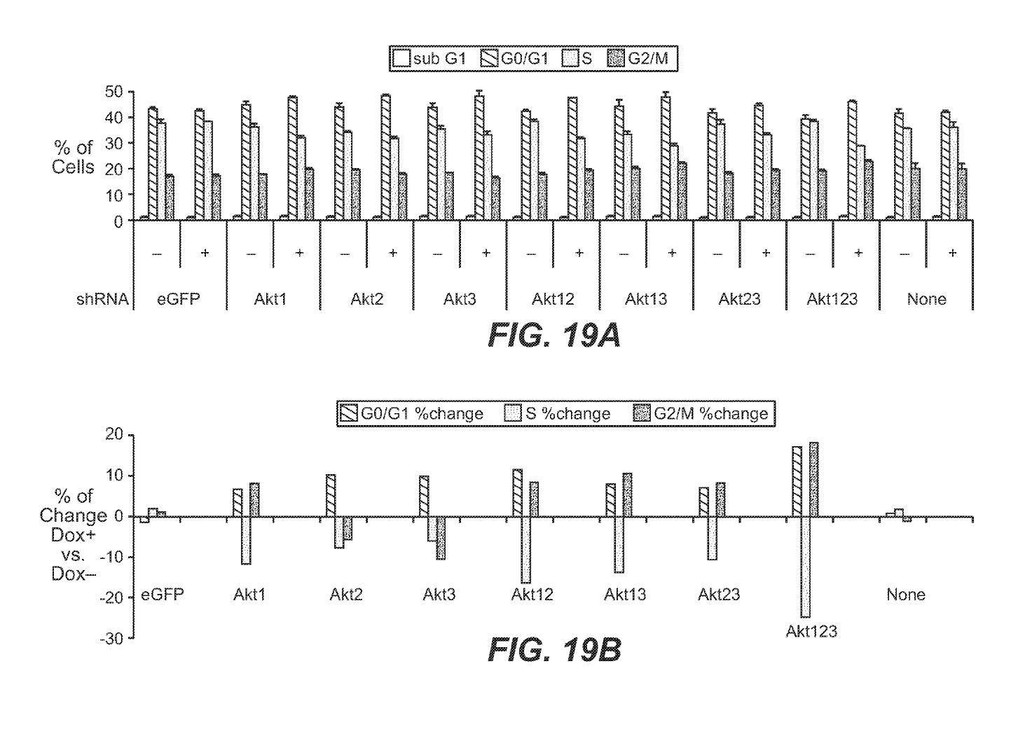

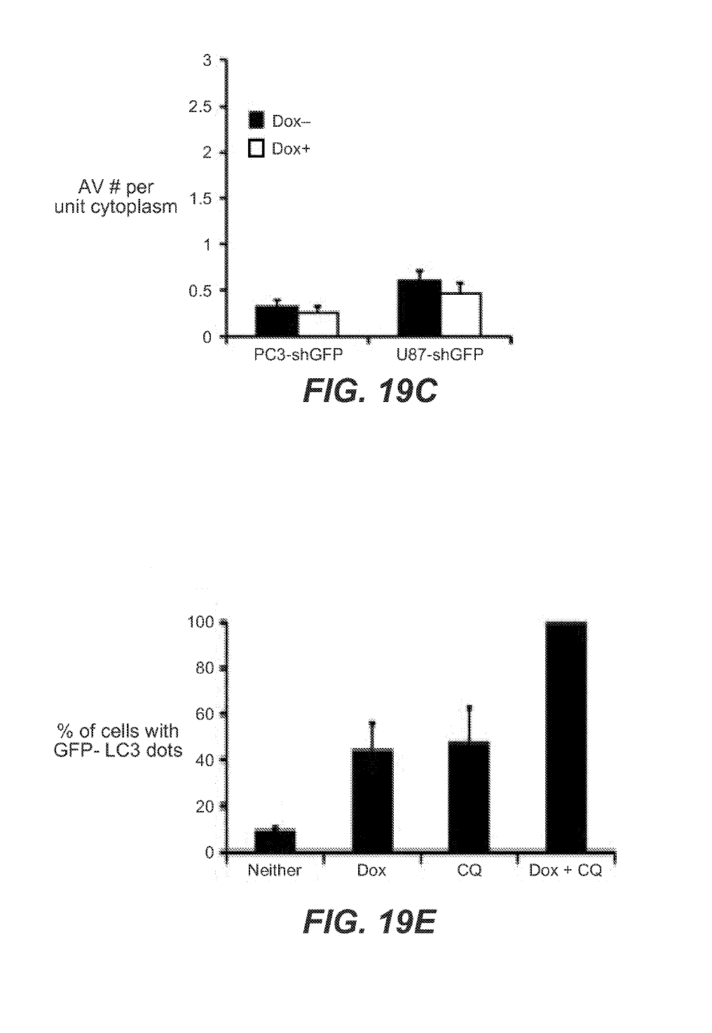

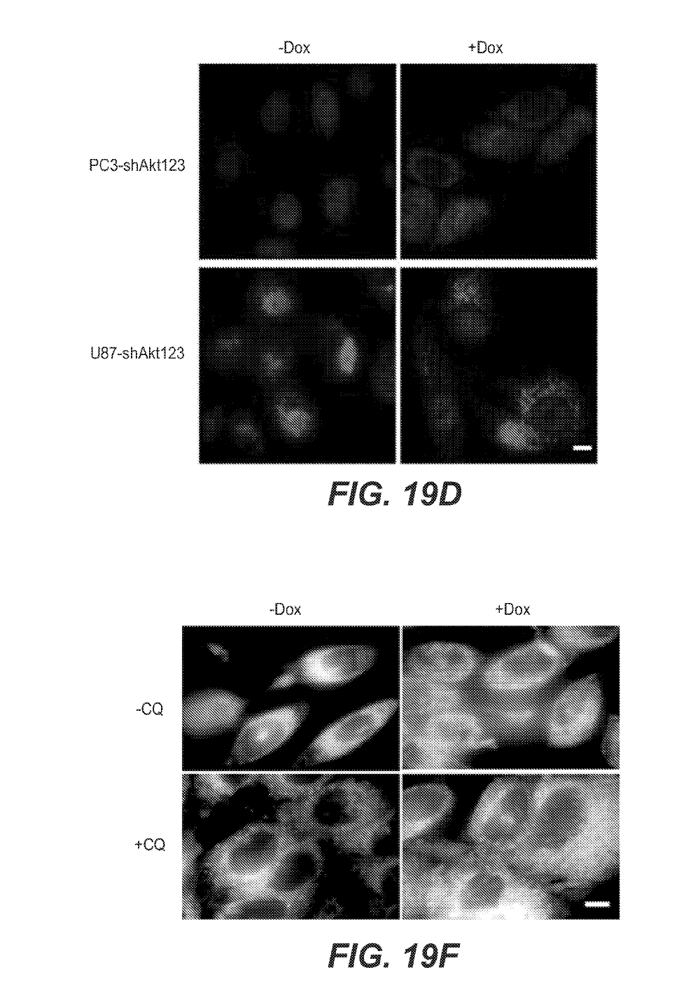

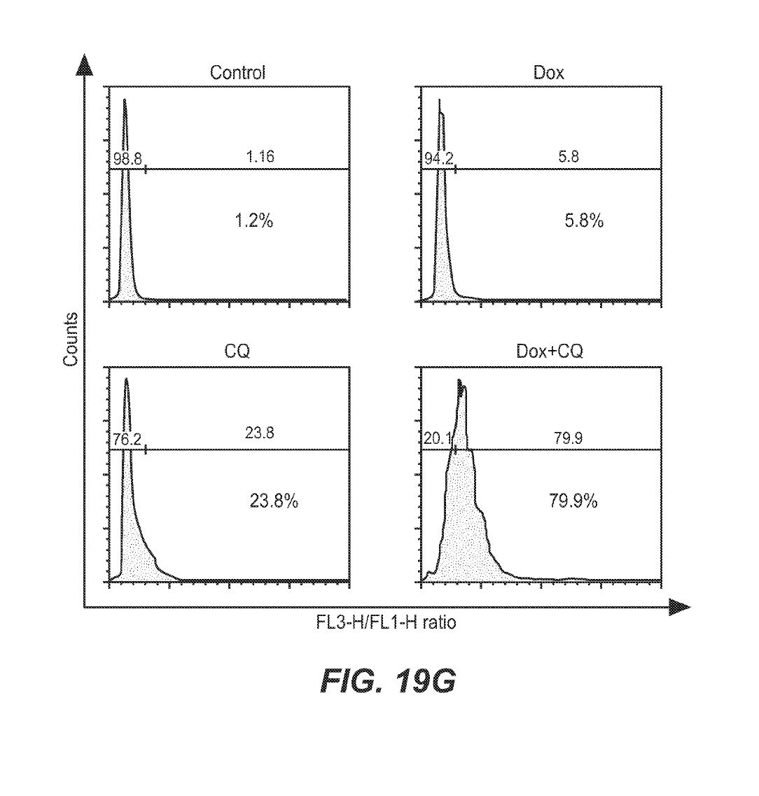

Analysis of PC3 tumors with Akt KDs revealed a mild decrease in the proliferation marker Ki-67 and no significant increase in TUNEL-positive cells compared with control tumors (FIG. 2 A). The lack of apoptosis was also observed in PC3 cells cultured in vitro. Under 10% FBS, a mild increase in G0/G I and a decrease in S phase was observed in cells expressing each shAkt construct. Slightly increased accumulation of cells in the G2/M phase was also observed in cells expressing shRNA for Akt1 alone and any combinations of two or three Akt isoforms, suggesting a cell cycle delay in both DNA replication and mitosis in these cells. However, no significant sub-G1 population was observed with any of the KDs (FIG. 19, A and B). Additional experiments also failed to detect significant caspase activation in response to Akt KDs in both PC3 and U87MG cells. To partially mimic the suboptimal growth condition in the in vivo environment, we starved the cells of serum in culture and asked whether the cells became more sensitive to Akt KD. Indeed, complete serum starvation or reducing serum to 0.5% resulted in markedly increased accumulation of cells in the G0/G1 phase. However, still no significant sub-G1 peak was observed for at least 2 d in 0% FBS and 5 d in 0.5% FBS (FIG. 2, B and C).

### Example 3

### Akt KD Promoted Autophagy in PC3 and U87MG Cells

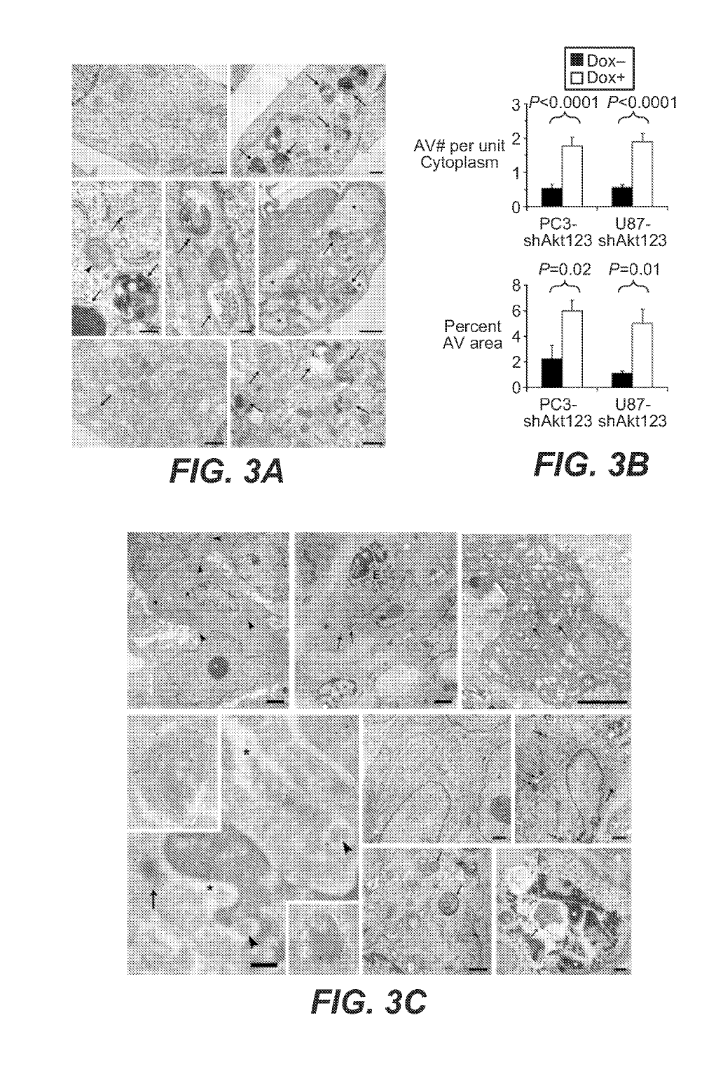

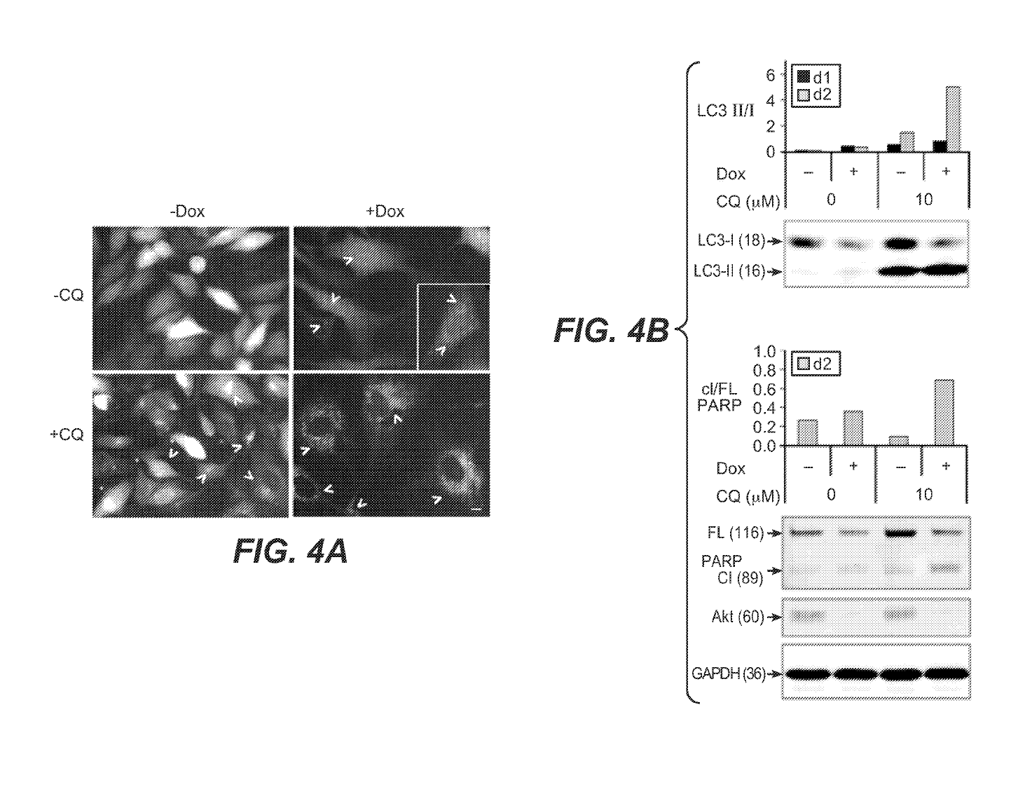

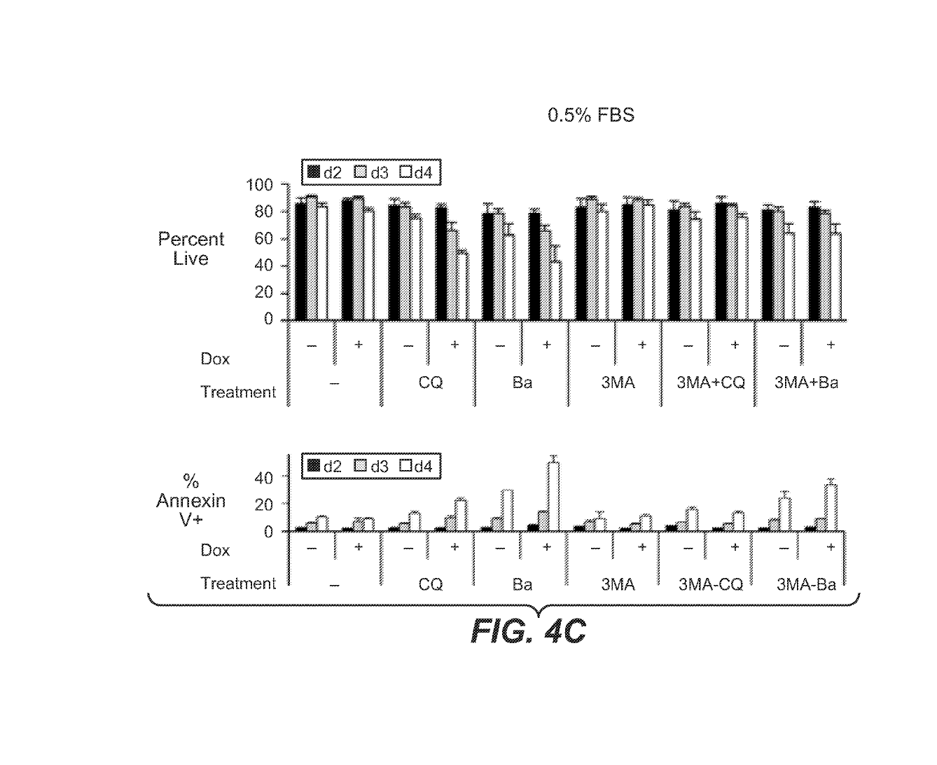

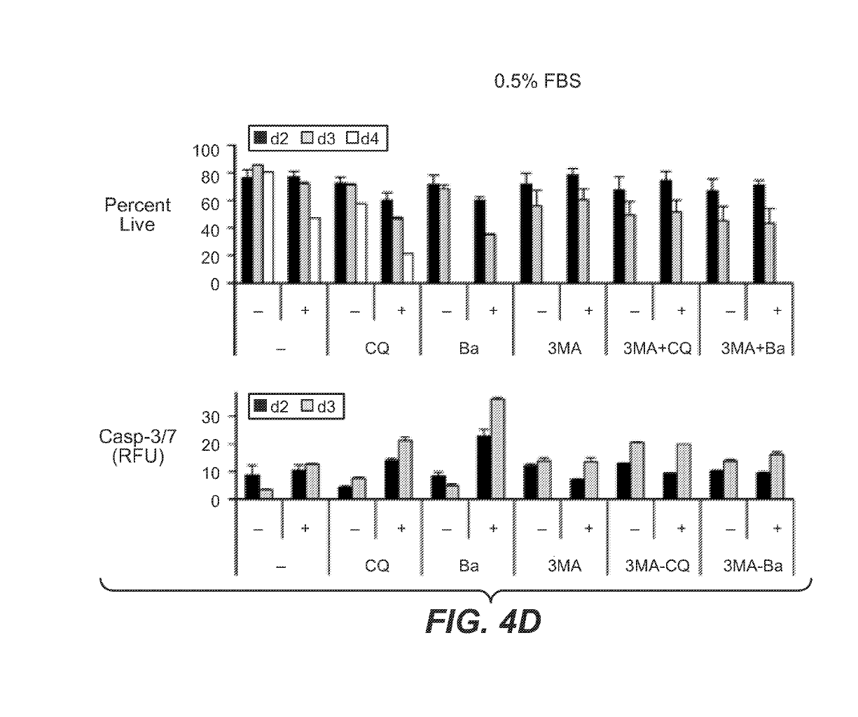

Because Akt has been shown to inhibit autophagy (Arico, S., A. Petiot, C. Bauvy, P. F. Dubbelhuis, A. J. Meijer, P. Codogno, and E. Ogier-Denis, (2001), The tumor suppressor PTEN positively regulates macroautophagy by inhibiting the phosphatidylinositol 3-kinase/protein kinase B pathway. J Biol Chem. 276:35243-6. Epub 2001 Jul. 26; Degenhardt et al., 2006), we asked whether specific KD of endogenous Akt could promote autophagy. Indeed, EM analysis revealed a significantly increased accumulation of AV s in both PC3 and U87MG cells induced to express shAkt123 (FIG. 3, A and B; and FIG. 19 C). The accumulation of AV and acidic vesicular organelles (AVOs) was further confirmed by localization of the autophagosome marker GFP-LC3, staining with an anti-LC3 antibody, and fluorescent dyes monodansylcadaverine (MDC) and acridine orange (AO; FIG. 4 A, FIG. 19, D-G).

We examined xenograft tumors expressing shAkt by EM. The control GFP-targeting shRNA-expressing PC3 tumors consist of healthy looking cells connected by cell-cell junctions (FIG. 3 C, a). In contrast, cells in the shAkt123-expressing tumors exhibit morphological signs of degeneration and loss of cell-cell contact after 10-15 d of Dox treatment (FIG. 3 C, b). Late AV s positive for human lysosome-associated membrane protein (LAMP)1 are found in degenerating tumor cells (FIG. 3 C, b-d). Also, these cells often contain swollen mitochondria and dilated RER that are drastically disorganized, suggesting a connection between energy metabolism, ER stress, and autophagy. Chromatin clumping and fragmentation characteristic of typical apoptosis are rarely observed in the degenerating tumor cells; instead, some AV-containing cells exhibit mild pyknosis typical of cells undergoing autophagic degeneration (FIG. 3 C, b and d).

To determine whether AV accumulation occurred in tumor cells before morphological signs of degeneration, we examined U87MG tumors with either 5 d or 3 wk of Akt KD. In tumors expressing shAkt 123 for 5 d, most cells showed similar gross morphology to vehicle-treated controls, but with an approximately two times increase in the percent AV area (from 0.78% in the control tumors to 1.53% in Dox-treated tumors; P<0.05; FIG. 3 C, e-h). After 3 wk of Akt KD, U87MG tumors show signs of degeneration in many cells similar to PC3 tumors treated for 15 d (unpublished data).

### Example 4

### Lysosomotropic Agents Accelerated Cell Death in PC3 Cells with Akt KD

Despite the elevated levels of autophagy and mild cell cycle delay, PC3 cells expressing shAkt123 can survive in culture for many passages under 10% FBS without appreciable increase in cell death. Even under reduced serum (0.5% FBS), there is only marginal decrease in viability over a prolonged period (unpublished data). Although the literature has been controversial on the effect of early stage autophagy inhibition on cell survival, blocking autophagy at a late stage has been more consistently shown to cause accelerated cell death under autophagy-inducing conditions (Kanzawa, T., I. M. Germano, T. Komata, H. Ito, Y. Kondo, and S. Kondo, (2004), Role of autophagy in temozolomide-induced cytotoxicity for malignant glioma cells. Cell Death Differ. 11:448-57; Boya, P., R. A. Gonzalez-Polo, N. Casares, J. L. Perfettini, P. Dessen, N. Larochette, D. Metivier, D. Meley, S. Souquere, T. Yoshimori, G. Pierron, P. Codogno, and G. Kroemer, (2005), Inhibition of macroautophagy triggers apoptosis. Mol Cell Biol. 25:1025-40; Gonzalez-Polo, R. A., P. Boya, A. L. Pauleau, A. Jalil, N. Larochette, S. Souquere, E. L. Eskelinen, G. Pierron, P. Saftig, and G. Kroemer, (2005), The apoptosis/autophagy paradox: autophagic vacuolization before apoptotic death. J Cell Sci. 118:3091-102. Epub 2005 Jun. 28; Kroemer and Jaattela, 2005; Yu, L., F. Wan, S. Dutta, S. Welsh, Z. Liu, E. Freundt, E. H. Baehrecke, and M. Lenardo, (2006), Autophagic programmed cell death by selective catalase degradation. Proc Natl Acad Sci USA. 103:4952-7). Therefore, we investigated the effect of blocking the completion of autophagy initiated by Akt KD on cell viability. In PC3-shAkt123 cells stably expressing GFP-LC3, Akt KD resulted in punctate GFP signals (FIG. 4 A and FIG. 19 E) with a corresponding reduction of the nonlipidated precursor form of the endogenous LC3 (LC3-I) and a slight increase in the lipidated auto phagosome-localized LC3-II, which is rapidly turned over in the autolysosomes (FIG. 4 B; Klionsky et al., 2008). The lysosomotropic agent chloroquine (CQ), a weak base amine widely used to inhibit the maturation of autophagosomes into degradative autolysosomes (Bova, P., R. A. Gonzalez-Polo, N. Casares, J. L. Perfettini, P. Dessen, N. Larochette, D. Metivier, D. Meley, S. Souquere, T. Yoshimori, G. Pierron, P. Codogno, and G. Kroemer, (2005), Inhibition of macroautophagy triggers apoptosis. Mol Cell Biol. 25:1025-40; Kroemer and Jaattela, 2005; Lum, J. J., D. E. Bauer, M. Kong, M. H. Harris, C. Li, T. Lindsten, and C. B. Thompson, (2005), Growth factor regulation of autophagy and cell survival in the absence of apoptosis. Cell. 120:237-48), caused the appearance of small GFP-LC3 clusters in the perinuclear region. The combination of CQ with Akt KD resulted in a much stronger accumulation of GFP-LC3 dots as well as augmented accumulation of LC3-II in the presence of continued LC3-I turnover, consistent with a defect in autolysosomal degradation. Similar accumulation of MDc+ vacuoles was also observed (FIG. 19 F). This was accompanied by an accelerated cell death in shAkt123-expressing cells treated with CQ under 0.5% and, more pronouncedly, 0% FBS (FIG. 4, C and D). A second lysosomotropic agent, bafilomycin A1 (Ba), which inhibits the vacuolar proton pump (VH+-ATPase) and prevents the proper acidification of lysosomal compartments (Yamamoto, A., Y. Tagawa, T. Yoshimori, Y. Moriyama, R. Masaki, and Y. Tashiro, (1998), Bafilomycin A1 prevents maturation of autophagic vacuoles by inhibiting fusion between autophagosomes and lysosomes in rat hepatoma cell line, H-4-II-E cells. Cell Struct Funct. 23:33-42), also promoted cell death in combination with shAkt 123. Increased annexin V-positive population and caspase-3,7 activity was observed in cells treated with either CQ or Ba in combination with Akt KD, correlating with an increase in poly-ADP-ribose polymerase (PARP) cleavage in these cells (FIG. 4 B). In contrast, pretreatment with 1 mM 3-MA, an inhibitor of the earliest stage of autophagosome formation, attributed to its inhibition of class III PI3K (Seglen, P. O., and P. B. Gordon, (1982), 3-Methyladenine: specific inhibitor of autophagic/lysosomal protein degradation in isolated rat hepatocytes. Proc Natl Acad Sci USA. 79:1889-92; Petiot, A., E. Ogier-Denis, E. F. Blommaart, A. J. Meijer, and P. Codogno, (2000), Distinct classes of phosphatidylinositol 3′-kinases are involved in signaling pathways that control macroautophagy in HT-29 cells. J Biol Chem. 275:992-8), suppressed the cell death-promoting effect of either CQ or Ba on shAkt expressing cells (FIG. 4, C and D). This suggests that the accelerated cell death caused by the lysosomotropic inhibitors is dependent on the accumulation of abnormal AVs.

### Example 5

### CQ Accelerated Cell Death in Combination with PI3K and Akt Inhibitors

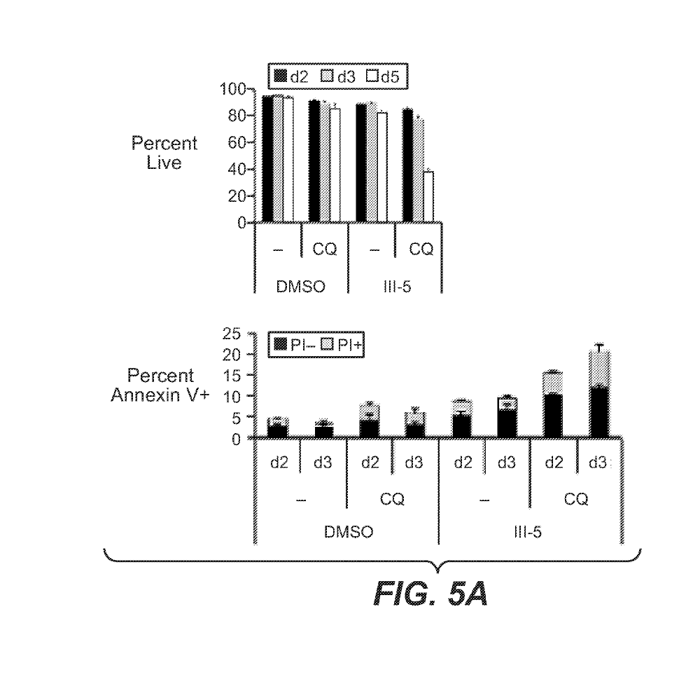

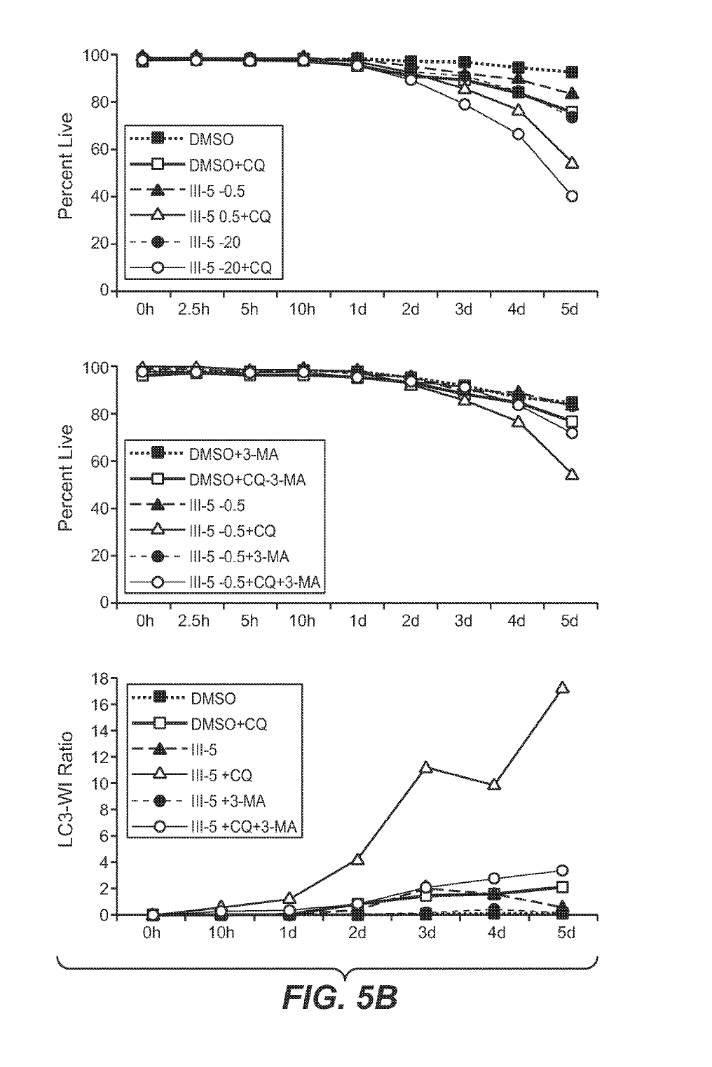

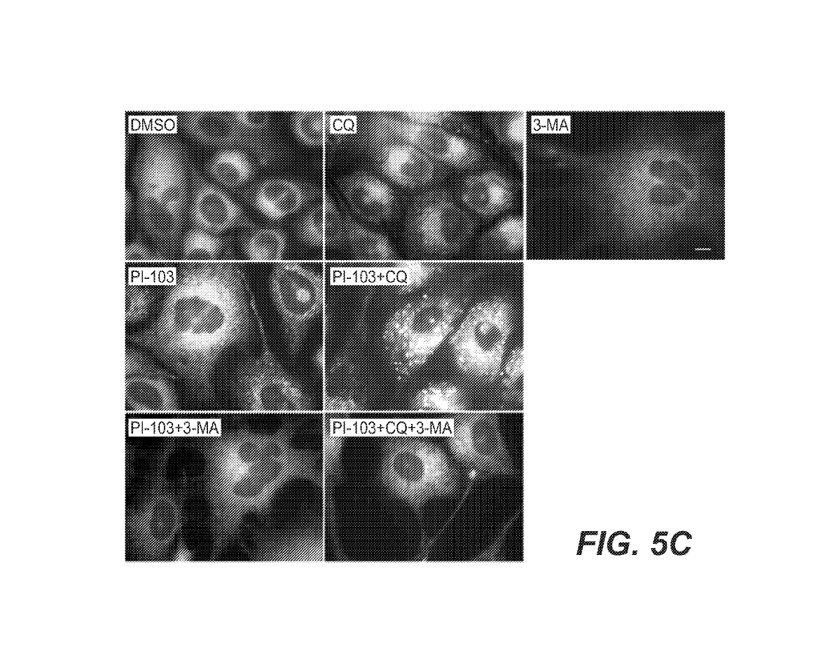

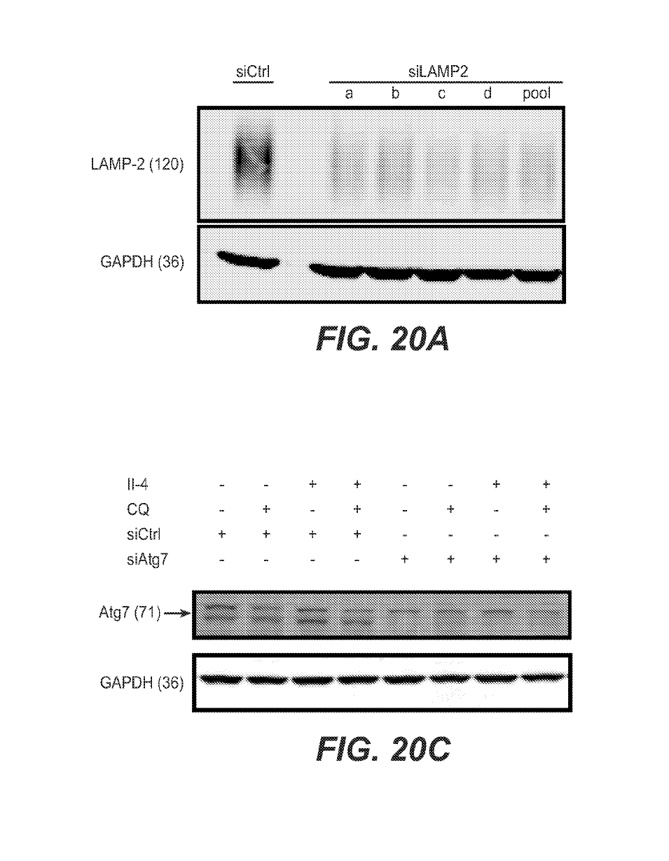

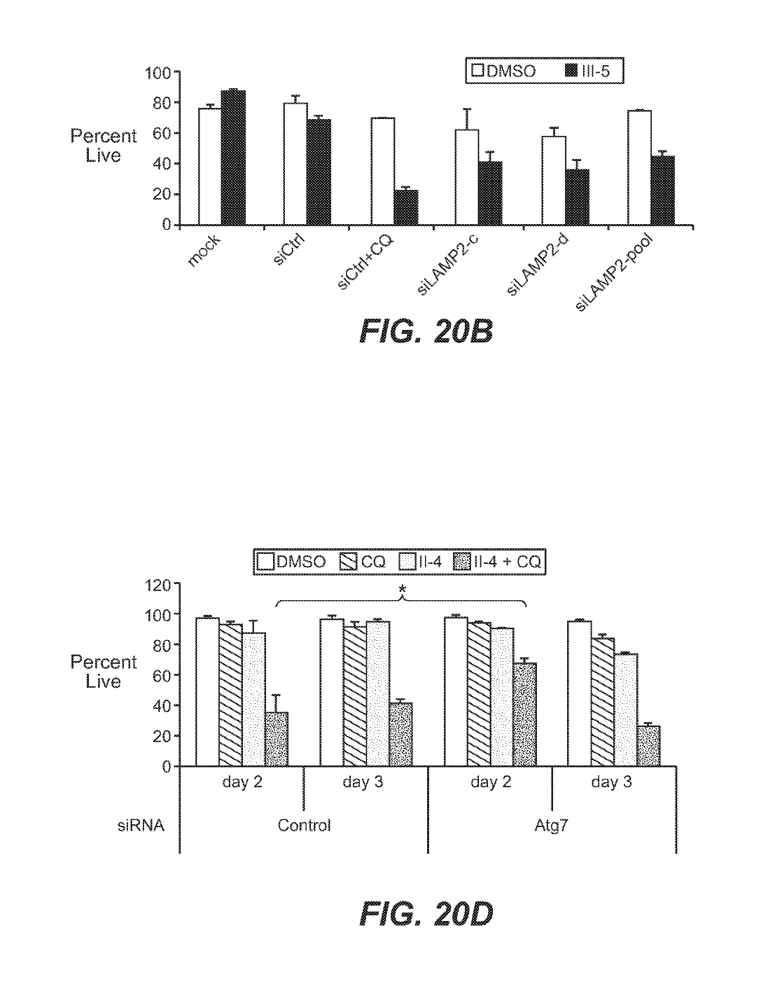

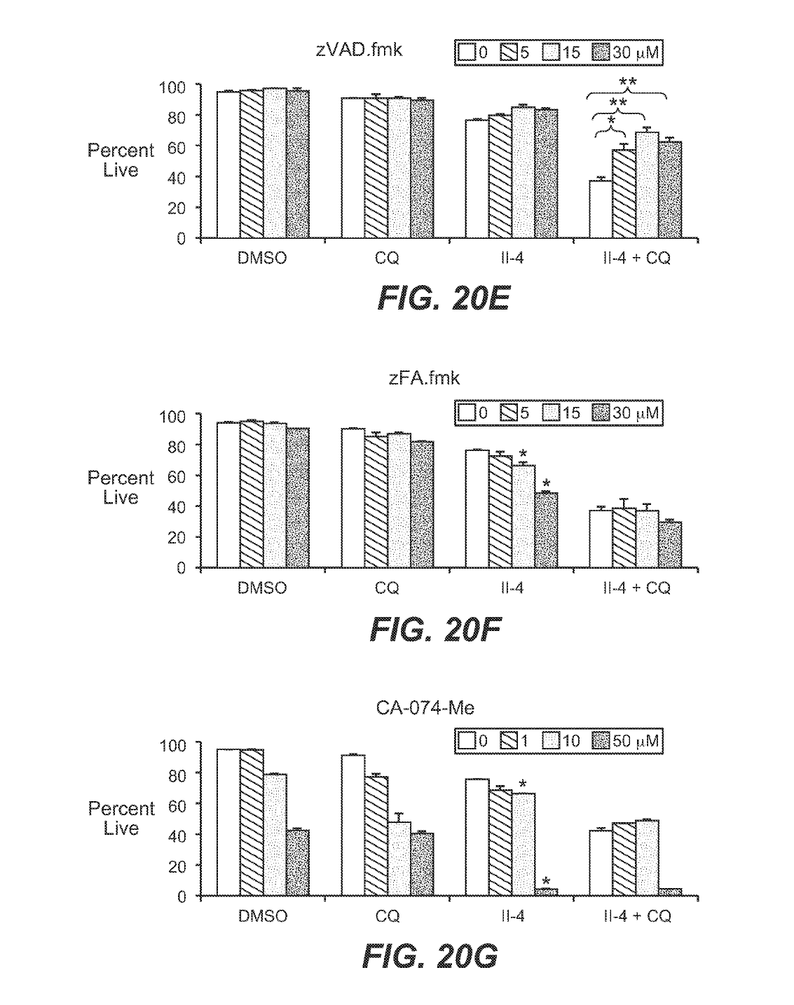

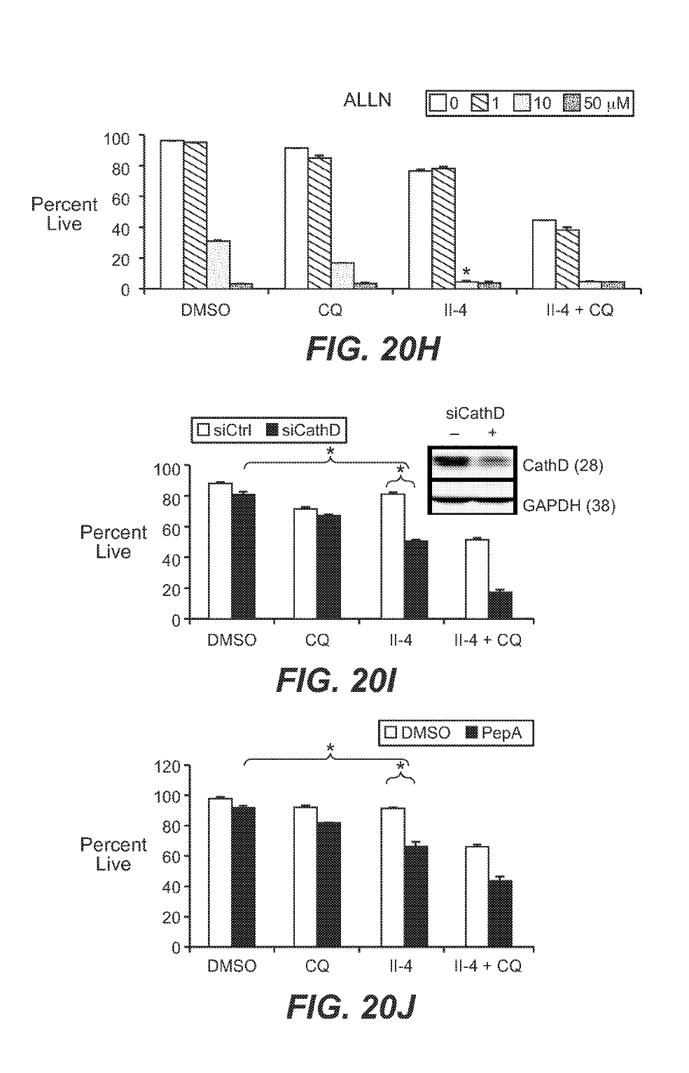

Recently, a phosphatidylinositol ether lipid analogue that inhibits Akt activation was reported to induce autophagy with radiosensitizing effect (Fujiwara et al., 2007). Because phosphatidylinositol ether lipid analogues are known to have additional cellular targets (Gills et al., 2006; Memmott et al., 2008), we asked whether other specific inhibitors of PI3K-Akt could also induce autophagy and sensitize cells to late stage autophagy inhibition. We first used a dual PI3K/mTOR inhibitor, compound III-5 (PI-103), which inhibits the class I PI3Ks and mTOR at nanomolar concentrations but is >1,000-fold less potent on the class III PI3K (Knight, Z. A., B. Gonzalez, M. E. Feldman, E. R. Zunder, D. D. Goldenberg, O. Williams, R. Loewith, D. Stokoe, A. Balla, B. Toth, T. Balla, W. A. Weiss, R. L. Williams, and K. M. Shokat, (2006), A pharmacological map of the PI3-K family defines a role for p110alpha in insulin signaling. Cell. 125:733-47). In contrast to the broad-spectrum PI3K inhibitors wortmannin or LY294002, which are equipotent at inhibiting both class I and III PI3Ks and inhibit autophagy caused by the latter activity (Petiot et al., 2000; Knight et al., 2006), III-5 is potent at inducing the accumulation of AVs (FIG. 5, B and C). Similar to Akt KD, combination with CQ accelerated the death of cells treated with III-5 (FIG. 5 A). The markedly increased LC3-II to LC3-I ratio and the appearance of enlarged vacuoles brightly stained by MDC was observed before the detection of overt cell death (FIG. 5, B and C). As observed with Akt KD, pretreatment with 3-MA reduced both LC3-II to -I ratios and the accumulation of MDC⁺ vacuoles and slowed down the rate of cell death (FIG. 5, B and C). In contrast, the cell death-promoting effect of CQ was partially mimicked by siRNA KD of LAMP2, a protein previously shown to be required for the maturation of autophagosomes into autolysosomes (FIG. 20, A and B); Gonzalez-Polo et al., 2005).

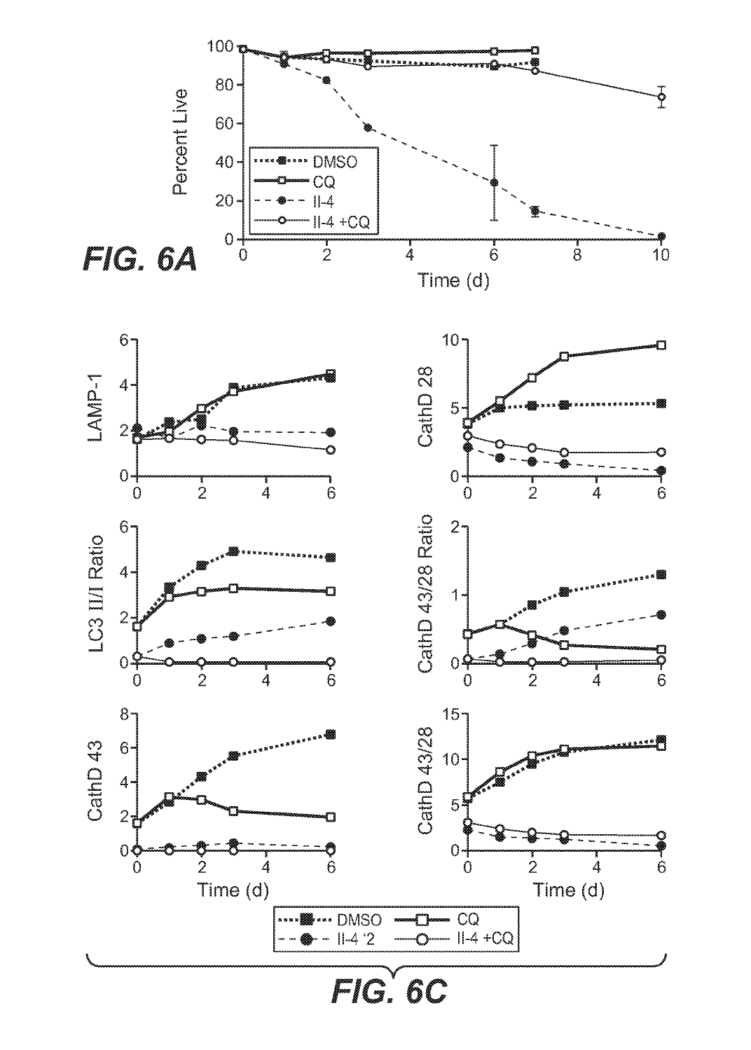

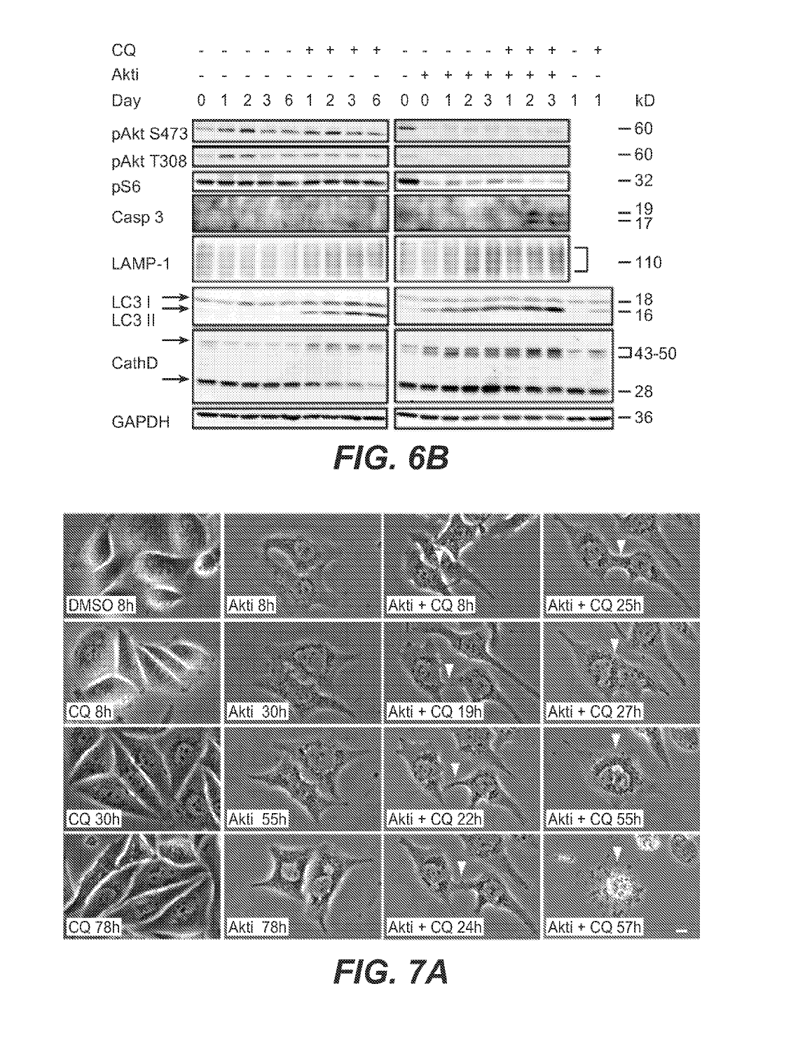

A similar effect of CQ was also obtained with one of the most selective Akt inhibitors reported, the dual Akt1,2 inhibitor compound II-4 (Akti-1/2) (Barnett et al., 2005). Treatment with II-4 alone effectively inhibited the phosphorylation of Akt on both Ser473 and Thr308 residues and significantly reduced the phosphorylation of downstream target S6 without causing significant cell death (FIG. 6, A and B). Cotreatment with CQ resulted in a rapid drop in cell viability with complete cell death observed by day 10. Immunoblot analysis revealed a significant accumulation of LC3-II within 24 h of II-4 treatment, which is further enhanced upon CQ cotreatment (FIG. 6, B and C).

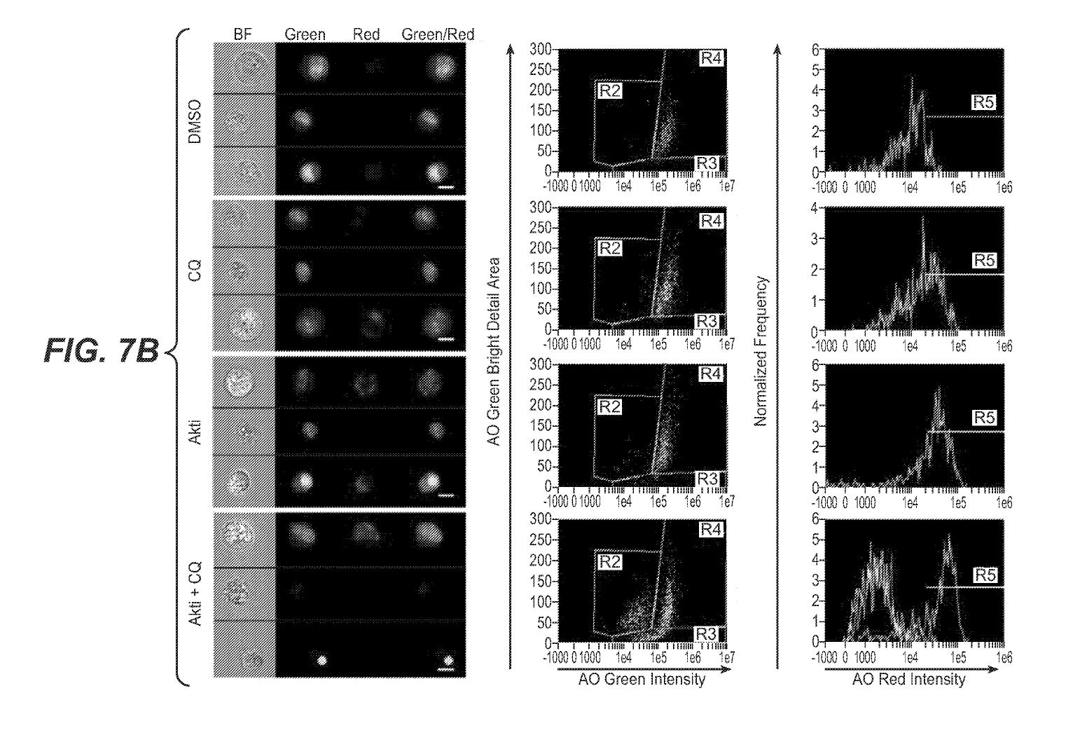

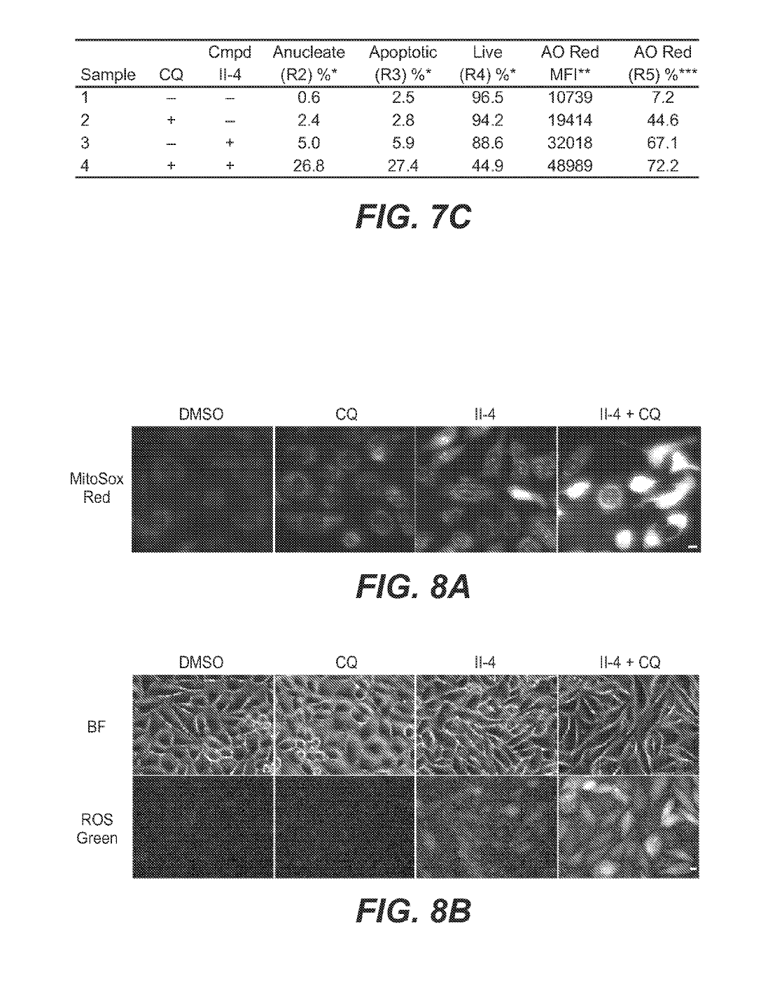

To follow the kinetics of cell death, we used time-lapse microscopy to image live cells treated with CQ and II-4 (FIG. 7 A). CQ treatment alone caused a mild decrease in cell division and a gradual accumulation of dark particles in the perinuclear region. AO staining indicates that these particles are AVOs whose formation is inhibited by 3-MA (unpublished data). Treatment with II-4 alone resulted in near-complete inhibition of cell division without overt cell death. These cells exhibited a flattened morphology with accumulation of AVOs that eventually filled the cytoplasm. Cells treated with both II-4 and CQ showed similar accumulation of AVOs, but cell shrinkage and plasma membrane rupture was observed within 48 h. On a few occasions, two neighboring cells were found to form a membrane junction that expanded into complete fusion between the two cells before rupture of the plasma membrane (FIG. 7 A, white arrowheads).

Similar correlation between AVO accumulation and cell death was observed using multispectral imaging flow cytometry (FIG. 7, B and C). Treatment with either CQ or II-4 alone induced AVO accumulation without significant loss of viability, whereas the combination of both resulted in a further increase in AVO accumulation in live cells and a concomitant increase in cells with condensed apoptotic nuclei and the appearance of anucleated population characteristics of necrotic cells.

### Example 6

### Autophagy Inhibition and Degradation Defective Autolysosome Accumulation Both Contribute to Accelerated Cell Death Induced by CG in Combination with II-4

To investigate whether autophagy inhibition by itself is sufficient to induce accelerated cell death in combination with Akt inhibition, we used siRNA to KD Atg7, a gene involved in the formation of autophagosomes (Ohsumi, Y. 2001. Molecular dissection of autophagy: two ubiquitin-like systems.). KD of Atg7 alone did not show a significant effect on cell death but induced a small drop in cell viability by day 3 when combined with Akti. However, when combined with both CQ and II-4, Atg7 KD resulted in a transient delay of cell death at day 2 (FIG. 20, C and D). Together with the aforementioned effect of 3-MA, these data suggest that autophagy inhibition and defective AV accumulation both contribute to the accelerated cell death induced by CQ in combination with Akt inhibition.

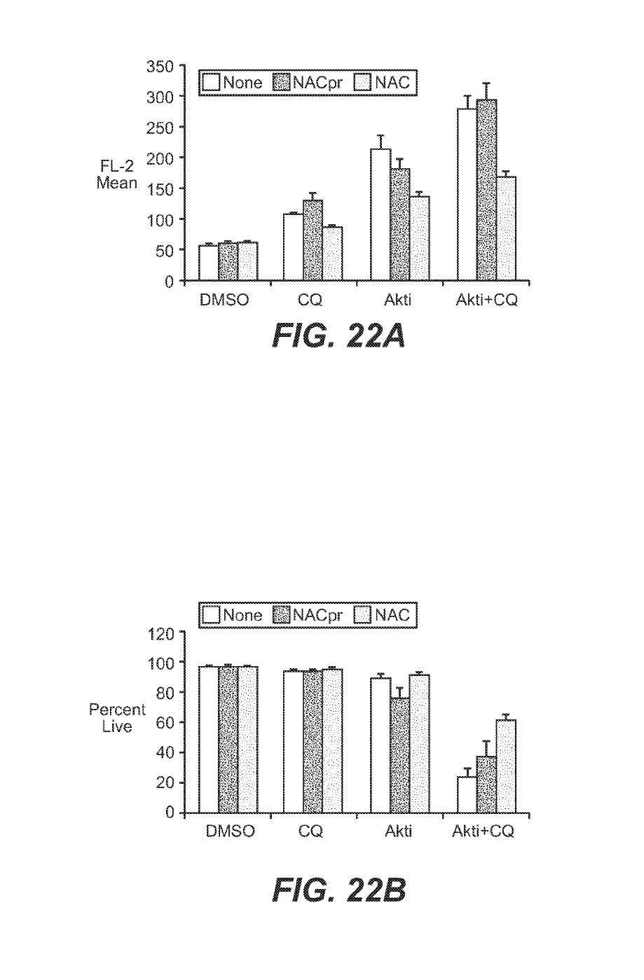

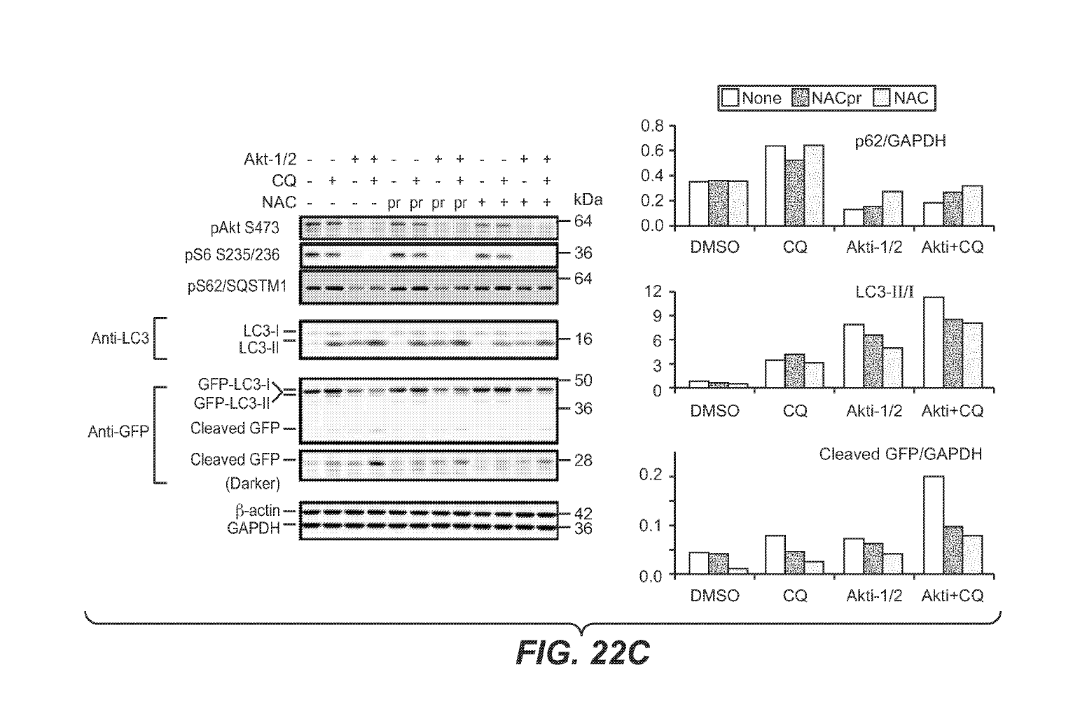

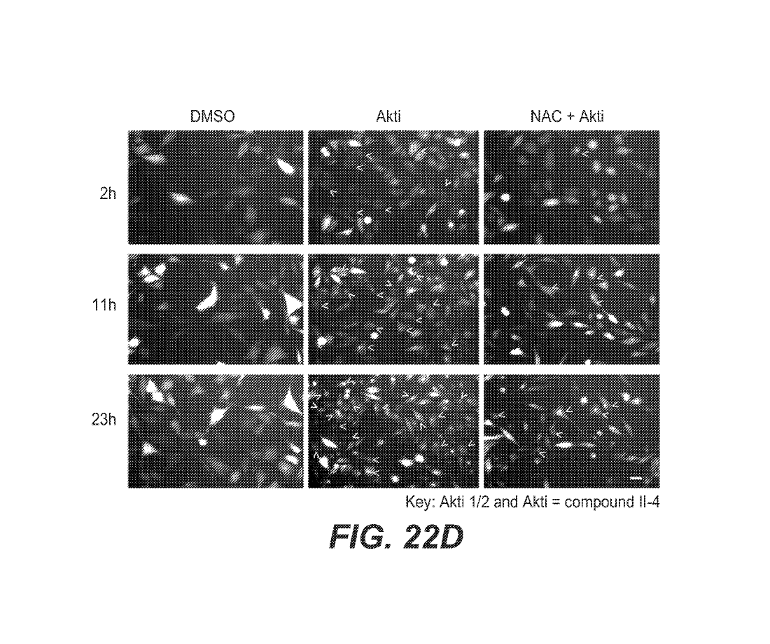

Because autophagy is a key function of the lysosomal compartment (Terman, A., B. Gustafsson, and U. T. Brunk, (2006), The lysosomal-mitochondrial axis theory of postmitotic aging and cell death. Chem Biol Interact. 163:29-37), we examined the lysosomal marker LAMPI and cathepsin D, the predominant lysosomal aspartic protease, by immunoblotting (FIG. 6, B and C). Compound II-4 alone induced an increase in LAMP1 levels, consistent with an elevated lysosomal activity. Cathepsin D is synthesized as a 43-kD preprocathepsin D that is cleaved cotranslationally and glycosylated to form a 46-kD procathepsin D, which is targeted to lysosomes yielding an intermediate that is further cleaved into a mature enzyme consisting of a 15-kD light chain and a 28-kD heavy chain. Using an antibody that detects both the 28-kD and the precursor forms of cathepsin D, an increase in the level of the premature forms of cathepsin D at 43-50 kD was first detected after II-4 treatment alone followed by an increase in the 28-kD heavy chain of the mature enzyme, again indicating an increased lysosomal activity. CQ caused accumulation of the precursor forms at the expense of the 28-kD chain, consistent with an inhibition of lysosomal cysteine protease activity required for the processing and maturation of cathepsin D (Liaudet-Coopman, E., M. Beaujouin, D. Derocq, M. Garcia, M. Glondu-Lassis, V. Laurent-Matha, C. Prebois, H. Rochefort, and F. Vignon, (2006), Cathepsin D: newly discovered functions of a long-standing aspartic protease in cancer and apoptosis. Cancer Lett. 237:167-79). In cells treated with both II-4 and CQ, the precursor forms of cathepsin D accumulated to even higher levels than either alone, whereas the mature 28-kD chain decreased gradually. Compound II-4 treatment also reduced the level of p62, another marker of autophagic activity that is degraded in the autolysosomes (Klionsky et al., 2008), whereas CQ blocked p62 degradation both with and without II-4 treatment (FIG. 22 C). Collectively, these data suggest that Akt inhibition causes an increased production and maturation of the lysosomal enzymes, whereas CQ cotreatment impairs the maturation of these enzymes in the final autolysosomal compartment, causing accumulation of defective AVOs. The latter is accompanied by an increased cleavage of caspase 3 into the active forms within 2 d (FIG. 6 B) with a corresponding increase in caspase activity and cleavage of its substrate PARP (unpublished data). zVAD.fmk, a pancaspase inhibitor, partially rescued cell death at all concentrations tested (FIG. 20 E). Although zVAD.fmk can also inhibit lysosomal cysteine proteases at higher concentrations, the latter have been reported to mediate caspase-independent cell death (Foghsgaard et al., 2001). Neither of the broad-spectrum cysteine protease inhibitors zFA.fmk and N-Acetyl-Leu-Leu-Nle-CHO nor a more specific cathepsin B inhibitor CA-074-Me showed significant rescue of cell death induced by II-4 and CQ. Instead, the cysteine protease inhibitors enhanced cell killing in combination with Akti at 10-50 μM concentrations, although they also showed cytotoxicity alone at higher concentrations (FIG. 20, F-H). These results suggest that cell death induced by II-4 and CQ is at least partially caspase dependent, whereas lysosomal protease activity may be required for the survival of cells under Akt inhibition.

To further ask whether impaired lysosomal degradation can accelerate cell death in combination with Akt inhibition, we knocked down cathepsin D using siRNA. Indeed, this significantly increased cell death when combined with II-4 and further enhanced the cell-killing effect of CQ when both are combined with II-4 (FIG. 20 I). Similarly, pepstatin A, an inhibitor of aspartic proteases including cathepsin D, also promoted cell death together with Akti-112 (FIG. 20 J)

### Example 7

### CQ Augmented Akti-Induced Mitochondrial Superoxide and Cellular Reactive Oxygen Species (ROS) Accumulation

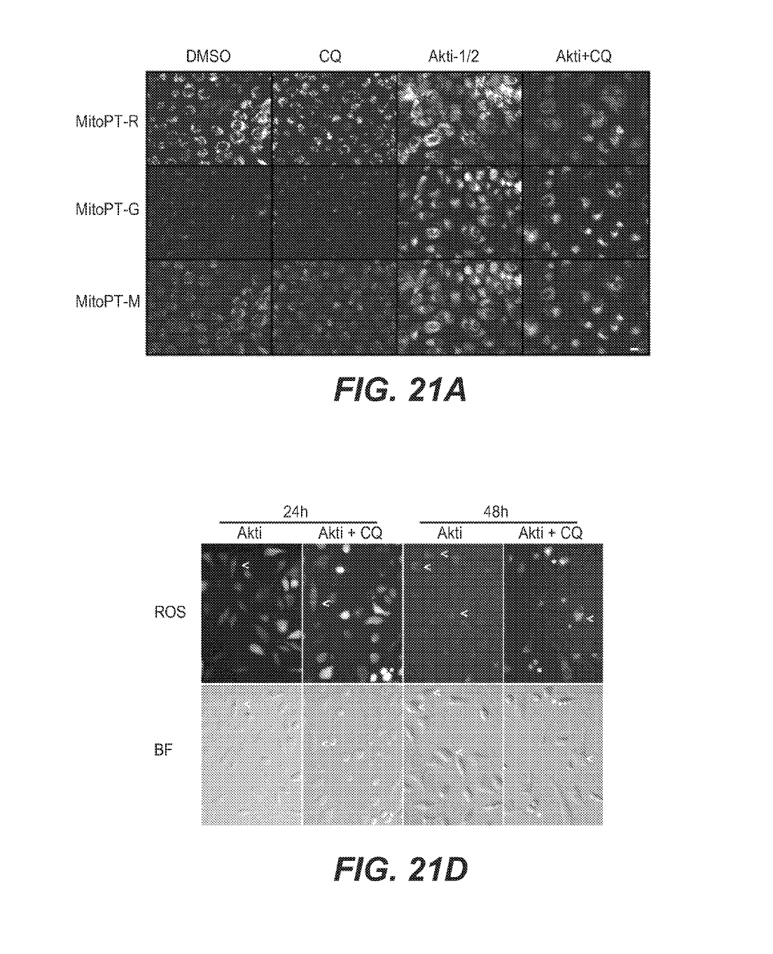

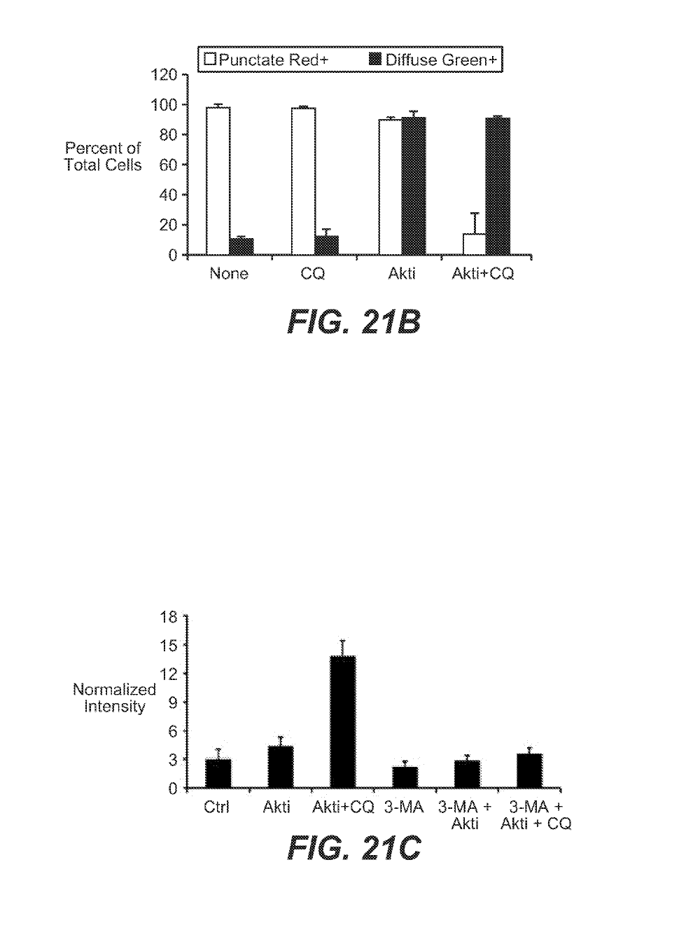

Increasing evidence has suggested an intimate relationship between lysosomes and mitochondria in the execution of programmed cell death (Bursch, W. (2001), The autophagosomal-lysosomal compartment in programmed cell death. Cell Death Differ. 8:569-81). The autophagosomal-lysosomal compartment in programmed cell death. Cell Death Differ. 8:569-81; Terman, A., T. Kurz, B. Gustafsson, and U. T. Brunk, (2006), Lysosomal labilization. IUBMB Life. 58:531-9). Therefore, we examined the effect of Akt inhibition and CQ on mitochondrial membrane potential. Consistent with Akt's function in maintaining mitochondrial integrity (Parcellier, A., L. A. Tintignac, E. Zhuravleva, and B. A. Hemmings, (2007), PKB and the mitochondria: AKTing on apoptosis. Cell Signal. 0:0), Akti-112 alone caused a decrease in mitochondrial membrane potential, although significant numbers of polarized mitochondria were still present in the majority of cells. Although CQ alone did not have a significant effect, cotreatment of CQ and II-4 caused an almost complete loss of mitochondrial potential, preceding the sharp drop in cell viability (FIG. 21, A and B).

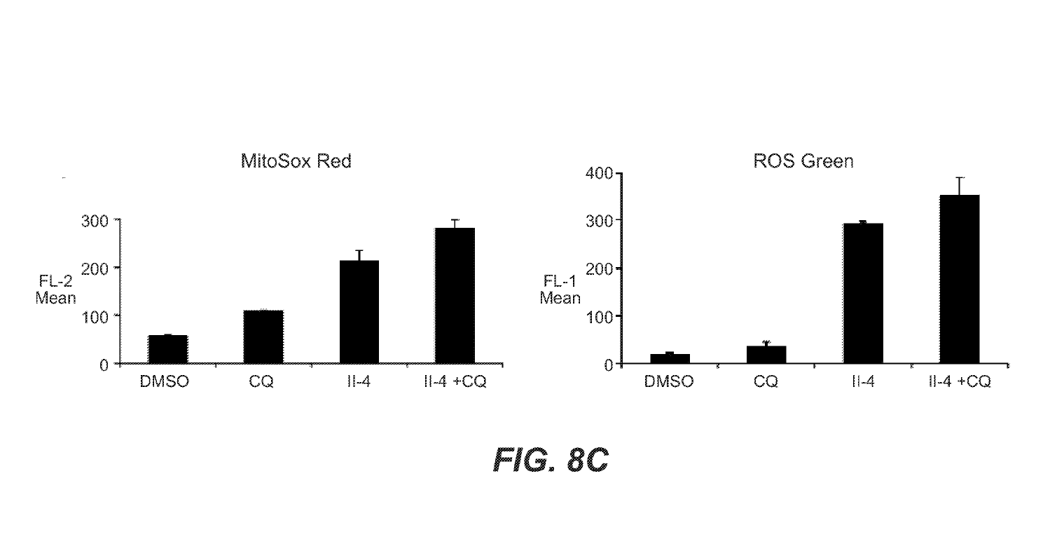

It has recently been reported that mitochondrial ROS is involved in autophagy induction (Scherz-Shouval et al., 2007). Because mitochondria are the primary intracellular source of superoxide (O₂⁻) generation, we analyzed O₂⁻ production using MitoSOX red, an O₂⁻ indicator that accumulates in the mitochondria as a function of membrane potential and fluoresces upon oxidation and subsequent binding to DNA. Compound II-4 alone increased MitoSOX fluorescence within 6 h (FIG. 8, A and C; and not depicted). Most of the fluorescence exhibited a mitochondrial localization pattern with a subpopulation of cells showing nuclear fluorescence, consistent with increased mitochondrial permeability in these cells. Although CQ alone only caused a mild increase in MitoSOX signal, the combination with II-4 resulted in a significant increase in fluorescence intensity with most cells exhibiting a strong nuclear staining pattern. The increase in O₂⁻ production was followed by an increase in overall cellular ROS levels within 24 h, as measured using a general ROS-sensitive probe (FIG. 8, B and C). Interestingly, cytoplasmic ROS signal induced by II-4 alone was attenuated within 48 h, whereas cotreatment with CQ caused a prolonged increase in ROS levels (FIG. 21 D and not depicted). This is consistent with the notion that limited mitochondrial depolarization caused by Akt inhibition induces a transient ROS signal to increase autophagy, which in sum removes the damaged mitochondria. Impaired digestion of cellular components caused by CQ results in autolysosomal aggregation of deleterious oxidative products such as ceroid/lipofuscin, which can further amplify the ROS damage (Moore et al., 2(06), leading to cell death. 3-MA pretreatment reduced ROS levels induced by II-4 (FIG. 21 C), suggesting a class III PI3K dependence similar to starvation-induced ROS production (Scherz-Shouval et al., 2007). Treatment with a general ROS scavenger N-acetylcysteine (NAC) rescued cell viability in the presence of II-4 and CQ (FIG. 22, A and B). In addition, NAC reduced Akti-induced LC3 and GFP-LC3 lipidation, p62 degradation, and GFP-LC3 puncta formation (FIG. SS, C and D), consistent with an essential role of ROS in autophagy induction (Scherz-Shouval et al., 2007).

### Example 8

### CQ Selectively Accelerated Cell Death in Akti-Treated PTEN-Null Cells In Vitro and Enhanced the Antitumor Efficacy of Akt KD In Vivo

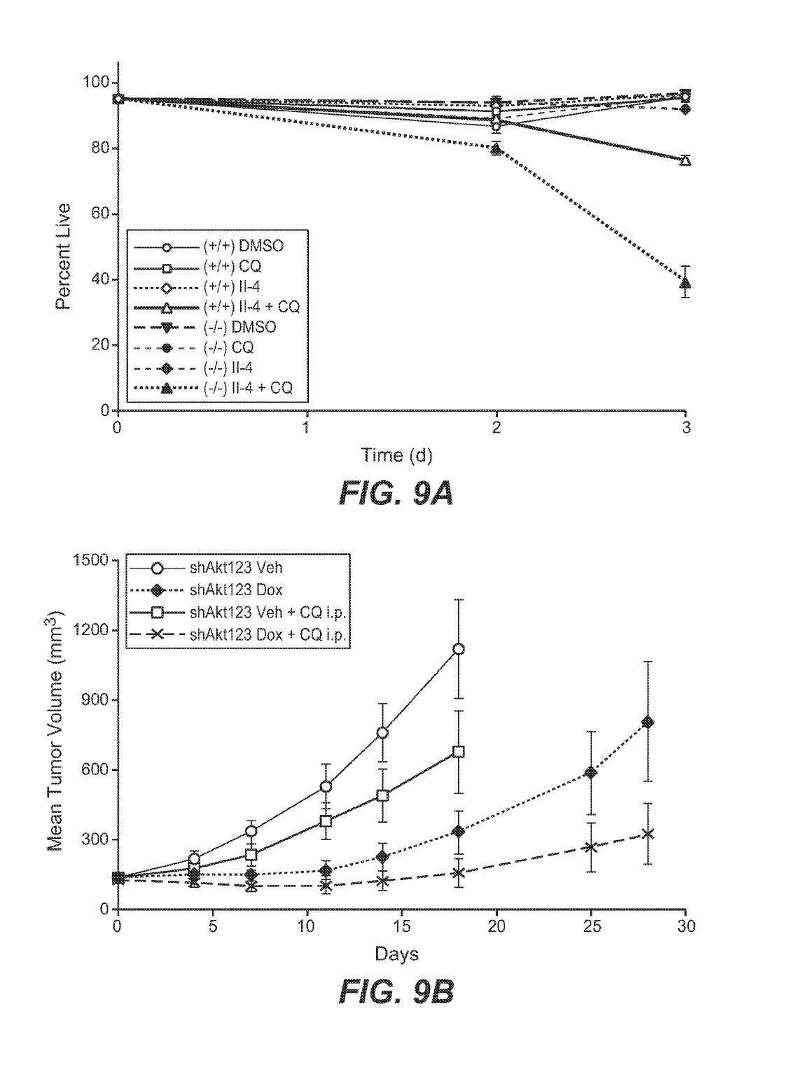

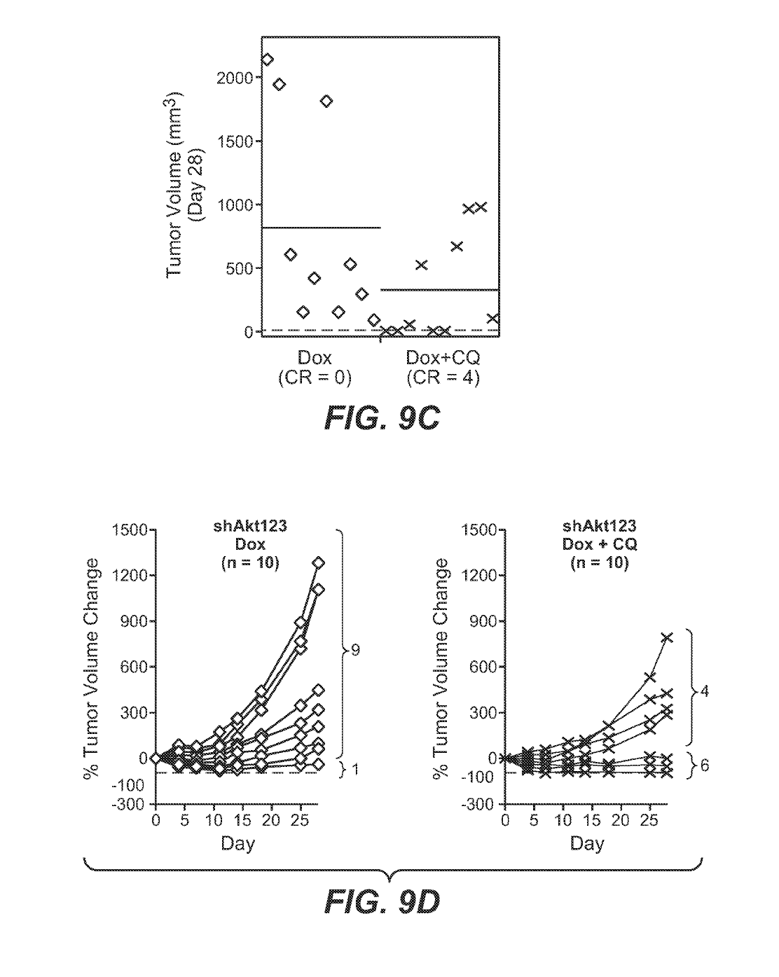

Because PC3 cells are PTEN null, we explored whether PTEN status might affect the sensitivity of cells to Akt inhibition alone or in combination with CQ using isogenic PTEN^{+/+} and PTEN^{-/-} mouse embryonic fibroblasts (MEFs). The PTEN^{-/-} MEFs were previously shown to have elevated Akt pathway activity and are more sensitive to the anti proliferative effect of mTOR inhibition than PTEN+t+ MEFs (Sun et al., 1999). As shown in FIG. 9 A, the PTEN^{-/-} MEFs were also significantly more sensitive to the cell-killing effect of combined CQ and II-4 than their PTEN^{+/+} counterparts. This suggests that PTEN^{-/-} cells may be more dependent on autophagic degradation for survival upon Akt inhibition, raising the possibility that a reasonable therapeutic index may be achievable by selective targeting of the malignant PTEN-null tumor cells using this strategy.

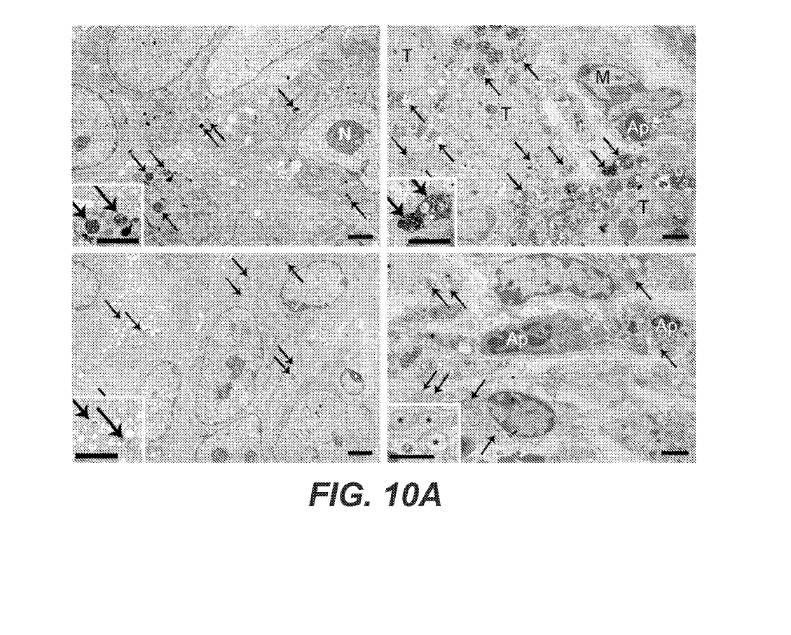

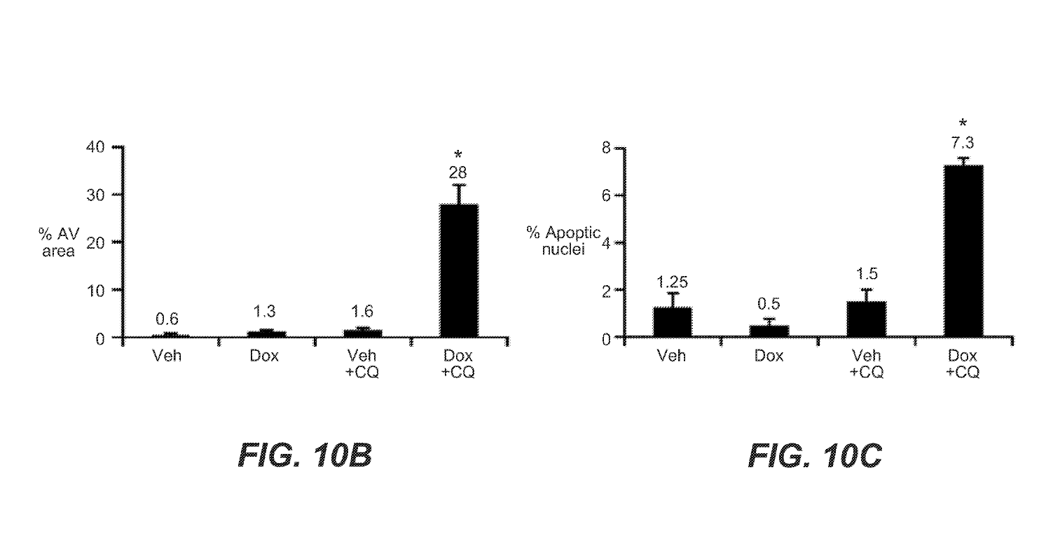

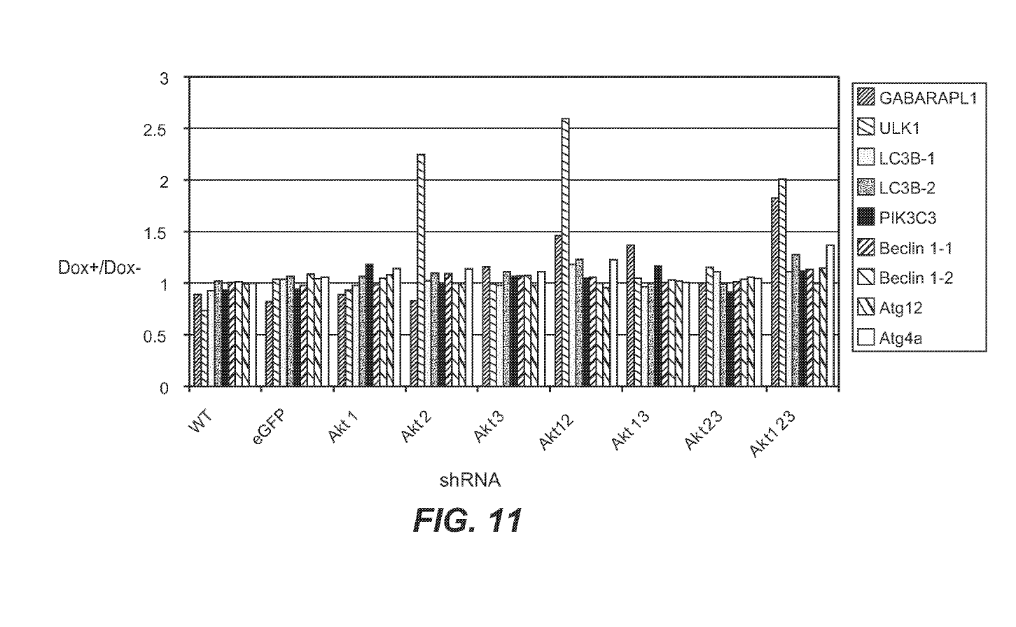

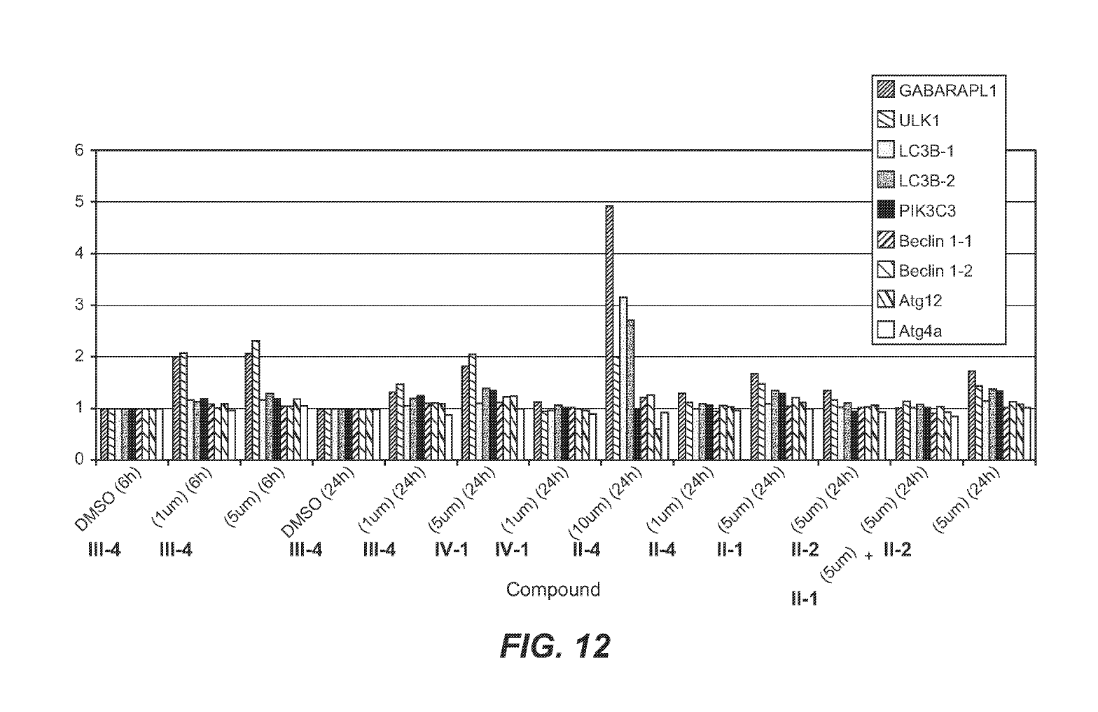

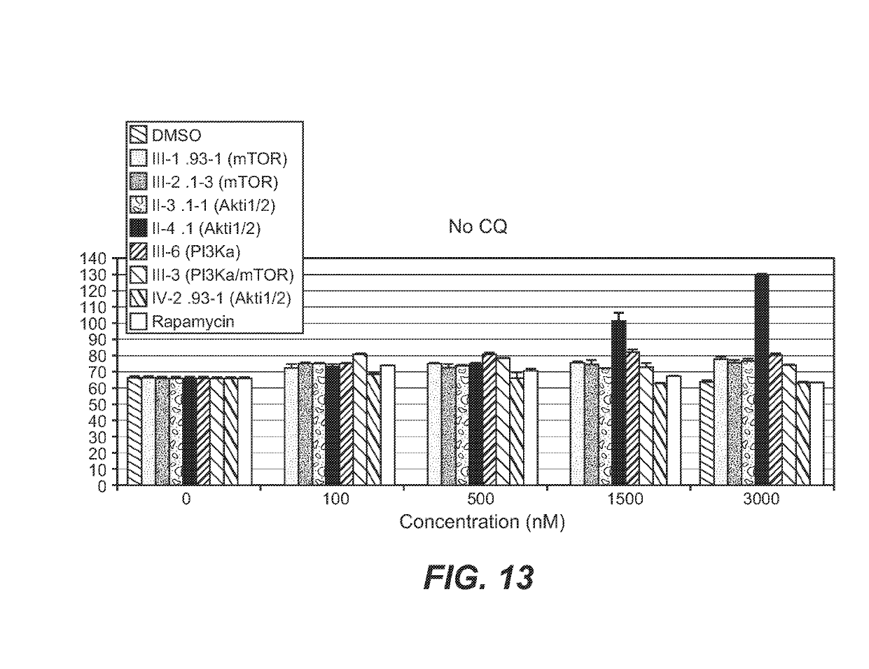

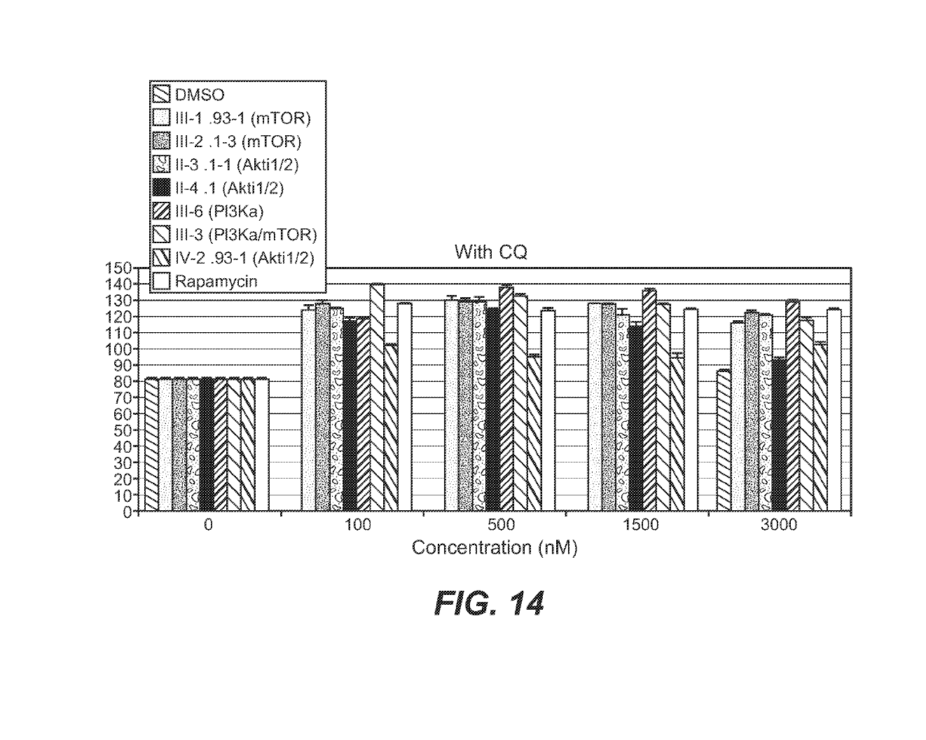

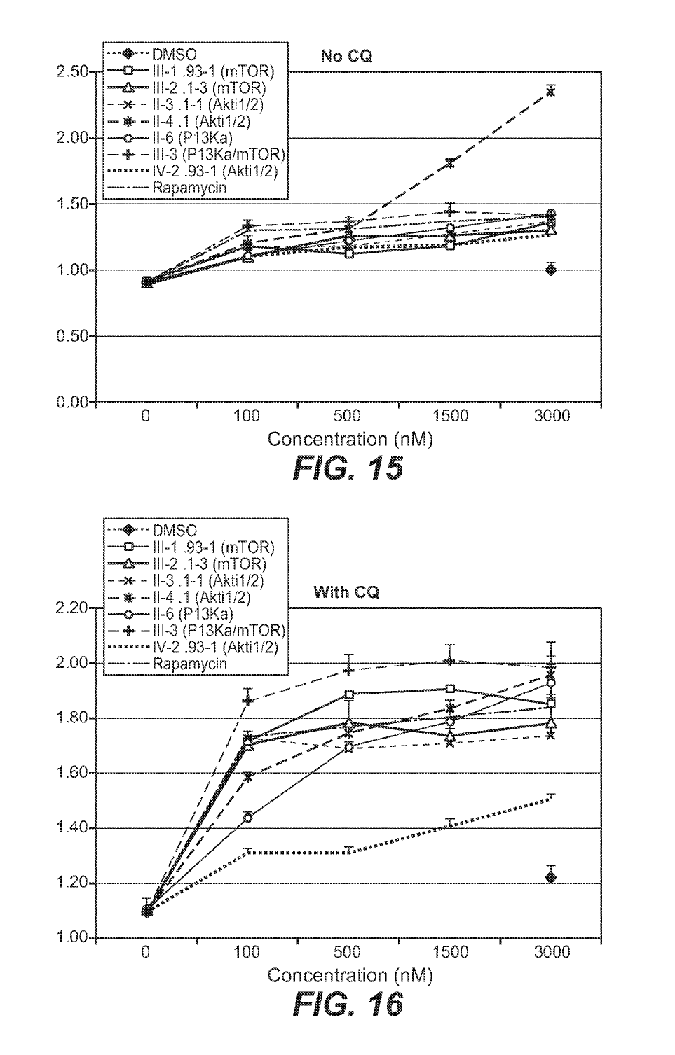

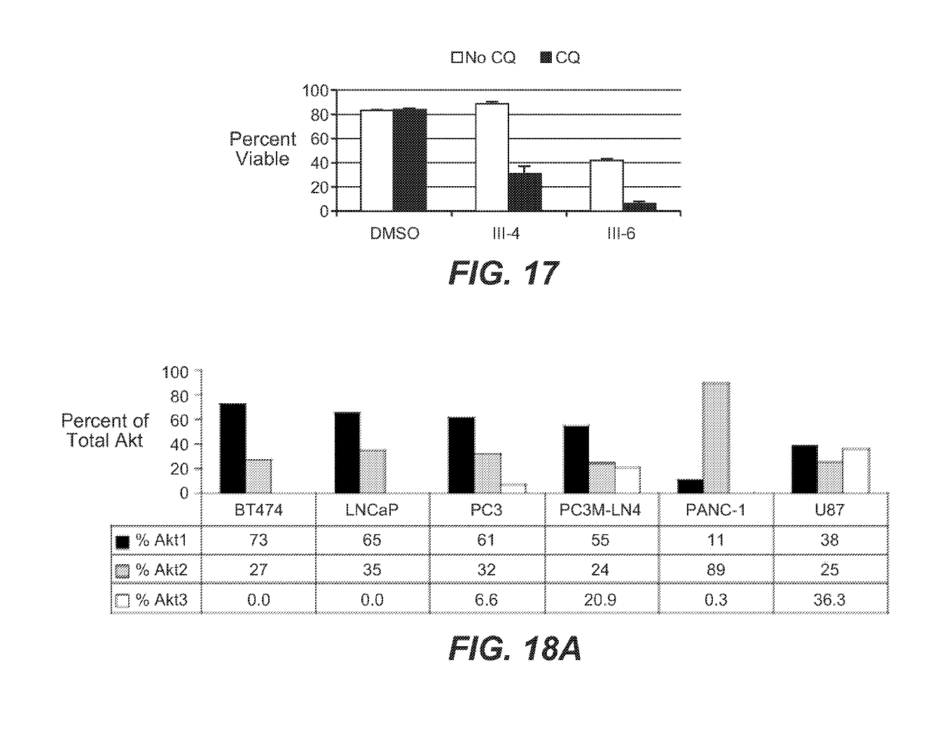

To ask whether PTEN-null tumors also rely on autophagic degradation upon Akt inhibition in vivo, we examined the effect of CQ on the survival of PC3 xenograft tumors expressing shAkt 123. As shown in FIG. 9 B, intraperitoneal injection of CQ alone caused a small but insignificant reduction in tumor growth rate. Akt KD alone resulted in significant tumor growth inhibition with an initial tumor stasis, but most tumors failed to regress completely, and rebound occurred in 90% of the tumors within 2-3 wk; no complete remission was achieved. In contrast, complete regression was observed in 40% of the tumors treated with both Dox and CQ with stasis maintained in another 20% of the tumors throughout the study (FIG. 9, C and D). Similar results were obtained with a subcutaneous peritumor injection of CQ (unpublished data). EM examination of tumor samples taken at day 5 revealed a mild increase in the AV area in tumors treated with either Dox or CQ alone, whereas a dramatic accumulation of AVs was observed in a tumor treated with both Dox and CQ that showed >50% regression. These AVs are larger than those found in the Dox- or CQ-alone tumors and contain dense undigested materials, but usually with a single-membrane auto lysosomal appearance and stained positive for human LAMP1, consistent with impaired degradation after autophagosome-lysosome fusions. This coincides with an increased number of tumor nuclei exhibiting apoptotic morphology as well as AV-containing cell debris with compromised plasma membrane integrity and abnormal mitochondria (FIG. 10). Thus, CQ not only accelerated cell death in combination with Akt inhibition in vitro but also increased the incidence of complete tumor remissions in vivo.

Using a Dox-inducible shRNA approach, we specifically knocked down each Akt isoform, both individually and in all possible combinations, to evaluate their requirement in the maintenance of tumor growth. Our results suggest that in the PTEN-null PC3 and U87MG cells, Akt1 is the most important isoform, whereas Akt2 and Akt3 activities could partially compensate for the reduced Akt1 activity in maintaining tumor growth. Taking together both the potential metabolic side effects of Akt2 inhibition and the reported increase in invasiveness associated with inhibiting Akt 1 alone that could be counteracted by simultaneous inhibition of Akt2 (Irie et al., 2005), it may be necessary to inhibit two or all three Akt isoforms simultaneously to achieve maximum tumor inhibition, but with different degrees of inactivation to preserve crucial levels of isoform activities to reduce side effects.

One of the most prominent functions of Akt is cell survival. Constitutively active Aid has been reported to protect cells from programmed cell death after various proapoptotic insults (Downward, 1998). However, whether apoptosis is a primary response to Akt inhibition is less clear, especially in cancer cells where apoptosis is often suppressed because of various genetic alterations. Previous experiments using small molecule inhibitors of the PI3K-Akt pathway often generate conflicting results that are obscured by their nonspecific effects. Our data indicate that specific KD of Akt can cause cell cycle delay without promoting significant apoptosis. This is consistent with a recent study that only a small portion of total Akt activity is required for apoptosis inhibition (Liu et al., 2006). In contrast, we found that autophagy is a more sensitive response to reduced Akt activity caused by either specific shRNA KD or selective inhibitors of the pathway.

Several mechanisms may contribute to autophagy induction by Akt inhibition. First, inhibiting Akt can lead to mTORC1 inhibition. mTOR is a known inhibitor of autophagy. Interestingly, a constitutively active form of Akt suppressed the induction of autophagy by rapamycin (Takeuchi et al., 2005), raising the possibility that the effect of rapamycin on autophagy may be mediated at least partially through inhibiting Akt via its long-term effect on mTORC2 (Sarbassov et al., 2006). Second, other signaling outputs of Akt, such as the FoxO proteins (Zhao et al., 2008) or glucose metabolism, can also contribute to autophagy regulation independently of mTOR. Third, our data indicate that Akt inhibition induces increased mitochondrial superoxide and cellular ROS signals that can activate autophagy.

Autophagy activation may lead to eventual cell death when allowed to reach its limit or may sensitize cells to additional death-inducing stimuli either through eventual autophagic cell death or switching to a more rapid death program such as apoptosis. For example, Akt inhibition may increase radiosensitivity through augmenting autophagic response (Fujiwara et al., 2007), whereas calpain-mediated cleavage of Atg5 may switch autophagy into apotosis (Yousefi et al., 2006). Here we show that inhibiting Akt alone is ineffective in cell killing in the PTEN-null cancer cells that we examined, but cell death can be accelerated through blocking autolysosomal degradation. Although autophagy may be a potential mechanism by which Akt inhibition restricts tumor growth, it may also provide temporary relief from the metabolic and oxidative stress imposed by Akt inhibition. Inhibiting autophagy at an early stage may prevent this temporary protective effect but may also counteract its tumor inhibitory effect while allowing early escape via alternative survival mechanisms. Blocking lysosomal function after tumor cells have become committed and reliant on autophagic degradation, however, might avoid this counteracting effect while amplifying the oxidative damage and cytotoxic effects through accumulation of deleterious oxidative aggregates (Seehafer and Pearce, 2006). Indeed, our data suggest that a compatible lysosomal degradation capacity is critical for cell survival in the presence of elevated autophagic activity induced by Akt inhibition such that inhibiting lysosomal function with lysosomotropic agents, cathepsin D KD or lysosomal protease inhibitors, can all precipitate cell death in combination with Akt inhibition. Autophagy, lysosomal changes, and oxidative stress have been associated with a lengthening list of anticancer treatments, and lysosomotropic agents have shown anticancer activity either alone or in combination with other therapeutic agents (Shoemaker and Dagher, 1979; Ohta et al., 1998; Ostenfeld et al., 2005; Amaravadi et al., 2007; Carew, J. S., S. T. Nawrocki, C. N. Kahue, H. Zhang, C. Yang, L. Chung, J. A. Houghton, P. Huang, F. J. Giles, and J. L. Cleveland, (2007), Targeting autophagy augments the anticancer activity of the histone deacetylase inhibitor SAHA to overcome Bcr-Abl-mediated drug resistance. Blood. 110:313-22; Fujiwara et al., 2007; GrothPedersen et al., 2007). Here we report for the first time that autophagy induced by Akt/PI3K/mTOR inhibition can also be exploited using lysosomotropic agents, such as the well-tolerated drug CQ, to promote the remission of PTEN-null human tumor xenografts. Because this effect is expected to correlate positively with the degree of autophagy induced by a given treatment, creative combination of these agents with potent autophagy inducers, such as inhibitors of the Akt pathway, may profoundly affect their anticancer efficacy.

Specific reference is made to U.S. provisional application No. 61/426,325, herein incorporated by reference in its entirety. Having now fully described this invention, it will be understood to those of ordinary skill in the art that the same can be performed within a wide and equivalent range of conditions, formulations, and other parameters without affecting the scope of the invention or any embodiment thereof. All patents, patent applications, and publications cited herein are fully incorporated by reference herein in their entirety.

1. A method of treating a neoplasm in a mammal comprising,
administering a combination of (i) an inhibitor of a kinase that induces autophagy and (ii) an inhibitor of autophagy in an amount effective to treat said neoplasm.

2. The method of claim 1, wherein said inhibitor of kinase that induces autophagy and said inhibitor of autophagy are present in synergistically effective amounts.

3. The method of claim 1, wherein said inhibitor of autophagy is a siRNA or antisense RNA.

4. The method of claim 1, wherein said inhibitor of autophagy inhibits the expression or function of LAMP2, LAMP1, or an autophagy (Atg) gene.

5. The method of claim 4, wherein said Atg gene is Atg1, Atg4, Atg8, Atg5, Atg7 or Atg12.

6. The method of claim 1, wherein said inhibitor of autophagy is an inhibitor of the induction, sequestration, fusion or degradation phase of autophagy.

7. The method of claim 6, wherein said inhibitor of autophagy is an inhibitor of the induction phase of autophagy.

8. The method of claim 7, wherein said inhibitor of autophagy is 3-methyladenine.

9. The method of claim 6, wherein said inhibitor of autophagy is an inhibitor of the degradation or fusion phase of autophagy.

10. The method of claim 9, wherein said inhibitor of the degradation phase of autophagy is a lysosomotropic agent.

11-16. (canceled)

17. The method of claim 10, wherein said lysosomotropic agent is a cytotoxic agent.

18-21. (canceled)

22. The method of claim 10, wherein said lysosomotropic agent is ammonium chloride, cAMP or methylamine.

23. The method of claim 1, wherein said inhibitor of a kinase that induces autophagy is selected from an Akt, PI3K, mTOR, PDK1 and p70S6K inhibitor.

24. The method of claim 23, wherein said inhibitor of a kinase that induces autophagy is an Akt or PI3K kinase inhibitor.

25-32. (canceled)

33. The method of claim 1, wherein said neoplasm is a sarcoma.

34. The method of claim 1, wherein said neoplasm is a carcinoma.

35. The method of claim 1, wherein said neoplasm is a squamous cell carcinoma.

36. The method of claim 1, wherein said neoplasm is an adenoma or adenocarcinoma.

37. The method of claim 1, wherein said cancer is selected from the group consisting of breast, ovary, cervix, prostate, testicular, penile, genitourinary tract, seminoma, esophageal, larynx, gastric, stomach, gastrointestinal, skin, keratoacanthoma, follicular carcinoma, melanoma, lung, small cell lung carcinoma, non-small cell lung carcinoma (NSCLC), lung adenocarcinoma, squamous carcinoma of the lung, colon, pancreas, thyroid, papillary, bladder, liver, biliary passage, kidney, bone, myeloid disorders, lymphoid disorders, hairy cells, buccal cavity and pharynx (oral), lip, tongue, mouth, salivary gland, pharynx, small intestine, colon, rectum, anal, renal, prostate, vulval, thyroid, large intestine, endometrial, uterine, brain, central nervous system, cancer of the peritoneum, hepatocellular cancer, head cancer, neck cancer, Hodgkin's and leukemia.

38. (canceled)

39. The method of claim 1, wherein said neoplasm is other than a glycolysis dependent cancer.

40-61. (canceled)


SEQUENCE LISTING (38 sequences)

SEQ ID NO: 1 - DNA, 65 bp, Human
gatcccctgaccatgaacgagtttgattcaagagatcaaactcgttcatggtcattttttggaaa

SEQ ID NO: 2 - DNA, 65 bp, Human
agcttttccaaaaaatgaccatgaacgagtttgatctcttgaatcaaactcgttcatggtcaggg

SEQ ID NO: 3 - DNA, 65 bp, Human
gatccccgtggaccactgtcatcgaattcaagagattcgatgacagtggtccacttttttggaaa

SEQ ID NO: 4 - DNA, 65 bp, Human
agcttttccaaaaaagtggaccactgtcatcgaatctcttgaattcgatgacagtggtccacggg

SEQ ID NO: 5 - DNA, 65 bp, Human
gatcccccctggaggccacggtacttttcaagagaaagtaccgtggcctccaggttttttggaaa

SEQ ID NO: 6 - DNA, 65 bp, Human
agcttttccaaaaaacctggaggccacggtactttctcttgaaaagtaccgtggcctccaggggg

SEQ ID NO: 7 - DNA, 65 bp, Human
gatcccctgacttcgactatctcaaattcaagagatttgagatagtcgaagtcattttttggaaa

SEQ ID NO: 8 - DNA, 65 bp, Human
agcttttccaaaaaatgacttcgactatctcaaatctcttgaatttgagatagtcgaagtcaggg

SEQ ID NO: 9 - DNA, 65 bp, Human
gatccccgaattgtagtccaacttcattcaagagatgaagttggactacaattcttttttggaaa

SEQ ID NO: 10 - DNA, 65 bp, Human
agcttttccaaaaaagaattgtagtccaacttcatctcttgaatgaagttggactacaattcggg

SEQ ID NO: 11 - DNA, 65 bp, Human
gatccccgcacttttgggaaagttatttcaagagaataactttcccaaaagtgcttttttggaaa

SEQ ID NO: 12 - DNA, 65 bp, Human
agcttttccaaaaaagcacttttgggaaagttattctcttgaaataactttcccaaaagtgcggg

SEQ ID NO: 13 - DNA, 65 bp, Human
gatccccgctactacgccatgaagatttcaagagaatcttcatggcgtagtagcttttttggaaa

SEQ ID NO: 14 - DNA, 65 bp, Human
agcttttccaaaaaagctactacgccatgaagattctcttgaaatcttcatggcgtagtagcggg

SEQ ID NO: 15 - DNA, 65 bp, Human
gatccccaggtgctggaggacaatgattcaagagatcattgtcctccagcacctttttttggaaa

SEQ ID NO: 16 - DNA, 65 bp, Human
agcttttccaaaaaaaggtgctggaggacaatgatctcttgaatcattgtcctccagcacctggg

SEQ ID NO: 17 - DNA, 65 bp, Human
gatccccctacaaccaggaccatgagttcaagagactcatggtcctggttgtagttttttggaaa

SEQ ID NO: 18 - DNA, 65 bp, Human
agcttttccaaaaaactacaaccaggaccatgagtctcttgaactcatggtcctggttgtagggg

SEQ ID NO: 19 - RNA, 19 nt, Human
ugaccaugaacgaguuuga

SEQ ID NO: 20 - RNA, 19 nt, Human
ucaaacucguucaugguca

SEQ ID NO: 21 - RNA, 19 nt, Human
guggaccacugucaucgaa

SEQ ID NO: 22 - RNA, 19 nt, Human
uucgaugacagugguccac

SEQ ID NO: 23 - RNA, 19 nt, Human
ccuggaggccacgguacuu

SEQ ID NO: 24 - RNA, 19 nt, Human
aaguaccguggccuccagg

SEQ ID NO: 25 - RNA, 19 nt, Human
ugacuucgacuaucucaaa

SEQ ID NO: 26 - RNA, 19 nt, Human
uuugagauagucgaaguca

SEQ ID NO: 27 - RNA, 19 nt, Human
gaauuguaguccaacuuca

SEQ ID NO: 28 - RNA, 19 nt, Human
ugaaguuggacuacaauuc

SEQ ID NO: 29 - RNA, 19 nt, Human
gcacuuuugggaaaguuau

SEQ ID NO: 30 - RNA, 19 nt, Human
auaacuuucccaaaagugc

SEQ ID NO: 31 - RNA, 19 nt, Human
gcacuuuugggaaaguuau

SEQ ID NO: 32 - RNA, 19 nt, Human
auaacuuucccaaaagugc

SEQ ID NO: 33 - RNA, 19 nt, Human
gcuacuacgccaugaagau

SEQ ID NO: 34 - RNA, 19 nt, Human
aucuucauggcguaguagc

SEQ ID NO: 35 - RNA, 19 nt, Human
aggugcuggaggacaauga

SEQ ID NO: 36 - RNA, 19 nt, Human
aggugcuggaggacaauga

SEQ ID NO: 37 - RNA, 19 nt, Human
cuacaaccaggaccaugag

SEQ ID NO: 38 - RNA, 19 nt, Homo sapiens
cucaugguccugguuguag